(19) United States
(12) Patent Application Publication
(10) Pub. No.: US 2016/0045531 A1
(43) Pub. Date: Feb. 18, 2016

(54) COMBINATION THERAPY FOR TREATING CANCER

(71) Applicant: EPIZYME, INC., Cambridge, MA (US)

(72) Inventors: Christine Klaus, Waban, MA (US); Maria Alejandra Raimondi, Jamaica Plain, MA (US); Scott Richard Daigle, Newburyport, MA (US); Roy MacFarlane Pollock, Medford, MA (US)

(21) Appl. No.: 14/776,669

(22) PCT Filed: Mar. 14, 2014
(86) PCT No.: PCT/US14/28609

§ 371 Date: Sep. 14, 2015

(51) Int. Cl. A61K 31/7076; C07H 19/044; A61K 31/7064; A61K 31/551; A61K 31/704; A61K 31/706; A61K 31/136; A61K 31/4745; C07H 19/16; A61K 31/7068

(52) U.S. Cl. CPC A61K 31/7076; C07H 19/16; C07H 19/044; A61K 31/7064; A61K 31/7068; A61K 31/704; A61K 31/706; A61K 31/136; A61K 31/4745; A61K 31/551

(57) ABSTRACT

The present invention relates to compositions comprising inhibitors of human histone methyltransferase DOT1L and one or more therapeutic agents, particularly anticancer agents, and methods of combination therapy for administering to subjects in need thereof for the treatment of cancer.

### RELATED APPLICATIONS

This application claims priority to, and the benefit of U.S. Provisional Application Nos. 61/785,446, field Mar. 14, 2013, 61/900,939, filed Nov. 6, 2013, 61/912,872, filed Dec. 6, 2013. The entire contents of each of these provisional applications are incorporated herein by reference in their entireties.

### FIELD OF INVENTION

This invention relates to compositions comprising inhibitors of human histone methyltransferase DOT1L and one or more other therapeutic agents, particularly anticancer agents, and methods of combination therapy for treating cancer.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., DOT1L), many of which are associated with specific genetic alterations that can cause human disease.

Disease-associated chromatin-modifying enzymes (e.g., DOT1L) play a role in diseases such as proliferative disorders, metabolic disorders, and blood disorders. Thus, there is a need for the development of compositions that are capable of modulating the activity of DOT1L.

### SUMMARY OF THE INVENTION

In one aspect, this present invention features a composition comprising a compound of Formula (I):

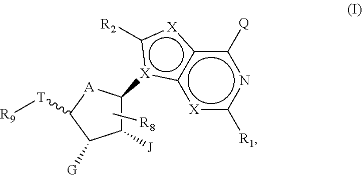

or pharmaceutically acceptable salts thereof, and one or more therapeutic agents, wherein,
T is a linker group of a 6-10 carbon atoms, in which one or more carbon atoms are optionally replaced with a heteroatom and T is optionally substituted;
R₉ comprises a C₆-C₁₀ aryl or 5 to 10-membered heteroaryl optionally substituted with one or more substituents selected from the group consisting of unsubstituted or substituted t-butyl, CF₃, cyclohexyl, C₆-C₁₀ aryl, and 5 to 10-membered heteroaryl;
A is O or CH₂;
each of G and J, independently, is H, halo, C(O)OH, C(O)O—C₁-C₆ alkyl or ORₐ, Rₐ being H, C₁-C₆ alkyl, C(O)—C₁-C₆ alkyl, or silyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;
each X independently is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
each of R₁ and R₂, independently is H, halo, hydroxyl, carboxyl, cyano, or R_{S2}, R_{S2} being amino, C₁-C₆ alkoxyl, C₁-C₆ alkyl, C₂-C₆ alkenyl, C₂-C₆ alkynyl, or C₃-C₈ cycloalkyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl; and
   Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl.

In some embodiments, the compound has formula (IV):

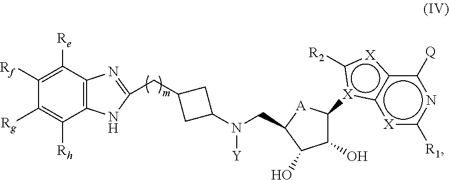

wherein each of Rₑ, R_{f}, R_{g}, and Rₕ, independently is -M₂-T₂, in which M₂ is a bond, SO₂, SO, S, CO, CO₂, O, O—C₁-C₄ alkyl linker, C₁-C₄ alkyl linker, NH, or N(Rₜ), Rₜ being C₁-C₆ alkyl, and T₂ is H, halo, or R_{S4}, R_{S4} being C₁-C₆ alkyl, C₂-C₆ alkenyl, C₂-C₆ alkynyl, C₃-C₈ cycloalkyl, C₆-C₁₀ aryl, 4 to 8-membered heterocycloalkyl, or 5 to 10-membered heteroaryl, and each of O—C₁-C₄ alkyl linker, C₁-C₄ alkyl linker, Rₜ, and R_{S4} being optionally substituted with one or more substituents selected from halo, hydroxyl, carboxyl, cyano, C₁-C₆ alkyl, C₂-C₆ alkenyl, C₂-C₆ alkynyl, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl, and
m is 0, 1, or 2.

In one aspect, the present invention provides a composition comprising any one of the compounds listed in Tables 1-4 or pharmaceutically acceptable salts thereof and one or more therapeutic agents.

In one aspect, the present invention provides a composition comprising Compound A2:

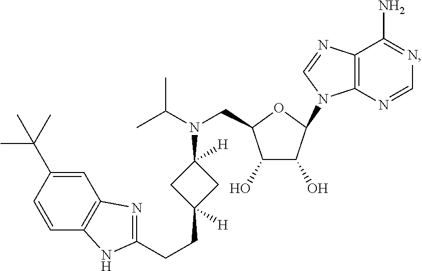 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or pharmaceutically acceptable salts thereof, and one or more therapeutic agents.

In one aspect, the present invention provides a composition comprising Compound D16:

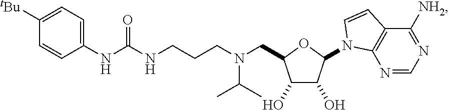

or pharmaceutically acceptable salts thereof, and one or more therapeutic agents.

In some embodiments, the one or more therapeutic agents are anti-cancer agents. The one or more therapeutic agents can be selected from Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, Navitoclax, and analogs, derivatives, or combinations thereof. Preferably, the therapeutic agent is Ara-C or Daunorubicin, or an analog or derivative thereof.

In one aspect, the present invention provides a pharmaceutical composition comprising a therapeutically effective amount of any composition described herein and a pharmaceutically acceptable carrier.

In one aspect, the present invention provides a method of treating or alleviating a symptom of a disease by administering to a subject in need thereof a therapeutically effective amount of a composition described herein. The disease is cancer or a precancerous condition. Alternatively, the disease can be influenced by modulating the methylation status of histones or other proteins. The methylation status is mediated at least in part by the activity of DOT1L.

In one aspect, the present invention provides a method of treating or alleviating a symptom of cancer by administering to a subject in need thereof a therapeutically effective dose of a compound of Formula (I) and one or more therapeutic agents, where a compound of Formula (I) and the one or more therapeutic agents are administered simultaneously or sequentially. Alternatively, a compound of Formula (I) is administered prior to administration of the one or more therapeutic agents. Alternatively, one or more therapeutic agents are administered/delivered prior to administration of a compound of Formula (I).

In one aspect, the present invention provides a method of treating or alleviating a symptom of cancer by administering to a subject in need thereof a therapeutically effective dose of a compound of Formula (I), or a pharmaceutically acceptable salt thereof, prior to administering a therapeutically effective dose of a composition described herein.

In one aspect, the present invention provides a method of treating or alleviating a symptom of cancer by administering to a subject in need thereof a therapeutically effective dose of one or more therapeutic agents prior to administering a therapeutically effective dose of a composition described herein.

In some embodiments, the composition described herein is administered to the subject in need thereof at a dosage of 0.01 mg/kg per day to about 1000 mg/kg per day.

In some embodiments, the compound of Formula (I) is administered at a dosage of 0.01 mg/kg per day to about 1000 mg/kg per day.

In some embodiments, each of the one or more therapeutic agents is administered at a dosage of 0.01 mg/kg per day to about 1000 mg/kg per day.

In some embodiments, the compound of Formula (I) or a pharmaceutically acceptable salt thereof is administered at a dose of at least 36 mg/m².

In some embodiments, the compound of Formula (I) or a pharmaceutically acceptable salt thereof is administered at a dose of at least 54 mg/m².

In some embodiments, the compound of Formula (I) or a pharmaceutically acceptable salt thereof is administered at a dose of at least 80 mg/m².

the compound of Formula (I) or a pharmaceutically acceptable salt thereof is administered continuously for at least 7, 14, 21, 28, 35, 42, 47, 56, or 64 days.

In some embodiments, continuous administration comprises administration without a drug holiday.

In some embodiments, the administration results in maturation or differentiation of leukemic blast cells. For example, at least 20% of leukemic blast cells have undergone maturation or differentiation. For example, at least 50% of leukemic blast cells have undergone maturation or differentiation. For example, at least 80% of leukemic blast cells have undergone maturation or differentiation.

In some embodiments, administration results in reduction of H3K79 methyl mark to at least 90%, 80%, 70%, 60%, 50%, 40%, 30%, 20%, 10% or less of untreated control levels.

In some embodiments, administration results in the suppression of H3K79 methyl mark rebound.

In some embodiments, administration results in at least 20%, 30%, 40%, 50%, 60%, 70%, 80%, or 90% of leukemic blast cells undergoing cell death or apoptosis.

In some embodiments, the method of treatment includes resolution of fevers, resolution of cachexia or resolution of leukemia cutis.

In some embodiments, the method of treatment includes restoration of normal haematopoiesis.

In some embodiments, the subject has demonstrated resistance to any one of the components of the composition of claim 1 when administered as a single agent.

In some embodiments, the subject is a pediatric patient aged 3 months to 18 years.

In one aspect, the present invention provides a method of inhibiting cancer cell proliferation by contacting a cancer cell with a composition described herein.

In one aspect, the present invention provides a method of inhibiting cancer cell proliferation by contacting a cancer cell with a compound of Formula (I) and one or more therapeutic agents, where the compound of Formula (I) and the therapeutic agents are delivered simultaneously or sequentially. Alternatively, a compound of Formula (I) is administered/delivered prior to administration of the therapeutic agents. Alternatively, one or more therapeutic agents are administered/delivered prior to administration of a compound of Formula (I).

In one aspect, the present invention provides a method of inhibiting cancer cell proliferation by contacting a cancer cell a therapeutically effective dose of a compound of Formula (I), or a pharmaceutically acceptable salt thereof, prior to administering/contacting a therapeutically effective dose of a composition described herein. Alternatively, one or more therapeutic agents are administered/delivered prior to administration of a composition described herein.

The present invention further provides a method of treating or alleviating a symptom of a disease by administering to a subject in need thereof a therapeutically effective amount of a compound of Formula I, where the therapeutically effective amount is an amount sufficient to sensitize the subject to subsequent treatment with a therapeutic agent. The method may further include a step of administering to the sensitized subject a therapeutically effective amount of a therapeutic agent.

The present invention further provides a method of treating or alleviating a symptom of a disease by administering to a subject in need thereof a therapeutically effective amount of one or more therapeutic agents, where the therapeutically effective amount is an amount sufficient to sensitize the subject to subsequent treatment with a compound of Formula I or a composition that includes one or more therapeutic agents and a compound of Formula I or a pharmaceutically acceptable salt thereof. The method may further include a step of administering to the sensitized subject a therapeutically effective amount of a compound of Formula I or a composition that includes one or more therapeutic agents and a compound of Formula I or a pharmaceutically acceptable salt thereof.

In certain embodiments, the therapeutic agent is administered at least after one, two, three or more hours following the administration of compound of Formula (I).

In certain embodiments, the therapeutic agent is administered at least one, two, three or more hours prior to the administration of compound of Formula (I).

In certain embodiments, the therapeutic agent is administered at least after one, two, three or more days following the administration of compound of Formula (I).

In certain embodiments, the therapeutic agent is administered at least one, two, three or more days prior to the administration of compound of Formula (I).

For example, the compound has the formula

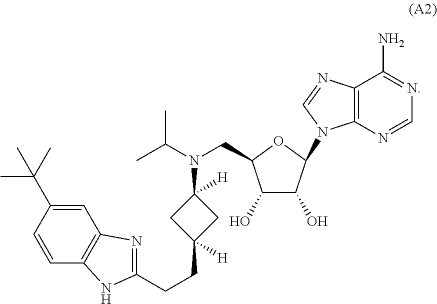 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

For example, the compound has the formula

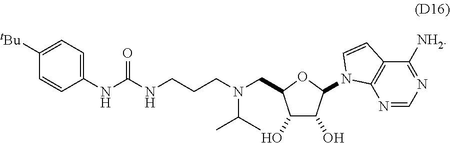

In certain embodiments, the sensitization is determined by the methylation status of histones or other proteins.

In certain embodiments, the sensitization is determined by decreased level of methylation of histones of other proteins.

In certain embodiments, the sensitization is determined by decreased methylation of H3K79.

In certain embodiments, the therapeutically effective amount of the therapeutic agent is lowered due to the sensitizing effect of compound of Formula (I).

In any methods described herein, the therapeutic agent may be Ara-C or Daunorubicin, or an analog or derivative thereof. Alternatively, the therapeutic agent is a standard of care agent.

In certain embodiments, the therapeutic agent is cytarabine.

The subject may have leukemia. The leukemia may be characterized by a chromosomal rearrangement. The chromosomal rearrangement is chimeric fusion of mixed lineage leukemia gene (MLL) or partial tandem duplication of MLL (MLL-PTD).

The subject may have an increased level of HOXA9, Fms-like tyrosine kinase 3 (FLT3), MEIS1, MEIS2, TBP, BCL, and/or DOT1L.

In any methods described herein, the compound may be Compound A2 or Compound D16.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF DRAWINGS

FIG. 1 is a diagram showing the overall experimental design and data analysis.

FIGS. 2A-2B are diagrams showing the steps of experimental design. FIG. 2A shows 4-day+3-day (“4+3”) treatment experimental design and FIG. 2B shows 7-day treatment experimental design.

FIG. 3 is diagram showing the experimental design about dosing of the compounds.

FIGS. 4A-4B are graphs showing combination index (CI) values for combinations of Compound A2 and Ara-C. FIG. 4A shows 4+3 treatment and FIG. 4B shows 7-day treatment experiments in MOLM-13 cell line.

FIGS. 5A-5B are graphs showing combination index (CI) values for combinations of Compound A2 and Daunorubicin. FIG. 5A shows 4+3 treatment and FIG. 5B shows 7-day treatment experiments in MOLM-13 cell line.

FIGS. 6A-6B are graphs showing combination index (CI) values for combinations of Compound A2 and hypomethylating agents. FIG. 6A shows combination of Compound A2 and Decitabine and FIG. 6B shows combination of Compound A2 and Vidaza in a 7-day treatment experiment in MOLM-13 cell line.

FIG. 7 is a graph showing combination index (CI) values for combinations of Compound A2 and topoisomerase inhibitor, Mitoxantrone, in MOLM-13 cell line.

FIG. 8 is a graph showing combination index (CI) values for combinations of Compound A2 and Bromodomain inhibitor, IBET-151, in a 7-day treatment experiment in MOLM-13 cell line.

FIGS. 9A-9B are graphs showing combination index (CI) values for combinations of Compound A2 and Ara-C. FIG. 9A shows 4+3 and FIG. 9B shows 7-day treatment experiments in MV4-11 cell line.

FIGS. 10A-10B are graphs showing combination index (CI) values for combinations of Compound A2 and Daunorubicin. FIG. 10A shows 4+3 and FIG. 10B shows 7-day treatment experiments in MV4-11 cell line.

FIG. 11 is a graph showing combination index (CI) values for combinations of Compound A2 and Vidaza in MV4-11 cell line.

FIG. 11 is a graph showing combination index (CI) values for combinations of Compound A2 and topoisomerase inhibitor, Mitoxantrone, in MV4-11 cell line.

FIG. 13 is a graph showing combination index (CI) values for combinations of Compound A2 and HDAC inhibitor, Panobinostat, in MV4-11 cell line.

FIGS. 14A-14B are graphs showing combination index (CI) values for combinations of Compound A2 and IBET-151. FIG. 14A shows 4+3 and FIG. 14B shows 7-day treatment experiments in MV4-11 cell line.

FIGS. 15A-15B are graphs showing combination index (CI) values for combinations of Compound A2 and Tranylcypromine in a 7-day treatment experiment. FIG. 15A shows MOLM-13 cell line and FIG. 15B shows MV4-11 cell line.

FIGS. 16A-16C are graphs showing combination index (CI) values for combinations of Compound A2 and Bcl-2 inhibitor, Navitoclax. FIG. 16A shows a 7-day treatment experiment in MOLM-13 cell line; FIG. 16B shows a 4+3 treatment experiment in MV4-11 cell line; and FIG. 16C shows a 7-day treatment experiment MV4-11 cell line.

FIG. 17 is a graph showing combination index (CI) values for combinations of Compound A2 and FLT inhibitor, Quizartinib, in a 7-day treatment experiment in MV4-11 cell line.

FIGS. 18A-18B are Fa-CI plots showing that Compound A2 and cytarabine act synergistically to induce an antiproliferative effect in the Molm-13 cell line in a pre-treatment model. FIG. 18A shows ten-day continuous dosing of Compound A2 with addition of cytarabine at day 7 showed a range of fractional effects with CI values <1 denoting synergy. FIG. 18B shows that Compound A2 was removed at day 7 prior to the addition of cytarabine showing durable combination benefit.

FIG. 19 shows three treatment models (A, B and C) for the study presented herein.

FIG. 20 shows the data analysis using Chou-Talalay method. Synergy quantification is performed using the Chou-Talalay method for drug combination. An Exemplary combination experiment is shown in A. The Combination Index (CI) equation offers a quantitative definition for additivity (CI=1), synergism (CI<1), and antagonism (CI>1). This equation (shown in graph B) used Fa values from a constant ratio of drug combination to determine CI values. The resulting plot (Fa-CI) plot (as shown in graph C) shows the resultant CI values bracketed by 95% confidence intervals. These Fa-CI plots are generated using the Calcusyn software. Statistically significant CI values for synergy are for example those CI value<1 with the confidence interval lines also below 1. Graph D shows an exemplary combination experiment result using this data analysis.

FIGS. 21A-21B are plots demonstrating synergistic and durable response with combination of Compound A2 and AML standard of care drugs in MLL-r leukemia cell lines. FIG. 21A shows that Compound A2 demonstrates synergistic antiproliferative activity in combination with standard of care (SOC) drugs for AML in MLL-rearranged leukemia cell lines MOLM-13 (panels a and b) and MV4-11 (panels c and d). Cells were treated according to the pre-treatment model described in the Methods Section A (no Compound A2 washout). Synergistic anti-proliferative activity of Compound A2 in combination with AML SOC agents was also observed when cells were treated according to the co-treatment model described in the Methods Section B (data not shown). FIG. 21B shows synergistic anti-proliferative activity between Compound A2 and AML SOC agents is maintained in MOLM-13 (panels a and b) and MV4-11 (panels c and d) MLL-rearranged cells following Compound A2 washout prior to the addition of the SOC agent. Cells were treated according to the pre-treatment model described in the Methods Section A (with Compound A2 washout).

FIGS. 22A-22D are plots showing that cotreatment of Compound A2 with standard of care agent Ara-C demonstrates increased fraction of apoptotic cells in a time and dose dependent manner. FIG. 22A shows that Compound A2 as a single agent induces a dose dependent increase in apoptotic cells after 7 days of treatment. FIG. 22B shows that Compound A2 and Ara-C act synergistically to enhance apoptosis in MLL-rearranged MOLM-13 cells. Compound treatments were performed as described in the Methods section under treatment for mechanism of cell death studies. In A and B, data represent mean of percentage of gated cells in each stage of apoptosis. * *Day 14 resulted in fewer cell events. Green stacks represent percentages of cells in early stage apoptosis (means+/−S.D., n=3). *** *P<0.0001 (ANOVA plus Bonferroni's post-test) Combination of Compound A2 with Ara-C compared with Compound A2 alone, #### P<0.0001 (ANOVA plus Bonferroni's post-test) combination of Compound A2 with Ara-C compared with Ara-C alone. FIG. 22C shows representative apoptosis dot plots of MOLM-13 cells on Day 10. Cells were treated with DMSO (panel a), Compound A2 (panel b), Ara-C (panel d) or the combination of Ara-C and Compound A2 (panel d). FIG. 22D shows a synergistic increase in apoptosis was detected by an increase in the percent of cells in sub-GI phase of the cell cycle and an increase in the percentage of cells staining positive for annexin-V. Similar results were observed when Compound A2 was combined with daunorubicin (data not shown).

FIGS. 23A-23B are plots demonstrating that Compound A2 increases expression of differentiation markers as single agent and in combination with Ara-C in the MOLM-13 cells. FIG. 23A shows that Compound A2 and Ara-C as single agents and in combination promote time and concentration dependent up-regulation of the differentiation markers CD11b and CD14 (data not shown) in MLL-rearranged MOLM-13 cells. FIG. 23B shows that IgG was utilized as a control. Cells were harvested at day 10 (panels a, b, and c) or day 14 (panels d, e, and f) for measuring the markers. Cells were treated with Compound A2 (panels a and d), Ara-C (panels b and e) or the combination (panels c and f). Cultures treated as described in the Methods section for mechanism of cell death studies.

FIGS. 24A-24B are plots showing that Compound A2 does not enhance anti-proliferative effect of standard of care drugs in non-MLL rearranged SKM-1 cells. Compound A2 has no single agent activity in non-MLL rearranged cell line SKM-1 and no augmentation of antileukemic activity was observed upon treatment with a combination of standard of care drugs and Compound A2 according to the co-treatment model described in the Methods section. FIG. 24A shows combination of Compound A2 and Ara-C and FIG. 24B shows combination of Compound A2 and Daunorubicin.

FIGS. 25A-25C are plots showing that Compound A2 demonstrates strong synergy with DNMT inhibitor Azacytidine in MLL-rearranged cell lines. Compound A2 and azacytidine synergistically induce an anti-proliferative effect in co-treatment models of MLL-rearranged leukemia. FIG. 25A shows MOLM-13 cell line and FIG. 25B shows MV4-11 cell line. FIG. 25C shows that Azacytidine single agent activity was not potentiated by Compound A2 in the non-rearranged SKM-1 cell line.

FIGS. 26A-26D are treatment schemes for the study presented herein. FIG. 26A shows a pre-treatment model. FIG. 26B shows a co-treatment model. FIG. 26C shows a treatment model for mechanism of action studies. FIG. 26D shows a pre-treatment model for reverse order of addition.

FIGS. 27A-27B are graphs showing combination therapy of Ara-C and Compound A2. Synergy is observed when cells are pretreated with Ara-C followed by cotreatment with Compound A2. Combination benefit is maintained when Ara-C is washed out prior to treatment with compound A2. FIG. 27A shows Ara-C Treatment for 3 Days followed by Compound A2 and Ara-C co-treatment for 7 Days. FIG. 27B shows Ara-C Treatment for 3 Days followed by Compound A2 Treatment for 7 Days (washout Ara-C).

FIGS. 28A-28D are graphs demonstrating that Compound A2 induces a synergistic and durable antiproliferative effect in combination with AML Standard of Care Drugs in MLL-rearranged leukemia cell lines. Cells were treated with Compound A2 continuously. FIG. 28A shows the combination of Compound A2 and Ara-C in MOLM-13 cells. FIG. 28B shows the combination of Compound A2 and Daunorubicin in MoLM-13 cells. FIG. 28C shows the combination of Compound A2 and Ara-C in MV4-11 cells. FIG. 28D shows the combination of Compound A2 and Daunorubicin in MV4-11 cells.

FIGS. 29A-29D are graphs showing that Compound A2 induces a synergistic and durable antiproliferative effect in combination with AML Standard of Care Drugs in MLL-rearranged leukemia cell lines. Compound A2 was washed out. FIG. 29A shows the combination of Compound A2 and Ara-C in MOLM-13 cells. FIG. 29B shows the combination of Compound A2 and Daunorubicin in MoLM-13 cells. FIG. 29C shows the combination of Compound A2 and Ara-C in MV4-11 cells. FIG. 29D shows the combination of Compound A2 and Daunorubicin in MV4-11 cells.

FIGS. 30A-30B are graphs showing that combination benefit is maintained when cells are pretreated with Ara-C prior to cotreatment with Compound A2 and durable upon removal of Ara-C after pretreatment in the MOLM-13 cell line. FIG. 30A shows Ara-C and Compound A2 co-treatment and FIG. 30B shows Ara-C washout before Compound A2 treatment.

FIGS. 31A-31B are graphs showing that Compound A2 (also called EPZ-5676 or 5676 in all the experiments described herein) does not enhance anti-proliferative effect of standard of care drugs in non-MLL rearranged SKM-1 cells. FIG. 31A shows the combination of Compound A2 and Ara-C and FIG. 31B shows the combination of Compound A2 and Daunorubicin.

FIGS. 32A-32D are graphs showing that Compound A2 increases expression of differentiation markers and apoptosis as single agent and in combination with standard of care drugs in the MOLM-13 cell line. FIG. 32A shows percent change of viable cells, early stage apoptosis, late stage apoptosis and nuclear debris in cells treated with DMSO or different dosage of Compound A2 alone. FIG. 32B show percent change of viable cells, early stage apoptosis, late stage apoptosis and nuclear debris in cells treated with DMSO or different combination of Compound A2 with standard care of drugs. FIG. 32C shows the distribution of cell cycle stages at various time points for MOLM-13 cells treated with DMSO (control), 156 nM Compound A2, 63 nM Ara-C or a combination of Compound A2 and Ara-C. FIG. 32D is a kinetic plot for the sub-G1 cell population.

FIGS. 33A-33D are graphs showing the same results of FIGS. 32A-32D in a different format. FIGS. 33A and 33B show the late and early apoptosis progress curves of cells treated with Compound A2 alone, Ara-C alone, or combination of Compound A2 and Ara-C. Cells in FIG. 33B received a pretreatment. FIGS. 33D and 33D show the cell cycle progress curves of cells treated with Compound A2 alone, Ara-C alone, or combination of Compound A2 and Ara-C. Cells in FIG. 33D received a pretreatment.

FIGS. 34A-34C are panels showing that Compound A2 increase expression of differentiation marker and apoptosis as single agent and in combination with standard of care drugs in the MOLM-13 cell line. FIG. 34A shows marker CD11b, FIG. 34B shows marker CD14 and FIG. 34C shows control marker IgG. Each small panel in each figure corresponds to a treatment regimen: cells in panel a were treated with Compound A2 alone and harvested at day 10; cells in panel b were treated with Compound A2 alone and harvested at day 14; cells in panel c were treated with Ara-C alone and harvested at day 10; cells in panel d were treated with Ara-C alone and harvested at day 14; cells in panel e were treated with Compound A2 and Ara-C and harvested at day 10; cells in panel f were treated with Compound A2 and Ara-C and harvested at day 14.

FIGS. 35A-35C are graphs showing that Compound A2 demonstrates strong synergy with DNMT inhibitor Azacytidine in MLL-rearranged cell lines and other chromatin modifying agents. FIG. 35A shows MOLM-13 cells. FIG. 35B shows MV4-11 cells. FIG. 35C shows SKM-1 cells.

### DETAILED DESCRIPTION OF THE INVENTION

The present invention is based upon the discovery that DOT1L histone methyltransferase inhibitors and anti-cancer agents can be used in combination to treat tumors with superior results than those achieved by treating tumors with DOT1L histone methyltransferase inhibitors alone or anti-cancer agents alone.

Accordingly, the present invention provides a composition comprising a DOT1L histone methyltransferase inhibitor and one or more therapeutic agents, and methods for their use to treat diseases the course of which can be influenced by modulating the methylation status of histones or other proteins, e.g., cancer. In particular, the present invention features a composition comprising Formula (I) and Ara-C or Daunorubicin.

The present invention also includes methods for combination therapies comprising DOT1L histone methyltransferase inhibitor and one or more therapeutic agents, such as a compound of Formula (I) and Ara-C or Daunorubicin, to treat cancer, e.g., leukemia. Specifically, the methods of the present invention are useful for treating or inhibiting cancer cell proliferation.

The present invention further provides uses of any composition described herein in the manufacture of medicament for treating diseases. Such diseases include, for example, cancer, a precancerous condition, or a disease influenced by modulating the methylation status of histones or other proteins.

Any compound (e.g., DOT1L inhibitor) disclosed herein can be used for the compositions or combination therapy of the invention.

In one aspect, a composition of the invention comprises a compound of Formula (I) or a pharmaceutically acceptable salt thereof, and one or more therapeutic agents. The compounds of Formula (I) are suitable for administration as part of a combination therapy with one or more therapeutic agents or treatment modality, suitable to be administered together, sequentially, or in alternation.

The invention provides the compounds of Formula (I):

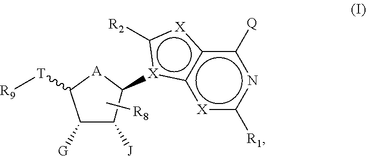

or a pharmaceutically acceptable salt or ester thereof,
wherein,
T is a linker group of a 6-10 carbon atoms, in which one or more carbon atoms are optionally replaced with a heteroatom and T is optionally substituted;
R₉ comprises a C₆-C₁₀ aryl or 5 to 10-membered heteroaryl optionally substituted with one or more substituents selected from the group consisting of unsubstituted or substituted t-butyl, CF₃, cyclohexyl, C₆-C₁₀ aryl, and 5 to 10-membered heteroaryl;
A is O or CH₂;
each of G and J, independently, is H, halo, C(O)OH, C(O)O—C₁-C₆ alkyl or ORₐ, Rₐ being H, C₁-C₆ alkyl, C(O)—C₁-C₆ alkyl, or silyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;
each X independently is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
each of R₁ and R₂, independently is H, halo, hydroxyl, carboxyl, cyano, or R_{S2}, R_{S2} being amino, C₁-C₆ alkoxyl, C₁-C₆ alkyl, C₂-C₆ alkenyl, C₂-C₆ alkynyl, or C₃-C₈ cycloalkyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl; and

Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl.

For example, in Formula (I), R₉ is selected from the group consisting of

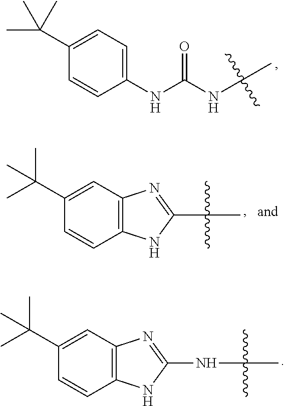 [SMILES: *NC(=O)Nc1ccc(C(C)(C)C)cc1 ; *c1nc2cc(C(C)(C)C)ccc2[nH]1 ; *Nc1nc2cc(C(C)(C)C)ccc2[nH]1]

For example, in Formula (I), T is —CH₂-L₁-L₂-L₃-, with L₃ connected to R₉, wherein:
L₁ is N(Y), S, SO, or SO₂;
L₂ is CO or absent when L₁ is N(Y), or L₂ is absent when L₁ is S, SO, or SO₂, in which Y is H, R_{d}, SO₂R_{d}, or COR_{d} when L₂ is absent, or Y is H or R_{d} when L₂ is CO, R_{d} being C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{d} being optionally substituted with one or more substituents selected from the group consisting of C₁-C₆ alkyl, C₂-C₆ alkenyl, C₂-C₆ alkynyl, halo, carboxyl, cyano, C₁-C₆ alkoxyl, C₁-C₆ alkylsulfonyl, C₃-C₈ cycloalkyl, C₆-C₁₀ aryl, 4 to 8-membered heterocycloalkyl, 5 to 6-membered heteroaryl, OR_{d′}, OCOR_{d′}, and N(R_{d′})₂, and with C₃-C₈ cycloalkyl, C₆-C₁₀ aryl, 4 to 6-membered heterocycloalkyl, or 5 to 6-membered heteroaryl further optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl; each R_{d}, independently being H, C₁-C₆ alkyl, silyl, C₁-C₆ alkyl-C₃-C₈ cycloalkyl, C₆-C₁₀ aryl, 5 to 6-membered heteroaryl, aralkyl, or heteroaralkyl;
L₃ is —(CR₄R₅)ₙ(CR₆R₇)ₘ— or —(CR₄R₅)ₙ-unsubstituted or substituted C₃-C₈ cycloalkyl-(CR₆R₇)ₘ—, with (CR₆R₇)ₘ connected to R₉;
each of R₄, R₅, R₆, and R₇, independently, is H, halo, hydroxyl, carboxyl, cyano, or R_{S2}, R_{S2} being amino, C₁-C₆ alkoxyl, C₁-C₆ alkyl, C₂-C₆ alkenyl, or C₂-C₆ alkynyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl; or two geminal R₄ and R₅ or two geminal R₆ and R₇ taken together are ethylene, propylene or butylene;
m is 0, 1, or 2; and
n is 0, 1, or 2.

For example, in Formula (I) R₉

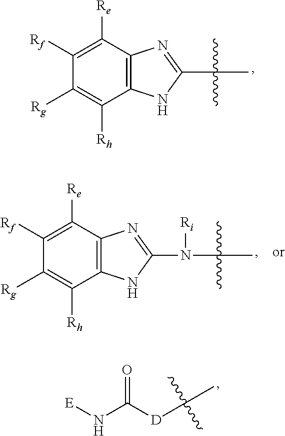

in which:
each of Rₑ, R_{f}, R_{g}, and Rₕ, independently is -M₂-T₂, in which M₂ is a bond, SO₂, SO, S, CO, CO₂, O, O—C₁-C₄ alkyl linker, C₁-C₄ alkyl linker, NH, or N(Rₜ), Rₜ being C₁-C₆ alkyl, and T₂ is H, halo, or R_{S4}, R_{S4} being C₁-C₆ alkyl, C₂-C₆ alkenyl, C₂-C₆ alkynyl, C₃-C₈ cycloalkyl, C₆-C₁₀ aryl, 4 to 8-membered heterocycloalkyl, or 5 to 10-membered heteroaryl, and each of O—C₁-C₄ alkyl linker, C₁-C₄ alkyl linker, Rₜ, and R_{S4} being optionally substituted with one or more substituents selected from the group consisting of halo, hydroxyl, carboxyl, cyano, C₁-C₆ alkyl, C₂-C₆ alkenyl, C₂-C₆ alkynyl, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl, Rᵢ is H or C₁-C₆ alkyl optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl,
D is O, NRⱼ, or CRⱼRₖ, each of Rⱼ and Rₖ independently being H or C₁-C₆ alkyl, or Rⱼ and Rₖ taken together, with the carbon atom to which they are attached, form a C₃-C₁₀ cycloalkyl ring, and

E is -M₃-T₃, M₃ being a bond or C₁-C₆ alkyl linker optionally substituted with halo or cyano, T₃ being C₃-C₁₄ carbocycle or 4 to 14-membered heterocycle, and T₃ being optionally substituted with one or more substituents selected from the group consisting of halo, hydroxyl, thiol, carboxyl, cyano, nitro, C₁-C₆ alkyl, C₂-C₆ alkenyl, C₂-C₆ alkynyl, C₁-C₆ alkoxyl, C₁-C₆ haloalkyl, C₁-C₆ haloalkoxyl, C₁-C₆ alkylthio, C₁-C₆ alkylsulfonyl, C₁-C₆ haloalkylsulfonyl, C₁-C₆ alkylcarbonyl, C₁-C₆ alkoxycarbonyl, oxo, amino, mono-C₁-C₆ alkylamino, di-C₁-C₆ alkylamino, C₃-C₈ cycloalkyl, C₄-C₁₂ alkylcycloalkyl, C₆-C₁₀ aryl, C₆-C₁₀ aryloxyl, C₇-C₁₄ alkylaryl, C₆-C₁₀ aminoaryloxyl, C₆-C₁₀ arylthio, 4 to 6-membered heterocycloalkyl optionally substituted with halo, C₁-C₄ alkyl, C₁-C₄ haloalkyl, 5 to 6-membered heteroaryl optionally substituted with halo, C₁-C₄ alkyl, and C₁-C₆ alkyl that is substituted with hydroxy, halo, C₁-C₆ alkoxycarbonyl, C₃-C₈ cycloalkyl, C₆-C₁₀ aryl, 4 to 6-membered heterocycloalkyl, or 5 to 6-membered heteroaryl optionally further substituted with halo, hydroxyl, or C₁-C₆ alkoxyl.

For example, the compound of Formula (I) is of formula (IIa) or (IIb):

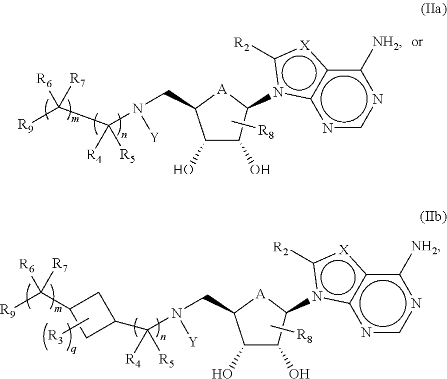

wherein R₃ is H, halo, hydroxyl, carboxyl, cyano, or R_{S2}, and q is 0, 1, 2, 3, or 4.

For example, the compound is of formula (IIa) and R₉ is

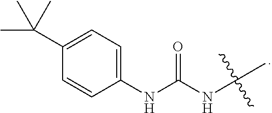 [SMILES: *NC(=O)Nc1ccc(C(C)(C)C)cc1]

For example, the compound is of formula (I %) and R₉ is

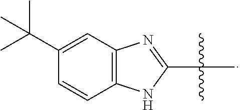 [SMILES: *c1nc2cc(C(C)(C)C)ccc2[nH]1]

Compounds of Formula (I) also include those of Formula (IIIa) or (IIIb)

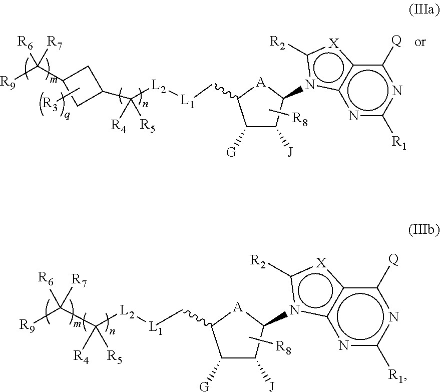

or a pharmaceutically acceptable salt or ester thereof, wherein:

A is O or CH₂;

each of G and J, independently, is H, halo, C(O)OH, C(O)O—C₁-C₆ alkyl or ORₐ, Rₐ being H, C₁-C₆ alkyl or C(O)—C₁-C₆ alkyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;

Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;

X is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;

L₁ is N(Y), S, SO, or SO₂;

L₂ is CO or absent when L₁ is N(Y) or L₂ is absent when L₁ is S, SO, or SO₂, in which Y is H, R_{d}, SO₂R_{d}, or COR_{d} when L₂ is absent, or Y is H or R_{d} when L₂ is CO, R_{d} being C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{d} being optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, C₁-C₆ alkylsulfonyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl and with C₃-C₈ cycloalkyl, C₆-C₁₀ aryl, 4 to 6-membered heterocycloalkyl, or 5 to 6-membered heteroaryl further optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl;

each of R₁, R₂, R₃, R₄, R₅, R₆, and R₇, independently, is H, halo, hydroxyl, carboxyl, cyano, R_{S2}, R_{S2} being amino, C₁-C₆ alkoxyl, C₁-C₆ alkyl, C₂-C₆ alkenyl, or C₂-C₆ alkynyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;

R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl;

R₉ is

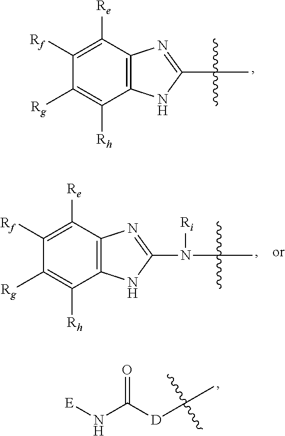

in which each of Rₑ, R_{f}, R_{g}, and Rₕ, independently is -M₂-T₂, in which M₂ is a bond, SO₂, SO, S, CO, CO₂, O, O—C₁-C₄ alkyl linker, C₁-C₄ alkyl linker, NH, or N(Rₜ), Rₜ being C₁-C₆ alkyl, and T₂ is H, halo, or R_{S4}, R_{S4} being C₁-C₆ alkyl, C₂-C₆ alkenyl, C₂-C₆ alkynyl, C₃-C₈ cycloalkyl, C₆-C₁₀ aryl, 4 to 8-membered heterocycloalkyl, or 5 to 10-membered heteroaryl, and each of O—C₁-C₄ alkyl linker, C₁-C₄ alkyl linker, Rₜ, and R_{S4} being optionally substituted with one or more substituents selected from the group consisting of halo, hydroxyl, carboxyl, cyano, C₁-C₆ alkyl, C₂-C₆ alkenyl, C₂-C₆ alkynyl, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl, Rᵢ is H or C₁-C₆ alkyl optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl, D is O, NRⱼ, or CRⱼRₖ, each of Rⱼ and Rₖ independently being H or C₁-C₆ alkyl, or Rⱼ and Rₖ taken together, with the carbon atom to which they are attached, form a C₃-C₁₀ cycloalkyl ring, and E is -M₃-T₃, M₃ being a bond or C₁-C₆ alkyl linker optionally substituted with halo or cyano, T₃ being C₃-C₁₀ cycloalkyl, C₆-C₁₀ aryl, 5 to 10-membered heteroaryl, or 4 to 10-membered heterocycloalkyl, and T₃ being optionally substituted with one or more substituents selected from the group consisting of halo, hydroxyl, thiol, carboxyl, cyano, nitro, C₁-C₆ alkyl, C₂-C₆ alkenyl, C₂-C₆ alkynyl, C₁-C₆ alkoxyl, C₁-C₆ haloalkyl, C₁-C₆ haloalkoxyl, C₁-C₆ alkylthio, C₁-C₆ alkylsulfonyl, C₁-C₆ haloalkylsulfonyl, C₁-C₆ alkylcarbonyl, C₁-C₆ alkoxycarbonyl, oxo, amino, mono-C₁-C₆ alkylamino, di-C₁-C₆ alkylamino, C₃-C₈ cycloalkyl, C₄-C₁₂ alkylcycloalkyl, C₆-C₁₀ aryl, C₆-C₁₀ aryloxyl, C₇-C₁₄ alkylaryl, C₆-C₁₀ aminoaryloxyl, C₆-C₁₀ arylthio, 4 to 6-membered heterocycloalkyl optionally substituted with halo, C₁-C₄ alkyl, C₁-C₄ haloalkyl, 5 to 6-membered heteroaryl optionally substituted with halo, C₁-C₄ alkyl, and C₁-C₆ alkyl that is substituted with hydroxy, halo, C₁-C₆ alkoxycarbonyl, C₃-C₈ cycloalkyl, C₆-C₁₀ aryl, 4 to 6-membered heterocycloalkyl, or 5 to 6-membered heteroaryl optionally further substituted with halo, hydroxyl, or C₁-C₆ alkoxyl;

q is 0, 1, 2, 3, or 4;

m is 0, 1, or 2; and

n is 0, 1, or 2.

For example, the sum of m and n is at least 1.

For example, m is 1 or 2 and n is 0.

For example, m is 2 and n is 0

For example, A is CH₂.

For example, A is O.

For example, L₁ is N(Y).

For example, L₁ is SO or SO₂.

For example, Y is R_{d}.

For example, R_{d} is C₁-C₆ alkyl.

For example, L₂ is absent.

For example, each of G and J independently is ORₐ.

For example, Rₐ is H.

For example, R₉ is

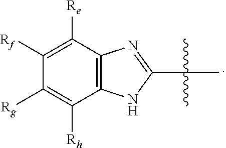

For example, R₉ is

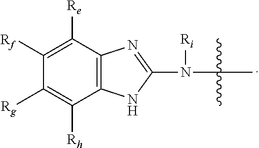

For example, at least one of Rₑ, R_{f}, R_{g}, and Rₕ is halo (such as F, Cl, and Br), C₁-C₆ alkoxyl optionally substituted with one or more halo (such as OCH₃, OCH₂CH₃, O-iPr, and OCF₃), C₁-C₆ alkylsulfonyl optionally substituted with one or more halo (such as SO₂CF₃), or C₁-C₆ alkyl optionally substituted with one or more halo (such as CH₃, i-propyl, n-butyl, and CF₃).

For example, Rᵢ is H or C₁-C₆ alkyl (e.g., methyl, ethyl, n-propyl, i-propyl, n-butyl, s-butyl, t-butyl, n-pentyl, s-pentyl and n-hexyl).

For example,

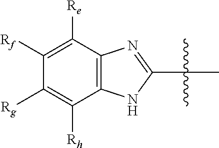

is unsubstituted benzimidazolyl or one of the following groups:

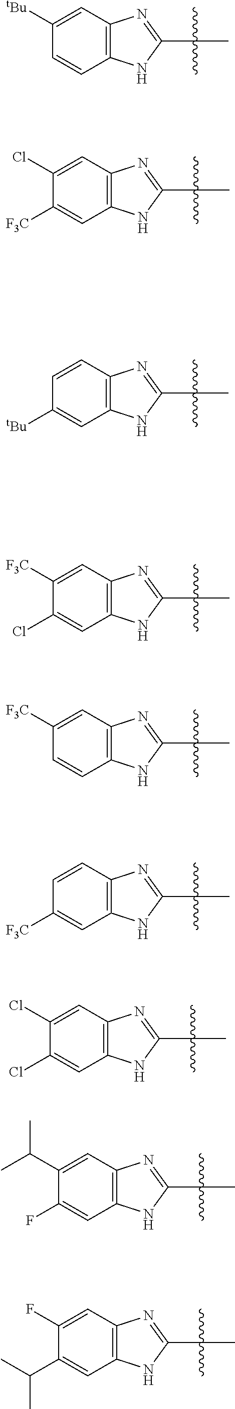

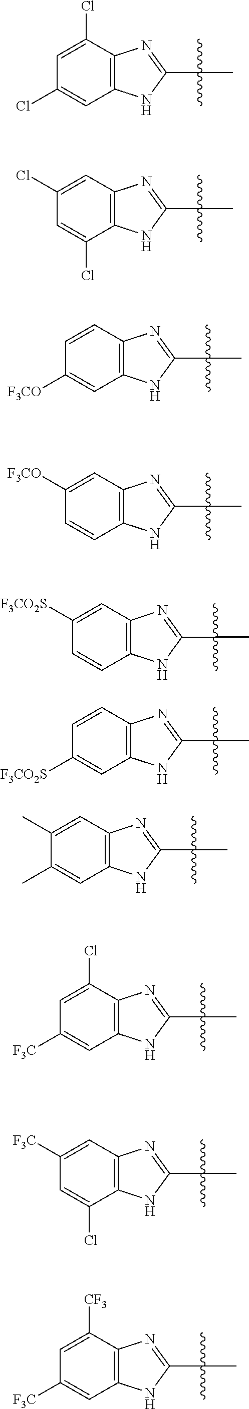

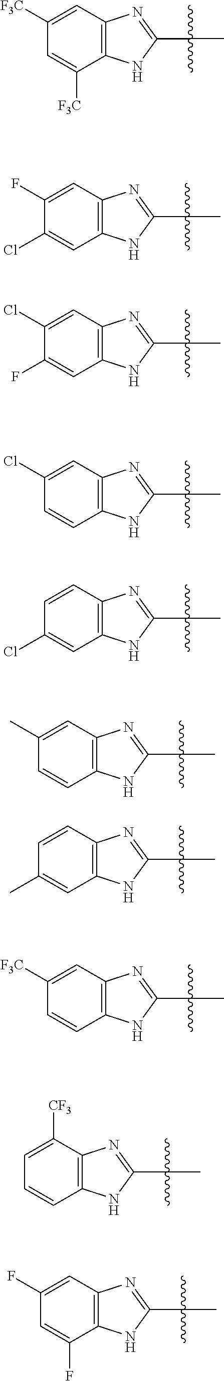

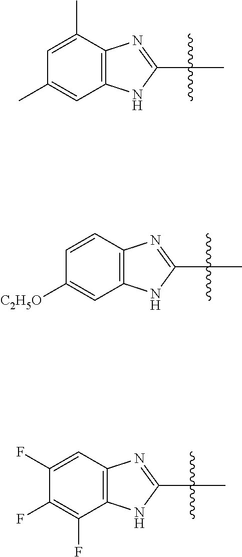

For example, R₉ is

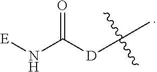

For example, D is O.

For example, D is NRⱼ.

For example, Rⱼ is H.

For example, D is CRⱼRₖ.

For example, each of Rⱼ and Rₖ is H.

For example, E is -M₃-T₃, in which M₃ is a bond or C₁-C₃ alkyl linker, T₃ is phenyl, naphthyl, thienyl, cyclopropyl, or cyclohexyl, and T₃ is optionally substituted with one or more substituents selected from the group consisting of halo, hydroxyl, thiol, carboxyl, cyano, nitro, C₁-C₆ alkyl, C₂-C₆ alkenyl, C₂-C₆ alkynyl, C₁-C₆ alkoxyl, C₁-C₆ haloalkyl, C₁-C₆ haloalkoxyl, C₁-C₆ alkylthio, C₁-C₆ alkylsulfonyl, C₁-C₆ alkylcarbonyl, C₁-C₆ alkoxycarbonyl, oxo, amino, mono-C₁-C₆ alkylamino, di-C₁-C₆ alkylamino, C₃-C₈ cycloalkyl, C₄-C₁₂ alkylcycloalkyl, C₆-C₁₀ aryl, C₆-C₁₀ aryloxyl, C₇-C₁₄ alkylaryl, C₆-C₁₀ aminoaryloxyl, C₆-C₁₀ arylthio, 4 to 6-membered heterocycloalkyl optionally substituted with C₁-C₄ alkyl, 5 to 6-membered heteroaryl optionally substituted with C₁-C₄ alkyl, and C₁-C₆ alkyl that is substituted with hydroxy, C₁-C₆ alkoxycarbonyl, C₃-C₈ cycloalkyl, C₆-C₁₀ aryl, 4 to 6-membered heterocycloalkyl, or 5 to 6-membered heteroaryl.

For example, T₃ is phenyl optionally substituted with one or more substituents selected from the group consisting of halo, hydroxyl, carboxyl, cyano, nitro, C₁-C₆ alkyl (e.g., methyl, ethyl, n-propyl, i-propyl, n-butyl, s-butyl, t-butyl, n-pentyl, s-pentyl and n-hexyl), C₁-C₆ alkoxyl, C₁-C₆ haloalkyl, C₁-C₆ haloalkoxyl, C₁-C₆ alkylsulfonyl, C₆-C₁₀ aryl (e.g., phenyl or naphthyl), and C₆-C₁₀ aryloxyl, and C₇-C₁₄ alkylaryl.

For example, E is

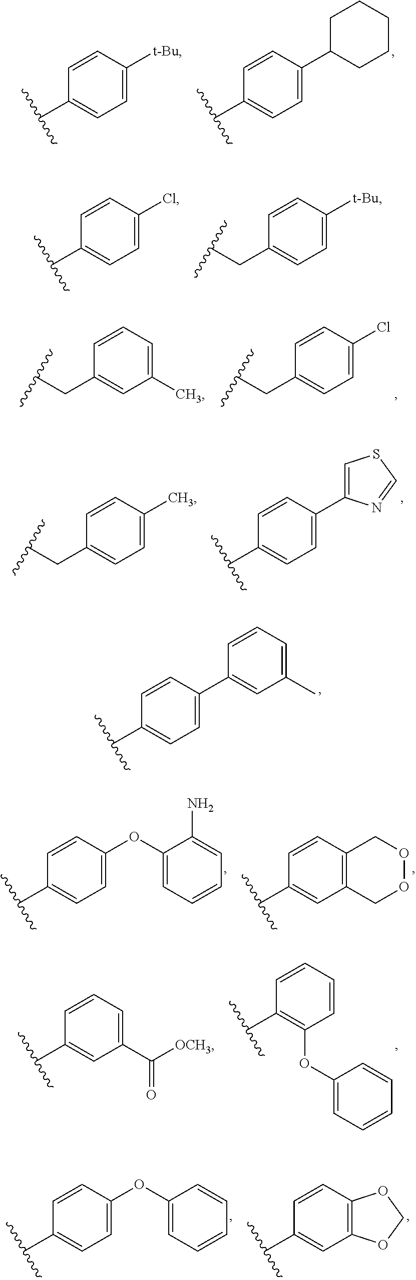

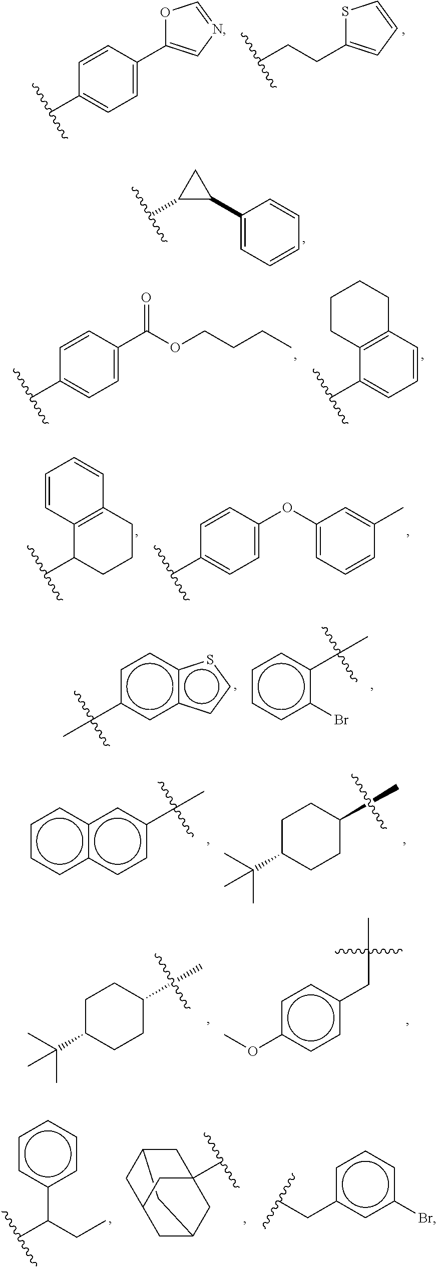

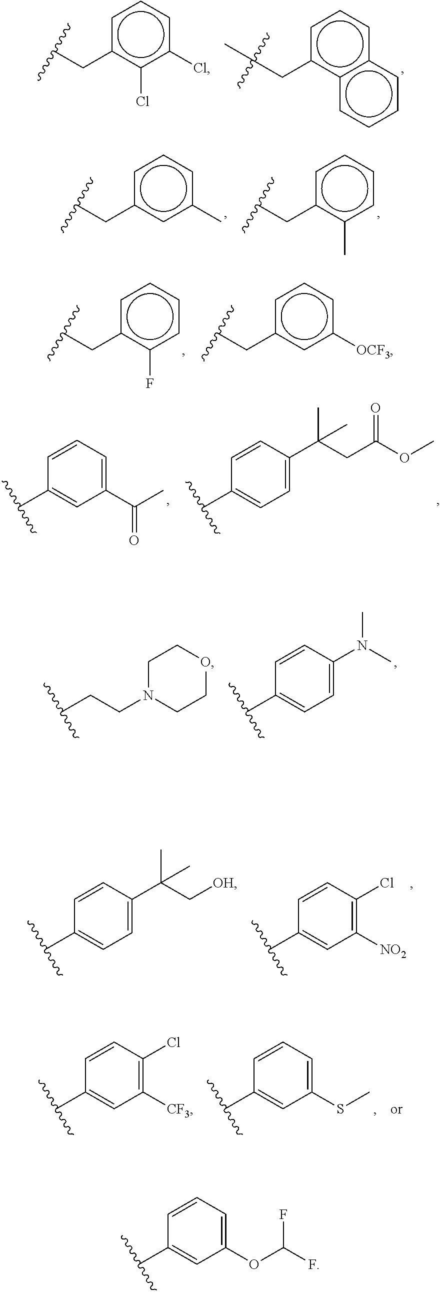

For example, X is N.

For example, X is CRₓ.

For example, X is CH.

For example, Q is NH₂ or NHR_{b}, in which R_{b} is -M₁-T₁, M₁ being a bond or C₁-C₆ alkyl linker and T₁ being C₃-C₈ cycloalkyl.

For example, Q is H.

For example, R₁, R₂, R₃, R₄, R₅, R₆, R₇, and R₈ are each H.

For example, when R₈ is halo and is attached to the same carbon atom as J, then J is not hydroxyl.

For example, when R₈ is halo and is attached to the same carbon atom as G, then G is not hydroxyl.

For example, T₂ is not halo when M₂ is SO₂, SO, S, CO or O.

For example, T₂ is a 4-8 membered heterocycloalkyl which is bound to M₂ via a heteroatom.

For example, T₂ is a 4-8 membered heterocycloalkyl which is bound to M₂ via a N atom.

For example, T₂ is a 4-8 membered heterocycloalkyl which is bound to M₂ via a C atom.

The present invention provides the compounds of Formula (IVa), (IVb), (IVd), or (IVe):

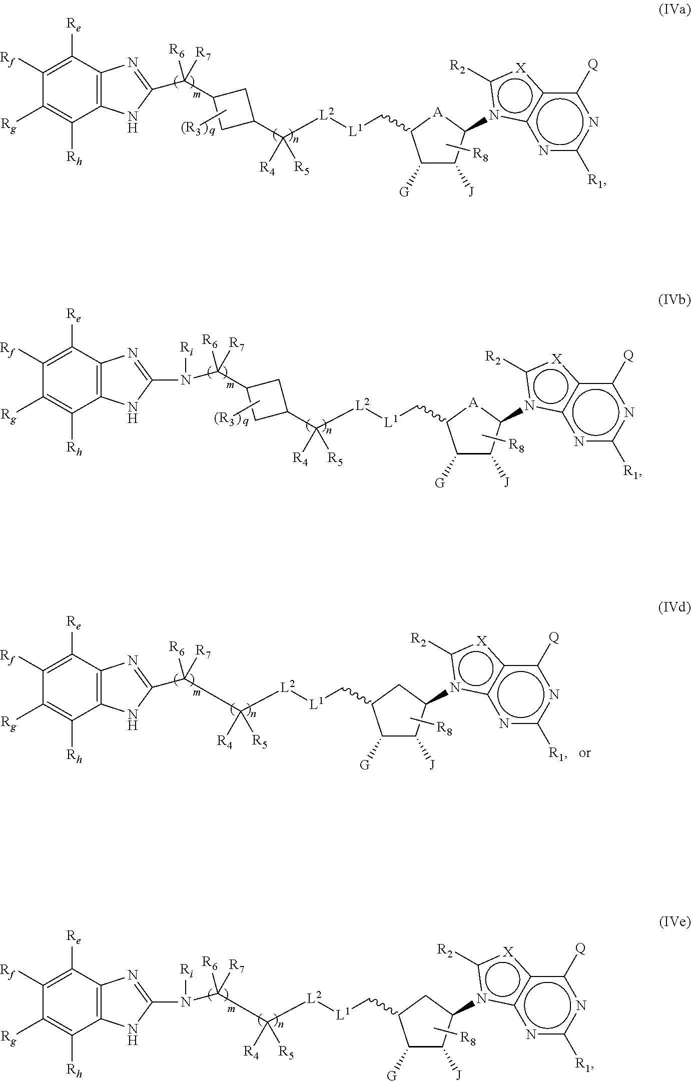

or a pharmaceutically acceptable salt or ester thereof, wherein:
A is O or CH₂;
each of G and J, independently, is H, halo, C(O)OH, C(O)O—C₁-C₆ alkyl or ORₐ, Rₐ being H, C₁-C₆ alkyl or C(O)—C₁-C₆ alkyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;
Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;

X is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
L₁ is N(Y), S, SO, or SO₂;
L₂ is CO or absent when L₁ is N(Y) or L₂ is absent when L₁ is S, SO, or SO₂, in which Y is H, R_{d}, SO₂R_{d}, or COR_{d} when L₂ is absent, or Y is H or R_{d} when L₂ is CO, R_{d} being C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{d} being optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, C₁-C₆ alkylsulfonyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl and with C₃-C₈ cycloalkyl, C₆-C₁₀ aryl, 4 to 6-membered heterocycloalkyl, or 5 to 6-membered heteroaryl further optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl;
each of R₁, R₂, R₃, R₄, R₅, R₆, and R₇, independently, is H, halo, hydroxyl, carboxyl, cyano, R_{S2}, R_{S2} being amino, C₁-C₆ alkoxyl, C₁-C₆ alkyl, C₂-C₆ alkenyl, or C₂-C₆ alkynyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl;
each of Rₑ, R_{f}, R_{g}, and Rₕ, independently is -M₂-T₂, in which M₂ is a bond, SO₂, SO, S, CO, CO₂, O, O—C₁-C₄ alkyl linker, C₁-C₄ alkyl linker, NH, or N(Rₜ), Rₜ being C₁-C₆ alkyl, and T₂ is H, halo, or R_{S4}, R_{S4} being C₁-C₆ alkyl, C₂-C₆ alkenyl, C₂-C₆ alkynyl, C₃-C₈ cycloalkyl, C₆-C₁₀ aryl, 4 to 8-membered heterocycloalkyl, or 5 to 10-membered heteroaryl, and each of O—C₁-C₄ alkyl linker, C₁-C₄ alkyl linker, Rₜ, and R_{S4} being optionally substituted with one or more substituents selected from the group consisting of halo, hydroxyl, carboxyl, cyano, C₁-C₆ alkyl, C₂-C₆ alkenyl, C₂-C₆ alkynyl, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl,
Rᵢ is H or C₁-C₆ alkyl optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
q is 0, 1, 2, 3, or 4;
m is 0, 1, or 2; and
n is 0, 1, or 2.

For example, the sum of m and n is at least 1.

For example, m is 1 or 2 and n is 0.

For example, m is 2 and n is 0

For example, A is CH₂.

For example, A is O.

For example, L₁ is N(Y).

For example, L₁ is SO or SO₂.

For example, Y is R_{d}.

For example, R_{d} is C₁-C₆ alkyl.

For example, L₂ is absent.

For example, each of G and J independently is ORₐ.

For example, Rₐ is H.

For example, at least one of Rₑ, R_{f}, R_{g}, and Rₕ is halo (such as F, Cl, and Br), C₁-C₆ alkoxyl optionally substituted with one or more halo (such as OCH₃, OCH₂CH₃, O-iPr, and OCF₃), C₁-C₆ alkylsulfonyl optionally substituted with one or more halo (such as SO₂CF₃), or C₁-C₆ alkyl optionally substituted with one or more halo (such as CH₃, i-propyl, n-butyl, and CF₃).

For example, Rᵢ is H or C₁-C₆ alkyl (e.g., methyl, ethyl, n-propyl, i-propyl, n-butyl, s-butyl, t-butyl, n-pentyl, s-pentyl and n-hexyl).

For example,

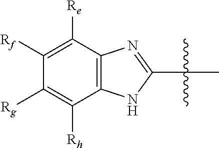

is unsubstituted benzimidazolyl or one of the following groups:

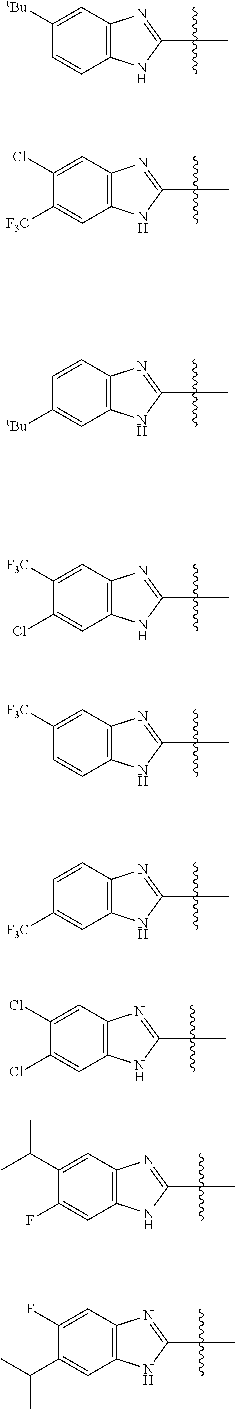

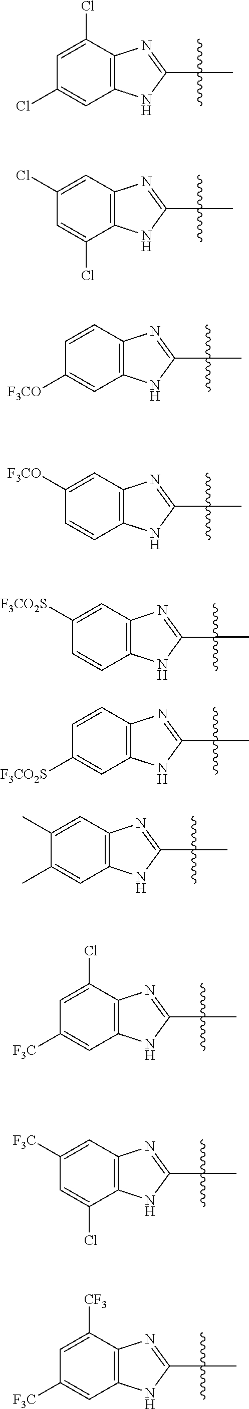

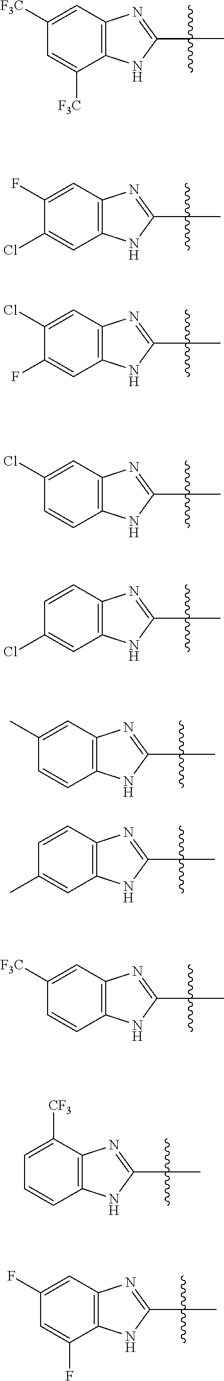

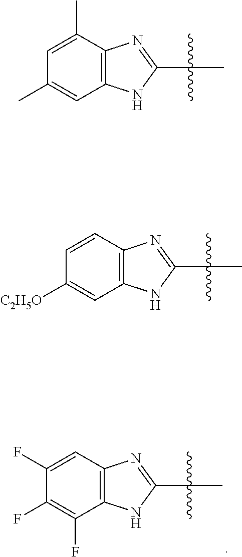

For example, X is N.

For example, X is CRₓ.

For example, X is CH.

For example, Q is NH₂ or NHR_{b}, in which R_{b} is -M₁-T₁, M₁ being a bond or C₁-C₆ alkyl linker and T₁ being C₃-C₈ cycloalkyl.

For example, Q is H.

For example, R₁, R₂, R₃, R₄, R₅, R₆, R₇, and R₈ are each H.

For example, when R₈ is halo and is attached to the same carbon atom as J, then J is not hydroxyl.

For example, when R₈ is halo and is attached to the same carbon atom as G, then G is not hydroxyl.

For example, T₂ is not halo when M₂ is SO₂, SO, S, CO or O.

For example, T₂ is a 4-8 membered heterocycloalkyl which is bound to M₂ via a heteroatom.

For example, T₂ is a 4-8 membered heterocycloalkyl which is bound to M₂ via a N atom.

For example, T₂ is a 4-8 membered heterocycloalkyl which is bound to M₂ via a C atom.

The present invention provides the DOT1L inhibitor compounds of Formula (IVc) or (IVf):

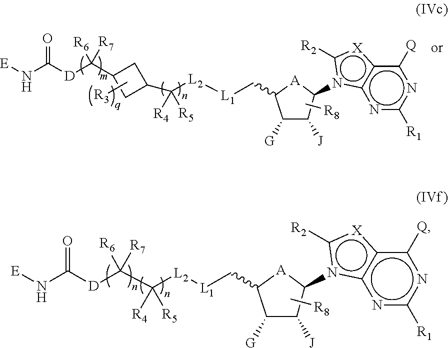

or
a pharmaceutically acceptable salt or ester thereof, wherein:
A is O or CH₂;
each of G and J, independently, is H, halo, C(O)OH, C(O)O—C₁-C₆ alkyl or ORₐ, Rₐ being H, C₁-C₆ alkyl or C(O)—C₁-C₆ alkyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;
Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
X is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
L₁ is N(Y), S, SO, or SO₂;
L₂ is CO or absent when L₁ is N(Y) or L₂ is absent when L₁ is S, SO, or SO₂, in which Y is H, R_{d}, SO₂R_{d}, or COR_{d} when L₂ is absent, or Y is H or R_{d} when L₂ is CO, R_{d} being C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{d} being optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, C₁-C₆ alkylsulfonyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl and with C₃-C₈ cycloalkyl, C₆-C₁₀ aryl, 4 to 6-membered heterocycloalkyl, or 5 to 6-membered heteroaryl further optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl;
each of R₁, R₂, R₃, R₄, R₅, R₆, and R₇, independently, is H, halo, hydroxyl, carboxyl, cyano, R_{S2}, R_{S2} being amino, C₁-C₆ alkoxyl, C₁-C₆ alkyl, C₂-C₆ alkenyl, or C₂-C₆ alkynyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl;
D is O, NRⱼ, or CRⱼRₖ, each of Rⱼ and Rₖ independently being H or C₁-C₆ alkyl, or Rⱼ and Rₖ taken together, with the carbon atom to which they are attached, form a C₃-C₁₀ cycloalkyl ring;
E is -M₃-T₃, M₃ being a bond or C₁-C₆ alkyl linker optionally substituted with halo or cyano, T₃ being C₃-C₁₀ cycloalkyl, C₆-C₁₀ aryl, 5 to 10-membered heteroaryl, or 4 to 10-membered heterocycloalkyl, and T₃ being optionally substituted with one or more substituents selected from the group consisting of halo, hydroxyl, thiol, carboxyl, cyano, nitro, C₁-C₆ alkyl, C₂-C₆ alkenyl, C₂-C₆ alkynyl, C₁-C₆ alkoxyl, C₁-C₆ haloalkyl, C₁-C₆ haloalkoxyl, C₁-C₆ alkylthio, C₁-C₆ alkylsulfonyl, C₁-C₆ haloalkylsulfonyl, C₁-C₆ alkylcarbonyl, C₁-C₆ alkoxycarbonyl, oxo, amino, mono-C₁-C₆ alkylamino, di-C₁-C₆ alkylamino, C₃-C₈ cycloalkyl, C₄-C₁₂ alkylcycloalkyl, C₆-C₁₀ aryl, C₆-C₁₀ aryloxyl, C₇-C₁₄ alkylaryl, C₆-C₁₀ aminoaryloxyl, C₆-C₁₀ arylthio, 4 to 6-membered heterocycloalkyl optionally substituted with halo, C₁-C₄ alkyl, C₁-C₄ haloalkyl, 5 to 6-membered heteroaryl optionally substituted with halo, C₁-C₄ alkyl, and C₁-C₆ alkyl that is substituted with hydroxy, halo, C₁-C₆ alkoxycarbonyl, C₃-C₈ cycloalkyl, C₆-C₁₀ aryl, 4 to 6-membered heterocycloalkyl, or 5 to 6-membered heteroaryl optionally further substituted with halo, hydroxyl, or C₁-C₆ alkoxyl;
q is 0, 1, 2, 3, or 4;
m is 0, 1, or 2; and
n is 0, 1, or 2.

For example, the sum of m and n is at least 1.

For example, m is 1 or 2 and n is 0.

For example, m is 2 and n is 0

For example, A is CH₂.

For example, A is O.

For example, L₁ is N(Y).

For example, L₁ is SO or SO₂.

For example, Y is R_{d}.

For example, R_{d} is C₁-C₆ alkyl.

For example, L₂ is absent.

For example, each of G and J independently is ORₐ.

For example, Rₐ is H.

For example, D is O.

For example, D is NRⱼ.

For example, Rⱼ is H.

For example, D is CRⱼRₖ.

For example, each of Rⱼ and Rₖ is H.

For example, E is -M₃-T₃, in which M₃ is a bond or C₁-C₃ alkyl linker, T₃ is phenyl, naphthyl, thienyl, cyclopropyl, or cyclohexyl, and T₃ is optionally substituted with one or more substituents selected from the group consisting of halo, hydroxyl, thiol, carboxyl, cyano, nitro, C₁-C₆ alkyl, C₂-C₆ alkenyl, C₂-C₆ alkynyl, C₁-C₆ alkoxyl, C₁-C₆ haloalkyl, C₁-C₆ haloalkoxyl, C₁-C₆ alkylthio, C₁-C₆ alkylsulfonyl, C₁-C₆ alkylcarbonyl, C₁-C₆ alkoxycarbonyl, oxo, amino, mono-C₁-C₆ alkylamino, di-C₁-C₆ alkylamino, C₃-C₈ cycloalkyl, C₄-C₁₂ alkylcycloalkyl, C₆-C₁₀ aryl, C₆-C₁₀ aryloxyl, C₇-C₁₄ alkylaryl, C₆-C₁₀ aminoaryloxyl, C₆-C₁₀ arylthio, 4 to 6-membered heterocycloalkyl optionally substituted with C₁-C₄ alkyl, 5 to 6-membered heteroaryl optionally substituted with C₁-C₄ alkyl, and C₁-C₆ alkyl that is substituted with hydroxy, C₁-C₆ alkoxycarbonyl, C₃-C₈ cycloalkyl, C₆-C₁₀ aryl, 4 to 6-membered heterocycloalkyl, or 5 to 6-membered heteroaryl.

For example, T₃ is phenyl optionally substituted with one or more substituents selected from the group consisting of halo, hydroxyl, carboxyl, cyano, nitro, C₁-C₆ alkyl (e.g., methyl, ethyl, n-propyl, i-propyl, n-butyl, s-butyl, t-butyl, n-pentyl, s-pentyl and n-hexyl), C₁-C₆ alkoxyl, C₁-C₆ haloalkyl, C₁-C₆ haloalkoxyl, C₁-C₆ alkylsulfonyl, C₆-C₁₀ aryl (e.g., phenyl or naphthyl), and C₆-C₁₀ aryloxyl, and C₇-C₁₄ alkylaryl.

For example, E is

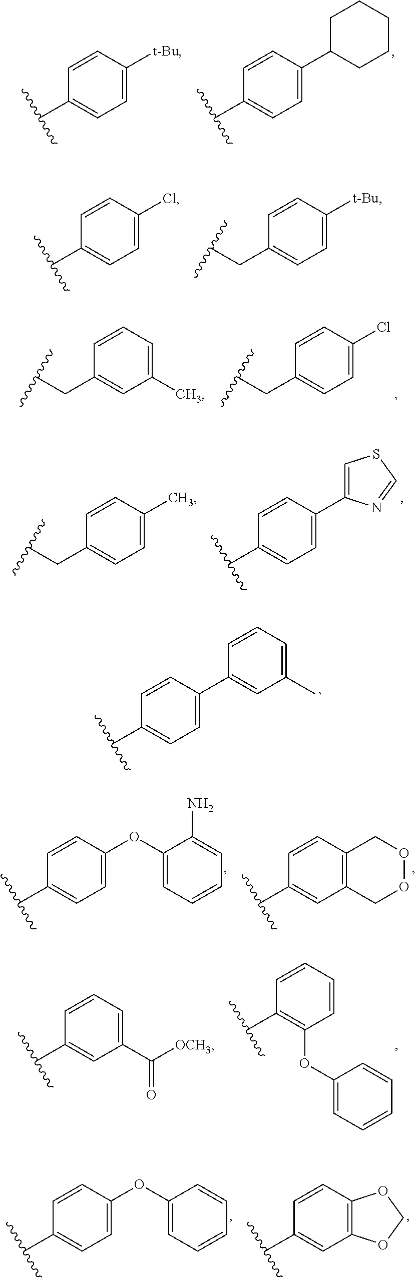

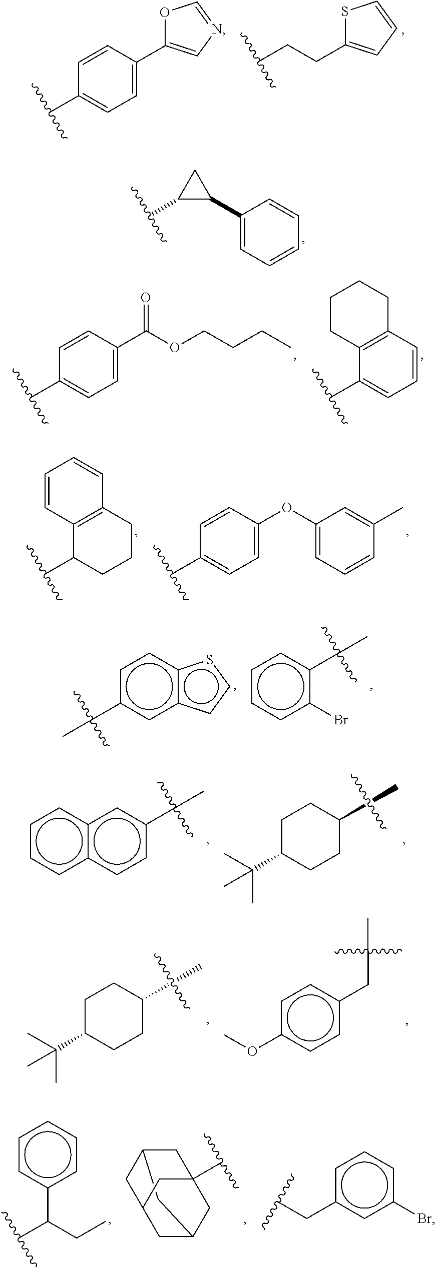

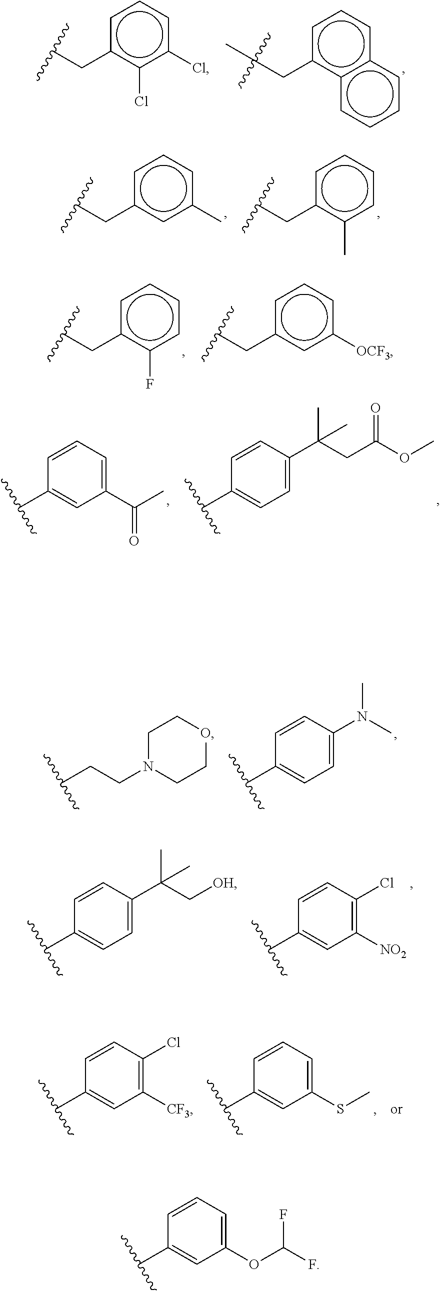

For example, X is N.

For example, X is CRₓ.

For example, X is CH.

For example, Q is NH₂ or NHR_{b}, in which R_{b} is -M₁-T₁, M₁ being a bond or C₁-C₆ alkyl linker and T₁ being C₃-C₈ cycloalkyl.

For example, Q is H.

For example, R₁, R₂, R₃, R₄, R₅, R₆, R₇, and R₈ are each H.

For example, when R₈ is halo and is attached to the same carbon atom as J, then J is not hydroxyl.

For example, when R₈ is halo and is attached to the same carbon atom as G, then G is not hydroxyl.

For example, T₂ is not halo when M₂ is SO₂, SO, S, CO or O.

For example, T₂ is a 4-8 membered heterocycloalkyl which is bound to M₂ via a heteroatom.

For example, T₂ is a 4-8 membered heterocycloalkyl which is bound to M₂ via a N atom.

For example, T₂ is a 4-8 membered heterocycloalkyl which is bound to M₂ via a C atom.

The invention also relates to a composition comprising one or more therapeutic agents and a compound of Formula (IV) or its N-oxide or a pharmaceutically acceptable salt thereof:

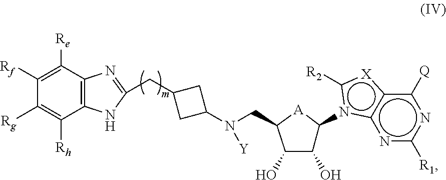

wherein A is O or CH₂;
Q is H, NH₂, NHR_{b}, NR_{b}R_{c}, R_{b}, ═O, OH, or OR_{b}, in which each of R_{b} and R_{c} independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
X is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
Y is H, R_{d}, SO₂R_{d}, or COR_{d}, R_{d} being C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{d} being optionally substituted with one or more substituents selected from C₁-C₆ alkyl, C₂-C₆ alkenyl, C₂-C₆ alkynyl, halo, hydroxyl, carboxyl, cyano, C₁-C₆ alkoxyl, C₁-C₆ alkylsulfonyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl and with C₃-C₈ cycloalkyl, C₆-C₁₀ aryl, 4 to 6-membered heterocycloalkyl, or 5 to 6-membered heteroaryl further optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl;
each of R₁ and R₂ independently, is H, halo, hydroxyl, carboxyl, cyano, R_{S2}, R_{S2} being amino, C₁-C₆ alkoxyl, C₁-C₆ alkyl, C₂-C₆ alkenyl, or C₂-C₆ alkynyl, and each R_{S2} being optionally substituted with one or more substituents selected from halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
each of Rₑ, R_{f}, R_{g}, and Rₕ, independently is -M₂-T₂, in which M₂ is a bond, SO₂, SO, S, CO, CO₂, O, O—C₁-C₄ alkyl linker, C₁-C₄ alkyl linker, NH, or N(Rₜ), Rₜ being C₁-C₆ alkyl, and T₂ is H, halo, or R_{S4}, R_{S4} being C₁-C₆ alkyl, C₂-C₆ alkenyl, C₂-C₆ alkynyl, C₃-C₈ cycloalkyl, C₆-C₁₀ aryl, 4 to 8-membered heterocycloalkyl, or 5 to 10-membered heteroaryl, and each of O—C₁-C₄ alkyl linker, C₁-C₄ alkyl linker, Rₜ, and R_{S4} being optionally substituted with one or more substituents selected from halo, hydroxyl, carboxyl, cyano, C₁-C₆ alkyl, C₂-C₆ alkenyl, C₂-C₆ alkynyl, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl, and
m is 0, 1, or 2.

For example, A is O. In certain compounds of Formula (IV), A is O and m is 2.

In certain compounds of Formula (IV), X is N.

For example, in certain compounds, Q is NH₂ or NHR_{b}, in which R_{b} is -M₁-T₁, M₁ being a bond or C₁-C₆ alkyl linker and T₁ being C₃-C₈ cycloalkyl

For example, in certain compounds of Formula (IV), R₁ and R₂ are each H.

In certain compounds of Formula (IV), Y is R_{d}. For example, R_{d} is C₁-C₆ alkyl optionally substituted with C₃-C₈ cycloalkyl or halo. For example, R_{d} is C₃-C₈ cycloalkyl optionally substituted with C₁-C₆ alkyl or halo.

The invention also relates to a compound of Formula (IV), wherein at least one of Rₑ, R_{f}, R_{g}, and Rₕ is halo, C₁-C₆ alkoxyl optionally substituted with one or more halo; C₁-C₆ alkylsulfonyl optionally substituted with one or more halo; C₁-C₆ alkyl optionally substituted with one or more substituents selected from CN, halo, C₃-C₈ cycloalkyl, hydroxy, and C₁-C₆ alkoxyl; C₃-C₈ cycloalkyl optionally substituted with one or more C₁-C₆ alkyl or CN; or 4 to 8-membered heterocycloalkyl optionally substituted with one or more substituents selected from CN, halo, hydroxy, C₁-C₆ alkyl and C₁-C₆ alkoxyl. For example, the compound of Formula (IV) has at least one of Rₑ, R_{f}, R_{g}, and Rₕ selected from F; Cl; Br; CF₃; OCF₃; SO₂CF₃; oxetanyl optionally substituted with one or more substituents selected from CN, halo, hydroxy, C₁-C₆ alkyl and C₁-C₆ alkoxyl; C₃-C₈ cycloalkyl optionally substituted with one or more substituents selected from C₁-C₄ alkyl; and C₁-C₄ alkyl optionally substituted with one or more substituents selected from halo, C₃-C₈ cycloalkyl, hydroxy and C₁-C₆ alkoxyl.

For example, the invention relates to DOT1L inhibitor compounds of Formula (IV) where at least one of R_{f} and R_{g} is alkyl, optionally substituted with hydroxyl. For example, the invention relates to compounds where at least one of R_{f} and R_{g} is t-butyl substituted with hydroxyl.

The invention relates to a composition comprising one or more therapeutic agents and i) a compound selected from Tables 1-4; ii) a salt of a compound selected from Tables 1-4; iii) an N-oxide of compound selected from Tables 1-4; or iv) a salt of an N-oxide of compound selected from Tables 1-4. For example, the invention relates to a composition comprising one or more therapeutic agents and a compound selected from Compounds A1-A7, A9-A109, and A111-A140.

In one embodiment, a composition comprises one or more therapeutic agents and Compound A2 (also called “Cpd A2” or “5676”) having the formula:

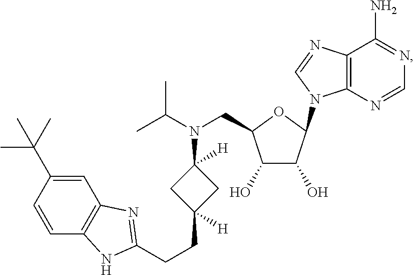 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or pharmaceutically acceptable salts thereof

In one embodiment, a composition comprises one or more therapeutic agents and Compound T (i.e., Compound D16) having the formula:

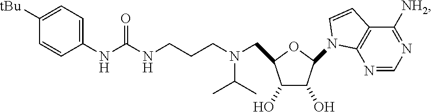 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or pharmaceutically acceptable salts thereof.

Other DOT1L inhibitor compounds suitable for this invention are described in, e.g., WO2012/075381, WO2012/075492, WO2012/082436, and WO2012/75500, the contents of each of which are hereby incorporated by reference in their entireties.

The invention also relates to a pharmaceutical composition of a therapeutically effective amount of any composition described herein and a pharmaceutically acceptable carrier.

The invention also relates to a pharmaceutical composition of a therapeutically effective amount of one or more therapeutic agents and a compound of any of the Formulae disclosed herein and a pharmaceutically acceptable carrier.

The invention also relates to a pharmaceutical composition of a therapeutically effective amount of one or more therapeutic agents and a salt of a compound of any of the Formulae disclosed herein and a pharmaceutically acceptable carrier.

The invention also relates to a pharmaceutical composition of a therapeutically effective amount of one or more therapeutic agents and a hydrate of a compound of any of the Formulae disclosed herein and a pharmaceutically acceptable carrier.

The invention also relates to a pharmaceutical composition of a therapeutically effective amount of one or more therapeutic agents and a compound selected from Tables 1-4 and a pharmaceutically acceptable carrier. The invention also relates to a pharmaceutical composition of a therapeutically effective amount of one or more therapeutic agents and a salt of a compound selected from Tables 1-4 and a pharmaceutically acceptable carrier. The invention also relates to a pharmaceutical composition of a therapeutically effective amount of one or more therapeutic agents and an N-oxide of a compound selected from Tables 1-4 and a pharmaceutically acceptable carrier. The invention also relates to a pharmaceutical composition of a therapeutically effective amount of one or more therapeutic agents and an N-oxide of salt of a compound selected from Tables 1-4 and a pharmaceutically acceptable carrier. The invention also relates to a pharmaceutical composition of a therapeutically effective amount of one or more therapeutic agents and a hydrate of a compound selected from Tables 1-4 and a pharmaceutically acceptable carrier.

In the formulae presented herein, the variables can be selected from the respective groups of chemical moieties later defined in the detailed description.

In addition, the invention provides methods of synthesizing the foregoing compounds. Following synthesis, a therapeutically effective amount of one or more of the compounds can be formulated with a pharmaceutically acceptable carrier for administration to a mammal, particularly humans, for use in modulating an epigenetic enzyme. In certain embodiments, the compounds of the present invention are useful for treating, preventing, or reducing the risk of cancer or for the manufacture of a medicament for treating, preventing, or reducing the risk of cancer. Accordingly, the compounds or the formulations can be administered, for example, via oral, parenteral, otic, ophthalmic, nasal, or topical routes, to provide an effective amount of the compound to the mammal.

Representative compounds of the present invention include compounds listed in Tables 1-4.

TABLE 1

| | | | Data |
|---|---|---|---|
| | | | (MS |
| Cmpd | | | or |
| No. | Structure | Chemical Name | NMR) |
| A1 | 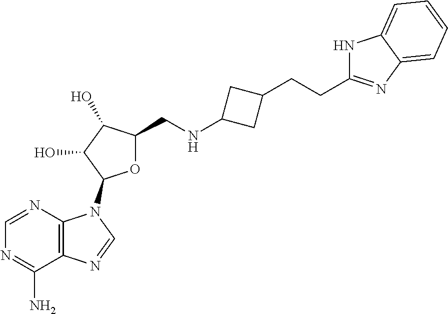 [SMILES: Nc1ncnc2c1ncn2[C@@H]1O[C@H](CNC2CC(CCc3nc4ccccc4[nH]3)C2)[C@@H](O)[C@H]1O] | (2R,3S,4R,5R)-2- (((3-(2-(1H- benzo[d]imidazol-2- yl)ethyl)cyclobutyl) amino)methyl)-5-(6- amino-9H-purin-9- yl)tetrahydrofuran- 3,4-diol | |
| A2 | 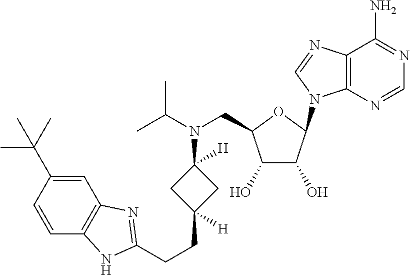 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1] | (2R,3R,4S,5R)-2-(6- amino-9H-purin-9- yl)-5-((((1r,3S)-3-(2- (5-(tert-butyl)-1H- benzo[d]imidazol-2- yl)ethyl)cyclobutyl) (isopropyl)amino)methyl) tetrahydrofuran- 3,4-diol | 563.4 (M + H)⁺ |
| A3 | 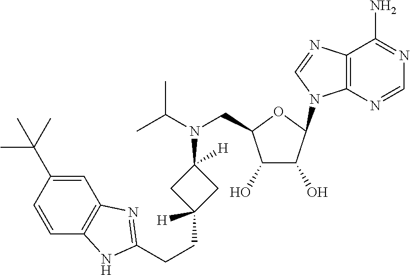 [SMILES: CC(C)N(C[C@H]1O[C@@H](n2cnc3c(N)ncnc32)[C@H](O)[C@@H]1O)[C@H]1C[C@H](CCc2nc3cc(C(C)(C)C)ccc3[nH]2)C1] | (2R,3R,4S,5R)-2-(6- amino-9H-purin-9-yl)- yl)-5-((((1s,3R)-3-(2- (5-(tert-butyl)-1H- benzo[d]imidazol-2- yl)ethyl)cyclobutyl) (isopropyl)amino)methyl) tetrahydrofuran-3,4-diol | 563.5 (M + H)⁺ |
| A4 | 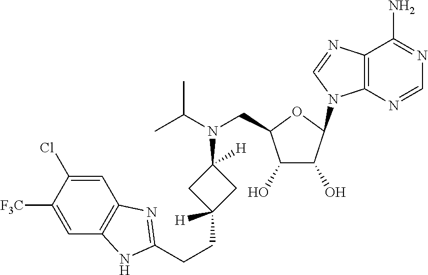 [SMILES: CC(C)N(C[C@H]1O[C@@H](n2cnc3c(N)ncnc32)[C@H](O)[C@@H]1O)[C@H]1C[C@H](CCc2nc3cc(Cl)c(C(F)(F)F)cc3[nH]2)C1] | (2R,3R,4S,5R)-2-(6- amino-9H-purin-9- yl)-5-((((1s,3R)-3-2- (5-chloro-6- (trifluoromethyl)-1H- benzo[d]imidazol-2- yl)ethyl)cyclobutyl) isopropyl)amino)methyl) tetrahydrofuran-3,4-diol | 609.2 (M + H)⁺ |
| A5 | 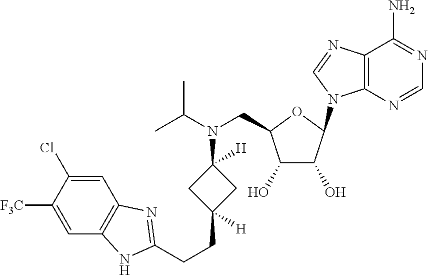 [SMILES: CC(C)N(C[C@H]1O[C@@H](n2cnc3c(N)ncnc32)[C@H](O)[C@@H]1O)[C@H]1C[C@@H](CCc2nc3cc(Cl)c(C(F)(F)F)cc3[nH]2)C1] | (2R,3R.4S,5R)-2-(6- amino-9H-purin-9- yl)-5-((((1r,3S)-3-(2- (5-chloro-6- (trifluoromethyl)-1H- benzo[d]imidazol-2- yl)ethyl(cyclobutyl) (isopropyl)amino)methyl) tetrahydrofuran-3,4-diol | 609.2 (M + H)⁺ |
| A6 | 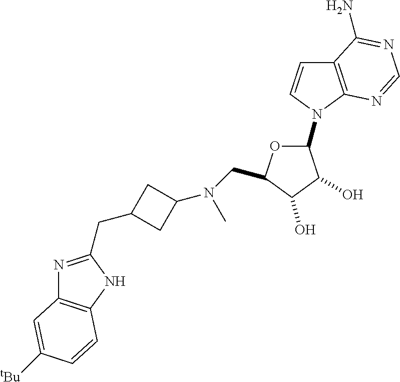 | (2R,3R,4S,5R)-2-(4- amino-7H- pyrrolo[2,3-d] pyrimidin-7-yl)-5- (((3-((5-(tert-butyl)- 1H- benzo[d]imidazol-2- yl)methyl)cyclobutyl) (methyl)amino)methyl) tetrahydrofuran-3,4-diol | 520.4 (M + H)⁺ |
| A7 | 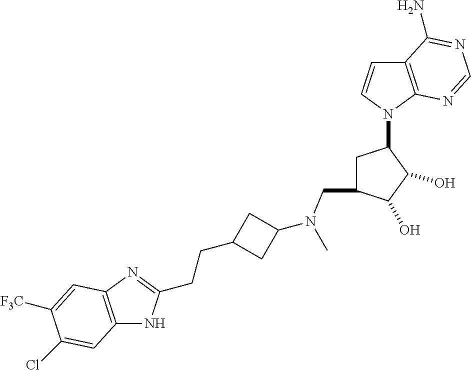 | (1R,2S,3R,5R)-3-(4- amino-7H- pyrrolo[2,3-d] pyrimidin-7-yl)-5- (((3-(2-(6-chloro-5- (trifluoromethyl)-1H- benzo[d]imidazol-2- yl)ethyl)cyclobutyl) (methyl)amino)methyl) cyclopentane-1,2-diol | 579.7 (M + H)⁺ |
| A8 | 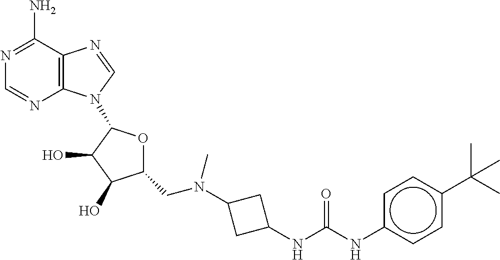 | 1-(3- ((((2R,3S,4R,5R)-5- (6-amino-9H-purin-9- yl)-3,4- dihydroxytetrahydro- furan-2- yl)methyl)(methyl) amino)cyclobutyl)-3- (4-tert- butylphenyl)urea | 525.5 (M + H)⁺ |
| A9 | 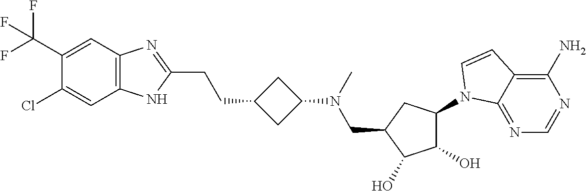 [SMILES: CN(C[C@H]1C[C@@H](n2ccc3c(N)ncnc32)[C@H](O)[C@@H]1O)[C@H]1C[C@@H](CCc2nc3cc(C(F)(F)F)c(Cl)cc3[nH]2)C1] | (1R,2S,3R,5R)-3-(4- amino-7H- pyrrolo[2,3-d] pyrimidin-7-yl)-5- ((((1R,3S)-3-(2-(6- chloro-5- (trifluoromethyl)-1H- benzo[d]imidazol-2- yl(ethyl)cyclobutyl) (methyl)amino)methyl) cyclopentane-1,2-diol | 578.3 (M + H)⁺ |
| A10 | 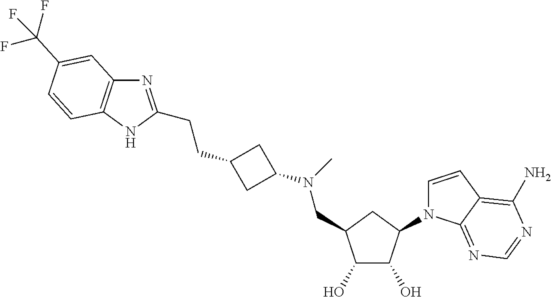 [SMILES: CN(C[C@H]1C[C@@H](n2ccc3c(N)ncnc32)[C@H](O)[C@@H]1O)[C@H]1C[C@@H](CCc2nc3cc(C(F)(F)F)ccc3[nH]2)C1] | (1R,2S,3R,5R)-3-(4- amino-7H- pyrrolo[2,3-d] pyrimidin-7-yl)-5- ((methyl((1R,3S)-3- (2-(5- (trifluoromethyl)-1H- benzo[d]imidazol-2- yl)ethyl(cyclobutyl) amino)methyl) cyclopentane-1,2-diol | 544.3 (M + H)⁺ |
| A11 | 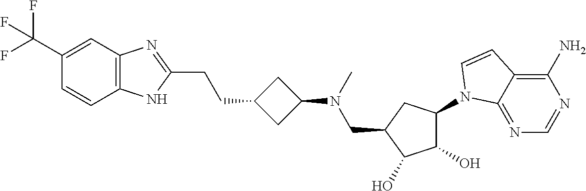 [SMILES: CN(C[C@H]1C[C@@H](n2ccc3c(N)ncnc32)[C@H](O)[C@@H]1O)[C@H]1C[C@H](CCc2nc3cc(C(F)(F)F)ccc3[nH]2)C1] | (1R,2S,3R,5R)-3-(4- amino-7H- pyrrolo[2,3-d] pyrimidin-7-yl)-5- ((methyl((1s,3R)-3- (2-(5- (trifluoromethyl)-1H- benzo[d]imidazol-2- yl)ethyl)cyclobutyl) amino)methyl) cyclopentane-1,2-diol | 544.3 (M + H)⁺ |
| A12 | 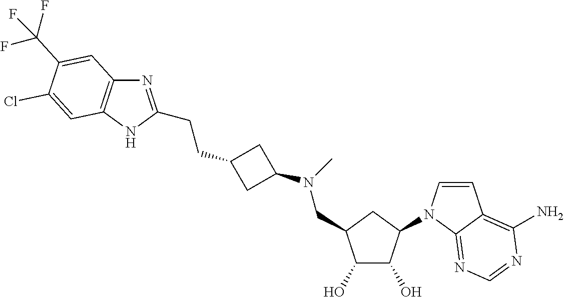 [SMILES: CN(C[C@H]1C[C@@H](n2ccc3c(N)ncnc32)[C@H](O)[C@@H]1O)[C@H]1C[C@H](CCc2nc3cc(C(F)(F)F)c(Cl)cc3[nH]2)C1] | (1R,2S,3R,5R)-3-(4- amino-7H- pyrrolo[2,3-d] pyrimidin-7-yl)-5- ((((1s,3R)-3-(2-(6- chloro-5- (trifluoromethyl)-1H- benzo[d]imidazol-2- yl)ethyl)cyclobutyl) (methyl)amino)methyl) cyclopentane-1,2-diol | 578.3 (M + H)⁺ |
| A13 | 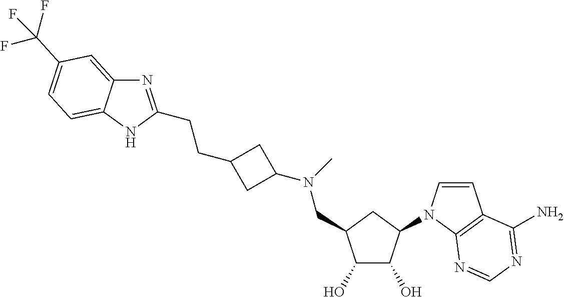 [SMILES: CN(C[C@H]1C[C@@H](n2ccc3c(N)ncnc32)[C@H](O)[C@@H]1O)C1CC(CCc2nc3cc(C(F)(F)F)ccc3[nH]2)C1] | (1R,2S,3R,5R)-3-(4- amino-7H- pyrrolo[2,3-d] pyrimidin-7-yl)-5- ((methyl(3-(2-(5- (trifluoromethyl)-1H- benzo[d]imidazol-2- yl)ethyl)cyclobutyl) amino)methyl) cyclopentane-1,2-diol | 544.5 (M + H)⁺ |
| A14 | 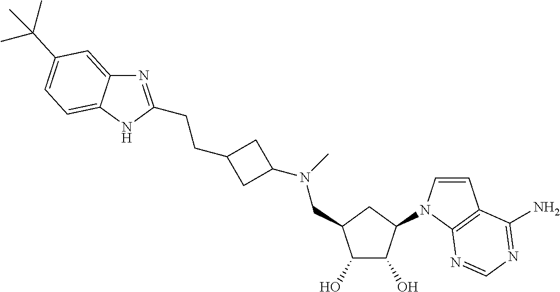 [SMILES: CN(C[C@H]1C[C@@H](n2ccc3c(N)ncnc32)[C@H](O)[C@@H]1O)C1CC(CCc2nc3cc(C(C)(C)C)ccc3[nH]2)C1] | (1R,2S,3R,5R)-3-(4- amino-7H- pyrrolo[2,3-d] pyrimidin-7-yl)-5- (((3-(2-(5-(tert- butyl)-1H- benzo[d]imidazol-2- yl(ethyl)cyclobutyl) (methyl)amino)methyl) cyclopentane-1,2-diol | 532.3 (M + H)⁺ |
| A15 | 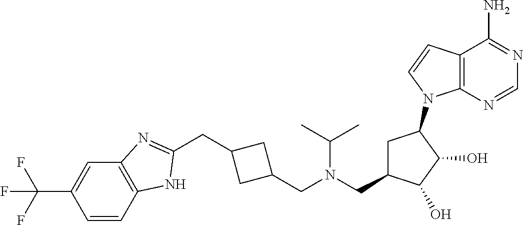 [SMILES: CC(C)N(CC1CC(Cc2nc3cc(C(F)(F)F)ccc3[nH]2)C1)C[C@H]1C[C@@H](n2ccc3c(N)ncnc32)[C@H](O)[C@@H]1O] | (1R,2S,3R,5R)-3-(4- amino-7H- pyrrolo[2,3-d] pyrimidin-7-yl)-5- ((isopropyl((3-((5- (trifluoromethyl)-1H- benzo[d]imidazol-2- yl)methyl)cyclobutyl) methyl)amino)methyl) cyclopentane-1,2-diol | 572.4 (M + H)⁺ |
| A16 | 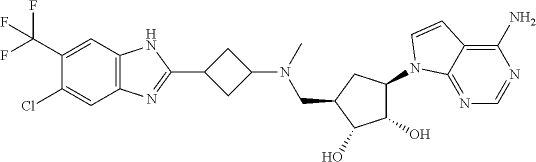 [SMILES: CN(C[C@H]1C[C@@H](n2ccc3c(N)ncnc32)[C@H](O)[C@@H]1O)C1CC(c2nc3cc(Cl)c(C(F)(F)F)cc3[nH]2)C1] | (1R,2S,3R,5R)-3-(4- amino-7H- pyrrolo[2,3-d] pyrimidin-7-yl)-5- (((3-(5-chloro-6- (trifluoromethyl)-1H- benzo[d]imidazol-2- yl)cyclobutyl)(methyl) amino)methyl) cyclopentane-1,2-diol | 550.3 (M + H)⁺ |
| A17 | 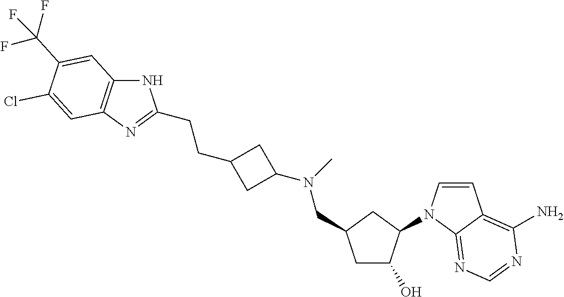 [SMILES: CN(C[C@@H]1C[C@@H](O)[C@H](n2ccc3c(N)ncnc32)C1)C1CC(CCc2nc3cc(Cl)c(C(F)(F)F)cc3[nH]2)C1] | (1R,2R,4S)-2-(4- amino-7H- pyrrolo[2,3-d] pyrimidin-7-yl)-4- (((3-(2-(5-chloro-6- (trifluoromethyl)-1H- benzo[d]imidazol-2- yl)ethyl)cyclobutyl) (methyl)amino)methyl) cyclopentanol | 562.3 (M + H)⁺ |
| A18 | 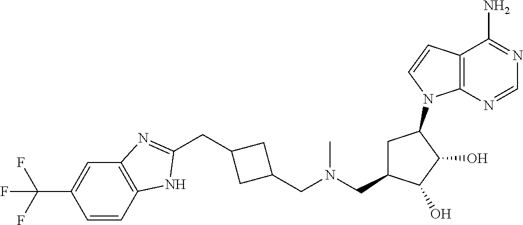 [SMILES: CN(CC1CC(Cc2nc3cc(C(F)(F)F)ccc3[nH]2)C1)C[C@H]1C[C@@H](n2ccc3c(N)ncnc32)[C@H](O)[C@@H]1O] | (1R,2S,3R,5R)-3-(4- amino-7H- pyrrolo[2,3-d] pyrimidin-7-yl)-5- ((methyl((3-((5- (trifluoromethyl)-1H- benzo[d]imidazol-2- yl)methyl)cyclobutyl) methyl)amino)methyl) cyclopentane-1,2-diol | 544 (M + H)⁺ |
| A19 | 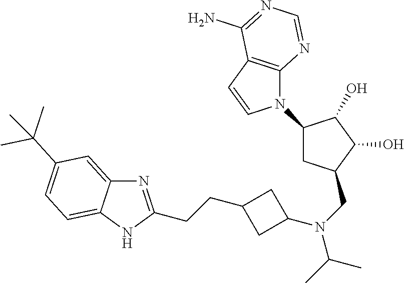 [SMILES: CC(C)N(C[C@H]1C[C@@H](n2ccc3c(N)ncnc32)[C@H](O)[C@@H]1O)C1CC(CCc2nc3cc(C(C)(C)C)ccc3[nH]2)C1] | (1R,2S,3R,5R)-3-(4- amino-7H- pyrrolo[2,3-d] pyrimidin-7-yl)-5- (((3-(2-(5-(tert- butyl)-1H- benzo[d]imidazol-2- yl)ethyl)cyclobutyl) (isopropyl)amino)methyl) cyclopentane-1,2-diol | NMR data |
| A20 | 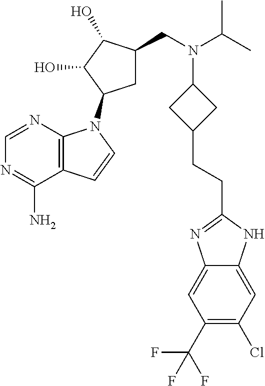 [SMILES: CC(C)N(C[C@H]1C[C@@H](n2ccc3c(N)ncnc32)[C@H](O)[C@@H]1O)C1CC(CCc2nc3cc(C(F)(F)F)c(Cl)cc3[nH]2)C1] | (1R,2S,3R,5R)-3-(4- amino-7H- pyrrolo[2,3-d] pyrimidin-7-yl)-5- (((3-(2-(6-chloro-5- (trifluoromethyl)-1H- benzo[d]imidazol-2- yl)ethyl)cyclobutyl) (isopropyl)amino)methyl) cyclopentane-1,2-diol | NMR data |
| A21 | 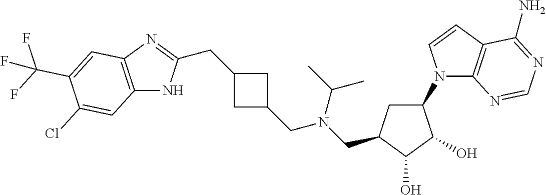 [SMILES: CC(C)N(CC1CC(Cc2nc3cc(C(F)(F)F)c(Cl)cc3[nH]2)C1)C[C@H]1C[C@@H](n2ccc3c(N)ncnc32)[C@H](O)[C@@H]1O] | (1R,2S,3R,5R)-3-(4- amino-7H- pyrrolo[2,3-d] pyrimidin-7-yl)-5- ((((3-((6-chloro-5- (trifluoromethyl)-1H- benzo[d]imidazol-2- yl)methyl)cyclobutyl) methyl)(isopropyl)amino) methyl)cyclopentane- 1,2-diol | 606.3 (M + H)⁺ |
| A22 | 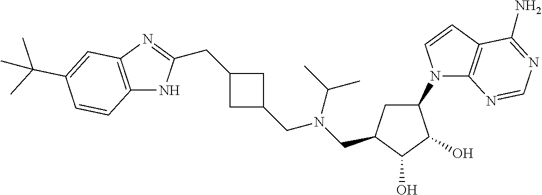 [SMILES: CC(C)N(CC1CC(Cc2nc3cc(C(C)(C)C)ccc3[nH]2)C1)C[C@H]1C[C@@H](n2ccc3c(N)ncnc32)[C@H](O)[C@@H]1O] | (1R,2S,3R,5R)-3-(4- amino-7H- pyrrolo[2,3-d] pyrimidin-7-yl)-5- ((((3-((5-(tert-butyl)- 1H- benzo[d]imidazol-2- yl)methyl)cyclobutyl) methyl(isopropyl)amino) methyl)cyclopentane- 1,2-diol | 560.4 (M +H)⁺ |
| A23 | 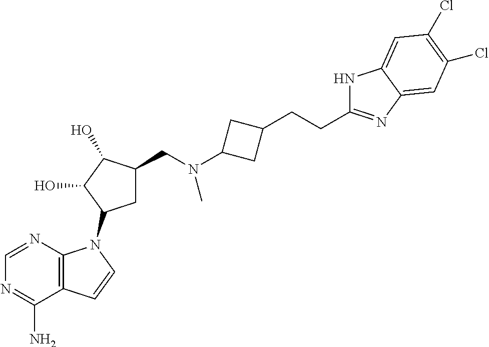 | (1R,2S,3R,5R)-3-(4- amino-7H- pyrrolo[2,3-d] pyrimidin-7-yl)-5- (((3-(2-(5,6-dichloro- 1H- benzo[d]imidazol-2- yl)ethyl)cyclobutyl) (methyl(amino)methyl) cyclopentane-1,2-diol | NMR data |
| A24 | 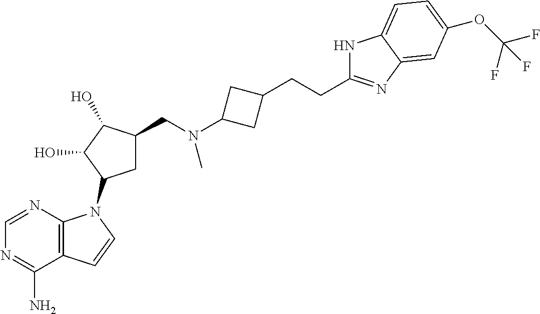 | (1R,2S,3R,5R)-3-(4- amino-7H- pyrrolo[2,3-d] pyrimidin-7-yl)-5- ((methyl(3-(2-(5- (trifluoromethoxy)- 1H- benzo[d]imidazol-2- yl)ethyl)cyclobutyl) amino)methyl) cyclopentane-1,2-diol | 558.2 (M − H)⁺ |
| A25 | 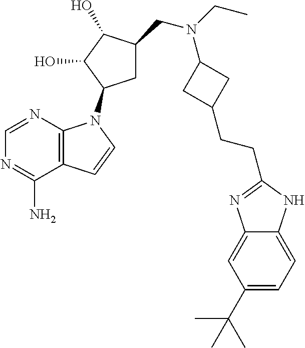 [SMILES: CCN(C[C@H]1C[C@@H](n2ccc3c(N)ncnc32)[C@H](O)[C@@H]1O)C1CC(CCc2nc3cc(C(C)(C)C)ccc3[nH]2)C1] | (1R,2S,3R,5R)-3-(4- amino-7H- pyrrolo[2,3-d] pyrimidin-7-yl)-5- (((3-(2-(5-(tert- butyl)-1H- benzo[d]imidazol-2- yl)ethyl)cyclobutyl) (ethyl)amino)methyl) cyclopentane-1,2-diol | 546.3 (M + H)⁺ |
| A26 | 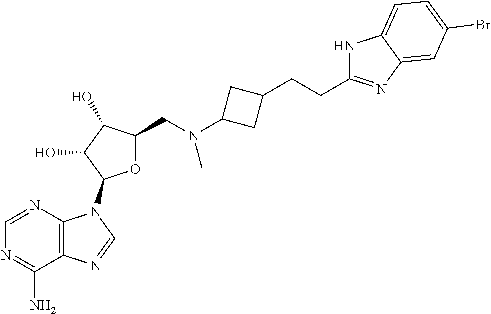 | (1R,2S,3R,5R)-3-(4- amino-7H- pyrrolo[2,3-d] pyrimidin-7-yl)-5- (((3-(2-(5-bromo-1H- benzo[d]imidazol-2- yl)ethyl)cyclobutyl) methyl)amino)methyl) cyclopentane-1,2-diol | 554.1 (M + H)⁺ |
| A27 | 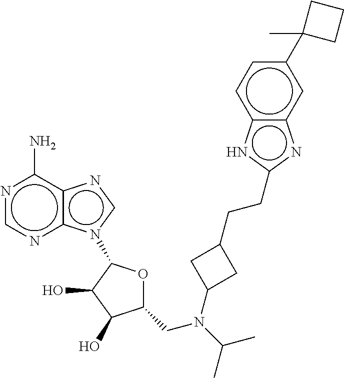 | (2R,3R,4S,5R)-2-(6- amino-9H-purin-9- yl)-5-((isopropyl(3- (2-(5-(1- methylcyclobutyl)- 1H- benzo[d]imidazol-2- yl)ethyl)cyclobutyl) amino)methyl) tetrahydrofuran-3,4-diol | 575.5 (M + H)⁺ |
| A28 | 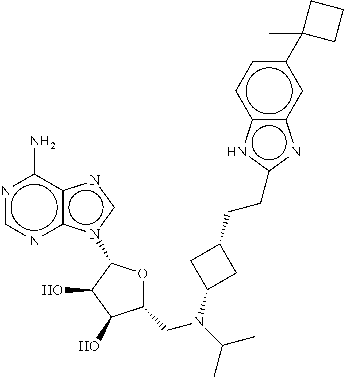 | (2R,3R,4S,5R)-2-(6- amino-9H-purin-9- yl)-5- ((isopropyl((1r,3S)-3- (2-(5-(1- methylcyclobutyl)- 1H- benzo[d]imidazol-2- yl(ethyl)cyclobutyl) amino)methyl) tetrahydrofuran-3.4-diol | 575.5 (M + H)⁺ |
| A29 | 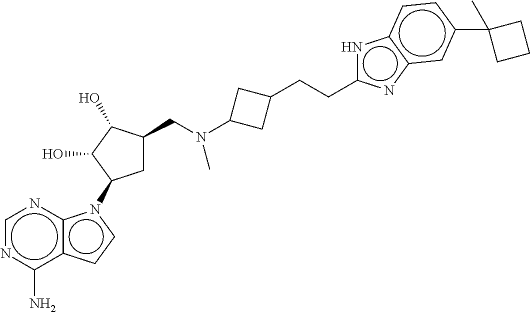 | (1R,2S,3R,5R)-3-(4- amino-7H- pyrrolo[2,3-d] pyrimidin-7-yl)-5- ((methyl(3-(2-(5-(1- methylcyclobutyl)- 1H- benzo[d]imidazol-2- yl)ethyl)cyclobutyl) amino)methyl) cyclopentane-1,2-diol | 544.4 (M + H)⁺ |
| A30 | 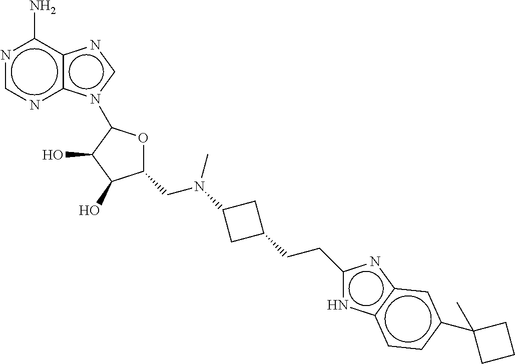 | (2R,3R,4S,5R)-2-(6- amino-9H-purin-9- yl)-5- ((methyl((1r,3S)-3- (2-(5-(1- methylcyclobutyl)- 1H- benzo[d]imidazol-2- yl)ethyl)cyclobutyl) amino)methyl) tetrahydrofuran-3,4-diol | 547.6 (M + H)⁺ |
| A31 | 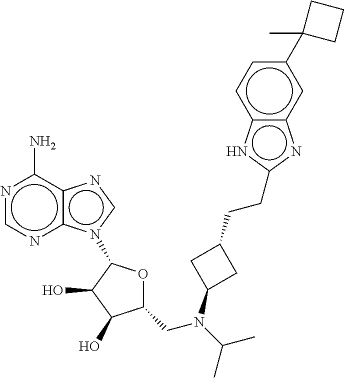 | (2R,3R,4S,5R)-2-(6- amino-9H-purin-9- yl)-5- ((isopropyl((1s,3R)- 3-(2-(5-(1- methylcyclobutyl)- 1H- benzo[d]imidazol-2- yl)ethyl)cyclobutyl) amino)methyl) tetrahydrofuran-3,4-diol | 575.6 (M + H)⁺ |
| A32 | 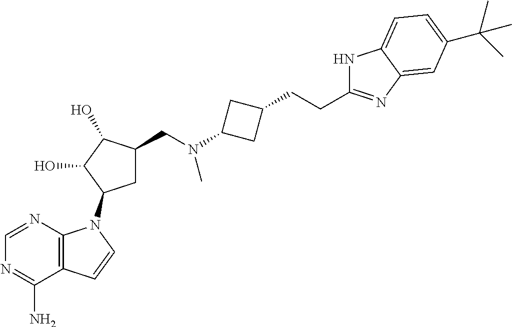 [SMILES: CN(C[C@H]1C[C@@H](n2ccc3c(N)ncnc32)[C@H](O)[C@@H]1O)[C@H]1C[C@@H](CCc2nc3cc(C(C)(C)C)ccc3[nH]2)C1] | (1R,2S,3R.5R)-3-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (methyl)amino)methyl) cyclopentane-1,2-diol | 532.4 (M + H)⁺ |
| A33 | 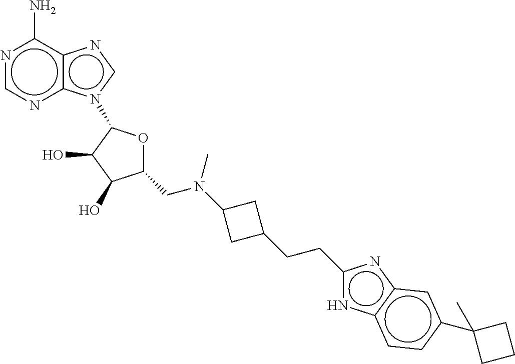 | (2R,3R,4S,5R)-2-(6- amino-9H-purin-9- yl)-5-((methyl(3-(2- (5-(1- methylcyclobutyl)- 1H- benzo[d]imidazol-2- yl)ethyl)cyclobutyl) amino)methyl) tetrahydrofuran-3,4-diol | 547.3 (M + H)⁺ |
| A34 | 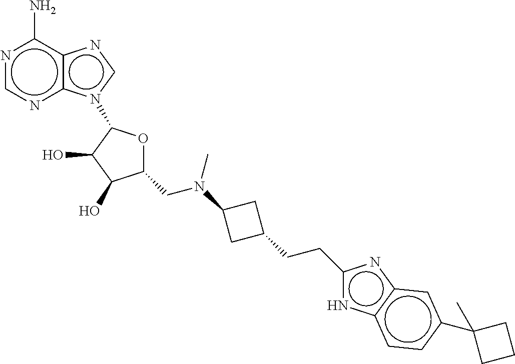 | (2R,3R,4S,5R)-2-(6- amino-9H-purin-9- yl)-5- ((methyl((1s,3R)-3- (2-(5-(l- methylcyclobutyl)- 1H- benzo[d]imidazol-2- yl)ethyl)cyclobutyl) amino)methyl) tetrahydrofuran-3,4-diol | 547.5 (M + H)⁺ |
| A35 | 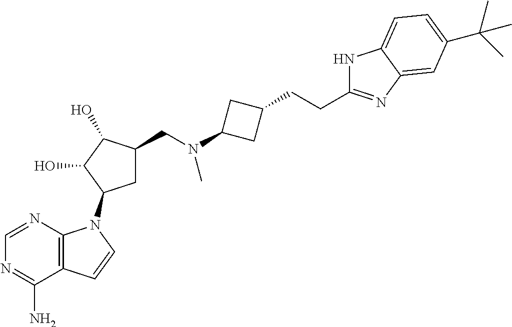 [SMILES: CN(C[C@H]1C[C@@H](n2ccc3c(N)ncnc32)[C@H](O)[C@@H]1O)[C@H]1C[C@H](CCc2nc3cc(C(C)(C)C)ccc3[nH]2)C1] | (1R,2S,3R,5R)-3-(4- amino-7H- pyrrolo[2,3-d] pyrimidin-7-yl)-5- ((((1s,3R)-3-(2-(5- (tert-butyl)-1H- benzo[d]imidazol-2- yl)ethyl)cyclobutyl) (methyl)amino)methyl) cyclopentane-1,2-diol | NMR data |
| A36 | 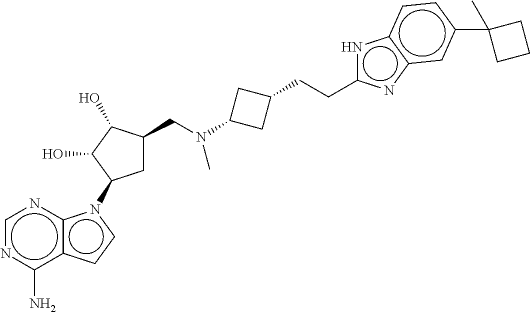 | (1R,2S,3R,5R)-3-(4- amino-7H- pyrrolo[2,3-d] pyrimidin-7-yl)-5- ((methyl((1r,3S)-3- (2-(5-(1- methylcyclobutyl)- 1H- benzo[d]imidazol-2- yl(ethyl(cyclobutyl) amino)methyl) cyclopentane-1,2-diol | 544.4 (M + H)⁺ |
| A37 | 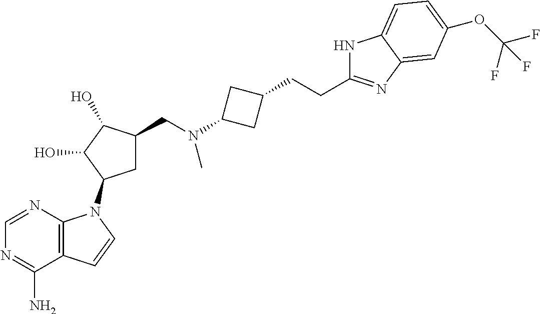 [SMILES: CN(C[C@H]1C[C@@H](n2ccc3c(N)ncnc32)[C@H](O)[C@@H]1O)[C@H]1C[C@@H](CCc2nc3cc(OC(F)(F)F)ccc3[nH]2)C1] | (1R,2S,3R,5R)-3-(4- amino-7H- pyrrolo[2,3-d] pyrimidin-7-yl)-5- ((methyl((1r,3S)-3- (2-(5- (trifluoromethoxy)- 1H- benzo[d]imidazol-2- yl)ethyl)cyclobutyl) amino(methyl) cyclopentane-1,2-diol | 558.3 (M − H)⁻ |
| A38 | 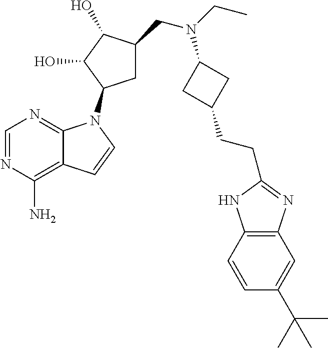 [SMILES: CCN(C[C@H]1C[C@@H](n2ccc3c(N)ncnc32)[C@H](O)[C@@H]1O)[C@H]1C[C@@H](CCc2nc3cc(C(C)(C)C)ccc3[nH]2)C1] | (1R,2S,3R,5R)-3-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) ethyl)amino)methyl) cyclopentane-1,2-diol | 546.3 (M + H)⁺ |
| A39 | 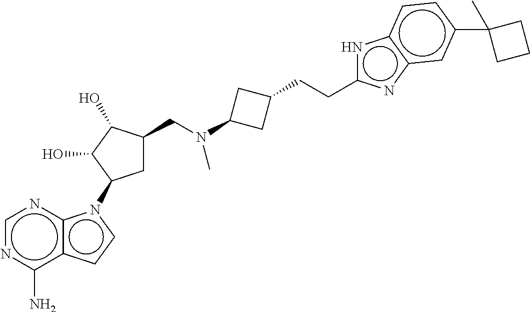 | (1R,2S,3R,5R)-3-(4- amino-7H- pyrrolo[2,3-d] pyrimidin-7-yl)-5- ((methyl((1s,3R)-3- (2-(5-(1- methylcyclobutyl)- 1H- benzo[d]imidazol-2- yl)ethyl)cyclobutyl) amino)methyl) cyclopentane-1,2-diol | 544.3 (M + H)⁺ |
| A40 | 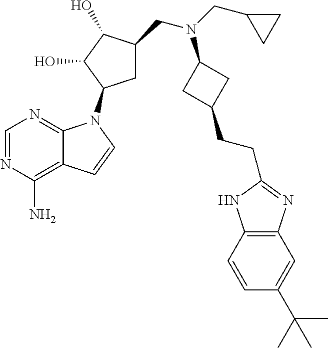 [SMILES: CC(C)(C)c1ccc2[nH]c(CC[C@H]3C[C@@H](N(CC4CC4)C[C@H]4C[C@@H](n5ccc6c(N)ncnc65)[C@H](O)[C@@H]4O)C3)nc2c1] | (1R,2S,3R,5R)-3-(4- amino-7H- pyrrolo[2,3-d] pyrimidin-7-yl)-5- ((((1r,3S)-3-(2-(5- (tert-butyl)-1H- benzo[d]imidazol-2- yl (ethyl)cyclobutyl) cyclopropylmethyl) amino)methyl) cyclopentane-1,2-diol | NMR data |
| A41 | 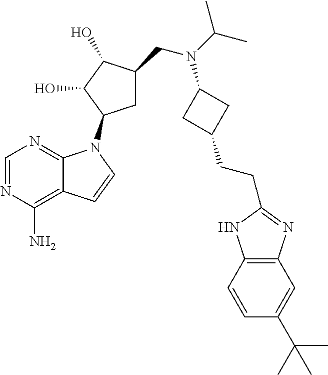 [SMILES: CC(C)N(C[C@H]1C[C@@H](n2ccc3c(N)ncnc32)[C@H](O)[C@@H]1O)[C@H]1C[C@@H](CCc2nc3cc(C(C)(C)C)ccc3[nH]2)C1] | (1R,2S,3R,5R)-3-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (isopropyl)amino)methyl) (cyclopentane-1,2-diol | NMR data |
| A42 | 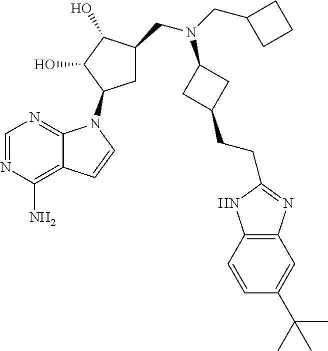 [SMILES: CC(C)(C)c1ccc2[nH]c(CC[C@H]3C[C@@H](N(CC4CCC4)C[C@H]4C[C@@H](n5ccc6c(N)ncnc65)[C@H](O)[C@@H]4O)C3)nc2c1] | (1R,2S,3R,5R)-3-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (cyclobutylmethyl) amino)methyl) cyclopentane-1,2-diol | 586.3 (M + H)⁺ |
| A43 | 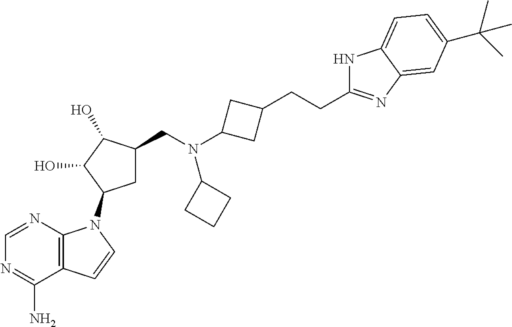 [SMILES: CC(C)(C)c1ccc2[nH]c(CCC3CC(N(C[C@H]4C[C@@H](n5ccc6c(N)ncnc65)[C@H](O)[C@@H]4O)C4CCC4)C3)nc2c1] | (1R,2S,3R,5R)-3-(4- amino-7H- pyrrolo[2,3-d] pyrimidin-7-yl)-5- ((((3-2-(5-(tert- butyl)-1H- benzo[d]imidazol-2- yl(ethyl)cyclobutyl) cyclobutyl)amino) methyl) cyclopentane-1,2-diol | 572.2 (M + H)⁺ |
| A44 | 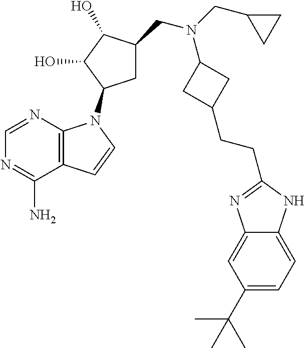 [SMILES: CC(C)(C)c1ccc2[nH]c(CCC3CC(N(CC4CC4)C[C@H]4C[C@@H](n5ccc6c(N)ncnc65)[C@H](O)[C@@H]4O)C3)nc2c1] | (1R,2S,3R,5R)-3-(4- amino-7H- pyrrolo[2,3-d] pyrimidin-7-yl)-5- (((3-(2-(5-(tert- butyl)-1H- benzo[d]imidazol-2- yl)ethyl)cyclobutyl) (cyclopropylmethyl) amino)methyl) cyclopentane-1,2-diol | 572.6 (M + H)⁺ |
| A45 | 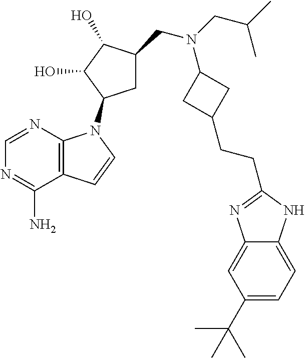 [SMILES: CC(C)CN(C[C@H]1C[C@@H](n2ccc3c(N)ncnc32)[C@H](O)[C@@H]1O)C1CC(CCc2nc3cc(C(C)(C)C)ccc3[nH]2)C1] | (1R,2S,3R,5R)-3-(4- amino-7H- pyrrolo[2,3-d] pyrimidin-7-yl)-5- (((3-(2-(5-(tert- butyl)-1H- benzo[d]imidazol-2- yl)ethyl)cyclobutyl) (isobutyl)amino)methyl) cyclopentane-1,2-diol | 574.6 (M + H)⁺ |
| A46 | 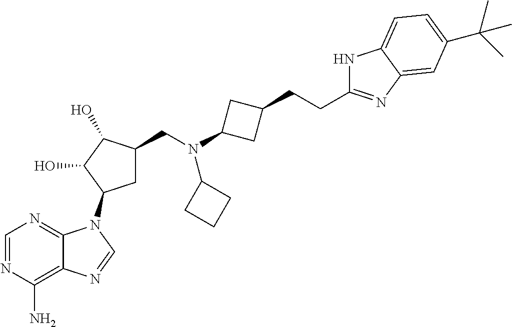 [SMILES: CC(C)(C)c1ccc2[nH]c(CC[C@H]3C[C@@H](N(C[C@H]4C[C@@H](n5cnc6c(N)ncnc65)[C@H](O)[C@@H]4O)C4CCC4)C3)nc2c1] | (1R,2S,3R,5R)-3-(4- amino-7H- pyrrolo[2,3-d] pyrimidin-7-yl)-5- ((((1r,3S)-3-(2-5- (tert-butyl)-1H- benzo[d]imidazol-2 - yl)ethyl)cyclobutyl) (cyclobutyl)amino)methyl) cyclopentane-1,2- diol | 572.6 (M + H)⁺ |
| A47 | 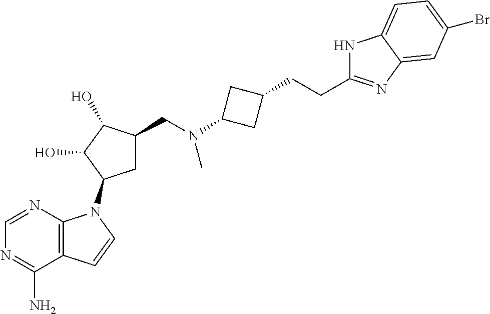 [SMILES: CN(C[C@H]1C[C@@H](n2ccc3c(N)ncnc32)[C@H](O)[C@@H]1O)[C@H]1C[C@@H](CCc2nc3cc(Br)ccc3[nH]2)C1] | (1R,2S,3R,5R)-3-(4- amino-7H- pyrrolo[2,3-d] pyrimidin-7-yl)-5- (((1r,3S)-3-(2-(5- bromo-1H- benzo[d]imidazol-2- yl)ethyl)cyclobutyl) methyl(amino)methyl) cyclopentane-1,2-diol | 556.0 (M + H)⁺ |
| A48 | 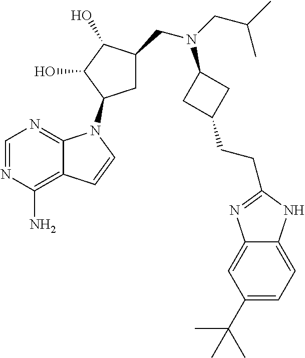 [SMILES: CC(C)CN(C[C@H]1C[C@@H](n2ccc3c(N)ncnc32)[C@H](O)[C@@H]1O)[C@H]1C[C@H](CCc2nc3cc(C(C)(C)C)ccc3[nH]2)C1] | (1R,2S,3R,5R)-3-(4- amino-7H- pyrrolo[2,3-d] pyrimidin-7-yl)-5- ((((1s,3R)-3-(2-(5- (tert-butyl)-1H- benzo[d)imidazol-2- yl)ethyl)cyclobutyl) (isobutyl)amino)methyl) cyclopentane-1,2-diol | 572.3 (M − H)⁻ |
| A49 | 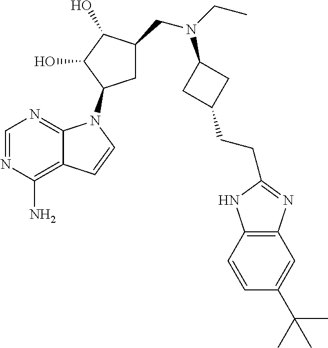 [SMILES: CCN(C[C@H]1C[C@@H](n2ccc3c(N)ncnc32)[C@H](O)[C@@H]1O)[C@H]1C[C@H](CCc2nc3cc(C(C)(C)C)ccc3[nH]2)C1] | (1R,2S,3R,5R)-3-(4- amino-7H- pyrrolo[2,3-d] pyrimidin-7-yl)-5- ((((1s,3R)-3-(2-(5- (tert-butyl)-1H- benzo[d]imidazol-2- yl)ethyl)cyclobutyl) (ethyl(amino)methyl) cyclopentane-1,2-diol | 546.3 (M + H)⁺ |
| A50 | 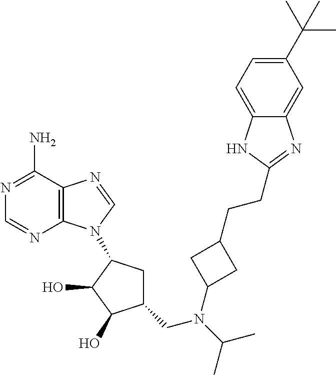 [SMILES: CC(C)N(C[C@H]1C[C@@H](n2cnc3c(N)ncnc32)[C@H](O)[C@@H]1O)C1CC(CCc2nc3cc(C(C)(C)C)ccc3[nH]2)C1] | (1R,2S,3R,5R)-3-(6- amino-9H-purin-9- yl)-5-(((3-(2-(5-(tert- butyl)-1H- benzo[d]imidazol-2- yl)ethyl)cyclobutyl) (isopropyl)amino)methyl) cyclopentane-1,2-diol | 561.4 (M + H)⁺ |
| A51 | 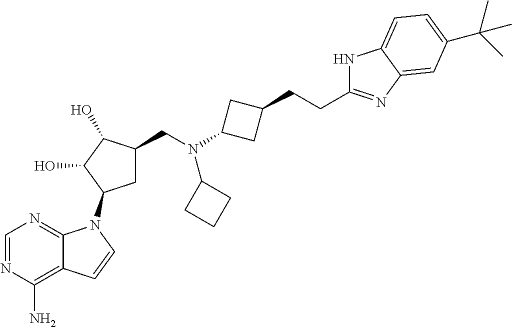 [SMILES: CC(C)(C)c1ccc2[nH]c(CC[C@H]3C[C@H](N(C[C@H]4C[C@@H](n5ccc6c(N)ncnc65)[C@H](O)[C@@H]4O)C4CCC4)C3)nc2c1] | (1R,2S,3R,5R)-3-(4- amino-7H- pyrrolo[2,3-d] pyrimidin-7-yl)-5- ((((1s,3R)-3-(2-(5- (tert-butyl)-1H- benzo[d]imidazol-2- yl)ethyl)cyclobutyl) (cyclobutyl)amino) methyl)cyclopentane- 1,2-diol | 572.7 (M + H)⁺ |
| A52 | 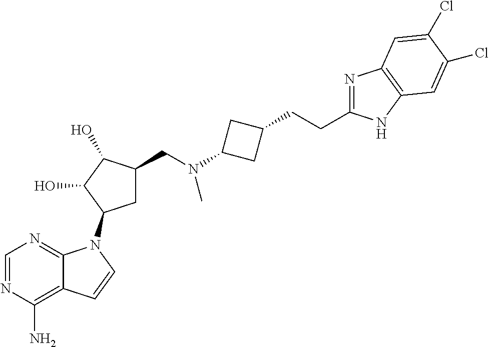 [SMILES: CN(C[C@H]1C[C@@H](n2ccc3c(N)ncnc32)[C@H](O)[C@@H]1O)[C@H]1C[C@@H](CCc2nc3cc(Cl)c(Cl)cc3[nH]2)C1] | (1R,2S,3R,5R)-3-(4- amino-7H- pyrrolo[2,3-d] pyrimidin-7-yl)-5- ((((1r,3S)-3-(2-(5,6- dichloro-1H- benzo[d]imidazol-2- yl)ethyl)cyclobutyl) (methyl)amino)rnethyl) cyclopentane-1,2-diol | NMR data |
| A53 | 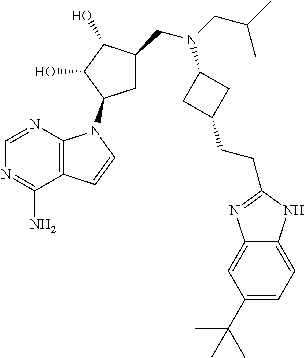 [SMILES: CC(C)CN(C[C@H]1C[C@@H](n2ccc3c(N)ncnc32)[C@H](O)[C@@H]1O)[C@H]1C[C@@H](CCc2nc3cc(C(C)(C)C)ccc3[nH]2)C1] | (1R,2S,3R,5R)-3-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (isobutyl)amino)methyl) cyclopentane-1,2-diol | 572.3 (M − H)⁻ |
| A54 | 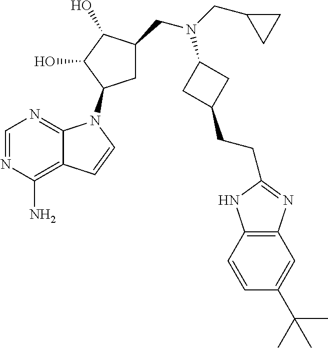 [SMILES: CC(C)(C)c1ccc2[nH]c(CC[C@H]3C[C@H](N(CC4CC4)C[C@H]4C[C@@H](n5ccc6c(N)ncnc65)[C@H](O)[C@@H]4O)C3)nc2c1] | (1R,2S,3R,5R)-3-(4- amino-7H- pyrrolo[2,3-d] pyrimidin-7-yl)-5- ((((1s,3R)-3-(2-(5- (tert-butyl)-1H- benzo[d]imidazol-2- yl)ethyl)cyclobutyl) (cyclopropylmethyl) amino)methyl) cyclopentanc-1,2-diol | NMR data |
| A55 | 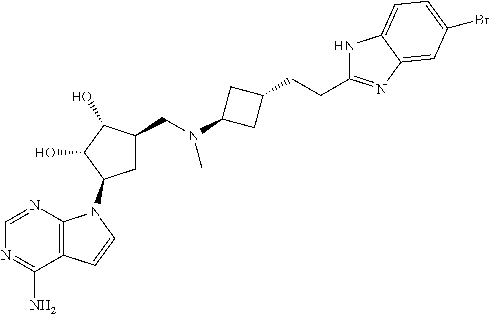 [SMILES: CN(C[C@H]1C[C@@H](n2ccc3c(N)ncnc32)[C@H](O)[C@@H]1O)[C@H]1C[C@H](CCc2nc3cc(Br)ccc3[nH]2)C1] | (1R,2S,3R,5R)-3-(4- amino-7H- pyrrolo[2,3- dlpyrimidin-7-yl)-5- ((((1s,3R)-3-(2-(5- bromo-1H- benzo[d]imidazol-2- yl(ethyl(cyclobutyl) (methyl(amino(methyl) cyclopentane-1,2-diol | NMR data |
| A56 | 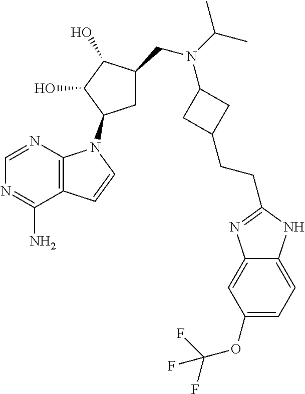 [SMILES: CC(C)N(C[C@H]1C[C@@H](n2ccc3c(N)ncnc32)[C@H](O)[C@@H]1O)C1CC(CCc2nc3cc(OC(F)(F)F)ccc3[nH]2)C1] | (1R,2S,3R,5R)-3-(4- amino-7H- pyrrolo[2,3-d] pyrimidin-7-yl)-5- ((isopropyl(3-(2-(5- (trifluoromethoxy)- 1H- benzo[d]imidazol-2- yl)ethyl)cyclobutyl) amino)methyl) cyclopentane-1,2-diol | 588.2 (M + H)⁺ |
| A57 | 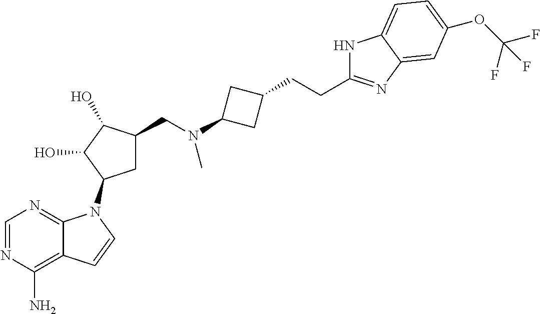 [SMILES: CN(C[C@H]1C[C@@H](n2ccc3c(N)ncnc32)[C@H](O)[C@@H]1O)[C@H]1C[C@H](CCc2nc3cc(OC(F)(F)F)ccc3[nH]2)C1] | (1R,2S,3R,5R)-3-(4- amino-7H- pyrrolo[2,3-d] pyrimidin-7-yl)-5- ((methyl((1s,3R)-3- (2-(5- (trifluoromethoxy)- 1H- benzo[d]imidazol-2- yl)ethyl)cyclobutyl) amino)methyl) cyclopentane-1,2-diol | 560.1 (M + H)⁺ |
| A58 | 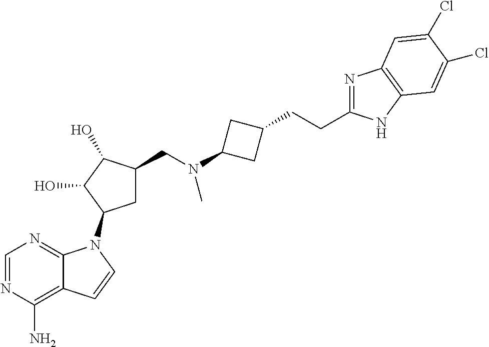 [SMILES: CN(C[C@H]1C[C@@H](n2ccc3c(N)ncnc32)[C@H](O)[C@@H]1O)[C@H]1C[C@H](CCc2nc3cc(Cl)c(Cl)cc3[nH]2)C1] | (1R,2S,3R,5R)-3-(4- amino-7H- pyrrolo[2,3-d] pyrimidin-7-yl)-5- ((((1s,3R)-3-(2-(5,6- dichloro-1H- benzo[d]imidazol-2- yl)ethyl)cyclobutyl) (methyl)amino)methyl) cyclopentane-1,2-diol | NMR data |
| A59 | 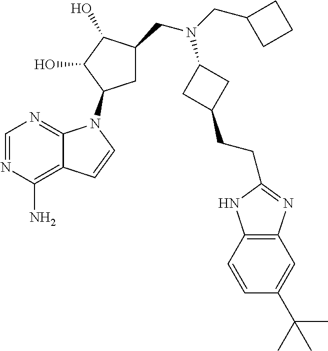 [SMILES: CC(C)(C)c1ccc2[nH]c(CC[C@H]3C[C@H](N(CC4CCC4)C[C@H]4C[C@@H](n5ccc6c(N)ncnc65)[C@H](O)[C@@H]4O)C3)nc2c1] | (1R,2S,3R,5R)-3-(4- amino-7H- pyrrolo[2,3-d] pyrimidin-7-yI)-5- ((((1s,3R)-3-(2-(5- (tert-butyl)-1H- benzo[d]imidazol-2- yl(ethyl)cyclobutyl) (cyclobutylmethyl) amino)methyl) cyclopentane-1,2-diol | 586.4 (M + H)⁺ |
| A60 | 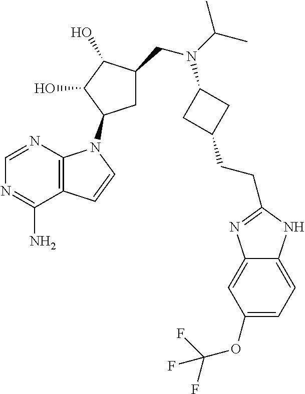 [SMILES: CC(C)N(C[C@H]1C[C@@H](n2ccc3c(N)ncnc32)[C@H](O)[C@@H]1O)[C@H]1C[C@@H](CCc2nc3cc(OC(F)(F)F)ccc3[nH]2)C1] | (1R,2S,3R,5R)-3-(4- amino-7H- pyrrolo[2,3-d] pyrimidin-7-yl)-5- ((isopropyl((1r,3S)-3- (2-(5- (trifluoromethoxy)- 1H- benzo[d]imidazol-2- yl)ethyl)cyclobutyl) amino)methyl) cyclopentane-1,2-diol | 588.2 (M + H)⁺ |
| A61 | 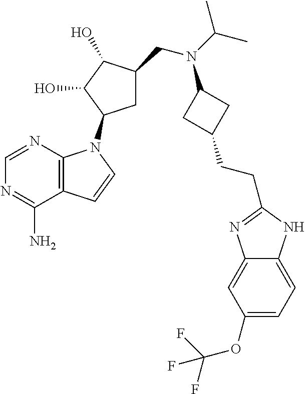 | (1R,2S,3R,5R)-3-(4- amino-7H- pyrrolo[2,3-d] pyrimidin-7-yl)-5- ((isopropyl((1s,3R)- 3-(2-(5- (trifluoromethoxy)- 1H- benzo[d]imidazol-2- yl)ethyl)cyclobutyl) amino)methyl) cyclopentane-1,2-diol | 588.7 (M + H)⁺ |
| A62 | 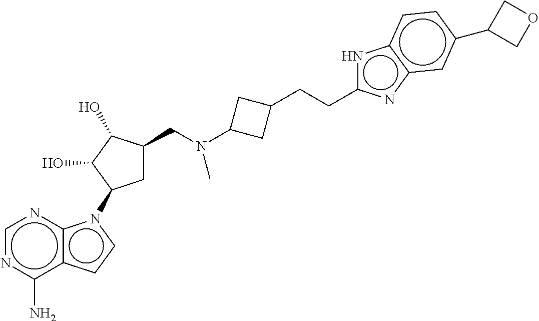 | (1R,2S,3R,5R)-3-(4- amino-7H- pyrrolo[2,3-d] pyrimidin-7-yl)-5- ((methyl(3-(2-(5- (oxetan-3-yl)-1H- benzo[d]imidazol-2- yl)ethyl)cyclobutyl) amino)methyl) cyclopentane-1,2-diol | NMR data |
| A63 | 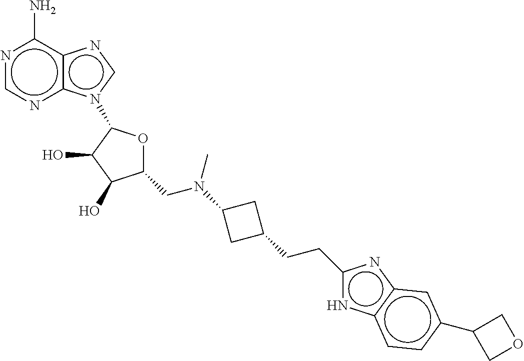 | (2R,3R,4S,5R)-2-(6- amino-9H-purin-9- yl)-5- ((methyl((1r,3S)-3- (2-(5-(oxetan-3-yl)- 1H- benzo[d]imidazol-2- yl)ethyl)cyclobutyl) amino)methyl) tetrahydrofuran-3,4- diol | 535.4 (M + H)⁺ |
| A64 | 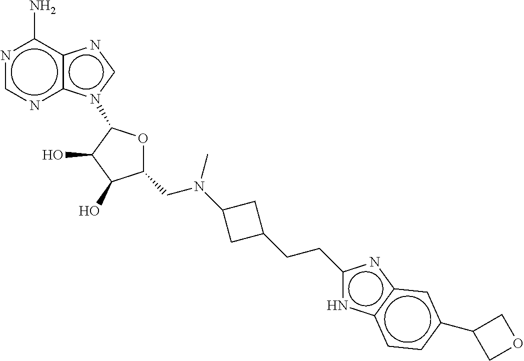 | (2R,3R,4S,5R)-2-(6- amino-9H-purin-9- yl)-5-((methyl(3-(2- (5-(oxetan-3-yl)-1H- benzo[d]imidazol-2- yl)ethyl)cyclobutyl) amino)mcthyl) tetrahydrofuran-3,4- diol | 535.3 (M + H)⁺ |
| A65 | 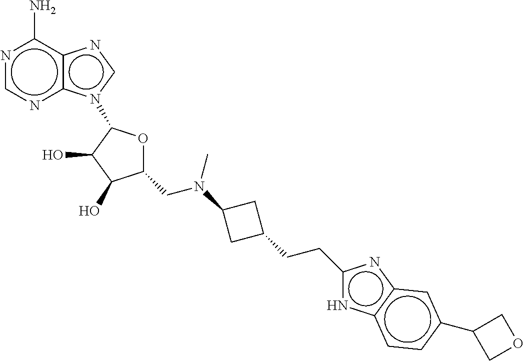 | (2R,3R,4S,5R)-2-(6- amino-9H-purin-9- yl)-5- ((methyl((1s,3R)-3- (2-(5-(oxetan-3-yl)- 1H- benzo[d]imidazol-2- yl)ethyl)cyclobutyl) amino)methyl) tetrahydrofuran-3,4- diol | 535.4 (M + H)⁺ |
| A66 | 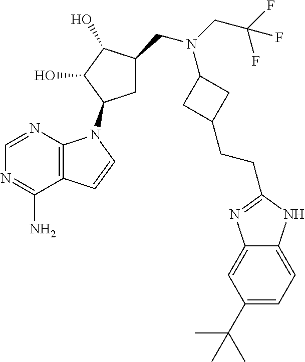 [SMILES: CC(C)(C)c1ccc2[nH]c(CCC3CC(N(C[C@H]4C[C@@H](n5ccc6c(N)ncnc65)[C@H](O)[C@@H]4O)CC(F)(F)F)C3)nc2c1] | (1R,2S,3R,5R)-3-(4- amino-7H- pyrrolo[2,3-d] pyrimidin-7-yl)-5- (((3-(2-(5-(tert- butyl)-1H- benzo[d]imidazol-2- yl)ethyl)cyclobutyl) (2,2,2- trifluoroethyl)amino) methyl)cyclopentane- 1,2-diol | 600.2 (M + H)⁺ |
| A67 | 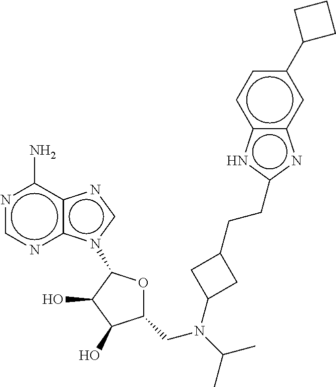 | (2R,3R,4S,5R)-2-(6- amino-9H-purin-9- yl)-5-(((3-(2-(5- cyclobutyl-1H- benzo[d]imidazol-2- yl)ethyl)cyclobutyl) (isopropyl)amino) methyl)tetrahydroturan- 3,4-diol | 561.5 (M + H)⁺ |
| A68 | 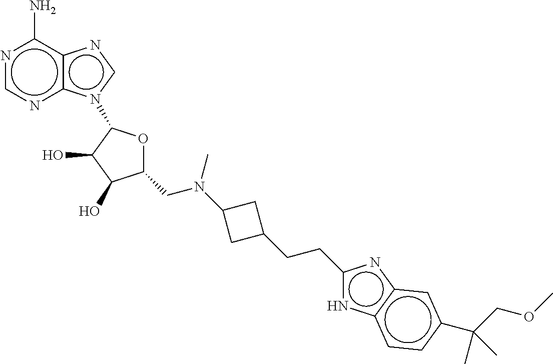 | (2R,3R,4S,5R)-2-(6- amino-9H-purin-9- yl)-5-(((3-(2-(5-(1- methoxy-2- methylpropan-2-yl)- 1H- benzo[d]imidazol-2- yl)ethyl)cyclobutyl) (methyl)amino)methyl) tetrahydrofuran-3,4- diol | 565.4 (M + H)⁺ |
| A69 | 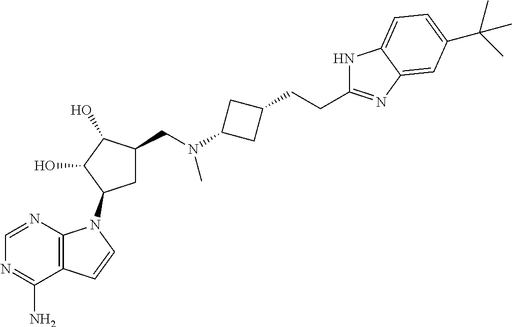 [SMILES: CN(C[C@H]1C[C@@H](n2ccc3c(N)ncnc32)[C@H](O)[C@@H]1O)[C@H]1C[C@@H](CCc2nc3cc(C(C)(C)C)ccc3[nH]2)C1] | (1R,2S,3R,5R)-3-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (methyl)amino)methyl) cyclopentane-1,2-diol | 532.3 (M + H)⁺ |
| A70 | 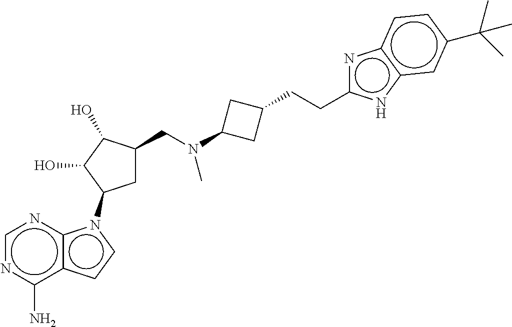 | (1R,2S,3R,5R)-3-(4- amino-7H- pyrrolo[2,3-d] pyrimidin-7-yl)-5- ((((1s,3R)-3-(2-(6- (tert-butyl)-1H- benzo[d]imidazol-2- yl)ethyl)cyclobutyl) (methyl)amino)methyl) cyclopentane-1,2-diol | 532.3 (M + H)⁺ |
| A71 | 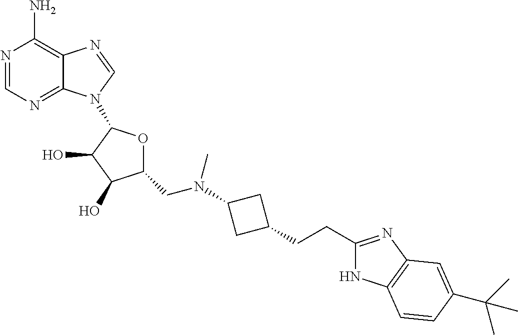 [SMILES: CN(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1] | (2R,3R,4S,5R)-2-(6- amino-9H-purin-9- yl)-5-((((1r,3S)-3-(2- (5-(tert-butyl)-1H- benzo[d]imidazol-2- yl)ethyl)cyclobutyl) (methyl)amino)rnethyl) tetrahydrofuran-3,4-diol | 535.3 (M + H)⁺ |
| A72 | 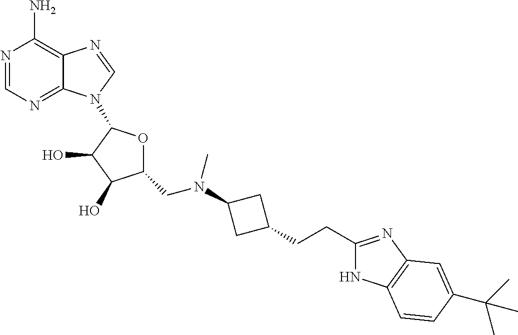 [SMILES: CN(C[C@H]1O[C@@H](n2cnc3c(N)ncnc32)[C@H](O)[C@@H]1O)[C@H]1C[C@H](CCc2nc3cc(C(C)(C)C)ccc3[nH]2)C1] | (2R,3R,4S,5R)-2-(6- amino-9H-purin-9- yl)-5-((((1s,3R)-3-(2- (5-(tert-butyl)-1H- benzo[d]imidazol-2- yl)cthyl)cyclobutyl) (methyl)amino)methyl) tetrahydrofuran-3,4- diol | 535.3 (M + H)⁺ |
| A73 | 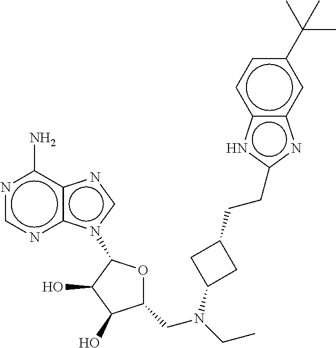 | (2R,3R,4S,5R)-2-(6- amino-9H-purin-9- yl)-5-((((1r,3S)-3-(2- (5-(tert-butyl)-1H- benzo[d]imidazol-2- yl)ethyl)cyclobutyl) (ethyl)amino)methyl) tetrahydrofuran-3-4-diol | 549.3 (M + H)⁺ |
| A74 | 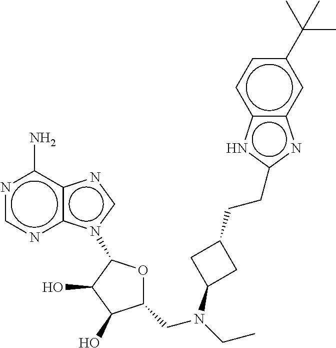 | (2R,3R,4S,5R)-2-(6- amino-9H-purin-9- yl)-5-((((1s,3R)-3-(2- (5-(tert-butyl)-1H- benzo[d]imidazol-2- yl)ethyl)cyclobutyl) (ethyl)amino)methyl) tetrahydrofuran-3,4-diol | 549.3 (M + H)⁺ |
| A75 | 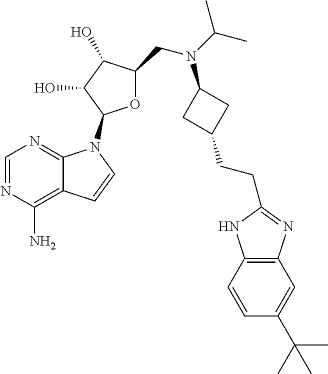 [SMILES: CC(C)N(C[C@H]1O[C@@H](n2ccc3c(N)ncnc32)[C@H](O)[C@@H]1O)[C@H]1C[C@H](CCc2nc3cc(C(C)(C)C)ccc3[nH]2)C1] | (2R,3R,4S,5R)-2-(4- amino-7H- pyrroIo[2,3-d] pyrimidin-7-yl)-5- ((((1s,3R)-3-(2-(5- (tert-butyl)-1H- benzo[d]imidazol-2- yl)ethyl)cyclobutyl) (isopropyl(amino)methyl) (tetrahydrofuran-3,4-diol | 562.5 (M + H)⁺ |
| A76 | 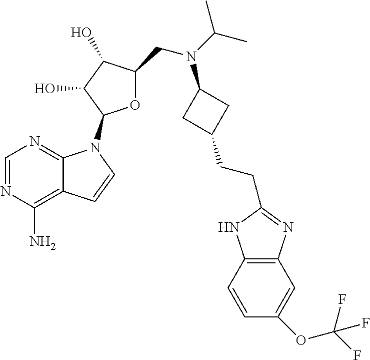 [SMILES: CC(C)N(C[C@H]1O[C@@H](n2ccc3c(N)ncnc32)[C@H](O)[C@@H]1O)[C@H]1C[C@H](CCc2nc3cc(OC(F)(F)F)ccc3[nH]2)C1] | (2R,3R,4S,5R)-2-(4- amino-7H- pyrrolo[2,3-d] pyrimidin-7-yl)-5- ((isopropyl((1s,3R)- 3-(2-(5- (trifluoromethoxy)- 1H- benzo[d]imidazol-2- yl)ethyl)cyclobutyl) amino(methyl) tctrahydrofuran-3,4-diol | 590.3 (M + H)⁺ |
| A77 | 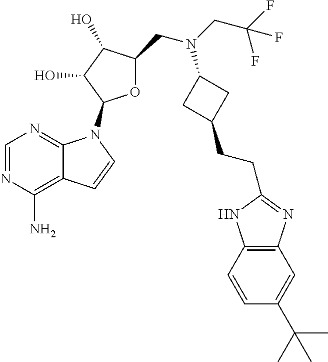 [SMILES: CC(C)(C)c1ccc2[nH]c(CC[C@H]3C[C@H](N(C[C@H]4O[C@@H](n5ccc6c(N)ncnc65)[C@H](O)[C@@H]4O)CC(F)(F)F)C3)nc2c1] | (2R,3R,4S,5R)-2-(4- amino-7H- pyrrolo[2,3-d] pyrimidin-7-yl)-5- ((((1s,3R)-3-(2-(5- (tert-butyl)-1H- benzo[d]imidazol-2- yl)ethyl)cyclobutyl) (2,2,2- trifluoroethyl)amino) methyl) tetrahydrofuran-3,4-diol | 602.3 (M + H)⁺ |
| A78 | 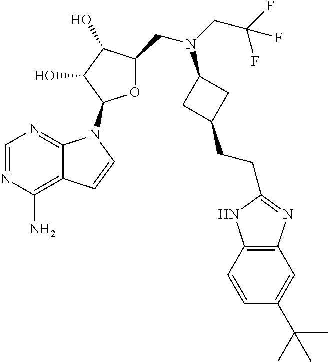 [SMILES: CC(C)(C)c1ccc2[nH]c(CC[C@H]3C[C@@H](N(C[C@H]4O[C@@H](n5ccc6c(N)ncnc65)[C@H](O)[C@@H]4O)CC(F)(F)F)C3)nc2c1] | (2R.,R,4S,5R)-2-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (2,2,2- trifluoroethyl)amino) methyl) tetrahydrofuran-3,4-diol | 602.3 (M + H)⁺ |
| A79 | 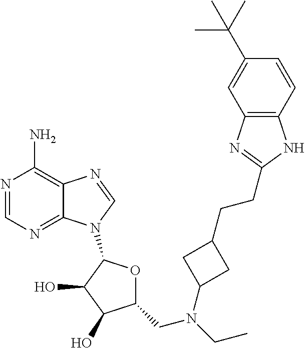 [SMILES: CCN(C[C@H]1O[C@@H](n2cnc3c(N)ncnc32)[C@H](O)[C@@H]1O)C1CC(CCc2nc3cc(C(C)(C)C)ccc3[nH]2)C1] | (2R,3R,4S,5R)-2-(6- amino-9H-purin-9- yl)-5-(((3-(2-(5-(tert- butyl)-1H- benzo[d]imidazol-2- yl)ethyl)cyclobutyl) (ethyl)amino)methyl) tetrahydrofuran-3,4-diol | 549.3 (M + H)⁺ |
| A80 | 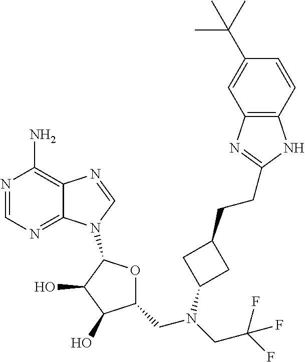 [SMILES: CC(C)(C)c1ccc2[nH]c(CC[C@H]3C[C@H](N(C[C@H]4O[C@@H](n5cnc6c(N)ncnc65)[C@H](O)[C@@H]4O)CC(F)(F)F)C3)nc2c1] | (2R,3R,4S,5R)-2-(6- amino-9H-purin-9- yl)-5-((((1s,3R)-3-(2- (5-(tert-butyl)-1H- benzo[d]imidazol-2- yl)ethyl)cyclobutyl) (2,2,2- trifluoroethyl)amino) methyl) tetrahydrofuran-3,4-diol | 603.3 (M + H)⁺ |
| A81 | 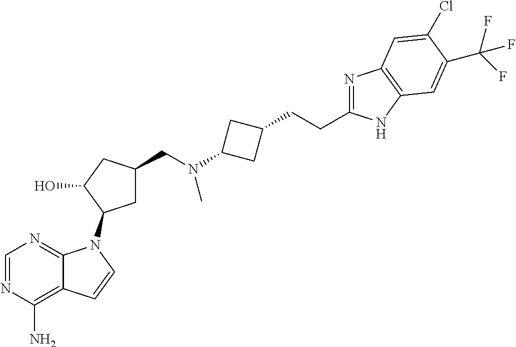 [SMILES: CN(C[C@@H]1C[C@@H](O)[C@H](n2ccc3c(N)ncnc32)C1)[C@H]1C[C@@H](CCc2nc3cc(Cl)c(C(F)(F)F)cc3[nH]2)C1] | (1R,2R,4S)-2-(4- amino-7H- pyrrolo[2,3-d] pyrimidin-7-yl)-4- ((((1r,3R)-3-(2-(5- chloro-6- (trifluoromethyl)-1H- benzo[d]imidazol-2- yl)ethyl)cyclobutyl) (mcthyl)amino)methyl) cyclopentanol | 562.3 (M + H)⁺ |
| A82 | 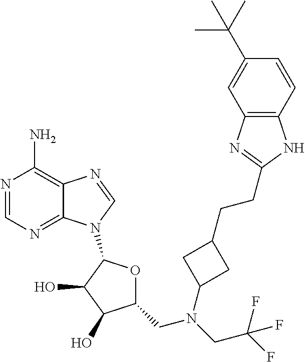 [SMILES: CC(C)(C)c1ccc2[nH]c(CCC3CC(N(C[C@H]4O[C@@H](n5cnc6c(N)ncnc65)[C@H](O)[C@@H]4O)CC(F)(F)F)C3)nc2c1] | (2R,3R,4S,5R)-2-(6- amino-9H-purin-9- yl)-5-(((3-(2-(5-(tert- butyl)-1H- benzo[d]imidazol-2- yl)ethyl)cyclobutyl) (2,2,2- trifluoroethyl)amino) methyl) tetrahydrofuran-3,4-diol | 603.3 (M + H)⁺ |
| A83 | 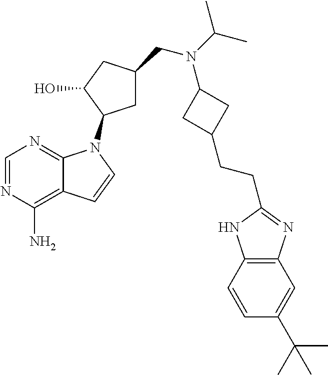 [SMILES: CC(C)N(C[C@@H]1C[C@@H](O)[C@H](n2ccc3c(N)ncnc32)C1)C1CC(CCc2nc3cc(C(C)(C)C)ccc3[nH]2)C1] | (1R,2R,4S)-2-(4- amino-7H- pyrrolo[2,3-d] pyrimidin-7-yl)-4- (((3-(2-(5-(tert- butyl)-1H- benzo[d]imidazol-2- yl)ethyl)cyclobutyl) (isopropyl)amino)methyl) cyclopentanol | 544.5 (M + H)⁺ |
| A84 | 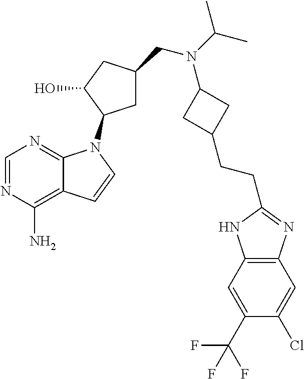 [SMILES: CC(C)N(C[C@@H]1C[C@@H](O)[C@H](n2ccc3c(N)ncnc32)C1)C1CC(CCc2nc3cc(Cl)c(C(F)(F)F)cc3[nH]2)C1] | (1R,2R,4S)-2-(4- amino-7H- pyrrolo[2,3-d] pyrimidin-7-yl)-4- (((3-(2-(5-chloro-6- (trifluoromethyl)-1H- benzo[d]imidazol-2- yl)ethyl)cyclobutyl) (isopropyl)amino)methyl) cyclopentanol | 590.3 (M + H)⁺ |
| A85 | 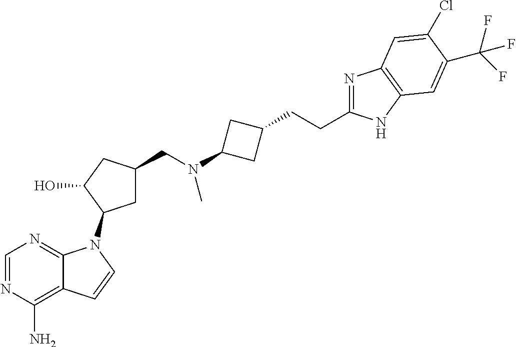 [SMILES: CN(C[C@@H]1C[C@@H](O)[C@H](n2ccc3c(N)ncnc32)C1)[C@H]1C[C@H](CCc2nc3cc(Cl)c(C(F)(F)F)cc3[nH]2)C1] | (1R,2R,4S)-2-(4- amino-7M- pyrrolo[2,3-d] pyrimidin-7-yl)-4- ((((1s,3S)-3-(2-(5- chloro-6- (trifluoromethyl)-1H- benzo[d]imidazol-2- yl)ethyl)cyclobutyl) (methyl)amino)methyl) cyclopentanol | 562.3 (M + H)⁺ |
| A86 | 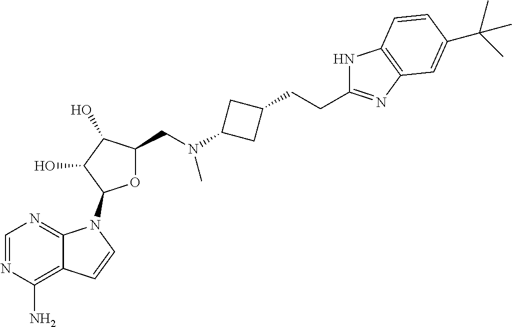 [SMILES: CN(C[C@H]1O[C@@H](n2ccc3c(N)ncnc32)[C@H](O)[C@@H]1O)[C@H]1C[C@@H](CCc2nc3cc(C(C)(C)C)ccc3[nH]2)C1] | (2R,3R,4S,5R)-2-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (methyl(amino)methyl) tetrahydrofuran-3,4-diol | 534.3 (M + H)⁺ |
| A87 | 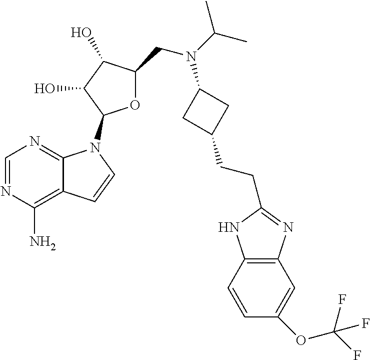 [SMILES: CC(C)N(C[C@H]1O[C@@H](n2ccc3c(N)ncnc32)[C@H](O)[C@@H]1O)[C@H]1C[C@@H](CCc2nc3cc(OC(F)(F)F)ccc3[nH]2)C1] | (2R,3R,4S,5R)-2-(4- amino-7H- pyrrolo[2,3-d] pyrimidin-7-yl)-5- ((isopropyl((1r,3S)-3- (2-(5- (trifluoromethoxy)- 1H- benzo[d]imidazol-2- yl)ethyl)cyclobutyl) amino)methyl) tetrahydrofuran-3,4-diol | 590.3 (M + H)⁺ |
| A88 | 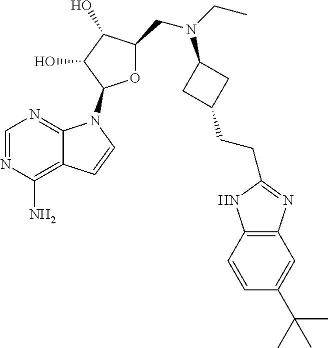 [SMILES: CCN(C[C@H]1O[C@@H](n2ccc3c(N)ncnc32)[C@H](O)[C@@H]1O)[C@H]1C[C@H](CCc2nc3cc(C(C)(C)C)ccc3[nH]2)C1] | (2R,3R,4S,5R)-2-(4- amino-7H- pyrrolo[2,3-d] pyrimidin-7-yl)-5- ((((1s,3R)-3-(2-(5- (tert-butyl)-1H- benzo[d]imidazol-2- yl)ethyl)cyclobutyl) (ethyl)amino)methyl) tetrahydrofuran-3-4-diol | 548.3 (M + H)⁺ |
| A89 | 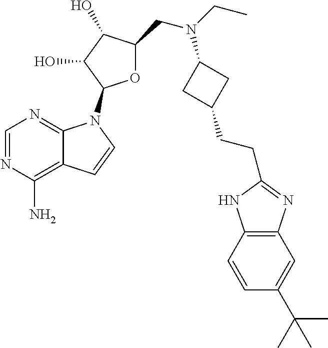 [SMILES: CCN(C[C@H]1O[C@@H](n2ccc3c(N)ncnc32)[C@H](O)[C@@H]1O)[C@H]1C[C@@H](CCc2nc3cc(C(C)(C)C)ccc3[nH]2)C1] | (2R,3R,4S,5R)-2-(4- amino-7H- pyrrolo[2,3-d] pyrimidin-7-yl)-5- ((((1r,3S)-3-(2-(5- (tert-butyl)-1H- benzo[d]imidazol-2- yl)ethyl)cyclobutyl) (ethyl)amino)methyl) tetrahydrofuran-3,4-diol | 548.3 (M + H)⁺ |
| A90 | 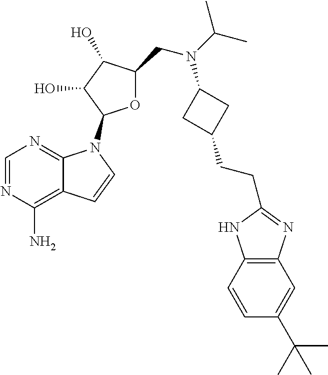 [SMILES: CC(C)N(C[C@H]1O[C@@H](n2ccc3c(N)ncnc32)[C@H](O)[C@@H]1O)[C@H]1C[C@@H](CCc2nc3cc(C(C)(C)C)ccc3[nH]2)C1] | (2R,3R,4S,5R)-2-(4- amino-7H- pyrrolo[2,3-d] pyrimidin-7-yl)-5- ((((1r,3S)-3-2-(5- (tert-butyl)-1H- benzo[d]imidazol-2- yl)ethyl)cyclobutyl) (isopropyl(amino)methyl (tetrahydrofuran-3,4-diol | 562.5 (M + H)⁺ |
| A91 | 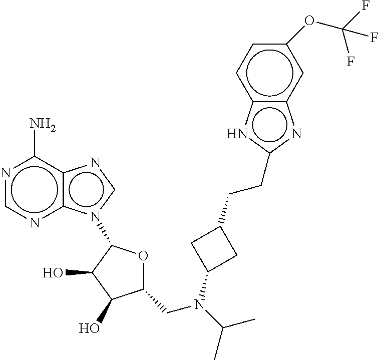 | (2R,3R,4S,5R)-2-(6- amino-9H-purin-9- yl)-5- ((isopropyl((1r,3S)-3- (2-(5- (trifluoromethoxy)- 1H- benzo[d]imidazol-2- yl)ethyl)cyclobutyl) amino(methyl) tetrahydrofuran-3,4-diol | 591.2 (M + H)⁺ |
| A92 | 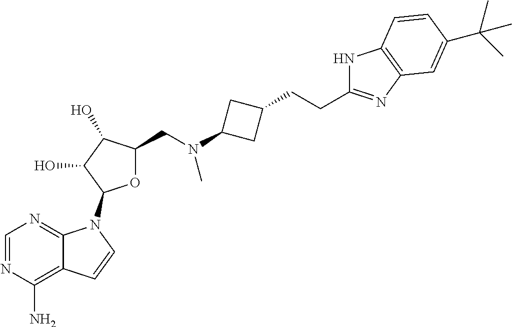 [SMILES: CN(C[C@H]1O[C@@H](n2ccc3c(N)ncnc32)[C@H](O)[C@@H]1O)[C@H]1C[C@H](CCc2nc3cc(C(C)(C)C)ccc3[nH]2)C1] | (2R,3R,4S,5R)-2-(4- amino-7H- pyrrolo[2,3-d] pyrimidin-7-yl)-5- ((((1s,3R)-3-(2-(5- (tert-butyl)-1H- benzo[d]imidazol-2- yl)ethyl)cyclobutyl) (methyl)amino)methyl) tetrahydrofuran-3,4-diol | 534.3 (M + H)⁺ |
| A93 | 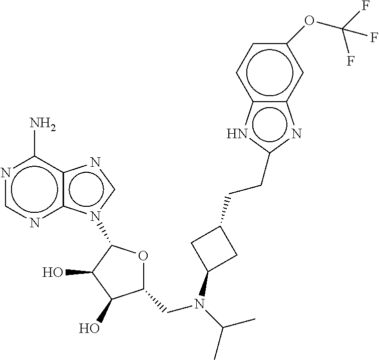 | (2R,3R,4S,5R)-2-(6- amino-9H-purin-9- yl)-5- ((isopropyl((1s,3R)- 3-(2-(5- (trifluoromethoxy)- 1H- benzo[d]imidazol-2- yl)ethyl)cyclobutyl) amino)methyl) tetrahydrofuran-3,4-diol | 591.3 (M + H)⁺ |
| A94 | 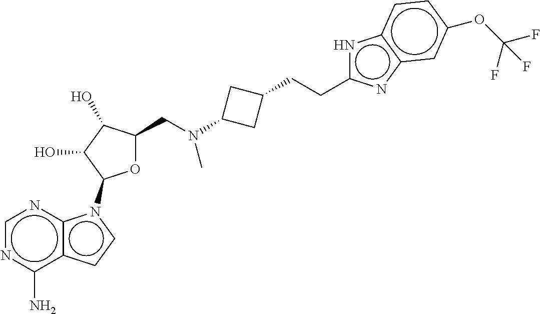 | (2R,3R,4S,5R)-2-(4- amino-7H- pyrrolo[2,3-d] pyrimidin-7-yl)-5- ((methyl((1r,3S)-3- (2-(5- (trifluoromethoxy)- 1H- benzo[d]imidazol-2- yl)ethyl)cyclobutyl) amino)methyl) tetrahydrofuran-3,4-diol | 562.2 (M + H)⁺ |
| A95 | 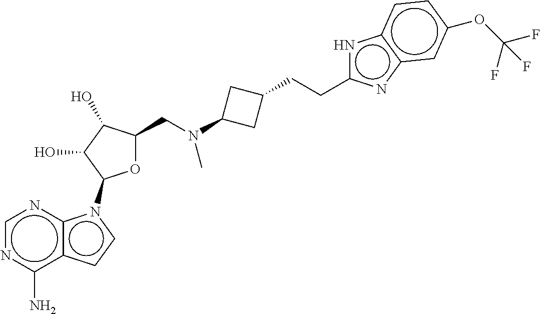 | (2R,3R,4S,5R)-2-(4- amino-7H- pyrrolo[2,3-d] pyrimidin-7-yl)-5- ((methyl((1s,3R)-3- (2-(5- (trifluoromethoxy)- 1H- benzo[d]imidazol-2- yl)ethyl)cyclobutyl) amino(methyl) tetrahydrofuran-3,4-diol | 562.3 (M + H)⁺ |
| A96 | 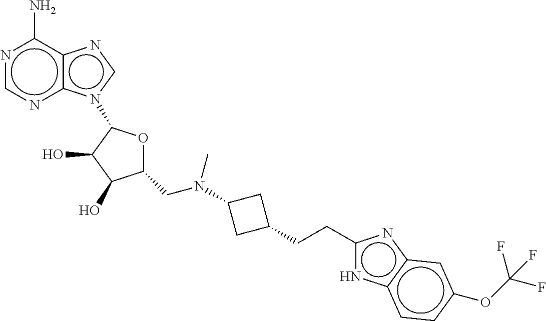 | (2R,3R,4S,5R)-2-(6- amino-9H-purin-9- yl)-5- ((methyl((1r,3S)-3- (2-(5- (trifluoromethoxy)- 1H- benzo[d]imidazol-2- yl)ethyl)cyclobutyl) amino(methyl) tetrahydrofuran-3,4-diol | 563.3 (M + H)⁺ |
| A97 | 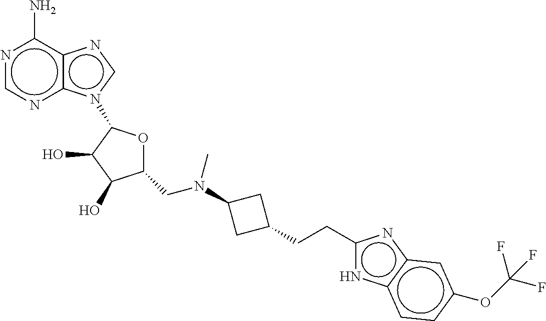 | (2R,3R,4S,5R)-2-(6- amino-9H-purin-9- yl)-5- ((methyl((1s,3R)-3- (2-(5- (trifluoromethoxy)- 1H- benzo[d]imidazol-2- yl)ethyl)cyclobutyl) amino)methyl) tetrahydrofuran-3,4-diol | 563.3 (M + H)⁺ |
| A98 | 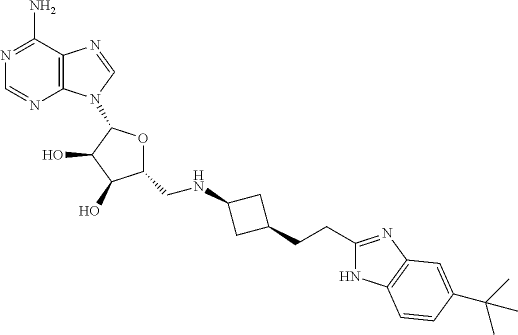 [SMILES: CC(C)(C)c1ccc2[nH]c(CC[C@H]3C[C@@H](NC[C@H]4O[C@@H](n5cnc6c(N)ncnc65)[C@H](O)[C@@H]4O)C3)nc2c1] | (2R,3R,4S,5R)-2-(6- amino-9H-purin-9- yl)-5-((((1r,3S)-3-(2- (5-(tert-butyl)-1H- benzo[d]imidazol-2- yl)ethyl)cyclobutyl) amino)methyl) tetrahydrofuran-3,4-diol | 521.3 (M + H)⁺ |
| A99 | 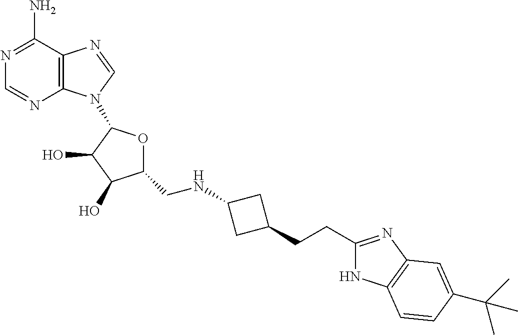 [SMILES: CC(C)(C)c1ccc2[nH]c(CC[C@H]3C[C@H](NC[C@H]4O[C@@H](n5cnc6c(N)ncnc65)[C@H](O)[C@@H]4O)C3)nc2c1] | (2R,3R,4S,5R)-2-(6- amino-9H-purin-9- yl)-5-((((1s,3R)-3-(2- (5-(tert-butyl)-1H- benzo[d]imidazol-2- yl)ethyl)cyclobutyl) amino)methyl) tetrahydrofuran-3,4-diol | 521.3 (M + H)⁺ |
| A100 | 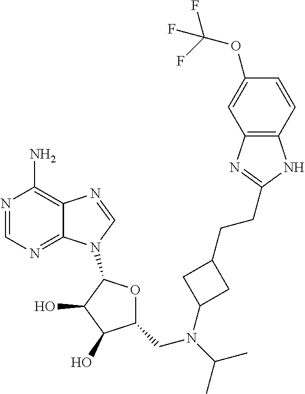 [SMILES: CC(C)N(C[C@H]1O[C@@H](n2cnc3c(N)ncnc32)[C@H](O)[C@@H]1O)C1CC(CCc2nc3cc(OC(F)(F)F)ccc3[nH]2)C1] | (2R,3R,4S,5R)-2-(6- amino-9H-purin-9- yl)-5-((isopropyl(3- (2-(5- (trifluoromethoxy)- 1H- benzo[d]imidazol-2- yl)ethyl)cyclobutyl) amino)methyl) tetrahydrofuran-3,4-diol | 591.3 (M + H)⁺ |
| A101 | 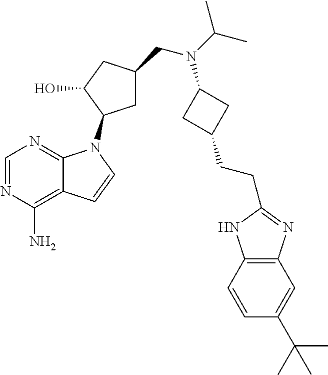 [SMILES: CC(C)N(C[C@@H]1C[C@@H](O)[C@H](n2ccc3c(N)ncnc32)C1)[C@H]1C[C@@H](CCc2nc3cc(C(C)(C)C)ccc3[nH]2)C1] | (1R,2R,4S)-2-(4- amino-7H- pyrrolo[2,3-d] pyrimidin-7-yl)-4- ((((1r,3R)-3-(2-(5- (tert-butyl)-1H- benzo[d]imidazol-2- yl)ethyl)cyclobutyl) (isopropyl)amino)methyl) cyclopentanol | 544.1 (M + H)⁺ |
| A102 | 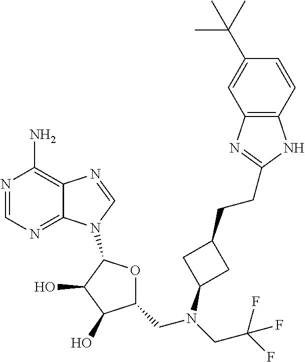 [SMILES: CC(C)(C)c1ccc2[nH]c(CC[C@H]3C[C@@H](N(C[C@H]4O[C@@H](n5cnc6c(N)ncnc65)[C@H](O)[C@@H]4O)CC(F)(F)F)C3)nc2c1] | (2R,3R,4S,5R)-2-(6- amino-9H-purin-9- yl)-5-((((1r,3S)-3-(2- (5-(tert-butyl)-1H- benzo[d]imidazol-2- yl)ethyl)cyclobutyl) (2,2,2- trifluoroethyl)amino) methyl) tetrahydrofuran-3,4-diol | 603.3 (M + H)⁺ |
| A103 | 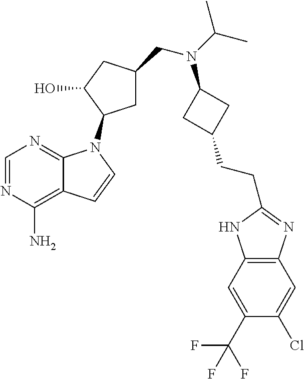 [SMILES: CC(C)N(C[C@@H]1C[C@@H](O)[C@H](n2ccc3c(N)ncnc32)C1)[C@H]1C[C@H](CCc2nc3cc(Cl)c(C(F)(F)F)cc3[nH]2)C1] | (1R,2R,4S)-2-(4- amino-7H- pyrrolo[2,3-d] pyrimidin-7-yl)-4- ((((1s,3S)-3-(2-(5- chloro-6- (trifluoromethyl)-1H- benzo[d]imidazol-2- yl)ethyl)cyclobutyl) (isopropyl(amino)methyl) cyclopentanol | 589.9 (M + H)⁺ |
| A104 | 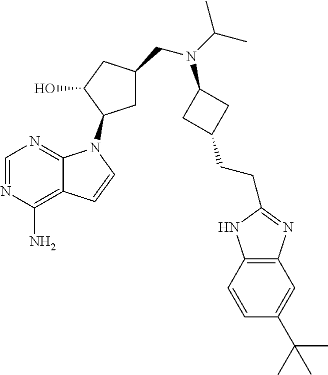 [SMILES: CC(C)N(C[C@@H]1C[C@@H](O)[C@H](n2ccc3c(N)ncnc32)C1)[C@H]1C[C@H](CCc2nc3cc(C(C)(C)C)ccc3[nH]2)C1] | (1R,2R,4S)-2-(4- amino-7H- pyrrolo[2,3-d] pyrimidin-7-yl)-4- ((((1s,3S)-3-(2-(5- (tert-butyl)-1H- benzo[d]imidazol-2- yl)ethyl)cyclobutyl) (isopropyl)amino)methyl) cyclopentanol | 544.1 (M + H)⁺ |
| A105 | 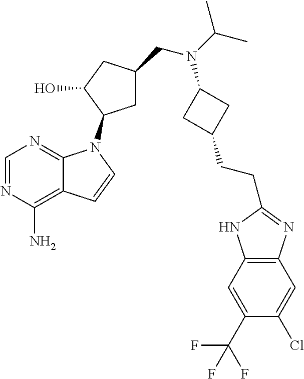 [SMILES: CC(C)N(C[C@@H]1C[C@@H](O)[C@H](n2ccc3c(N)ncnc32)C1)[C@H]1C[C@@H](CCc2nc3cc(Cl)c(C(F)(F)F)cc3[nH]2)C1] | (1R,2R,4S)-2-(4- amino-7H- pyrrolo[2,3-d] pyrimidin-7-yl)-4- ((((1r,3R)-3-(2-(5- chloro-6- (trifluoromethyl)-1H- benzo[d]imidazol-2- yl)ethyl)cyclobutyl) (isopropyl)amino)methyl) cyclopentanol | 589.9 (M + H)⁺ |
| A106 | 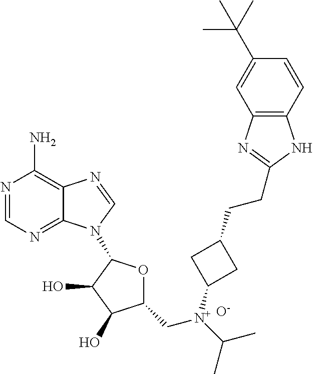 | (1r,3S)-N- (((2R,3S,4R,5R)-5- (6-amino-9H-purin-9- yl)-3,4- dihydroxytetrahydro- furan-2-yl)methyl)-3- (2-(5-(tert-butyl)-1H- benzo[d]imidazol-2- yl)ethyl)-N- isopropylcyclobutan- amine oxide | 579.4 (M + H)⁺ |
| A107 | 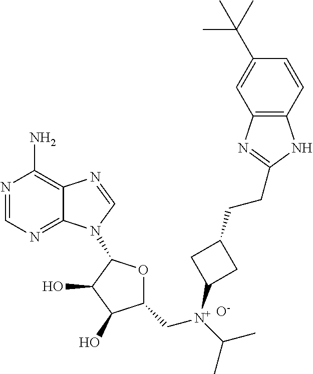 | (R,1s,3R)-N- (((2R,3S,4R,5R)-5- (6-amino-9H-purin-9- yl)-3,4- dihydroxytetrahydro- furan-2-yl)methyl)-3- (2-(5-(tert-butyl)-1H- benzo[d]imidazol-2- yl)ethyl)-N- isopropylcyclobutan- amine oxide | 579.4 (M + H)⁺ |
| A108 | 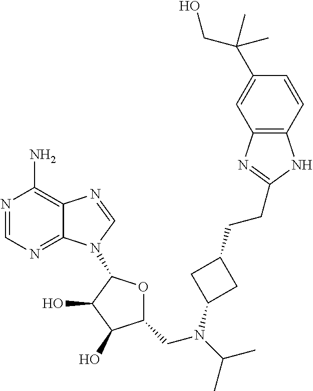 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)CO)ccc3[nH]2)C1] | (2R,3R,4S,5R)-2-(6- amino-9H-purin-9- yl)-5-((((1r,3S)-3-(2- (5-(1-hydroxy-2- methylpropan-2-yl)- 1H- benzo[d]imidazol-2- yl)ethyl)cyclobutyl) (isopropyl(amino)methyl) tetrahydrofuran-3,4-diol | 579.4 (M + H)⁺ |
| A109 | 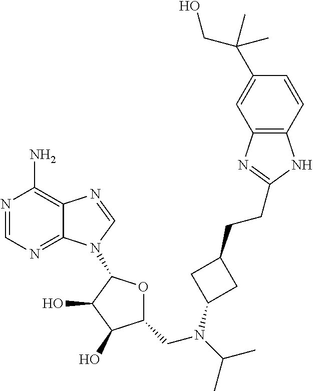 [SMILES: CC(C)N(C[C@H]1O[C@@H](n2cnc3c(N)ncnc32)[C@H](O)[C@@H]1O)[C@H]1C[C@H](CCc2nc3cc(C(C)(C)CO)ccc3[nH]2)C1] | (2R,3R,4S,5R)-2-(6- amino-9H-purin-9- yl)-5-((((1s,3R)-3-(2- (5-(1-hydroxy-2- methylpropan-2-yl)- 1H- benzo[d]imidazol-2- yl(ethyl)cyclobutyl) (isopropyl)amino)methyl) tctrahydrofuran-3,4-diol | 579.4 (M + H)⁺ |
| A110 | 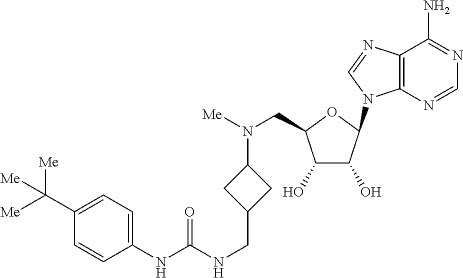 [SMILES: CN(C[C@H]1O[C@@H](n2cnc3c(N)ncnc32)[C@H](O)[C@@H]1O)C1CC(CNC(=O)Nc2ccc(C(C)(C)C)cc2)C1] | 1-((3- ((((2R,3S,4R,5R)-5- (6-amino-9H-purin-9- yl)-3,4- dihydroxytetrahydro- furan-2- yl)methyl)(methyl)amino) cyclobutyl) methyl)-3-(4-(tert- butyl)phenyl)urea | 539.3 (M + H)⁺ |
| A111 | 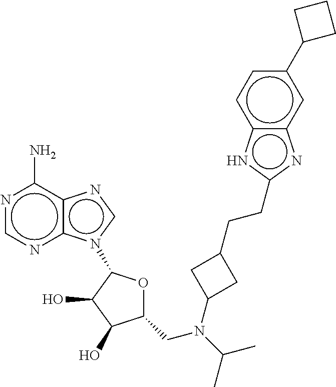 | (2R,3R,4S,5R)-2-(6- amino-9H-purin-9- yl)-5-(((3-(2-(5- cyclobutyl-1H- benzo[d]imidazol-2- yl)ethyl)cyclobutyl) (issopropyl)amino)methyl) tctrahydrofuran-3,4-diol | 561 (M + H)⁺ |
| A112 | 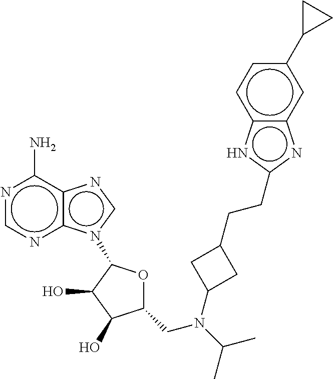 | (2R,3R,4S,5R)-2-(6- amino-9H-purin-9- yl)-5-(((3-(2-(5- cyclopropyl-1H- benzo[d]imidazol-2- yl(ethyl)cyclobutyl) (isopropyl)amino)methyl) tetrahydrofuran-3,4-diol | 547 (M + H)⁺ |
| A113 | 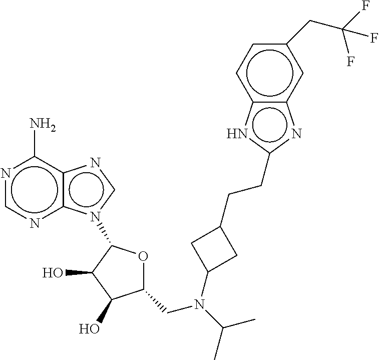 | (2R,3R,4S,5R)-2-(6- amino-9H-purin-9- yl )-5-((isopropyl(3- (2-(5-(2,2,2- trifluoroethyl)-1H- benzo[d]imidazol-2- yl)ethyl)cyclobutyl) amino)methyl) tetrahydrofuran-3,4-diol | 589 (M + H)⁺ |
| A114 | 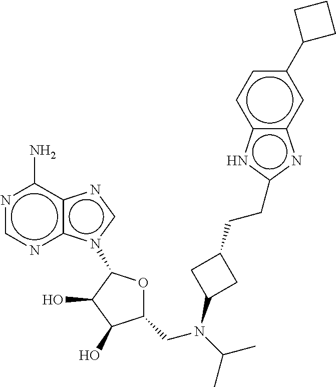 | (2R,3R,4S,5R)-2-(6- amino-9H-purin-9- yl)-5-((((1s,3R)-3-(2- (5-cyclobutyl-1H- benzo[d]imidazol-2- yl)ethyl)cyclobutyl) (isopropyl)amino)methyl) tetrahydrofuran-3,4-diol | 561 (M + H)⁺ |
| A115 | 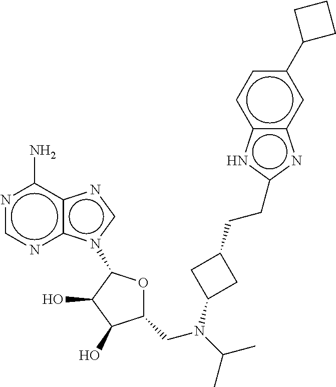 | (2R,3R,4S,5R)-2-(6- amino-9H-purin-9- yl)-5-(((1r,3S)-3-(2- (5-cyclobutyl-1H- benzo[d]imidazol-2- yl)ethyl)cyclobutyl) (isopropyl)amino)methyl) tetrahydrofuran-3,4-diol | 561 (M + H)⁺ |
| A116 | 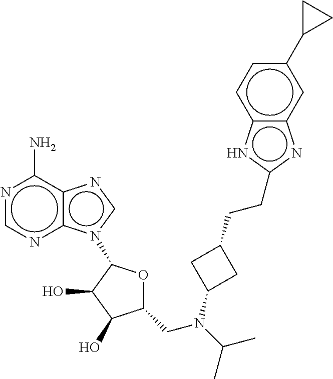 | (2R,3R,4S,5R)-2-(6- amino-9H-purin-9- yl)-5-((((1r,3S)-3-(2- (5-cyclopropyl-1H- benzo[d]imidazol-2- yl)ethyl)cyclobutyl) (isopropyl(amino)methyl) tetrahydrofuran-3,4-diol | 547 (M + H)⁺ |
| A117 | 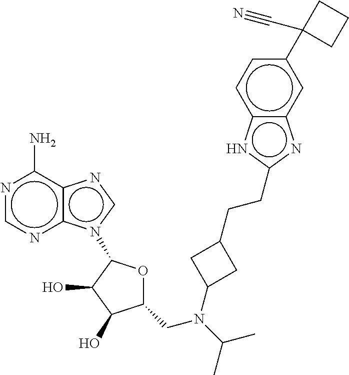 | 1-(2-(2-(3- ((((2R,3S,4R,5R)-5- (6-amino-9H-purin-9- yl)-3,4- dihydroxytetrahydro- furan-2- yl)methyl)(isopropyl) amino)cyclobutyl) ethyl)-1H- benzo[d]imidazol-5- yl)cyclobutanecarboni- trile | 586 (M + H)⁺ |
| A118 | 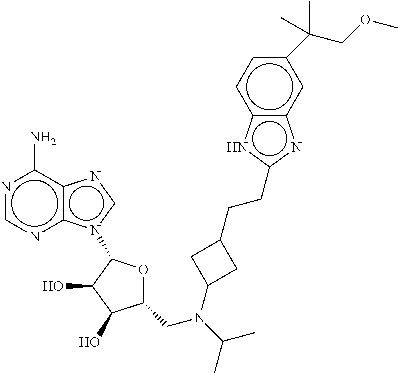 | (2R,3R,4S,5R)-2-(6- amino-9H-purin-9- yl)-5-((isopropyl(3- (2-(5-(1-methoxy-2- methylpropan-2-yl)- 1H- benzo[d]imidazol-2- yl)ethyl)cyclobulyl) amino(methyl) tetrahydrofuran-3,4-diol | 593 (M + H)⁺ |
| A119 | 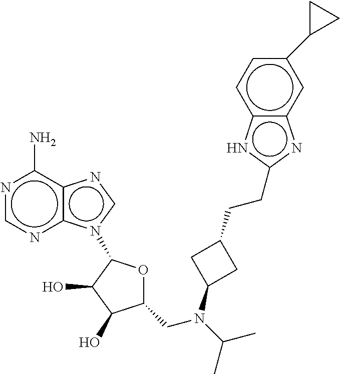 | (2R,3R,4S,5R)-2-(6- amino-9H-purin-9- yl)-5-((((1s,3R)-3-(2- (5-cyclopropyl-1H- benzo[d]imidazol-2- yl)ethyl)cyclobutyl) (isopropyl(amino)methyl) tetrahydrofuran-3,4-diol | 547 (M + H)⁺ |
| A120 | 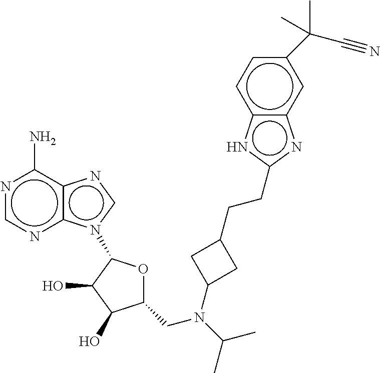 | 2-(2-(2-(3- ((((2R,3S,4R,5R)-5- (6-amino-9H-purin-9- yl)-3,4- dihydroxytetrahydro- furan-2- yl)methyl)(isopropyl) amino)cyclobutyl) ethyl)-1H- benzo[d]imidazol-5- yl)-2- methylpropanenitrile | 574 (M + H)⁺ |
| A121 | 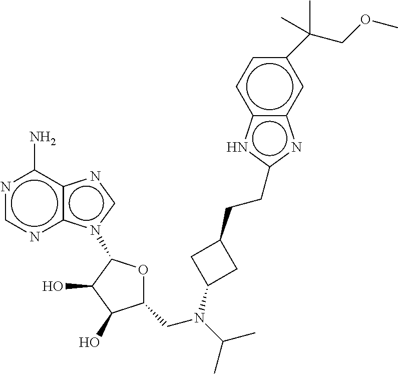 | (2R,3R,4S,5R)-2-(6- amino-9H-purin-9- yl)-5- ((isopropyl((1s,3R)- 3-(2-(5-(1-methoxy- 2-methylpropan-2- yl)-1H- benzo[d]imidazol-2- yl)ethyl)cyclobutyl) amino)methyl) tetrahydrofuran-3,4-diol | 593 (M + H)⁺ |
| A122 | 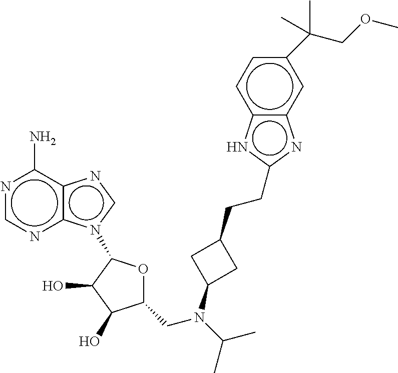 | (2R,3R,4S,5R)-2-(6- amino-9H-purin-9- yl)-5- ((isopropyl((1r,3S)-3- (2-(5-(1-methoxy-2- methylpropan-2-yl)- 1H- benzo[d]imidazol-2- yl)ethyl)cyclobutyl) amino)methyl) tetrahydrofuran-3,4-diol | 593 (M + H)⁺ |
| A123 | 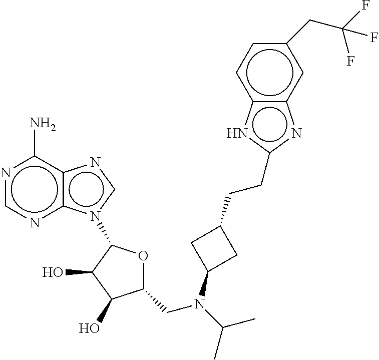 | (2R,3R,4S,5R)-2-(6- amino-9H-purin-9- yl)-5- ((isopropyl((1s,3R)- 3-(2-(5-(2,2,2- trifluoroethyl)-1H- benzo[d]imidazol-2- yl)ethyl)cyclobutyl) amino)methyl) tetrahydrofuran-3,4-diol | 589 (M + H)⁺ |
| A124 | 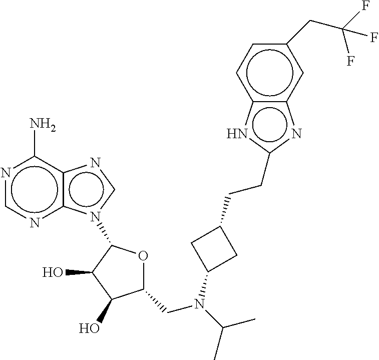 | (2R,3R,4S,5R)-2-(6- amino-9H-purin-9- yl)-5- ((isopropyl((1r,3S)-3- (2-(5-(2,2,2- trifluoroethyl)-1- benzo[d]imidazol-2- yl)ethyl)cyclobutyl) amino)methyl) tetrahydrofuran-3,4-diol | 589 (M + H)⁺ |
| A125 | 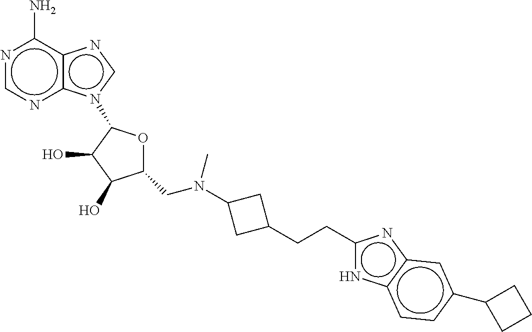 | (2R,3R,4S,5R)-2-(6- amino-9H-purin-9- yl)-5-((((3-(2-(5- cyclobutyl-1H- benzo[d]imidazol-2- yl)ethyl)cyclobutyl) (methyl)amino)methyl) tetrahydrofuran-3,4-diol | 533 (M + H)⁺ |
| A126 | 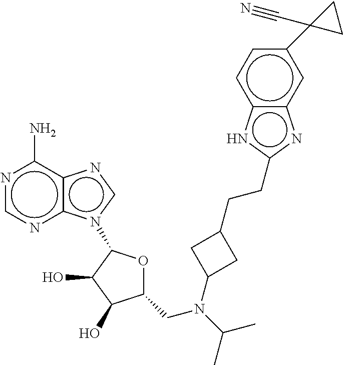 | 1-(2-(2-(3- ((((2R,3S,4R,5R)-5- (6-amino-9H-purin-9- yl)-3,4- dihydroxytetrahydro- furan-2- yl)methyl)(isopropyl) amino)cyclobutyl) ethyl)-1H- benzo[d]imidazol-5- yl)cyclopropanecar- bonitrilc | 572 (M + H)⁺ |
| A127 | 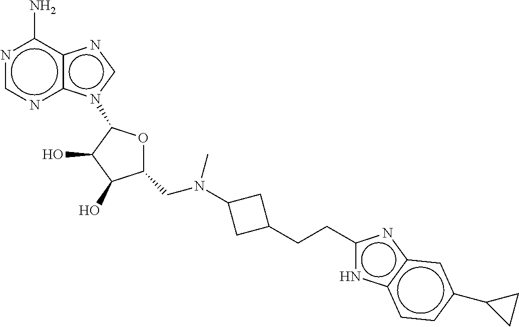 | (2R,3R,4S,5R)-2-(6- amino-9H-purin-9- yl)-5-(((3-(2-(5- cyclopropyl-1H- benzo[d]imidazol-2- yl)ethyl)cyclobutyl) (methyl)amino)methyl) tetrahydrofuran-3,4-diol | 519 (M + H)⁺ |
| A128 | 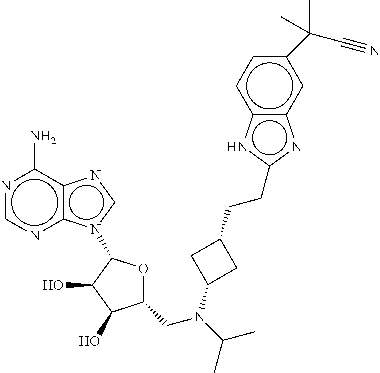 | 2-(2-(2-((1S,3r)-3- ((((2R,3S,4R,5R)-5- (6-amino-9H-purin-9- yl)-3,4- dihydroxytetrahydro- furan-2- yl)methyl)(isopropyl) amino)cyclobutyl) ethyl)-1H- benzo[d]imidazol-5- yl)-2- methylpropanenitrilc | 574 (M + H)⁺ |
| A129 | 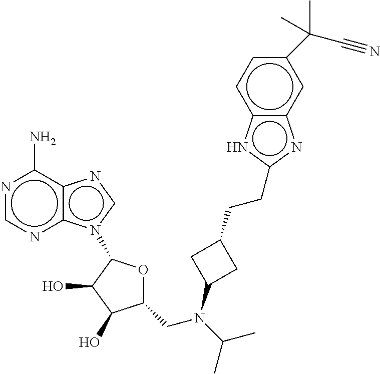 | 2-(2-(2-((1R,3s)-3- ((((2R,3S,4R,5R)-5- (6-amino-9H-purin-9- yl)-3,4- dihydroxytetrahydro- furan-2- yl)methyl)(isopropyl) amino)cyclobutyl) ethyl)-1H- benzo[d]imidazol-5- yl)-2- methylpropanenitrile | 574 (M + H)⁺ |
| A130 | 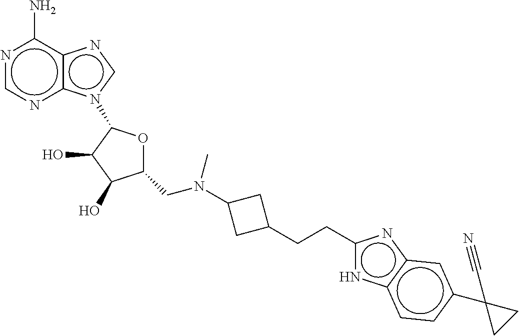 | 1-(2-(2-(3- ((((2R,3S,4R,5R)-5- (6-amino-9H-purin-9- yl)-3,4- dihydroxytetrahydro- furan-2- yl)methyl)(methyl) amino)cyclobutyl) ethyl)-1H- benzo[d]imidazol-5- yl)cyclopropanecar- bonitrile | 544 (M + H)⁺ |
| A131 | 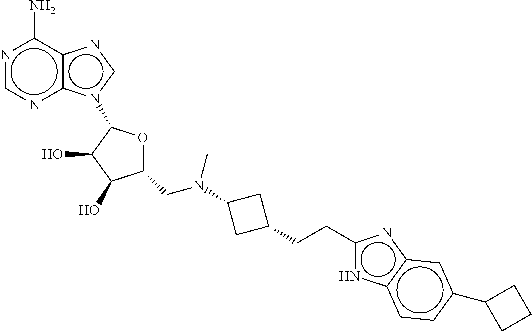 | (2R,3R,4S,5R)-2-(6- amino-9H-purin-9- yl)-5-((((1r,3S)-3-(2- (5-cyclobutyl-1H- benzo[d]imidazol-2- yl)ethyl)cyclobutyl) (methyl)amino)methyl) tetrahydrofuran-3,4- diol | 533 (M + H)⁺ |
| A132 | 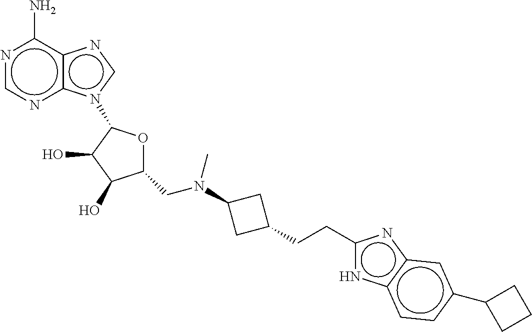 | (2R,3R,4S,5R)-2-(6- amino-9H-purin-9- yl)-5-((((1s,3R)-3-(2- (5-cyclobutyl-1H- benzo[d]imidazol-2- yl)ethyl)cyclobutyl) (methyl)amino)methyl) tetrahydrofuran-3,4-diol | 533 (M + H)⁺ |
| A133 | 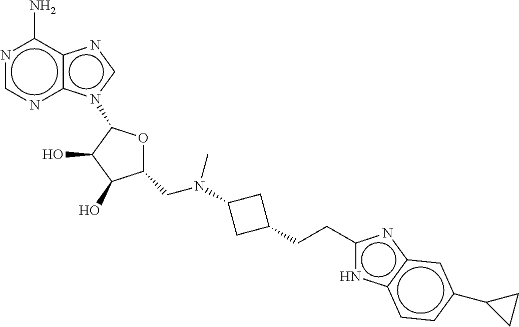 | (2R,3R,4S,5R)-2-(6- amino-9H-purin-9- yl)-5-((((1r,3S)-3-(2- (5-cyclopropyl-1H- benzo[d]imidazol-2- yl)ethyl)cyclobutyl) (methyl)amino)methyl) tetrahydrofuran-3,4-diol | 519 (M + H)⁺ |
| A134 | 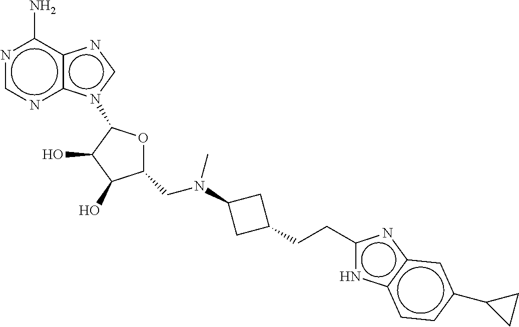 | (2R,3R,4S,5R)-2-(6- amino-9H-purin-9- yl)-5-((((1s,3R)-3-(2- (5-cyclopropyl-1H- benzo[d]imidazol-2- yl)ethyl)cyclobutyl) (methyl)amino)methyl) tetrahydrofuran-3,4-diol | 519 (M + H)⁺ |
| A135 | 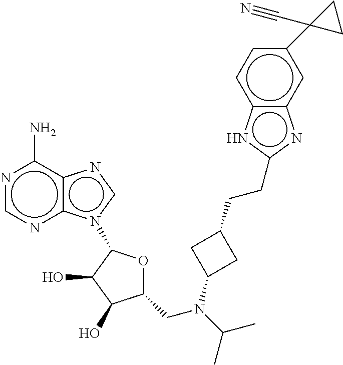 | 1-(2-(2-((1S,3r)-3- ((((2R,3S,4R,5R)-5- (6-amino-9H-purin-9- yl)-3,4- dihydroxytetrahydro- furan-2- yl)methyl)(isopropyl) amino)cyclobutyl) ethyl)-1H- benzo[d]imidazol-5- yl)cyclopropanecar- bonitrile | 572 (M + H)⁺ |
| A136 | 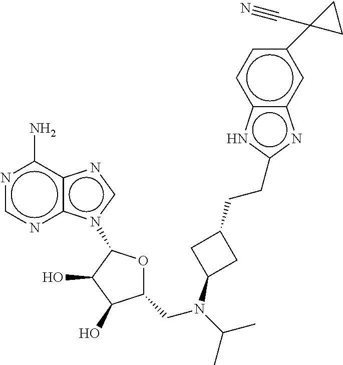 | 1-(2-(2-((1R,3s)-3- ((((2R,3S,4R,5R)-5- (6-amino-9H-purin-9- yl)-3,4- dihydroxytetrahydro- furan-2- yl)methyl)(isopropyl) amino)cyclobutyl) ethyl)-1H- benzo[d]imidazol-5- yl)cyclopropanecar- bonitrile | 572 (M + H)⁺ |
| A137 | 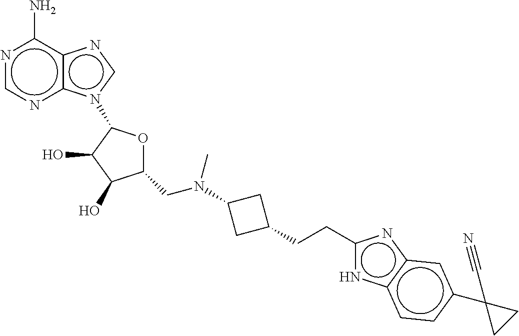 | 1-(2-(2-((1S,3r)-3- ((((2R,3S,4R,5R)-5- (6-amino-9H-purin-9- yl)-3,4- dihydroxytetrahydro- furan-2- yl)methyl((methyl) amino)cyclobutyl) ethyl)-1H- benzo[d]imidazol-5- yl(cyclopropanecar- bonitrile | 544 (M + H)⁺ |
| A138 | 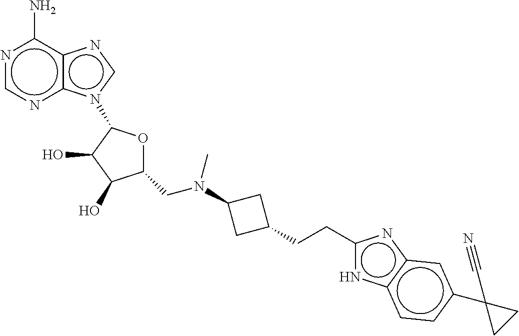 | 1-(2-(2-((1R,3s)-3- ((((2R,3S,4R,5R)-5- (6-amino-9H-purin-9- yl)-3,4- dihydroxytetrahydro- furan-2- yl)methyl)(methyl) amino)cyclobutyl) ethyl)-1H- benzo[d]imidazol-5- yl)cyclopropanecar- bonitrilc | 544 (M + H)⁺ |
| A139 | 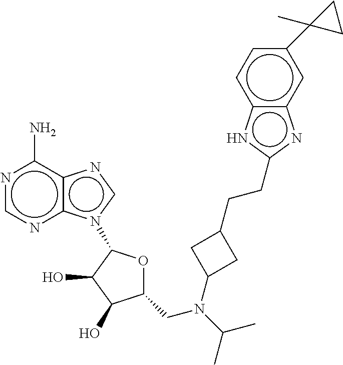 | (2R,3R,4S,5R)-2-(6- amino-9H-purin-9- yl)-5-((isopropyl(3- (2-(5-(1- methylcyclopropyl)- 1H- benzo[d]imidazol-2- yl)ethyl)cyclobutyl) amino)methyl) tetrahydrofuran-3,4-diol | 561 (M + H)⁺ |
| A140 | 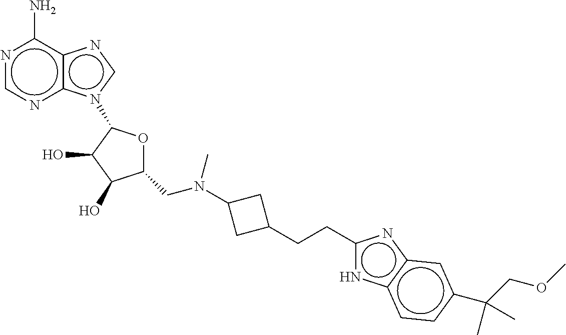 | (2R,3R,4S,5R)-2-(6- amino-9H-purin-9- yl)-5-(((3-(2-(5-(1- methoxy-2- methylpropan-2-yl)- 1H- benzo[d]imidazol-2- yl)ethyl)cyclobutyl) (methyl)amino)methyl) tetrahydrofuran-3,4-diol | 565 (M + H)⁺ |

TABLE 2

| Cmpd. | | |
|---|---|---|
| No. | Structures | Chemical Name |
| B1 | 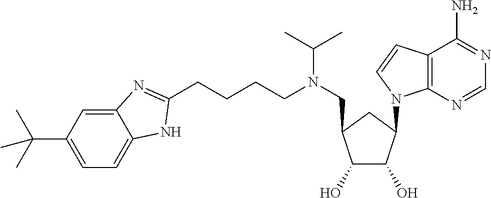 [SMILES: CC(C)N(CCCCc1nc2cc(C(C)(C)C)ccc2[nH]1)C[C@H]1C[C@@H](n2ccc3c(N)ncnc32)[C@H](O)[C@@H]1O] | (1R,2S,3R,5R)-3-(4-amino-7H- pyrrolo[2,3-d]pyrimidin-7-yl)-5- (((4-(5-tert-butyl-1H- benzo[d]imidazol-2- yl)butyl)(isopropyl)amino)- methyl)cyclo- pentane-1,2-diol |
| B2 | 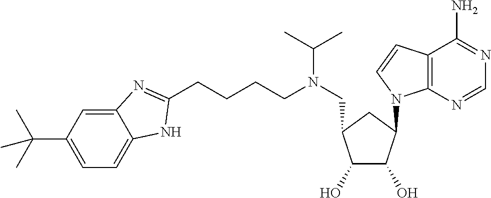 [SMILES: CC(C)N(CCCCc1nc2cc(C(C)(C)C)ccc2[nH]1)C[C@@H]1C[C@@H](n2ccc3c(N)ncnc32)[C@H](O)[C@@H]1O] | (1R,2S,3R,5S)-3-(4-amino-7H- pyrrolo[2,3-d]pyrimidin-7-yl)-5- (((4-(5-test-butyl-1H- benzo[d]imidazol-2- yl)butyl)(isopropyl)amino)- methyl)cyclo- pentane-1,2-diol |
| B3 | 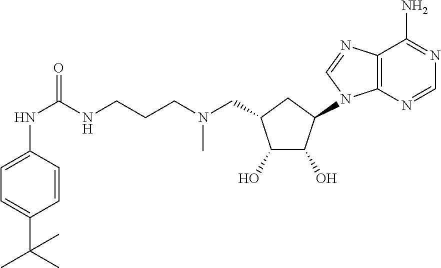 [SMILES: CN(CCCNC(=O)Nc1ccc(C(C)(C)C)cc1)C[C@@H]1C[C@@H](n2cnc3c(N)ncnc32)[C@H](O)[C@@H]1O] | 1-(3-((((1S,2R,3S,4R)-4-(6-amino- 9H-purin-9-yl)-2,3- dihydroxycyclopentyl)methyl)(meth- yl)amino)propyl)-3-(4-(tert- butyl)phenyl)urea |
| B4 | 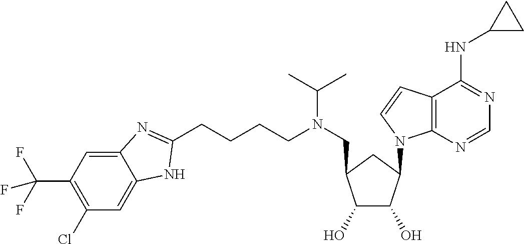 [SMILES: CC(C)N(CCCCc1nc2cc(C(F)(F)F)c(Cl)cc2[nH]1)C[C@H]1C[C@@H](n2ccc3c(NC4CC4)ncnc32)[C@H](O)[C@@H]1O] | (1S,2R,3R,5R)-3-(((4-(6-chloro-5- (trifluoromethyl)-1H- benzo[d]imidazol-2- yl)butyl)(isopropyl)amino)methyl)- 5-(4-(cyclopropylamino)-7H- pyrrolo[2,3-d]pyrimidin-7- yl)cyclopentane-1,2-diol |
| B5 | 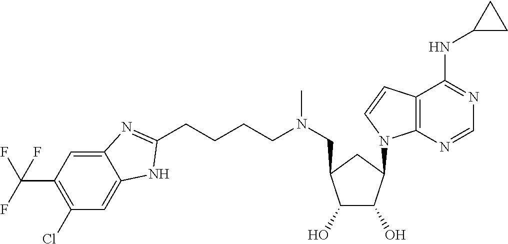 [SMILES: CN(CCCCc1nc2cc(C(F)(F)F)c(Cl)cc2[nH]1)C[C@H]1C[C@@H](n2ccc3c(NC4CC4)ncnc32)[C@H](O)[C@@H]1O] | (1S,2R,3R,5R)-3-(((4-(6-chloro-5- (trifluoromethyl)-1H- benzo[d]imidazol-2- yl)butyl)(methyl)amino)methyl)-5- (4-(cyclopropylamino)-7H- pyrrolo[2,3-d]pyrimidin-7- yl)cyclopentane-1,2-diol |
| B6 | 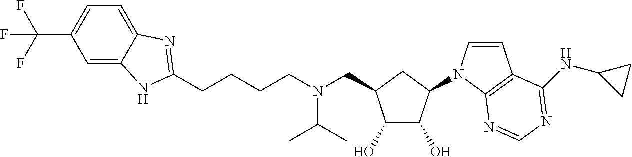 [SMILES: CC(C)N(CCCCc1nc2ccc(C(F)(F)F)cc2[nH]1)C[C@H]1C[C@@H](n2ccc3c(NC4CC4)ncnc32)[C@H](O)[C@@H]1O] | (1R,2S,3R,5R)-3-(4- (cyclopropylamino)-7H-pyrrolo[2,3- d]pyrimidin-7-yl)-5-((isopropyl(4- (6-(trifluoromethyl)-1H- benzo[d]imidazol-2- yl)butyl)amino)methyl)cyclopentane- 1,2-diol |
| B7 | 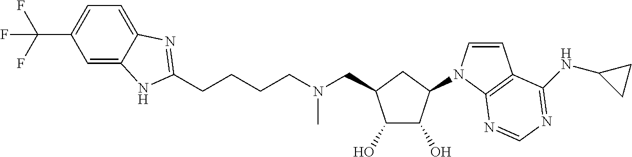 [SMILES: CN(CCCCc1nc2ccc(C(F)(F)F)cc2[nH]1)C[C@H]1C[C@@H](n2ccc3c(NC4CC4)ncnc32)[C@H](O)[C@@H]1O] | (1R,2S,3R,5R)-3-(4- (cyclopropylamino)-7H-pyrrolo[2,3- d]pyrimidin-7-yl)-5-((methyl(4-(6- (trifluoromethyl)-1H- benzo[d]imidazol-2- yl)butyl)amino)methyl)cyclopentane- 1,2-diol |
| B8 | 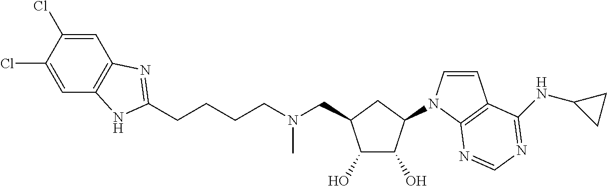 [SMILES: CN(CCCCc1nc2cc(Cl)c(Cl)cc2[nH]1)C[C@H]1C[C@@H](n2ccc3c(NC4CC4)ncnc32)[C@H](O)[C@@H]1O] | (1R,2S,3R,5R)-3-(4- (cyclopropylamino)-7H-pyrrolo[2,3- d]pyrimidin-7-yl)-5-(((4-(5,6- dichloro-1H-benzo[d]imidazol-2- yl)butyl)(methyl)- amino)methyl)cyclo- pentane-1,2-diol |
| B9 | 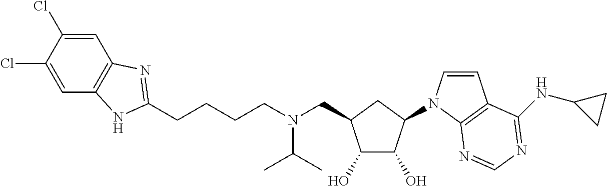 [SMILES: CC(C)N(CCCCc1nc2cc(Cl)c(Cl)cc2[nH]1)C[C@H]1C[C@@H](n2ccc3c(NC4CC4)ncnc32)[C@H](O)[C@@H]1O] | (1R,2S,3R,5R)-3-(4- (cyclopropylamino)-7H-pyrrolo[2,3- d]pyrimidin-7-yl)-5-(((4-(5,6- dichloro-1H-benzo[d]imidazol-2- yl)butyl)(isopropyl)- amino)methyl)cyclo- pentane-1,2-diol trihydrochloride |
| B10 | 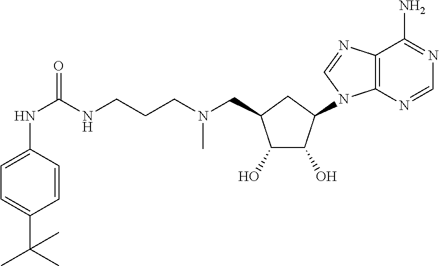 [SMILES: CN(CCCNC(=O)Nc1ccc(C(C)(C)C)cc1)C[C@H]1C[C@@H](n2cnc3c(N)ncnc32)[C@H](O)[C@@H]1O] | 1-(3-((((1R,2R,3S,4R)-4-(6-amino- 9H-purin-9-yl)-2,3- dihydroxycyclopentyl)methyl)(meth- yl)amino)propyl)-3-(4-(tert- butyl)phenyl)urea |
| B11 | 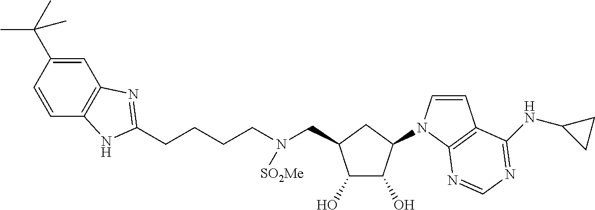 [SMILES: CC(C)(C)c1ccc2[nH]c(CCCCN(C[C@H]3C[C@@H](n4ccc5c(NC6CC6)ncnc54)[C@H](O)[C@@H]3O)S(C)(=O)=O)nc2c1] | N-(4-(5-(tert-butyl)-1H- benzo[d]imidazol-2-yl)butyl)-N- (((1R,2R,3S,4R)-4-(4- (cyclopropylamino)-7H-pyrrolo[2,3- d]pyrimidin-7-yl)-2,3- dihydroxycyclopentyl)- methyl)methane- sulfonamide |
| B12 | 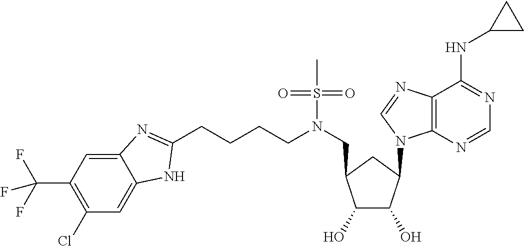 [SMILES: CS(=O)(=O)N(CCCCc1nc2cc(C(F)(F)F)c(Cl)cc2[nH]1)C[C@H]1C[C@@H](n2cnc3c(NC4CC4)ncnc32)[C@H](O)[C@@H]1O] | N-(4-(6-chloro-5-(trifluoromethyl)- 1H-benzo[d]imidazol-2-yl)butyl)-N- (((1R,2R,3S,4R)-4-(4- (cyclopropylamino)-7H-pyrrolo[2,3- d]pyrimidin-7-yl)-2,3- dihydroxycyclopentyl)- methyl)methane- sulfonamide |
| B13 | 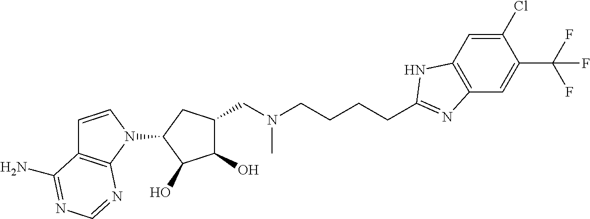 [SMILES: CN(CCCCc1nc2cc(C(F)(F)F)c(Cl)cc2[nH]1)C[C@H]1C[C@@H](n2ccc3c(N)ncnc32)[C@H](O)[C@@H]1O] | (1R,2S,3R,5R)-3-(4-amino-7H- pyrrolo[2,3-d]pyrimidin-7-yl)-5- (((4-(6-chloro-5-(trifluoromethyl)- 1H-benzo[d]imidazol-2- yl)butyl)(methyl)amino)- methyl)cyclo- pentane-1,2-diol |
| B14 | 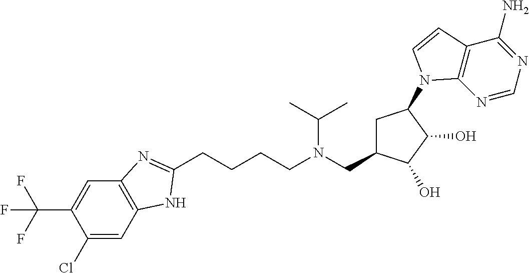 [SMILES: CC(C)N(CCCCc1nc2cc(C(F)(F)F)c(Cl)cc2[nH]1)C[C@H]1C[C@@H](n2ccc3c(N)ncnc32)[C@H](O)[C@@H]1O] | (1R,2S,3R,5R)-3-(4-amino-7H- pyrrolo[2,3-d]pyrimidin-7-yl)-5- (((4-(6-chloro-5-(trifluoromethyl)- 1H-benzo[d]imidazol-2- yl)butyl)(isopropyl)- amino)methyl)cyclo- pentane-1,2-diol |
| B15 | 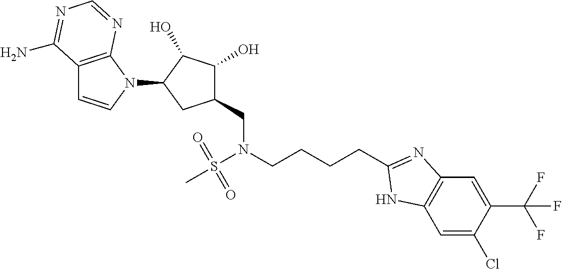 [SMILES: CS(=O)(=O)N(CCCCc1nc2cc(C(F)(F)F)c(Cl)cc2[nH]1)C[C@H]1C[C@@H](n2ccc3c(N)ncnc32)[C@H](O)[C@@H]1O] | N-(((1R,2R,3S,4R)-4-(4-amino-7H- pyrrolo[2,3-d]pyrimidin-7-yl)-2,3- dihydroxycyclopentyl)methyl)-N- (4-(6-chloro-5-(trifluoromethyl)- 1H-benzo[d]imidazol-2- yl)butyl)methanesulfonamide |
| B16 | 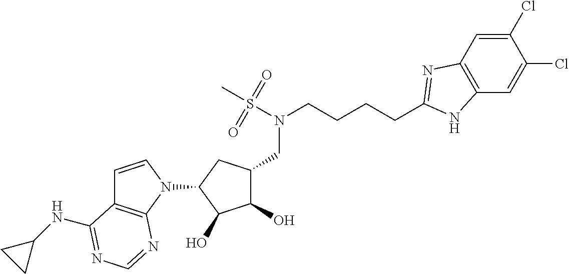 [SMILES: CS(=O)(=O)N(CCCCc1nc2cc(Cl)c(Cl)cc2[nH]1)C[C@H]1C[C@@H](n2ccc3c(NC4CC4)ncnc32)[C@H](O)[C@@H]1O] | N-(((1R,2R,3S,4R)-4-(4- (cyclopropylamino)-7H-pyrrolo[2,3- d]pyrimidin-7-yl)-2,3- dihydroxycyclopentyl)methyl)-N- (4-(5,6-dichloro-1H- benzo[d]imidazol-2- yl)butyl)methanesulfonamide |
| B17 | 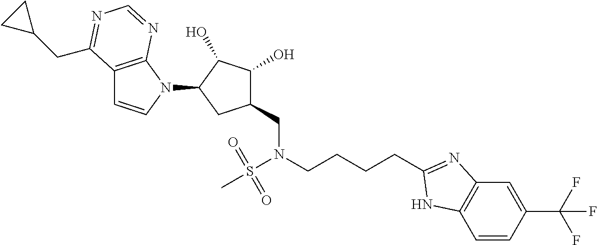 [SMILES: CS(=O)(=O)N(CCCCc1nc2cc(C(F)(F)F)ccc2[nH]1)C[C@H]1C[C@@H](n2ccc3c(CC4CC4)ncnc32)[C@H](O)[C@@H]1O] | N-(((1R,2R,3S,4R)-4-(4- (cyclopropylamino)-7H-pyrrolo[2,3- d]pyrimidin-7-yl)-2,3- dihydroxycyclopentyl)methyl)-N- (4-(5-(trifluoromethyl)-1H- benzo[d]imidazol-2- yl)butyl)methanesulfonamide |

TABLE 3

| | |
|---|---|
| SAH | 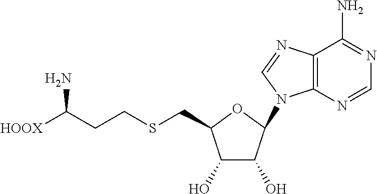 |
| C1 | 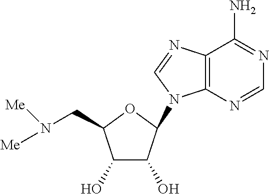 [SMILES: CN(C)C[C@H]1O[C@@H](n2cnc3c(N)ncnc32)[C@H](O)[C@@H]1O] |
| C2 | 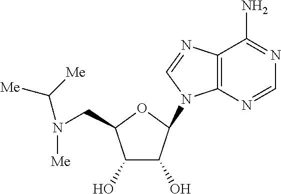 [SMILES: CC(C)N(C)C[C@H]1O[C@@H](n2cnc3c(N)ncnc32)[C@H](O)[C@@H]1O] |
| C64 | 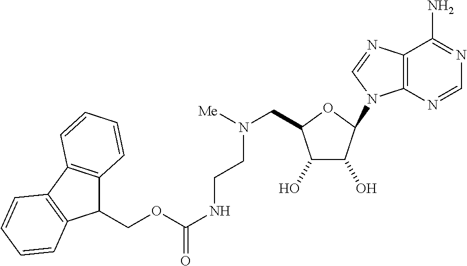 |
| C79 | 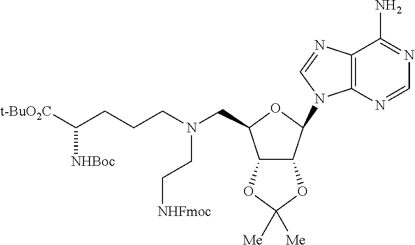 |
| C80 | 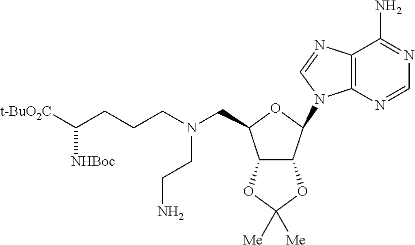 |
| C81 | 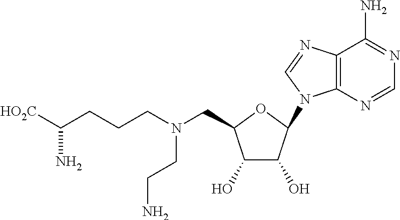 [SMILES: NCCN(CCC[C@H](N)C(=O)O)C[C@H]1O[C@@H](n2cnc3c(N)ncnc32)[C@H](O)[C@@H]1O] |
| C83 | 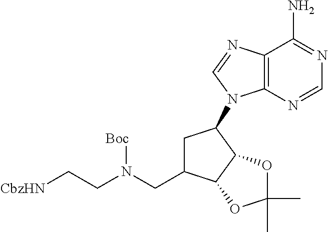 [SMILES: CC(C)(C)OC(=O)N(CCNC(=O)OCc1ccccc1)CC1C[C@@H](n2cnc3c(N)ncnc32)[C@@H]2OC(C)(C)O[C@H]12] |
| C84 | 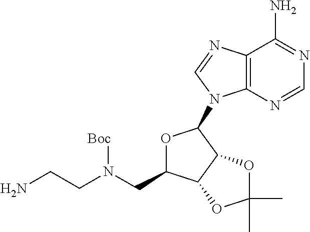 [SMILES: CC(C)(C)OC(=O)N(CCN)C[C@H]1O[C@@H](n2cnc3c(N)ncnc32)[C@@H]2OC(C)(C)O[C@@H]21] |
| C85 | 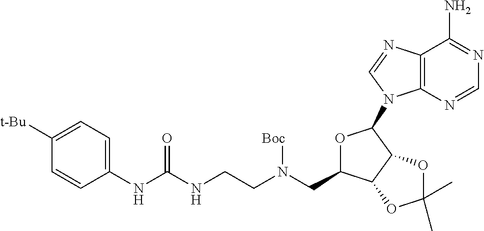 [SMILES: CC(C)(C)OC(=O)N(CCNC(=O)Nc1ccc(C(C)(C)C)cc1)C[C@H]1O[C@@H](n2cnc3c(N)ncnc32)[C@@H]2OC(C)(C)O[C@@H]21] |
| C86 | 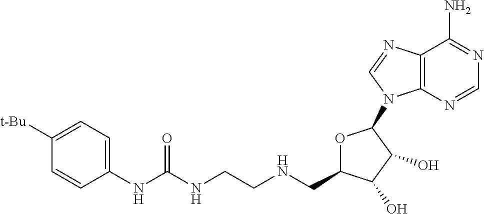 [SMILES: CC(C)(C)c1ccc(NC(=O)NCCNC[C@H]2O[C@@H](n3cnc4c(N)ncnc43)[C@H](O)[C@@H]2O)cc1] |
| C89 | 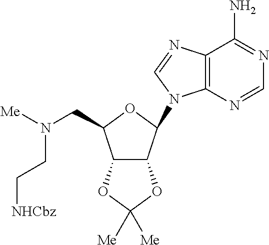 [SMILES: CN(CCNC(=O)OCc1ccccc1)C[C@H]1O[C@@H](n2cnc3c(N)ncnc32)[C@@H]2OC(C)(C)O[C@@H]21] |
| C90 | 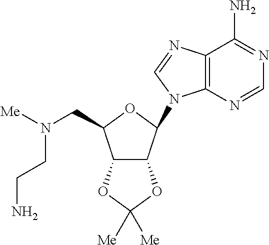 [SMILES: CN(CCN)C[C@H]1O[C@@H](n2cnc3c(N)ncnc32)[C@@H]2OC(C)(C)O[C@@H]21] |
| C91 | 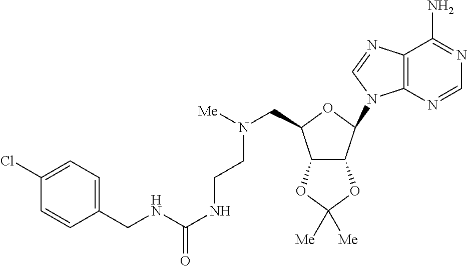 [SMILES: CN(CCNC(=O)NCc1ccc(Cl)cc1)C[C@H]1O[C@@H](n2cnc3c(N)ncnc32)[C@@H]2OC(C)(C)O[C@@H]21] |
| C92 | 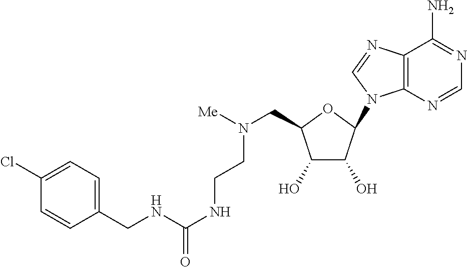 [SMILES: CN(CCNC(=O)NCc1ccc(Cl)cc1)C[C@H]1O[C@@H](n2cnc3c(N)ncnc32)[C@H](O)[C@@H]1O] |
| C93 | 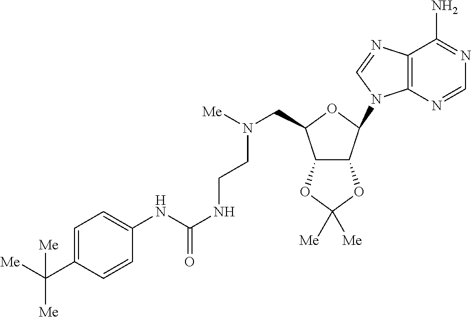 [SMILES: CN(CCNC(=O)Nc1ccc(C(C)(C)C)cc1)C[C@H]1O[C@@H](n2cnc3c(N)ncnc32)[C@@H]2OC(C)(C)O[C@@H]21] |
| C94 | 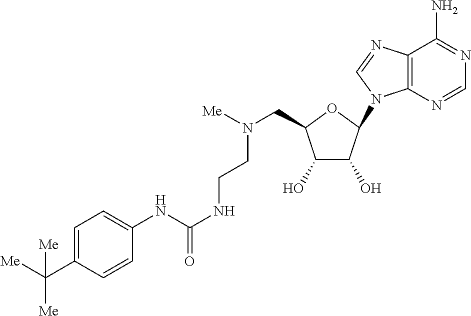 [SMILES: CN(CCNC(=O)Nc1ccc(C(C)(C)C)cc1)C[C@H]1O[C@@H](n2cnc3c(N)ncnc32)[C@H](O)[C@@H]1O] |
| C95 | 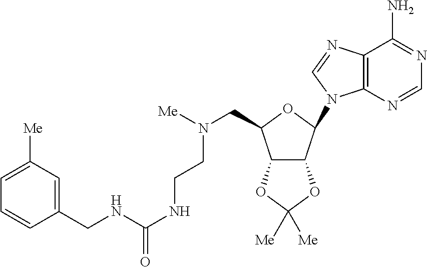 [SMILES: Cc1cccc(CNC(=O)NCCN(C)C[C@H]2O[C@@H](n3cnc4c(N)ncnc43)[C@@H]3OC(C)(C)O[C@@H]32)c1] |
| C96 | 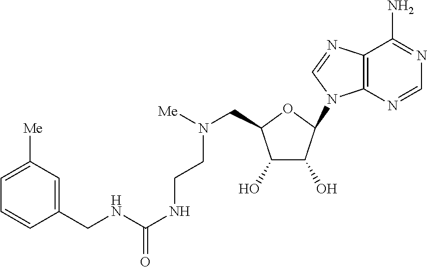 [SMILES: Cc1cccc(CNC(=O)NCCN(C)C[C@H]2O[C@@H](n3cnc4c(N)ncnc43)[C@H](O)[C@@H]2O)c1] |
| C97 | 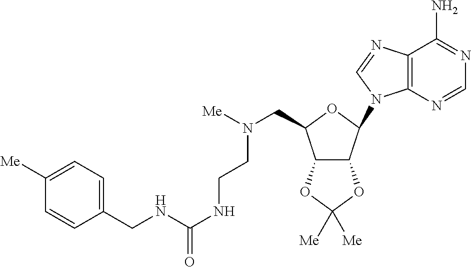 [SMILES: Cc1ccc(CNC(=O)NCCN(C)C[C@H]2O[C@@H](n3cnc4c(N)ncnc43)[C@@H]3OC(C)(C)O[C@@H]32)cc1] |
| C98 | 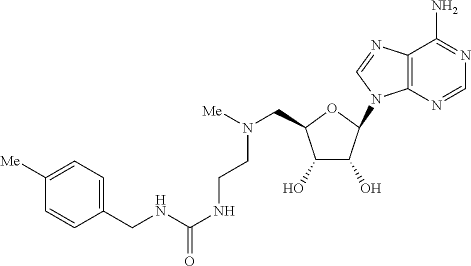 [SMILES: Cc1ccc(CNC(=O)NCCN(C)C[C@H]2O[C@@H](n3cnc4c(N)ncnc43)[C@H](O)[C@@H]2O)cc1] |
| C112 | 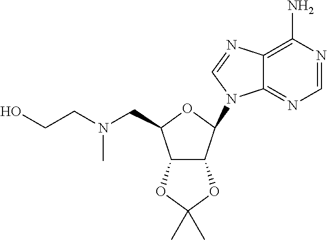 [SMILES: CN(CCO)C[C@H]1O[C@@H](n2cnc3c(N)ncnc32)[C@@H]2OC(C)(C)O[C@@H]21] |
| C113 | 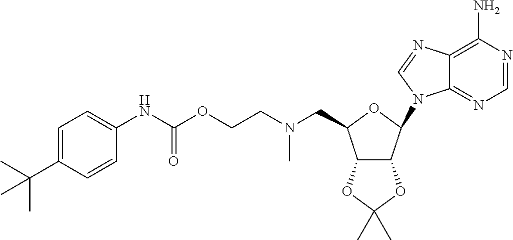 [SMILES: CN(CCOC(=O)Nc1ccc(C(C)(C)C)cc1)C[C@H]1O[C@@H](n2cnc3c(N)ncnc32)[C@@H]2OC(C)(C)O[C@@H]21] |
| C114 | 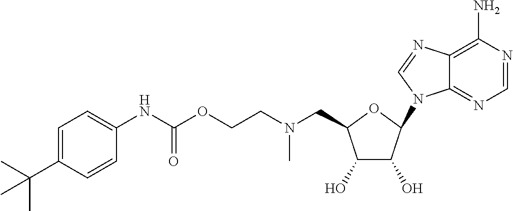 [SMILES: CN(CCOC(=O)Nc1ccc(C(C)(C)C)cc1)C[C@H]1O[C@@H](n2cnc3c(N)ncnc32)[C@H](O)[C@@H]1O] |
| C115 | 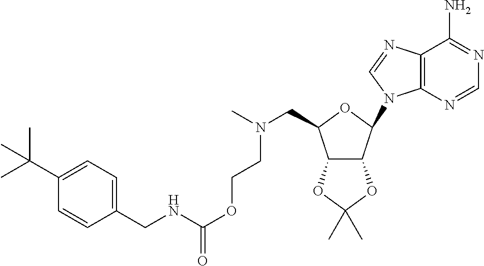 [SMILES: CN(CCOC(=O)NCc1ccc(C(C)(C)C)cc1)C[C@H]1O[C@@H](n2cnc3c(N)ncnc32)[C@@H]2OC(C)(C)O[C@@H]21] |
| C116 | 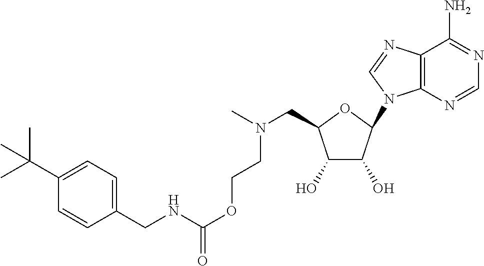 [SMILES: CN(CCOC(=O)NCc1ccc(C(C)(C)C)cc1)C[C@H]1O[C@@H](n2cnc3c(N)ncnc32)[C@H](O)[C@@H]1O] |
| C117 | 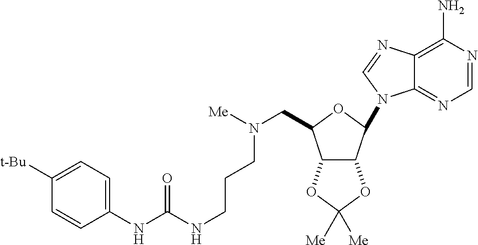 |
| C118 | 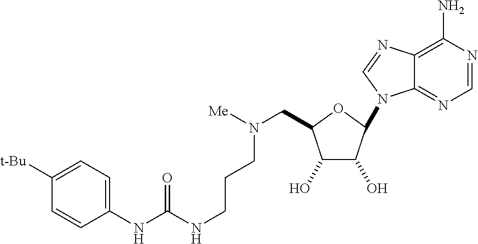 [SMILES: CN(CCCNC(=O)Nc1ccc(C(C)(C)C)cc1)C[C@H]1O[C@@H](n2cnc3c(N)ncnc32)[C@H](O)[C@@H]1O] |
| C122 | 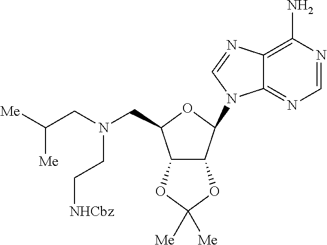 [SMILES: CC(C)CN(CCNC(=O)OCc1ccccc1)C[C@H]1O[C@@H](n2cnc3c(N)ncnc32)[C@@H]2OC(C)(C)O[C@@H]21] |
| C123 | 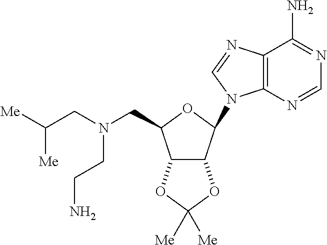 |
| C124 | 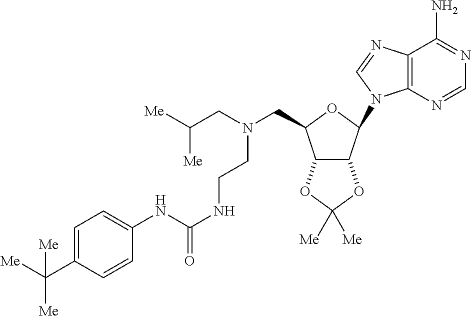 |
| C125 | 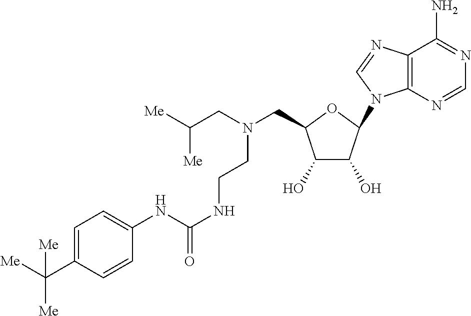 [SMILES: CC(C)CN(CCNC(=O)Nc1ccc(C(C)(C)C)cc1)C[C@H]1O[C@@H](n2cnc3c(N)ncnc32)[C@H](O)[C@@H]1O] |
| C126 | 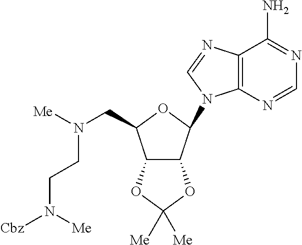 [SMILES: CN(CCN(C)C(=O)OCc1ccccc1)C[C@H]1O[C@@H](n2cnc3c(N)ncnc32)[C@@H]2OC(C)(C)O[C@@H]21] |
| C127 | 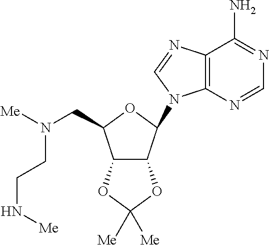 [SMILES: CNCCN(C)C[C@H]1O[C@@H](n2cnc3c(N)ncnc32)[C@@H]2OC(C)(C)O[C@@H]21] |
| C128 | 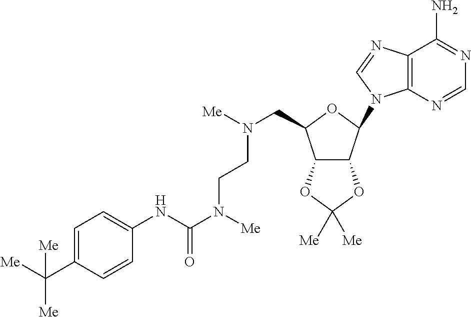 [SMILES: CN(CCN(C)C(=O)Nc1ccc(C(C)(C)C)cc1)C[C@H]1O[C@@H](n2cnc3c(N)ncnc32)[C@@H]2OC(C)(C)O[C@@H]21] |
| C129 | 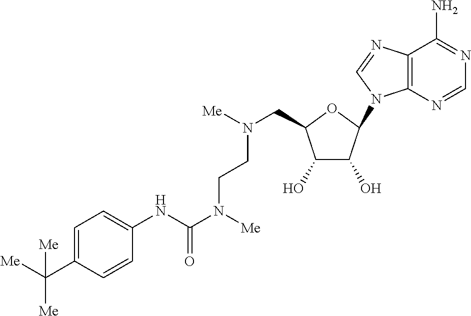 [SMILES: CN(CCN(C)C(=O)Nc1ccc(C(C)(C)C)cc1)C[C@H]1O[C@@H](n2cnc3c(N)ncnc32)[C@H](O)[C@@H]1O] |
| C130 | 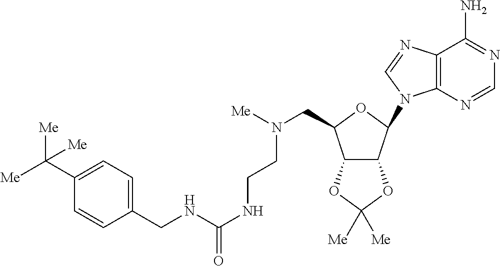 [SMILES: CN(CCNC(=O)NCc1ccc(C(C)(C)C)cc1)C[C@H]1O[C@@H](n2cnc3c(N)ncnc32)[C@@H]2OC(C)(C)O[C@@H]21] |
| C131 | 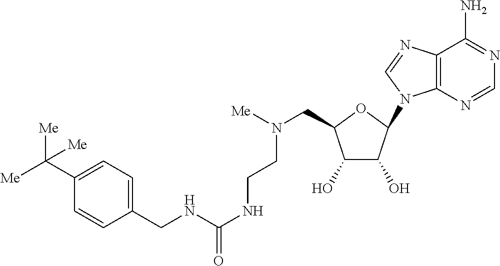 [SMILES: CN(CCNC(=O)NCc1ccc(C(C)(C)C)cc1)C[C@H]1O[C@@H](n2cnc3c(N)ncnc32)[C@H](O)[C@@H]1O] |
| C140 | 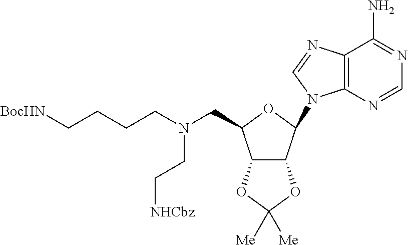 [SMILES: CC(C)(C)OC(=O)NCCCCN(CCNC(=O)OCc1ccccc1)C[C@H]1O[C@@H](n2cnc3c(N)ncnc32)[C@@H]2OC(C)(C)O[C@@H]21] |
| C141 | 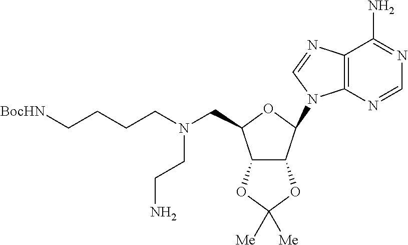 [SMILES: CC(C)(C)OC(=O)NCCCCN(CCN)C[C@H]1O[C@@H](n2cnc3c(N)ncnc32)[C@@H]2OC(C)(C)O[C@@H]21] |
| C142 | 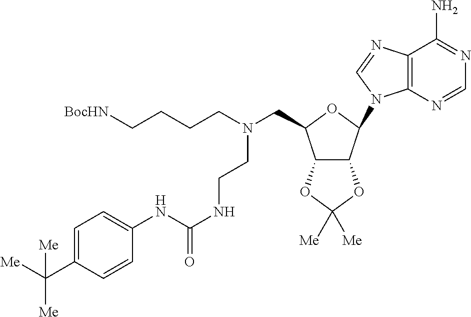 [SMILES: CC(C)(C)OC(=O)NCCCCN(CCNC(=O)Nc1ccc(C(C)(C)C)cc1)C[C@H]1O[C@@H](n2cnc3c(N)ncnc32)[C@@H]2OC(C)(C)O[C@@H]21] |
| C143 | 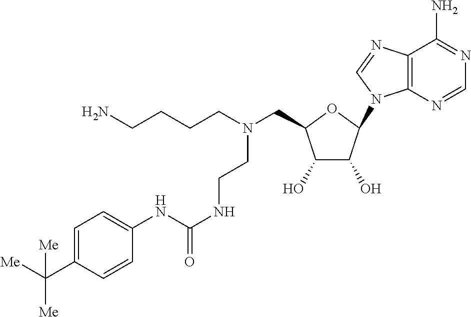 [SMILES: CC(C)(C)c1ccc(NC(=O)NCCN(CCCCN)C[C@H]2O[C@@H](n3cnc4c(N)ncnc43)[C@H](O)[C@@H]2O)cc1] |

TABLE 4

| | |
|---|---|
| D1 | 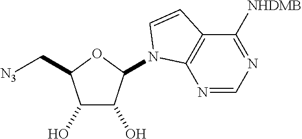 |
| D2 | 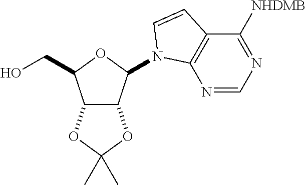 |
| D3 | 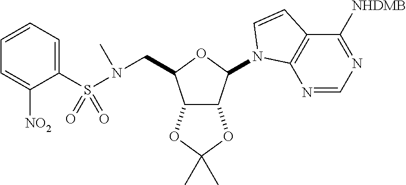 |
| D4 | 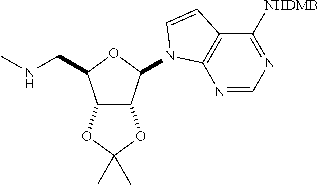 |
| D5 | 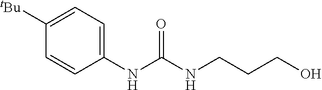 [SMILES: CC(C)(C)c1ccc(NC(=O)NCCCO)cc1] |
| D6 | 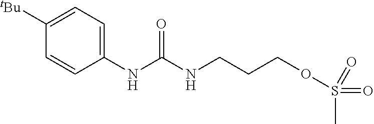 [SMILES: CC(C)(C)c1ccc(NC(=O)NCCCOS(C)(=O)=O)cc1] |
| D7 | 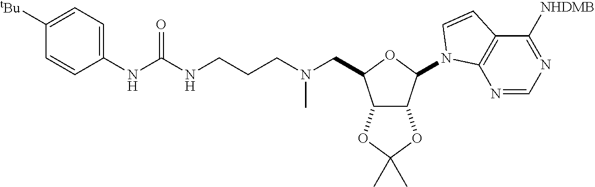 |
| D8 | 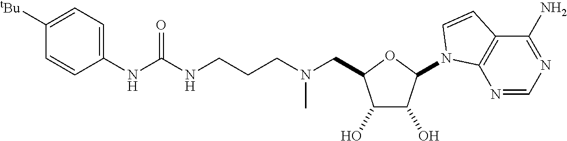 |
| D9 | 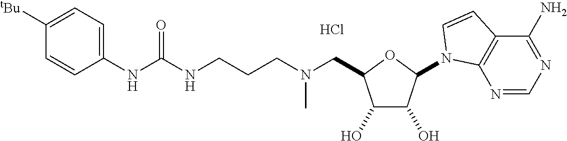 |
| D10 | 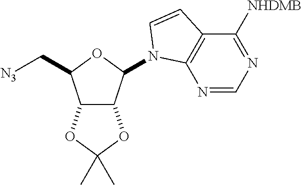 |
| D11 | 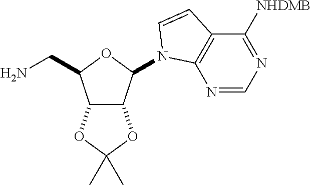 |
| D12 | 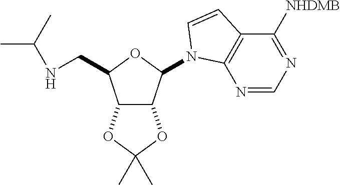 |
| D13 | 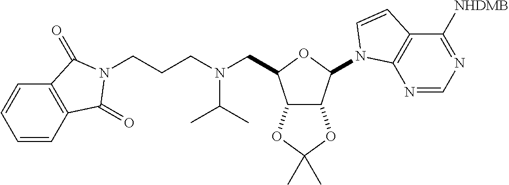 |
| D14 | 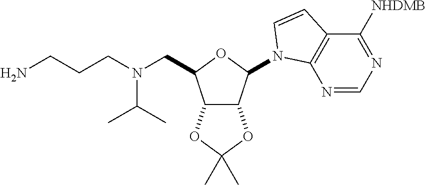 |
| D15 | 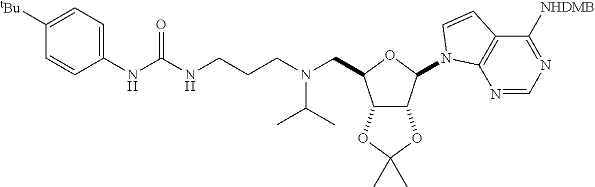 |
| D16 | 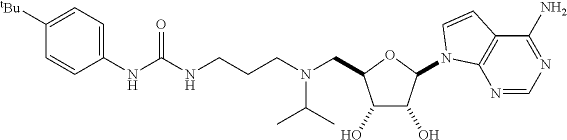 |
| D17 | 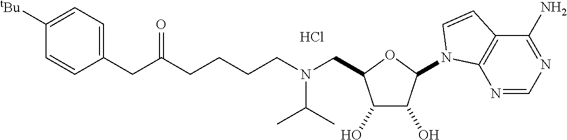 |

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon mono- or multi-ring system having 3 to 30 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, and adamantyl. The term “heterocycloalkyl” refers to a saturated or unsaturated nonaromatic 5-8 membered monocyclic, 8-12 membered bicyclic, or 11-14 membered tricyclic ring system having one or more heteroatoms (such as O, N, S, or Se). Examples of heterocycloalkyl groups include, but are not limited to, piperazinyl, pyrrolidinyl, dioxanyl, morpholinyl, and tetrahydrofuranyl.

The term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl (benzyl)). An “alkylaryl” moiety is an aryl substituted with an alkyl (e.g., methylphenyl).

As used herein, “alkyl linker” is intended to include C₁, C₂, C₃, C₄, C₅ or C₆ straight chain (linear) saturated divalent aliphatic hydrocarbon groups and C₃, C₄, C₅ or C₆ branched saturated aliphatic hydrocarbon groups. For example, C₁-C₆ alkyl linker is intended to include C₁, C₂, C₃, C₄, C₅ and C₆ alkyl linker groups. Examples of alkyl linker include, moieties having from one to six carbon atoms, such as, but not limited to, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂—), n-pentyl (—CH₂CH₂CH₂CH₂CH₂—), s-pentyl (—CHCH₃CH₂CH₂CH₂—) or n-hexyl (—CH₂CH₂CH₂CH₂CH₂CH₂—).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents.

“Aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5- or 6-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The aryl or heteroaryl aromatic ring can be substituted at one or more ring positions with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl).

As used herein, “carbocycle” or “carbocyclic ring” is intended to include any stable monocyclic, bicyclic or tricyclic ring having the specified number of carbons, any of which may be saturated, unsaturated, or aromatic. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In one embodiment, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” includes any ring structure (saturated or partially unsaturated) which contains at least one ring heteroatom (e.g., N, O or S). Examples of heterocycles include, but are not limited to, morpholine, pyrrolidine, tetrahydrothiophene, piperidine, piperazine and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., =0), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R₃) occurs more than one time in any constituent or formula for a compound, its definition at each occurrence is independent of its definition at every other occurrence. Thus, for example, if a group is shown to be substituted with 0-2 R₃ moieties, then the group may optionally be substituted with up to two R₃ moieties and R₃ at each occurrence is selected independently from the definition of R₃. Also, combinations of substituents and/or variables are permissible, but only if such combinations result in stable compounds.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —COOH or its C₁-C₆ alkyl ester.

“Acyl” includes moieties that contain the acyl radical (R—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen bonded to two carbon atoms or heteroatoms. For example, the term includes “alkoxyalkyl,” which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls, and alkthioalkynyls. The term “alkthioalkyls” include moieties with an alkyl, alkenyl, or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” refers to unsubstituted or substituted —NH₂. “Alkylamino” includes groups of compounds wherein nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

Compounds of the present invention (i.e., DOT1L inhibitors) that contain nitrogens can be converted to N-oxides by treatment with an oxidizing agent (e.g., 3-chloroperoxybenzoic acid (mCPBA) and/or hydrogen peroxides) to afford other compounds of the present invention. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present invention can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present invention includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present invention. Furthermore, so-called metabolite which is produced by degradation of the present compound in vivo is included in the scope of the present invention.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present invention may be depicted as different chiral isomers or geometric isomers. It should also be understood that when compounds have chiral isomeric or geometric isomeric forms, all isomeric forms are intended to be included in the scope of the present invention, and the naming of the compounds does not exclude any isomeric forms.

For example, compounds of Formula (I) include those of the following chiral isomers and geometric isomers.

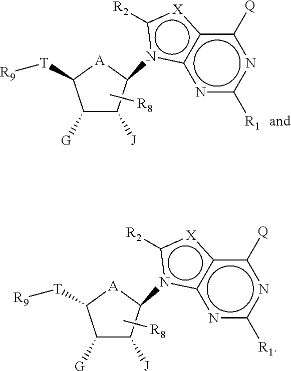

Furthermore, the structures and other compounds discussed in this invention include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. Benzimidazoles also exhibit tautomerism, when the benzimidazole contains one or more substituents in the 4, 5, 6 or 7 positions, the possibility of different isomers arises. For example, 2,5-dimethyl-1H-benzo[d]imidazole can exist in equilibrium with its isomer 2,6-dimethyl-1H-benzo[d]imidazole via tautomerization.

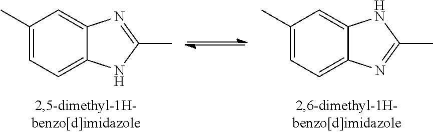

Another example of tautomerism is shown below.

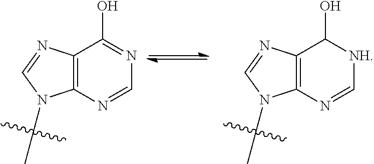 [SMILES: *n1cnc2c(O)ncnc21 ; *n1cnc2c1N=CNC2O]

It is to be understood that the compounds of the present invention may be depicted as different tautomers. It should also be understood that when compounds have tautomeric forms, all tautomeric forms are intended to be included in the scope of the present invention, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Compounds of the invention may be crystalline, semi-crystalline, non-crystalline, amorphous, mesomorphous, etc.

The compounds of any of the Formulae disclosed herein include the compounds themselves, as well as their N-oxides, salts, their solvates, and their prodrugs, if applicable. A salt, for example, can be formed between an anion and a positively charged group (e.g., amino) on the compound or inhibitor (e.g., a substituted nucleoside compound such as a substituted purine or 7-deazapurine compound). Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate. Likewise, a salt can also be formed between a cation and a negatively charged group (e.g., carboxylate) on the compound or inhibitor (e.g., a substituted nucleoside compound such as a substituted purine or 7-deazapurine compound). Suitable cations include sodium ion, potassium ion, magnesium ion, calcium ion, and an ammonium cation such as tetramethylammonium ion. The compound or inhibitor (e.g., a substituted nucleoside compound such as a substituted purine or 7-deazapurine compound) also include those salts containing quaternary nitrogen atoms. Examples of prodrugs include esters and other pharmaceutically acceptable derivatives, which, upon administration to a subject, are capable of providing active substituted nucleoside compound such as a substituted purine or 7-deazapurine.

Additionally, the compounds of the present invention, for example, the salts of the compounds, can exist in either hydrated or unhydrated (the anhydrous) form or as solvates with other solvent molecules. Nonlimiting examples of hydrates include hemihydrates, monohydrates, dihydrates, trihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hemihydrate is formed by the combination of one molecule of water with more than one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (I) are substituted purine compounds or substituted 7-deazapurine compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present invention is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The present invention also provides methods for the synthesis of the compounds of any of the Formulae disclosed herein. The present invention also provides detailed methods for the synthesis of various disclosed compounds of the present invention according to the schemes and the Examples described in WO2012/075381, WO2012/075492, WO2012/082436, WO2012/75500, and U.S. Provisional Application No. 61/682,090, the contents of which are hereby incorporated by reference in their entireties.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial unless otherwise specified so long as the invention remains operable. Moreover, two or more steps or actions can be conducted simultaneously.

The synthetic processes of the invention can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester, or prodrug thereof

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present invention.

One of ordinary skill in the art will note that, during the reaction sequences and synthetic schemes described herein, the order of certain steps may be changed, such as the introduction and removal of protecting groups.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

Preferred protecting groups include, but are not limited to:

For the hydroxyl moiety: TBS, benzyl, THP, Ac

For carboxylic acids: benzyl ester, methyl ester, ethyl ester, allyl ester

For amines: Cbz, BOC, DMB

For diols: Ac (×2) TBS (×2), or when taken together acetonides

For thiols: Ac

For benzimidazoles: SEM, benzyl, PMB, DMB

For aldehydes: di-alkyl acetals such as dimethoxy acetal or diethyl acetyl.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

The following abbreviations are used throughout the specification and are defined below:

AA ammonium acetate

Ac acetyl

ACN acetonitrile

AcOH acetic acid

atm atmosphere

Bn benzyl

BOC tert-butoxy carbonyl

BOP (benzotriazol-1-yloxy)tris(dimethylamino)phosphonium hexafluorophosphate

Cbz benzyloxycarbonyl

COMU (1-cyano-2-ethoxy-2-oxoethylidenaminooxy)dimethylamino-morpholino-carbenium hexafluorophosphate

d days

DBU 1,8-diazabicyclo[5.4.0]undec-7-ene

DCE 1,2 dichloroethane

DCM dichloromethane

DEA diethylamine

DEAD diethyl azodicarboxylate

DIAD diisopropyl azodicarboxylate

DiBAL-H diisobutylalumininium hydride

DIPEA N,N-diisopropylethylamine (Hunig's base)

DMAP N,N-dimethyl-4-aminopyridine

DMB 2,4 dimethoxybenzyl

DMF dimethylformamide

DMSO dimethylsulfoxide

DPPA diphenylphosphoryl azide

EA or EtOAc ethylacetate

EDC or EDCI N-(3-dimethylaminopropyl)-N′-ethylcarbodiimide

ELS Evaporative Light Scattering

ESI− Electrospray negative mode

ESI+ Electrospray positive mode

Et₂O diethyl ether

Et₃N or TEA triethylamine

EtOH ethanol

FA formic acid

FC flash chromatography

h hours

H₂O water

HATU O-(7-azabenzotriazol-1-yl)-N,N,N′,N′-tetramethyluronium hexafluorophosphate

HCl hydrochloric acid

HOAT 1-hydroxy-7-azabenzotriazole

HOBt 1-hydroxybenzotriazole

HOSu N-hydroxysuccinimide

HPLC high performance liquid chromatography

Inj. Vol. injection volume

I.V. or IV intravenous

KHMDs potassium hexamethyldisilazide

LC/MS or LC-MS liquid chromatography mass spectrum

LDA lithium diisopropylamide

LG leaving group

LiHMs lithium hexamethyldisilazide

M Molar

m/z mass/charge ratio

m-CPBA meta-chloroperbenzoic acid

MeCN acetonitrile

MeOD d₄-methanol

MeOH methanol

MgSO₄ magnesium sulfate

min minutes

MS mass spectrometry or mass spectrum

Ms mesyl

MsCl methanesulfonyl chloride

MsO mesylate

MWI microwave irradiation

Na₂CO₃ sodium carbonate

NaHCO₃ sodium bicarbonate

NaHMDs sodium hexamethyldisilazide

NaOH sodium hydroxide

NIS N-iodosuccinimide

NMR Nuclear Magnetic Resonance

o/n or O/N overnight

PE petroleum ether

PG protecting group

PKMT protein lysine methyltransferase

PMB para-methoxybenzyl

PMT protein methyltransferase

PPAA 1-propanephosphonic acid cyclic anhydride

ppm parts per million

prep HPLC preparative high performance liquid chromatography

prep TLC preparative thin layer chromatography

p-TsOH para-toluenesulfonic acid

rt or RT room temperature

SAH S-adenosylhomocysteine

SAM S-adenosylmethionine

SAR structure activity relationship

SEM 2-(trimethylsilyl)ethoxymethyl

SEMC1 (trimethylsilyl)ethoxymethyl chloride

SFC supercritical chromatography

SGC silica gel chromatography

SPR surface plasmon resonance

STAB sodium triacetoxyborohydride

TBAF tetra-n-butylammonium fluoride

TFA trifluoroacetic acid

TfO triflate

THF tetrahydrofuran

THP tetrahydropyran

TLC thin layer chromatography

Ts tosyl

TsOH tosic acid

UV ultraviolet

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions can be conducted simultaneously.

Compounds suitable for the methods of the invention, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described herein.

To further assess a compound's drug-like properties, measurements of inhibition of cytochrome P450 enzymes and phase II metabolizing enzyme activity can also be measured either using recombinant human enzyme systems or more complex systems like human liver microsomes. Further, compounds can be assessed as substrates of these metabolic enzyme activities as well. These activities are useful in determining the potential of a compound to cause drug-drug interactions or generate metabolites that retain or have no useful antimicrobial activity.

To get an estimate of the potential of the compound to be orally bioavailable, one can also perform solubility and Caco-2 assays. The latter is a cell line from human epithelium that allows measurement of drug uptake and passage through a Caco-2 cell monolayer often growing within wells of a 24-well microtiter plate equipped with a 1 micron membrane. Free drug concentrations can be measured on the basolateral side of the monolayer, assessing the amount of drug that can pass through the intestinal monolayer. Appropriate controls to ensure monolayer integrity and tightness of gap junctions are needed. Using this same system one can get an estimate of P-glycoprotein mediated efflux. P-glycoprotein is a pump that localizes to the apical membrane of cells, forming polarized monolayers. This pump can abrogate the active or passive uptake across the Caco-2 cell membrane, resulting in less drug passing through the intestinal epithelial layer. These results are often done in conjunction with solubility measurements and both of these factors are known to contribute to oral bioavailability in mammals. Measurements of oral bioavailability in animals and ultimately in man using traditional pharmacokinetic experiments will determine the absolute oral bioavailability.

Experimental results can also be used to build models that help predict physical-chemical parameters that contribute to drug-like properties. When such a model is verified, experimental methodology can be reduced, with increased reliance on the model predictability.

A composition of the present invention comprises a compound of Formula (I), or a pharmaceutically acceptable salt thereof, and one or more therapeutic agents. The present invention provides for the administration of a compound of Formula (I) or a pharmaceutically acceptable salt thereof, and one or more therapeutic agents as a co-formulation or separate formulations, wherein the administration of formulations is simultaneous, sequential, or in alternation. In one embodiment, the one or more therapeutic agents can be an agent that is recognized in the art as being useful to treat the disease or condition being treated by the composition of the present invention. In another embodiment, the one or more therapeutic agents can be an agent that is not recognized in the art as being useful to treat the disease or condition being treated by the composition of the present invention. In one aspect, the other therapeutic agents can be an agent that imparts a beneficial attribute to the composition of the present invention (e.g., an agent that affects the viscosity of the composition). The beneficial attribute to the composition of the present invention includes, but is not limited to, pharmacokinetic or pharmacodynamic co-action resulting from the combination of a compound of Formula (I) and one or more therapeutic agents.

In some embodiments, the one or more therapeutic agents can be anticancer agents or chemotherapeutic agents. For example, the one or more therapeutic agents can be selected from Ara-C, Daunorubicin, Azacitidine, Decitabine, Panobinostat, Vidaza, Mitoxantrone, Methotrexate, Mafosfamide, Prednisolone, Vincristine, Lenalidomide, Hydroxyurea, Menin-MLL inhibitor MI-2, JQ1, IBET151, Panobinostat, Vorinostat, Quizartinib, Midostaurin, Tranylcypromine, LSD1 inhibitor II, Navitoclax, Velcade or functional analogs, derivatives, prodrugs, and metabolites thereof. Preferably, the therapeutic agent is Ara-C or Daunorubicin or functional analogs, derivatives, produgs, and metabolites thereof

In some embodiments, the therapeutic agents are topoisomerase inhibitors (e.g., Mitoxantrone), hypomethylating agents (e.g., Decitabine or Vidaza), Menin inhibitors (e.g., MI-2), Bromodomain inhibitors (e.g., IBET-151), HDAC inhibitors (e.g., Panobinostat), Bcl-2 inhibitors (e.g., Navitoclax) or FLT inhibitors (e.g., Quizartinib).

In some embodiments, the therapeutic agents are Bromodomain inhibitors (e.g., IBET-151) or Menin inhibitors (e.g., MI-2).

The therapeutic agents set forth below are for illustrative purposes and not intended to be limiting. The present invention includes at least one therapeutic agent selected from the lists below. The present invention can include more than one therapeutic agent, e.g., two, three, four, or five therapeutic agents such that the composition of the present invention can perform its intended function.

In one embodiment, the other therapeutic agent is an anticancer agent. In one embodiment, the anticancer agent is a compound that affects histone modifications, such as an HDAC inhibitor. In certain embodiments, an anticancer agent is selected from the group consisting of chemotherapeutics (such as 2CdA, 5-FU, 6-Mercaptopurine, 6-TG, Abraxane™, Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Tykerb®, Velcade® and Zevalin™); corticosteroids, (such as dexamethasone sodium phosphate, DeltaSone® and Delta-Cortef®); hormonal therapies (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); and radiopharmaceuticals (such as IodotopeO, Metastron®, Phosphocol® and Samarium SM-153).

In another embodiment, the other therapeutic agent is a chemotherapeutic agent (also referred to as an anti-neoplastic agent or anti-proliferative agent), selected from the group including an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, anti-neoplastic or anti-proliferative agent listed in www.cancer.org/docroot/cdg/cdg_{—}0.asp.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine131 tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; Pkc412; bryostatin; KAI-9803; SF1126; or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristine, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexalen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine—1 M potassium oxonate), or lovastatin.

In another aspect, the other therapeutic agent is a chemotherapeutic agent or a cytokine such as G-CSF (granulocyte colony stimulating factor).

In yet another aspect, the other therapeutic agents can be standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), or CMFP (cyclophosphamide, methotrexate, 5-fluorouracil and prednisone).

In another aspect, the other therapeutic agents can be an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-B, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

In one embodiment, a composition of the present invention includes a compound of Formula (I) or a pharmaceutically acceptable salt thereof, and one or more anticancer agents. Anticancer agents include, for example, Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, Navitoclax, or functional analogs, derivatives, produgs, and metabolites thereof

The present invention provides methods for combination therapy in which a composition comprising a compound of Formula (I) or a pharmaceutically acceptable salt thereof, and one or more other therapeutic agents are administered to a subject in need for treatment of a disease or cancer. The combination therapy can also be administered to cancer cells to inhibit proliferation or induce cell death.

The present invention includes the combination therapy of administering a compound of Formula (I) or a pharmaceutically acceptable salt thereof, and anticancer agents, where the anticancer agents are Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, Navitoclax.

In one aspect, a compound of Formula (I) or a pharmaceutically acceptable salt thereof and one or more therapeutic agents are administered simultaneously or sequentially.

In one aspect, a compound of Formula (I) or a pharmaceutically acceptable salt thereof is administered prior to administration of the composition of the invention comprising a compound of Formula (I) or a pharmaceutically acceptable salt thereof, and one or more therapeutic agents.

In one aspect, one or more therapeutic agents are administered prior to administration of a composition of the invention comprising a compound of Formula (I) or a pharmaceutically acceptable salt thereof and one or more therapeutic agents. The one or more therapeutic agents are administered either in a single composition or in two or more compositions, e.g. administered simultaneously, sequentially, or in alternation.

In one aspect, a compound of Formula (I) or a pharmaceutically acceptable salt thereof is administered prior to administration of one or more therapeutic agents, such that the one or more therapeutic agents are administered either in a single composition or in two or more compositions, e.g. administered simultaneously, sequentially, or in alternation.

In one aspect, one or more therapeutic agents are administered prior to administration of a compound of Formula (I) or a pharmaceutically acceptable salt thereof. The one or more therapeutic agents are administered either in a single composition or in two or more compositions, e.g. administered simultaneously, sequentially, or in alternation.

In one aspect, a compound of Formula (I) or a pharmaceutically acceptable salt thereof and the one or more therapeutic agents are administered sequentially. It should be appreciated that the one or more therapeutic agents can be administered one or more hours, or one or more days after a compound of Formula (I) or a pharmaceutically acceptable salt thereof is administered. Alternatively, the one or more therapeutic agents can be administered one or more hours, or one or more days prior to a compound of Formula (I) or a pharmaceutically acceptable salt thereof is administered.

In some embodiments, the one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) or a pharmaceutically acceptable salt thereof. In some embodiments, the one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more prior to the administration of a compound of Formula (I) or a pharmaceutically acceptable salt thereof.

In some embodiments, the composition comprising a compound of Formula (I) or a pharmaceutically acceptable salt thereof and one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) or a pharmaceutically acceptable salt thereof.

In some embodiments, the composition comprising a compound of Formula (I) or a pharmaceutically acceptable salt thereof and one or more therapeutic agents are administered 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of the one or more therapeutic agents.

In some embodiments, the one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more after the administration of a compound of Formula (I) or a pharmaceutically acceptable salt thereof. In some embodiments, the one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more prior to the administration of a compound of Formula (I) or a pharmaceutically acceptable salt thereof. In some embodiments, the composition comprising a compound of Formula (I) or a pharmaceutically acceptable salt thereof and one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more after the administration of a compound of Formula (I) or a pharmaceutically acceptable salt thereof

In some embodiments, the composition comprising a compound of Formula (I) or a pharmaceutically acceptable salt thereof and one or more therapeutic agents are administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or more after the administration of the one or more therapeutic agents.

It should be appreciated that the one or more therapeutic agents or the composition comprising a compound of Formula (I) or a pharmaceutically acceptable salt thereof and one or more therapeutic agents, can be administered to a subject after the level in a subject of a compound of Formula (I) or a pharmaceutically acceptable salt thereof that has been administered to the subject has decreased. Thus, for instance, a compound of Formula (I) or a pharmaceutically acceptable salt thereof is administered to a subject and the one or more therapeutic agents are administered after the level of administered compound of Formula (I) or a pharmaceutically acceptable salt thereof is less than 90% of the initial level, less than 80% of the initial level, less than 70% of the initial level, less than 60% of the initial level, less than 50% of the initial level, less than 40% of the initial level, less than 30% of the initial level, less than 20% of the initial level or less than 10% of the initial level. In some embodiments, a compound of Formula (I) or a pharmaceutically acceptable salt thereof that has been administered to a subject can no longer be detected in a subject prior to administration of the one or more therapeutic agents.

It should be appreciated that a compound of Formula (I) or a pharmaceutically acceptable salt thereof or the composition comprising a compound of Formula (I) or a pharmaceutically acceptable salt thereof and one or more therapeutic agents, can be administered to a subject after the level(s) in a subject one or more therapeutic agents that have been administered to the subject has decreased. For example, one or more therapeutic agents are administered to a subject and a compound of Formula (I) or a pharmaceutically acceptable salt thereof is administered after the level of administered one or more therapeutic agents is less than 90% of the initial level, less than 80% of the initial level, less than 70% of the initial level, less than 60% of the initial level, less than 50% of the initial level, less than 40% of the initial level, less than 30% of the initial level, less than 20% of the initial level or less than 10% of the initial level. In some embodiments, one or more therapeutic agents that have been administered to a subject can no longer be detected in a subject prior to administration of a compound of Formula (I) or a pharmaceutically acceptable salt thereof.

For example, the compound has the formula

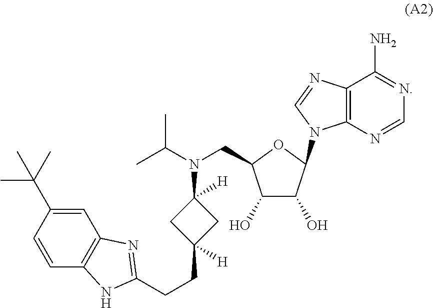 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

For example, the compound has the formula

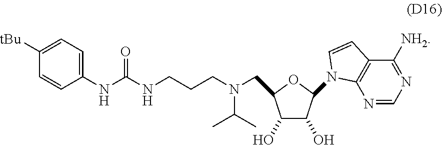 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

In one aspect, the disclosure provides methods for sensitizing or priming a subject to administration of one or more therapeutic agents (e.g., anti-cancer agents). In some embodiments, a subject is sensitized or primed to one or more therapeutic agents (e.g., anti-cancer agents) by administering a compound of Formula (I) or a pharmaceutically acceptable salt thereof. Thus, in one aspect, a compound of Formula (I) or a pharmaceutically acceptable salt thereof is administered to a subject resulting in the sensitization or priming of the subject after which the one or more therapeutic agents (e.g., anti-cancer agents) or the composition comprising a compound of Formula (I) or a pharmaceutically acceptable salt thereof and one or more therapeutic agents, are administered. While not being limited to a specific mechanism it is thought that a subject is sensitized by the administration of a compound of Formula (I) or a pharmaceutically acceptable salt thereof, through a durable altered chromatin state caused by the administration of administering a compound of Formula (I) or a pharmaceutically acceptable salt thereof. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) or a pharmaceutically acceptable salt thereof

In one aspect, the disclosure provides methods for sensitizing or priming a subject to administration of a compound of Formula (I) or a pharmaceutically acceptable salt thereof. In some embodiments, a subject is sensitized or primed for responding to a compound of Formula (I) or a pharmaceutically acceptable salt thereof by administering one or more therapeutic agents (e.g., anti-cancer agents). Thus, in one aspect, one or more therapeutic agents or the composition comprising a compound of Formula (I) or a pharmaceutically acceptable salt thereof and one or more therapeutic agents, are administered to a subject prior to the administration of a compound of Formula (I) or a pharmaceutically acceptable salt thereof, resulting in the sensitization or priming of the subject. Consequently the subject is more sensitive to a compound of Formula (I) or a pharmaceutically acceptable salt thereof

In some embodiments, the administration of a compound of Formula (I) or a pharmaceutically acceptable salt thereof results in a biological effect prior to the administration of the one or more therapeutic agents (e.g., anti-cancer agents) or the composition comprising a compound of Formula (I) or a pharmaceutically acceptable salt thereof and one or more therapeutic agents. In some embodiments, the one or more therapeutic agents (e.g., anti-cancer agents) are not administered until 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) or a pharmaceutically acceptable salt thereof has resulted in a biological effect. In some embodiments, the biological effect is a reduction of H3K79 methylmark, maturation or induction of blast cells, apoptosis of leukemic blast cells, resolution of fevers, cachexia or leukemia cutis and/or restoration of normal haemoatopoiesis. It should be appreciated that more than one biological effect may result from the administration of a compound of Formula (I) or a pharmaceutically acceptable salt thereof. In some embodiments, the biological effect is a reduction of H3K79 methyl mark. In some embodiments, the biological effect is a reduction of H3K79 methyl mark to at least 90%, 80%, 70%, 60%, 50%, 40%, 30%, 20%, 10% or less compared to untreated control levels. In some embodiments, the H3K79 methyl mark must be at least 90%, 80%, 70%, 60%, 50%, 40%, 30%, 20%, 10% or less compared to untreated control levels prior to the addition of the one or more therapeutic agents. In some embodiments, the biological effect is the maturation or differentiation of leukemic blast cells. In some embodiments, at least 20% of leukemic blast cells have undergone maturation or differentiation, at least 50% of leukemic blast cells have undergone maturation or differentiation, or at least 80% of leukemic blast cells have undergone maturation or differentiation prior to the addition of the one or more therapeutic agents. In some embodiments, the biological effect is the apoptosis of leukemic blast cells. In some embodiments, at least 20%, 30%, 40%, 50%, 60%, 70%, 80%, or 90% of the leukemic blast cells undergo cell death or apoptosis prior to administration of the one or more therapeutic agents. In some embodiments, the biological effect is the resolution of fever, resolution of cachexia and/or resolution of leukemia cutis. In some embodiments, fever, cachexia and/or leukemia cutis is resolved prior to administration of the one or more therapeutic agents. In some embodiments, the biological effect is the restoration of normal haematopoiesis. In some embodiments, normal haematopoiesis is restored prior to administration of the one or more therapeutic agents.

In some embodiments, the administration of one or more therapeutic agents (e.g., anti-cancer agents) results in a biological effect prior to the administration of a compound of Formula (I) or a pharmaceutically acceptable salt thereof or the composition comprising a compound of Formula (I) or a pharmaceutically acceptable salt thereof and one or more therapeutic agents. In some embodiments, a compound of Formula (I) or a pharmaceutically acceptable salt thereof is not administered until 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of one or more therapeutic agents have resulted in a biological effect. In some embodiments, the biological effect is a reduction of H3K79 methylmark, maturation or induction of blast cells, apoptosis of leukemic blast cells, resolution of fevers, cachexia or leukemia cutis and/or restoration of normal haemoatopoiesis. It should be appreciated that more than one biological effect may result from the administration of one or more therapeutic agents. In some embodiments, the biological effect is a reduction of H3K79 methyl mark. In some embodiments, the biological effect is a reduction of H3K79 methyl mark to at least 90%, 80%, 70%, 60%, 50%, 40%, 30%, 20%, 10% or less compared to untreated control levels. In some embodiments, the H3K79 methyl mark must be at least 90%, 80%, 70%, 60%, 50%, 40%, 30%, 20%, 10% or less compared to untreated control levels prior to the addition of a compound of Formula (I) or a pharmaceutically acceptable salt thereof

In some embodiments, the biological effect is the maturation or differentiation of leukemic blast cells. In some embodiments, at least 20% of leukemic blast cells have undergone maturation or differentiation, at least 50% of leukemic blast cells have undergone maturation or differentiation, or at least 80% of leukemic blast cells have undergone maturation or differentiation prior to the addition of a compound of Formula (I) or a pharmaceutically acceptable salt thereof

In some embodiments, the biological effect is the apoptosis of leukemic blast cells. In some embodiments, at least 20%, 30%, 40%, 50%, 60%, 70%, 80%, or 90% of the leukemic blast cells undergo cell death or apoptosis prior to administration of a compound of Formula (I) or a pharmaceutically acceptable salt thereof. In some embodiments, the biological effect is the resolution of fever, resolution of cachexia and/or resolution of leukemia cutis. In some embodiments, fever, cachexia and/or leukemia cutis is resolved prior to administration of a compound of Formula (I) or a pharmaceutically acceptable salt thereof. In some embodiments, the biological effect is the restoration of normal haematopoiesis. In some embodiments, normal haematopoiesis is restored prior to administration of a compound of Formula (I) or a pharmaceutically acceptable salt thereof.

In some embodiments, a subject is evaluated after the administration of a compound of Formula (I) or a pharmaceutically acceptable salt thereof for any biological effects prior to administration of one or more therapeutic agents (e.g., anti-cancer agents) or the composition comprising a compound of Formula (I) or a pharmaceutically acceptable salt thereof and one or more therapeutic agents. In some embodiments, the one or more therapeutic agents are administered only if the evaluated biological effect has reached a certain predetermined level or activity. In some embodiments, the biological effect is maturation or induction of blast cells, apoptosis of leukemic blast cells, resolution of fever, cachexia or leukemia cutis and/or restoration of normal haemoatopoiesis. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of a compound of Formula (I) or a pharmaceutically acceptable salt thereof.

In some embodiments, a subject is evaluated after the administration of one or more therapeutic agents (e.g., anti-cancer agents) for any biological effects prior to administration of a compound of Formula (I) or a pharmaceutically acceptable salt thereof or the composition comprising a compound of Formula (I) or a pharmaceutically acceptable salt thereof and one or more therapeutic agents, In some embodiments, a compound of Formula (I) or a pharmaceutically acceptable salt thereof is administered only if the evaluated biological effect has reached a certain predetermined level or activity. In some embodiments, the biological effect is maturation or induction of blast cells, apoptosis of leukemic blast cells, resolution of fever, cachexia or leukemia cutis and/or restoration of normal haemoatopoiesis. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the administration of one or more therapeutic agents.

In certain aspects of the invention, the sensitization or priming by a compound of Formula (I) results in the need for lower therapeutically effective amounts of the sequential therapeutic agent. It should be appreciated that in certain embodiments the sensitization would result in a synergistic effect as described herein between the compound of Formula (I) and the therapeutic agent, such as a standard of care agent.

In certain aspects of the invention, the sensitization or priming by one or more therapeutic agents results in the need for lower therapeutically effective amounts of the sequential administration of a compound of Formula (I) or a pharmaceutically acceptable salt thereof or a composition of the invention. It should be appreciated that in certain embodiments the sensitization would result in a synergistic effect as described herein between the compound of Formula (I) and the therapeutic agent, such as a standard of care agent.

In one aspect, a compound of Formula (I) or a pharmaceutically acceptable salt thereof is administered continuously. In some embodiments, the compound of Formula (I) or a pharmaceutically acceptable salt thereof is administered continuously for at least 7, 14, 21, 28, 35, 42, 47, 56 or 64 days. In some embodiments, the compound of Formula (I) or a pharmaceutically acceptable salt thereof is administered without a drug holiday.

In one aspect, a compound of Formula (I) or a pharmaceutically acceptable salt thereof and one or more therapeutic agents are administered simultaneously or sequentially. In some embodiments, a compound of Formula (I) or a pharmaceutically acceptable salt thereof and the one or more therapeutic agents are administered continuously. In some embodiments, the compound of Formula (I) or a pharmaceutically acceptable salt thereof and the one or more therapeutic agents are administered continuously for at least 7, 14, 21, 28, 35, 42, 47, 56 or 64 days. In some embodiments, the compound of Formula (I) or a pharmaceutically acceptable salt thereof and the one or more therapeutic agents are administered without a drug holiday.

In one aspect, a compound of Formula (I) or a pharmaceutically acceptable salt thereof is administered continuously while the one or more therapeutic agents are not administered continuously. In some embodiments, the compound of Formula (I) or a pharmaceutically acceptable salt thereof is administered continuously for at least 7, 14, 21, 28, 35, 42, 47, 56 or 64 days while the one or more therapeutic agents is not administered continuously for at least 7, 14, 21, 28, 35, 42, 47, 56 or 64 days. In some embodiments, the compound of Formula (I) or a pharmaceutically acceptable salt thereof is administered without a drug holiday while the one or more therapeutic agents are administered with a drug holiday. It should be appreciated that the compound of Formula (I) or a pharmaceutically acceptable salt thereof and the one or more therapeutic agents can be administered using different regimens. Thus, for instance, the compound of Formula (I) or a pharmaceutically acceptable salt thereof may be administered continuously while the one or more therapeutic agents may be administered as one dose or a defined number of multiple doses. The administration regimen of the one or more therapeutic agents may be as indicated on a label (e.g., if the therapeutic agent is a regulated drug) and/or may be modified to optimize the biological effect of the one or more therapeutic agents and/or the biological effect of the combination of the one or more therapeutic agents and the compound of Formula (I) or a pharmaceutically acceptable salt thereof

In one aspect, a compound of Formula (I) or a pharmaceutically acceptable salt thereof and one or more therapeutic agents are administered sequentially (either compound first or agent first). It should be appreciated that the compound of Formula (I) or a pharmaceutically acceptable salt thereof may be administered according to any of the methods described herein, such as by continuous administration, and/or administration without a drug holiday, prior to or after the administration of the one or more therapeutic agents. As also described above, a subject may be sensitized or primed by the administration of the compound of Formula (I) or a pharmaceutically acceptable salt thereof by any of the administration regimes described herein such as by continuous administration, and/or administration without a drug holiday, prior to the administration of the one or more therapeutic agents. Alternatively, a subject may be sensitized or primed by the administration of one or more therapeutic agents. In some embodiments, the compound of Formula (I) or a pharmaceutically acceptable salt thereof is administered with continuous administration, and/or administration without a drug holiday and the one or more therapeutic agents are administered one or more days after or prior to the administration of the compound of Formula (I) or a pharmaceutically acceptable salt thereof.

In some embodiments, the compound of Formula (I) or a pharmaceutically acceptable salt thereof is administered with continuous administration, and/or administration without a drug holiday until a desirable biological effect is achieved (e.g., altered chromatin state, reduction of H3K79 methyl mark, and/or cell differentiation) prior to administration of the one or more therapeutic agents.

In some embodiments, one or more therapeutic agents are administered as indicated on label until a desirable biological effect is achieved (e.g., altered chromatin state, reduction of H3K79 methyl mark, and/or cell differentiation) prior to administration of the compound of Formula (I) or a pharmaceutically acceptable salt thereof or the composition comprising a compound of Formula (I) or a pharmaceutically acceptable salt thereof and one or more therapeutic agents.

In some embodiments, a subject is evaluated after one treatment regimen described herein for any biological effects. In some embodiments, no further treatment is required if the evaluated biological effect has reached a certain predetermined level or activity. In some embodiments, the biological effect is maturation or induction of blast cells, apoptosis of leukemic blast cells, resolution of fever, cachexia or leukemia cutis, restoration of normal haemoatopoiesis, and/or complete remission. In some embodiments, the biological effect is a durable altered chromatin state. In some embodiments, the durable altered chromatin state is decreased histone methylation. In some embodiments the decreased chromatin methylation is decreased methylation of H3K79. In some embodiments, the durable altered chromatin state is present at 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more after the treatment.

“Combination therapy” is intended to embrace administration of these therapeutic agents in a sequential manner, wherein each therapeutic agent is administered at a different time, as well as administration of these therapeutic agents, or at least two of the therapeutic agents concurrently, or in a substantially simultaneous manner. Simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. Therapeutic agents may also be administered in alternation.

The combination therapies featured in the present invention can result in a synergistic effect in the treatment of a disease or cancer. A “synergistic effect” is defined as where the efficacy of a combination of therapeutic agents is greater than the sum of the effects of any of the agents given alone. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

As provided herein, the administration of the combination of a compound of Formula (I) and one or more therapeutic agents provides synergistic effects. As provided herein, the combination of a compound of Formula (I) and therapeutic agents result in a synergistic antiproliferative response, a synergistic induction of apoptosis in leukemic cells and a synergistic induction of differentiation of leukemic cells. As provided herein synergistic effects also result when leukemic cells are sensitized by the administration of a compound of Formula (I) prior to the administration of therapeutic agents.

“Combination therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In another aspect, a composition of the present invention may be administered in combination with radiation therapy. Radiation therapy can also be administered in combination with a composition of the present invention and another chemotherapeutic agent described herein as part of a multiple agent therapy.

The present invention also provides pharmaceutical compositions comprising a compound of Formula (I) or pharmaceutically acceptable salts thereof, and one or more other therapeutic agent disclosed herein, mixed with pharmaceutically suitable carriers or excipient(s) at doses to treat or prevent a disease or condition as described herein.

In one aspect, the present invention also provides pharmaceutical compositions comprising any compound of Tables 1-4 or pharmaceutically acceptable salts thereof, and one or more therapeutic agents, mixed with pharmaceutically suitable carriers or excipient(s) at doses to treat or prevent a disease or condition as described herein.

In another aspect, the present invention also provides pharmaceutical compositions comprising Compound A2 having the formula:

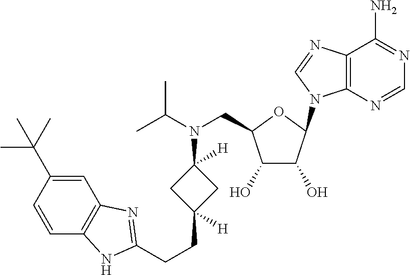 [SMILES: CC(C)N(C[C@H]1O[C@@H](n2cnc3c(N)ncnc32)[C@H](O)[C@@H]1O)[C@H]1C[C@@H](CCc2nc3cc(C(C)(C)C)ccc3[nH]2)C1]

or pharmaceutically acceptable salts thereof, and one or more therapeutic agents, mixed with pharmaceutically suitable carriers or excipient(s) at doses to treat or prevent a disease or condition as described herein.

In another aspect, the present invention also provides pharmaceutical compositions comprising Compound D16 having the formula

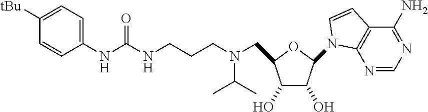 [SMILES: CC(C)N(CCCNC(=O)Nc1ccc(C(C)(C)C)cc1)C[C@H]1O[C@@H](n2ccc3c(N)ncnc32)[C@H](O)[C@@H]1O]

or pharmaceutically acceptable salts thereof, and one or more therapeutic agents, mixed with pharmaceutically suitable carriers or excipient(s) at doses to treat or prevent a disease or condition as described herein.

The pharmaceutical compositions of the present invention can also be administered in combination with other therapeutic agents or therapeutic modalities simultaneously, sequentially, or in alternation.

Mixtures of compositions of the present invention can also be administered to the patient as a simple mixture or in suitable formulated pharmaceutical compositions.

A “pharmaceutical composition” is a formulation containing the compounds of the present invention in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug interaction(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

In some embodiments, the compound of Formula (I) or pharmaceutically acceptable salt thereof is administered continuously for at least 7, 14, 21, 28, 35, 42, 47, 56, or 64 days. In some embodiments, the compound of Formula (I) or pharmaceutically acceptable salt thereof is administered continuously for at least 7, 14, 21, 28, 35, 42, 47, 56, or 64 days without a drug holiday.

In some embodiments, the compound of Formula (I) or pharmaceutically acceptable salt thereof is administered at a dose of at least 36 mg/m², at least 54 mg/m² or at least 80 mg/m². In some embodiments, the compound of Formula (I) or pharmaceutically acceptable salt thereof is administered at a dose of at least 36 mg/m², at least 54 mg/m² or at least 80 mg/m² continuously for at least 7, 14, 21, 28, 35, 42, 47, 56, or 64 days. In some embodiments, the compound of Formula (I) or pharmaceutically acceptable salt thereof is administered at a dose of at least 36 mg/m², at least 54 mg/m² or at least 80 mg/m² continuously without a drug holiday. In some embodiments, the compound of Formula (I) or pharmaceutically acceptable salt thereof is administered at a dose of at least 36 mg/m², at least 54 mg/m² or at least 80 mg/m² continuously for at least 7, 14, 21, 28, 35, 42, 47, 56, or 64 days without a drug holiday.

In some embodiments, the compound of Formula (I) or pharmaceutically acceptable salt thereof is administered continuously for at least 7, 14, 21, 28, 35, 42, 47, 56, or 64 days in combination with one or more therapeutic agents. In some embodiments, the compound of Formula (I) or pharmaceutically acceptable salt thereof is administered continuously for at least 7, 14, 21, 28, 35, 42, 47, 56, or 64 days without a drug holiday in combination with one or more therapeutic agents.

In some embodiments, the compound of Formula (I) or pharmaceutically acceptable salt thereof is administered at a dose of at least 36 mg/m², at least 54 mg/m² or at least 80 mg/m² in combination with one or more therapeutic agents. In some embodiments, the compound of Formula (I) or pharmaceutically acceptable salt thereof is administered at a dose of at least 36 mg/m², at least 54 mg/m² or at least 80 mg/m² continuously for at least 7, 14, 21, 28, 35, 42, 47, 56, or 64 days in combination with the one or more therapeutic agents. In some embodiments, the compound of Formula (I) or pharmaceutically acceptable salt thereof is administered at a dose of at least 36 mg/m², at least 54 mg/m² or at least 80 mg/m² continuously without a drug holiday in combination with one or more therapeutic agents. In some embodiments, the compound of Formula (I) or pharmaceutically acceptable salt thereof is administered at a dose of at least 36 mg/m², at least 54 mg/m² or at least 80 mg/m² continuously for at least 7, 14, 21, 28, 35, 42, 47, 56, or 64 days without a drug holiday in combination with one or more therapeutic agents.

The pharmaceutical compositions can be included in a container, pack, or dispenser together with instructions for administration.

The compounds of the present invention are capable of further forming salts. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds of the present invention can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs in the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the invention, and the like, See Bundegaard, H., Design of Prodrugs, p 1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention.

Based on the present disclosure the skilled artisan can identify and employ other components and methodology useful for practicing the present invention.

In the synthetic schemes described herein, compounds may be drawn with one particular configuration for simplicity. Such particular configurations are not to be construed as limiting the invention to one or another isomer, tautomer, regioisomer or stereoisomer, nor does it exclude mixtures of isomers, tautomers, regioisomers or stereoisomers.

Compounds described herein are assayed for modulation of activity, for example, histone methylation, modulation of cell growth and/or IC₅₀, described in the examples below. IC₅₀ values for DOT1L inhibition for select DOT1L inhibitors were determined as described in Example 1 and are listed below.

| | DOT1L |
|---|---|
| | IC₅₀ |
| Compound | (μM) |
| A2 | 0.00074 |
| A3 | 0.00073 |
| A5 | 0.00059 |
| A69 | 0.00251 |
| A75 | 0.00059 |
| A86 | 0.00062 |
| A87 | 0.0008 |
| A91 | 0.00218 |
| A93 | 0.00292 |

Diseases such as cancers and neurological disease can be treated by administration of modulators of protein (e.g., histone) methylation, e.g., modulators of histone methyltransferase, or histone demethylase enzyme activity. Histone methylation has been reported to be involved in aberrant expression of certain genes in cancers, and in silencing of neuronal genes in non-neuronal cells. The composition of this invention, e.g. a composition comprising any compound of Formula (I) or pharmaceutically acceptable salt thereof and one or more therapeutic agents described herein can be used to treat such diseases, i.e., to decrease or inhibit methylation of histones in affected cells or restore methylation to roughly its level in counterpart normal cells.

The present invention provides compositions and methods for treating or alleviating a symptom of conditions and diseases the course of which can be influenced by modulating the methylation status of histones or other proteins, wherein said methylation status is mediated at least in part by the activity of DOT1L. Modulation of the methylation status of histones can in turn influence the level of expression of target genes activated by methylation, and/or target genes suppressed by methylation. The method includes administering to a subject in need of such treatment, a therapeutically effective amount of a composition of the present invention or a pharmaceutically acceptable salt, prodrug, metabolite, polymorph or solvate thereof, to a subject in need of such treatment.

Modulators of methylation can be used for modulating cell proliferation, generally. For example, in some cases excessive proliferation may be reduced with agents that decrease methylation, whereas insufficient proliferation may be stimulated with agents that increase methylation. Accordingly, diseases that may be treated include hyperproliferative diseases, such as benign cell growth and malignant cell growth (cancer).

The disorder in which DOT1L-mediated protein methylation plays a part can be cancer, a cell proliferative disorder, or a precancerous condition. Exemplary cancers that may be treated include brain and CNS cancer, kidney cancer, ovarian cancer, pancreatic cancer, lung cancer, breast cancer, colon cancer, prostate cancer, or a hematological cancer. For example, the hematological cancer is leukemia or lymphoma. Preferably the cancer is leukemia. The leukemia can be acute or chronic leukemia. In some embodiments, the leukemia is acute myeloid leukemia or acute lymphocytic leukemia. In some embodiments, leukemia that may be treated is leukemia characterized by a chromosomal rearrangement on chromosome 11q23, including chimeric fusion of mixed lineage leukemia gene (MLL) or partial tandem duplication of MLL (MLL-PTD). In some embodiments, leukemia that may be treated is leukemia characterized by the presence of a genetic lesion of MLL. Such genetic lesions include chromosomal rearrangements, such as translocations, deletions, and/or duplications of the MLL gene. MLL has been categorized or characterized as having a chimeric fusion of MLL, partial tandem duplication of the MLL gene (MLL-PTD), or nonrearranged MLL.

The disorder that can be treated by the combination therapy described herein can be a disorder medicated by translocation, deletion and/or duplication of a gene on chromosome 11q23.

In general, compounds that are methylation modulators can be used for modulating cell proliferation. For example, in some cases excessive proliferation may be reduced with agents that decrease methylation, whereas insufficient proliferation may be stimulated with agents that increase methylation. Accordingly, diseases that may be treated by the compounds of the invention include hyperproliferative diseases, such as benign cell growth and malignant cell growth.

As used herein, a “subject in need thereof” is a subject having a disorder in which DOT1L-mediated protein methylation plays a part, or a subject having an increased risk of developing such disorder relative to the population at large. A subject in need thereof can have a precancerous condition. Preferably, a subject in need thereof has cancer. A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

In some embodiments, the subject is child. In some embodiments, the subject is younger than 18 years of age. In some embodiments, the subject is younger than 18, 17, 16, 15, 14, 13, 12, 11, 10, 9, 8, 7, 6, 5, 4, 3, 2, or 1 year of age. In some embodiments, the subject is between 3 months and 18 years of age.

The subject of the present invention includes any human subject who has been diagnosed with, has symptoms of, or is at risk of developing a cancer or a precancerous condition.

A subject in need thereof may be a subject having a disorder associated DOT1L. A subject in need thereof can have a precancerous condition. Preferably, a subject in need thereof has cancer. A subject in need thereof can have cancer associated with DOT1L. In a preferred aspect, a subject in need thereof has one or more cancers selected from the group consisting of brain and central nervous system (CNS) cancer, head and neck cancer, kidney cancer, ovarian cancer, pancreatic cancer, leukemia, lung cancer, lymphoma, myeloma, sarcoma, breast cancer, prostate cancer and a hematological cancer. Preferably, a subject in need thereof has a hematologic cancer, wherein the hematologic cancer is leukemia or lymphoma. Exemplary leukemia is MLL. Other hematologic cancers of the present invention can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A subject in need thereof can be one who has been previously diagnosed or identified as having cancer or a precancerous condition. A subject in need thereof can also be one who is having (suffering from) cancer or a precancerous condition. Alternatively, a subject in need thereof can be one who is having an increased risk of developing such disorder relative to the population at large (i.e., a subject who is predisposed to developing such disorder relative to the population at large).

A subject in need thereof can have cancer associated with increased expression (mRNA or protein) and/or activity level of at least one protein selected from the group consisting of HOXA9, FLT3, MEIS1, MEIS2, TBP, BCL, and DOT1L. A subject in need thereof may have increased mRNA, protein, and/or activity level of at least of at least one signaling component downstream of at least one protein selected from the group consisting of HOXA9, FLT3, MEIS1, MEIS2, TBP, BCL, and DOT1L. Such downstream components are readily known in the art, and can include other transcription factors, or signaling proteins. As used herein, the term “increase in activity” refers to increased or a gain of function of a gene product/protein compared to the wild type. Accordingly, an increase in mRNA or protein expression and/or activity levels can be detected using any suitable method available in the art.

Optionally a subject in need thereof has already undergone, is undergoing or will undergo, at least one therapeutic intervention for the cancer or precancerous condition.

A subject in need thereof may have refractory cancer on most recent therapy. “Refractory cancer” means cancer that does not respond to treatment. The cancer may be resistant at the beginning of treatment or it may become resistant during treatment. Refractory cancer is also called resistant cancer. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy.

In some embodiments, a subject in need thereof may have a secondary cancer as a result of a previous therapy. “Secondary cancer” means cancer that arises due to or as a result from previous carcinogenic therapies, such as chemotherapy. In some embodiments, the secondary cancer is a hematologic cancer, such as leukemia.

The subject may exhibit resistance to DOT1L histone methyltransferase inhibitors or any other therapeutic agent.

The invention also features a method of selecting a combination therapy for a subject having leukemia. The method includes the steps of: detecting the level of HOXA9, FLT3, MEIS1, MEIS2, TBP, BCL, and/or DOT1L in a sample from the subject; and selecting, based on the presence of the increased level of HOXA9, FLT3, MEIS1, MEIS2, TBP, BCL, and/or DOT1L, a combination therapy for treating leukemia. In one embodiment, the therapy includes administering to the subject a composition of the invention. In one embodiment, the method further includes administrating to the subject a therapeutically effective amount of a composition of the invention. In one embodiment, the leukemia is characterized by partial tandem duplication of the MLL gene (MLL-PTD)n. In another embodiment, the leukemia is characterized by overexpression of HOXA9, FLT3, MEIS1, MEIS2, TBP, BCL, and/or DOT1L.

The methods and uses described herein may include steps of detecting the mRNA, protein and/or activity (function) level of HOXA9, FLT3, MEIS1, MEIS2, TBP, BCL, and/or DOT in a sample from a subject in need thereof prior to and/or after the administration of a composition of the invention (e.g., a composition comprising a compound of Formula (I) or pharmaceutically acceptable salts thereof, and one or more therapeutic agents) to the subject. The presence of the increased level of HOXA9, FLT3, MEIS1, MEIS2, TBP, BCL, and/or DOT1L in the tested sample indicates the subject is responsive to the combination therapy described herein.

The present invention provides personalized medicine, treatment and/or cancer management for a subject by genetic screening of increased gene expression (mRNA or protein), and/or increased function or activity level of at least one protein selected from the group consisting of HOXA9, FLT3, MEIS1, MEIS2, TBP, BCL, and DOT1L in the subject. For example, the present invention provides methods for treating or alleviating a symptom of cancer or a precancerous condition in a subject in need thereof by determining responsiveness of the subject to a combination therapy and when the subject is responsive to the combination therapy, administering to the subject a composition of the invention. The responsiveness is determined by obtaining a sample from the subject and detecting increased mRNA or protein, and/or increased activity level of at least one protein selected from the group consisting of HOXA9, FLT3, MEIS1, MEIS2, TBP, BCL, and DOT1L, and the presence of such gain of expression and/or function indicates that the subject is responsive to the composition of the invention. Once the responsiveness of a subject is determined, a therapeutically effective amount of a composition, for example, a composition comprising a compound of Formula (I) or pharmaceutically acceptable salts thereof, and one or more therapeutic agents, can be administered. The therapeutically effective amount of a composition can be determined by one of ordinary skill in the art.

As used herein, the term “responsiveness” is interchangeable with terms “responsive”, “sensitive”, and “sensitivity”, and it is meant that a subject is showing therapeutic responses when administered a composition of the invention, e.g., tumor cells or tumor tissues of the subject undergo apoptosis and/or necrosis, and/or display reduced growing, dividing, or proliferation. This term is also meant that a subject will or has a higher probability, relative to the population at large, of showing therapeutic responses when administered a composition of the invention, e.g., tumor cells or tumor tissues of the subject undergo apoptosis and/or necrosis, and/or display reduced growing, dividing, or proliferation.

By “sample” it means any biological sample derived from the subject, includes but is not limited to, cells, tissues samples, body fluids (including, but not limited to, mucus, blood, plasma, serum, urine, saliva, and semen), tumor cells, and tumor tissues. Preferably, the sample is selected from bone marrow, peripheral blood cells, blood, plasma and serum. Samples can be provided by the subject under treatment or testing. Alternatively samples can be obtained by the physician according to routine practice in the art.

An increase in mRNA or protein expression and/or activity levels can be detected using any suitable method available in the art. For example, an increase in activity level can be detected by measuring the biological function of a gene product, such as the histone methyltransferase activity of DOT1L (i.e., methylation of histone substrates such as H3K79 by immunoblot); transcriptional activity of HOXA9, MEIS2 or MEIS1 (i.e., expression levels of HOXA9, MEIS2 or MEIS1 target genes by RT-PCR); or phosphorylation activity of FLT3 (i.e., phosphorylation status of FLT3 targets by immunoblot or radioimmunoassay). Alternatively, a gain of function mutation can be determined by detecting any alternation in a nucleic acid sequence encoding a protein selected from the group consisting of HOXA9, FLT3, MEIS1, MEIS2, TBP, BCL, and DOT1L. For example, a nucleic acid sequence encoding HOXA9, FLT3, MEIS1, MEIS2, TBP, BCL, and/or DOT1L having a gain of function mutation can be detected by whole-genome resequencing or target region resequencing (the latter also known as targeted resequencing) using suitably selected sources of DNA and polymerase chain reaction (PCR) primers in accordance with methods well known in the art. The method typically and generally entails the steps of genomic DNA purification, PCR amplification to amplify the region of interest, cycle sequencing, sequencing reaction cleanup, capillary electrophoresis, and/or data analysis. Alternatively or in addition, the method may include the use of microarray-based targeted region genomic DNA capture and/or sequencing. Kits, reagents, and methods for selecting appropriate PCR primers and performing resequencing are commercially available, for example, from Applied Biosystems, Agilent, and NimbleGen (Roche Diagnostics GmbH). Detection of mRNA expression can be detected by methods known in the art, such as Northern blot, nucleic acid PCR, and quantitative RT-PCR. Detection of polypeptide expression (i.e., wild-type or mutant) can be carried out with any suitable immunoassay in the art, such as Western blot analysis.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue.

A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute lymphocytic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Preferably, compositions of the present invention may be used to treat a cancer selected from the group consisting of a hematologic cancer of the present invention or a hematologic cell proliferative disorder of the present invention. A hematologic cancer of the present invention can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, compositions of the present invention may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer. Preferably, compositions of the present invention may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructual heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions of the present invention may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Preferably, compositions of the present invention may be used to treat breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructual heterogeneity (e.g., mixed cell types).

Preferably, compound of the present invention, or a pharmaceutically acceptable salt, prodrug, metabolite, polymorph, or solvate thereof, may be used to treat breast cancer. A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich, or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound of the present invention, or a pharmaceutically acceptable salt, prodrug, metabolite, polymorph or solvate thereof, may be used to treat or prevent a cell proliferative disorder of the breast, or to treat or prevent breast cancer, in a subject having an increased risk of developing breast cancer relative to the population at large. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can histologically graded according to the Scarff-Bloom-Richardson system, wherein a breast tumor has been assigned a mitosis count score of 1, 2, or 3; a nuclear pleiomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the present invention, or a pharmaceutically acceptable salt, prodrug, metabolite, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present invention, or a pharmaceutically acceptable salt, prodrug, metabolite, polymorph or solvate thereof. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

For example, an in vitro biological assay that can be used includes the steps of (1) mixing a histone substrate (e.g., an isolated histone sample for a histone or modified histone of interest, or an isolated oligonucleosome substrate) with recombinant DOT1L enzyme (e.g., recombinant protein containing amino acids 1-416); (2) adding a candidate compound of the invention to this mixture; (3) adding non-radioactive and ³H-labeled S-Adenosyl methionine (SAM) to start the reaction; (4) adding excessive amount of non-radioactive SAM to stop the reaction; (4) washing off the free non-incorporated ³H-SAM; and (5) detecting the quantity of ³H-labeled histone substrate by any methods known in the art (e.g., by a PerkinElmer TopCount platereader).

For example, an in vitro cell viability assay that can be used includes the steps of (1) culturing cells (e.g., EOL-1 cells) in the presence of increasing concentration of candidate compound (e.g., Compound A2, Compound D16); (2) determining viable cell number every 3-4 days by methods known in the art (e.g., using the Millipore Guava Viacount assay); (3) plotting concentration-dependence growth curves; and optionally (4) calculating IC₅₀ values from the concentration-dependence growth curves using methods known in the art (e.g., using GraphPad Prism Software).

For example, a histone methylation assay that can be used includes the steps of (1) culturing cells (e.g., EOL-1 cells) in the presence of candidate compound (e.g., Compound A2 or Compound D16); (2) harvesting the cells; (3) extracting histone proteins, using methods known in the art (e.g., sulfuric acid precipitation); (4) fractionating histone extracts by SDS-PAGE electrophoresis and transferring to a filter; (5) probing the filter with antibodies specific to a protein or methylated-protein of interest (e.g., H3K79me2-specific antibody and total histone H3-specific antibody); and (6) detecting the signal of the antibodies using methods known in the art (e.g., Li-cor Odyssey infrared imager).

For example, a gene expression assay that can be used includes the steps of (1) culturing cells (e.g., EOL-1, Molm13, MV411, LOUCY, SemK2, Reh, HL60, BV173, or Jurkat cells) in the presence or absence of candidate compound (e.g., Compound A2 or Compound D16); (2) harvesting the cells; (3) extracting the RNA using methods known in the art (e.g., Qiagen RNeasy Kit); (4) synthesizing cDNA from the extracted RNA (e.g., Applied Biosystems reverse transcriptase kit); (5) preparing qPCR reactions using, for example, primers and probes (e.g., predesigned labeled primer and probe sets for HOXA9, FLT3, MEIS1, MEIS2, TBP, BCL, DOT1L, and β2-microglobulin from Applied Biosystems), synthesized sample cDNA, and qPCR master mix reagent (e.g., Applied Biosystems Taqman universal PCR master mix); (6) running samples on PCR machine (e.g., Applied Biosystems); (7) analysis of the data and calculation of relative gene expression.

As used herein, “monotherapy” refers to the administration of a single active or therapeutic compound to a subject in need thereof. Preferably, monotherapy will involve administration of a therapeutically effective amount of a single active compound. For example, cancer monotherapy with one of the compound of the present invention, or a pharmaceutically acceptable salt, prodrug, metabolite, analog or derivative thereof, to a subject in need of treatment of cancer. In one aspect, the single active compound is a compound of the present invention, or a pharmaceutically acceptable salt, prodrug, metabolite, polymorph or solvate thereof

As used herein, “treating” or “treat” describes the management and care of a patient for the purpose of combating a disease, condition, or disorder and includes the administration of a compound of the present invention, or a pharmaceutically acceptable salt, prodrug, metabolite, polymorph or solvate thereof, to alleviate the symptoms or complications of a disease, condition or disorder, or to eliminate the disease, condition or disorder.

A compound of the present invention, or a pharmaceutically acceptable salt, prodrug, metabolite, polymorph or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant invention.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, a compound of the present invention, or a pharmaceutically acceptable salt, prodrug, metabolite, polymorph or solvate thereof, acts selectively on a cancer or precancerous cell but not on a normal cell. Preferably, a compound of the present invention, or a pharmaceutically acceptable salt, prodrug, metabolite, polymorph or solvate thereof, acts selectively to modulate one molecular target (e.g., a target protein methyltransferase) but does not significantly modulate another molecular target (e.g., a non-target protein methyltransferase). The invention also provides a method for selectively inhibiting the activity of an enzyme, such as a protein methyltransferase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A composition of the present invention e.g., a composition comprising a compound of Formula (I) or a pharmaceutically acceptable salt, prodrug, metabolite, polymorph or solvate thereof and one or more therapeutic agents, can modulate the activity of a molecular target (e.g., a target protein methyltransferase). Modulating refers to stimulating or inhibiting an activity of a molecular target. Preferably, a composition of the invention modulates the activity of a molecular target if it stimulates or inhibits the activity of the molecular target by at least 2-fold relative to the activity of the molecular target under the same conditions but lacking only the presence of said compound. More preferably, a composition of the present invention modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene.

As used herein, the term “isozyme selective” means preferential inhibition or stimulation of a first isoform of an enzyme in comparison to a second isoform of an enzyme (e.g., preferential inhibition or stimulation of a protein methyltransferase isozyme alpha in comparison to a protein methyltransferase isozyme beta). Preferably, a composition of the present invention demonstrates a minimum of a fourfold differential, preferably a tenfold differential, more preferably a fifty fold differential, in the dosage required to achieve a biological effect. Preferably, a composition of the present invention demonstrates this differential across the range of inhibition, and the differential is exemplified at the IC₅₀, i.e., a 50% inhibition, for a molecular target of interest.

Administering a composition of the present invention to a cell or a subject in need thereof can result in modulation (i.e., stimulation or inhibition) of an activity of a protein methyltransferase of interest. Several intracellular targets can be modulated with the compounds of the present invention, including, but not limited to, protein methyltransferase.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. A cell cycle checkpoint regulator can be a protein or not a protein.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

Preferably, an effective amount of a composition of the present invention is not significantly cytotoxic to normal cells. A therapeutically effective amount of a composition is not significantly cytotoxic to normal cells if administration of the composition in a therapeutically effective amount does not induce cell death in greater than 10% of normal cells. A therapeutically effective amount of a composition does not significantly affect the viability of normal cells if administration of the composition in a therapeutically effective amount does not induce cell death in greater than 10% of normal cells. In an aspect, cell death occurs by apoptosis.

Contacting a cell with a composition of the invention can induce or activate cell death selectively in cancer cells. Administering to a subject in need thereof a composition of the present invention can induce or activate cell death selectively in cancer cells. Contacting a cell with a composition of the present invention can induce cell death selectively in one or more cells affected by a cell proliferative disorder. Preferably, administering to a subject in need thereof a composition of the present invention induces cell death selectively in one or more cells affected by a cell proliferative disorder.

The present invention relates to a method of treating or alleviating a symptom of cancer by administering a composition of the present invention to a subject in need thereof, where administration of the composition results in one or more of the following: accumulation of cells in G1 and/or S phase of the cell cycle, cytotoxicity via cell death in cancer cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the invention

The composition of the instant invention can also be utilized to treat or alleviate a symptom of neurologic diseases or disorders. Neurologic diseases or disorders that may be treated with the compounds of this invention include epilepsy, schizophrenia, bipolar disorder or other psychological and/or psychiatric disorders, neuropathies, skeletal muscle atrophy, and neurodegenerative diseases, e.g., a neurodegenerative disease. Exemplary neurodegenerative diseases include: Alzheimer's, Amyotrophic Lateral Sclerosis (ALS), and Parkinson's disease. Another class of neurodegenerative diseases includes diseases caused at least in part by aggregation of poly-glutamine. Diseases of this class include: Huntington's Diseases, Spinalbulbar Muscular Atrophy (SBMA or Kennedy's Disease) Dentatorubropallidoluysian Atrophy (DRPLA), Spinocerebellar Ataxia 1 (SCAT), Spinocerebellar Ataxia 2 (SCA2), Machado-Joseph Disease (MJD; SCA3), Spinocerebellar Ataxia 6 (SCA6), Spinocerebellar Ataxia 7 (SCAT), and Spinocerebellar Ataxia 12 (SCA12).

Any other disease in which epigenetic methylation, which is mediated by DOT1, plays a role may be treatable or preventable using compounds and methods described herein.

The present invention provides use of a composition disclosed herein for inhibiting DOT1L activity in a cell. Still another aspect of the invention relates to a use of a composition disclosed herein for reducing the level of methylation of histone H3 lysine residue 79 (H3-K79) in a cell.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Example 1

### DOT1L Combination Studies in MLL-Rearranged Cell Lines Methods

The acute myelogenous leukemia cell lines MV4-11 (MLL-AF4) and MOLM-13 (MLL-AF9) were obtained from American Type Culture Collection (ATCC; Rockville, Md.) and Deutsche Sammlung von Mikroorganismen and Zellkulturen (DSMZ; Braunschweig, Germany) respectively. MV4-11 cells were maintained in IMDM (Invitrogen, supplemented with 10% heat inactivated fetal bovine serum (Life Technologies, Grand Island, N.Y.). MOLM-13 cells were maintained in RPMI-1640 supplemented with 10% fetal bovine serum (Life Technologies, Grand Island, N.Y.). Cultures were maintained in a humidified atmosphere including 5% CO₂.

Studies were performed using MLL-rearranged cell lines in vitro to evaluate the anti-proliferative effect of a combination of two agents together on cell growth. Initial proliferation studies were performed to determine the IC₅₀ of a given compound in each cell line. The cell counts were measured by ATP quantitation using the Promega Cell Titer Glo kit and luminescence values corresponded to the amount of ATP in a given well.

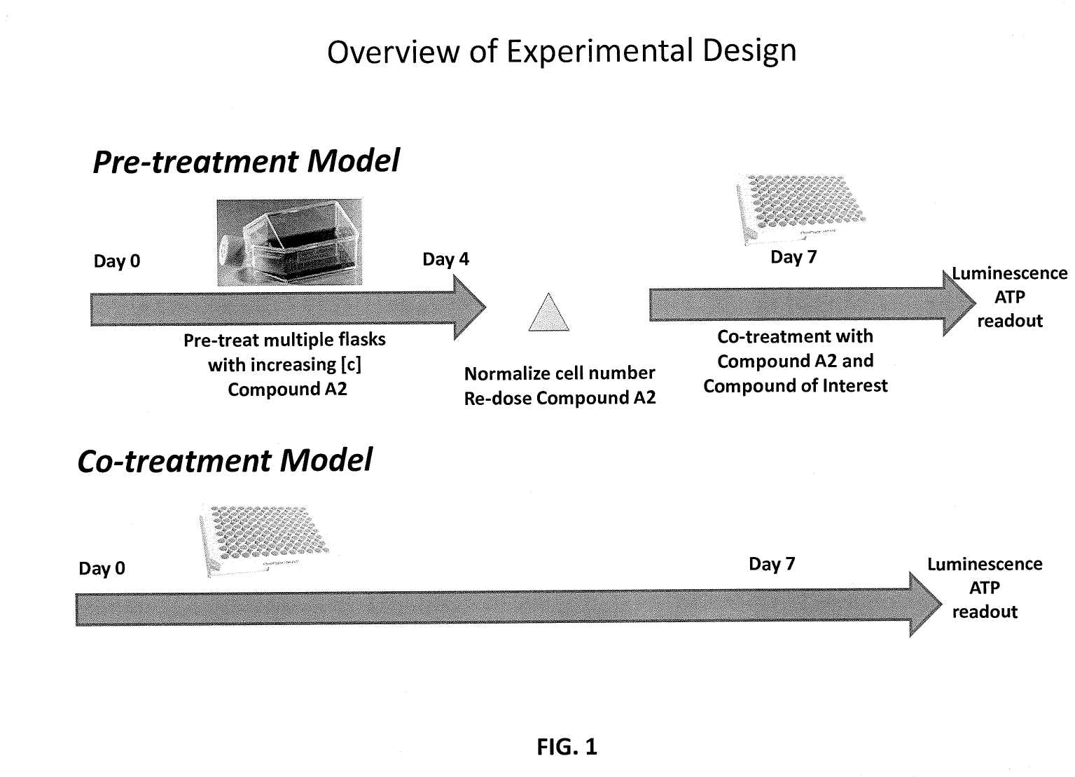

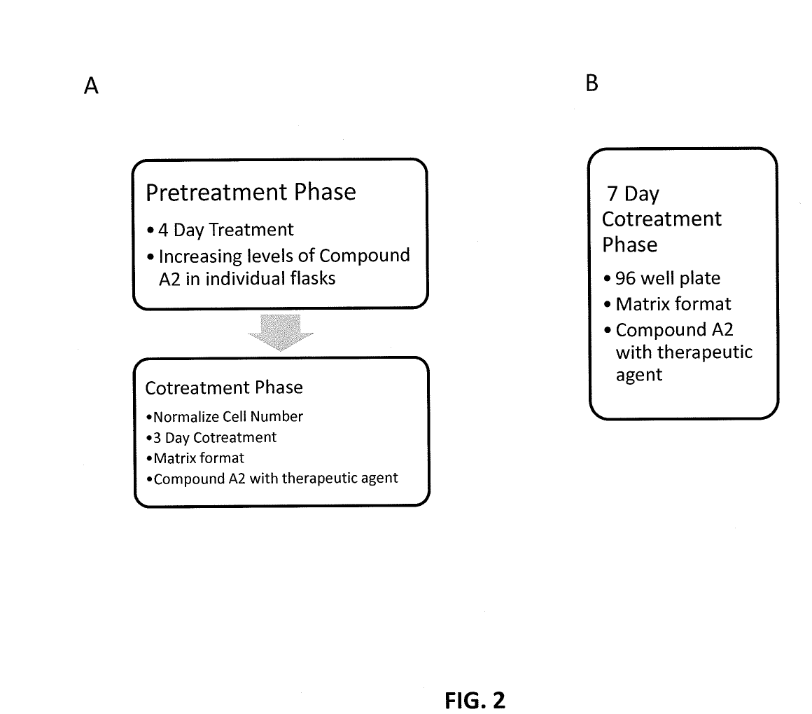

Compounds were tested in combination with Compound A2 to study their effect on cell proliferation in either a 4+3 model (cells were pretreated with increasing concentrations of Compound A2 for 4 days, followed by a co-treatment with Compound A2 with test article for 3 days) or a 7 day co-treatment model (FIGS. 1 and 2).

### Results

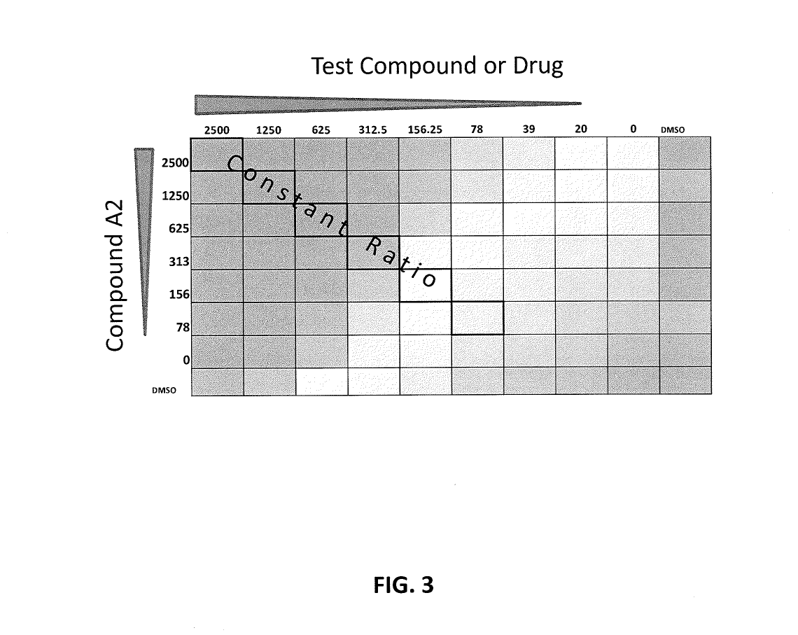

Compounds were evaluated for synergy in the co-treatment phase by testing the compounds in a concentration range which was bracketed around their IC₅₀ values. The compounds were plated to a 96 well plate in a matrix format (FIG. 3) which includes increasing concentrations of each drug in the combination in a constant ratio, in addition to the effect of each compound alone in the study. Cells were seeded and grown in the log-linear phase for 3 or 7 days in the co-treatment phase. Minimum inhibition (DMSO alone) controls were used in each plate to calculate fraction affected (Fa) of a test well. DMSO concentration was kept at 0.1% v/v.

The drug combination analysis was performed utilizing the Chou-Talalay method (Ref 1). Synergy was determined using the software package Calcusyn by Biosoft. The combination index (CI) is a quantitative term used to describe the level of synergy or antagonism in a given test system. A combination index less than one indicates synergy, and a CI greater than one indicates antagonism. Further, strong synergism is achieved when the CI value falls below 0.3.

Pretreatment with Compound A2 followed by cotreatment with either Ara-C or Daunorubicin demonstrated synergy in both MV4-11 and MOLM-13 cell lines.

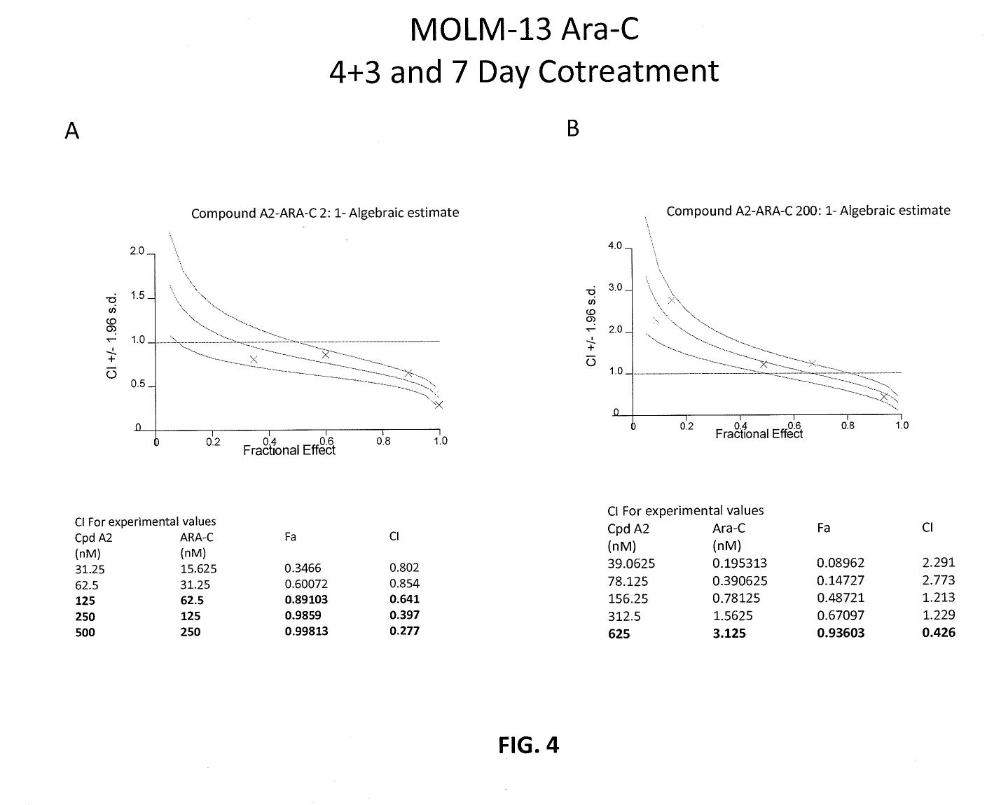

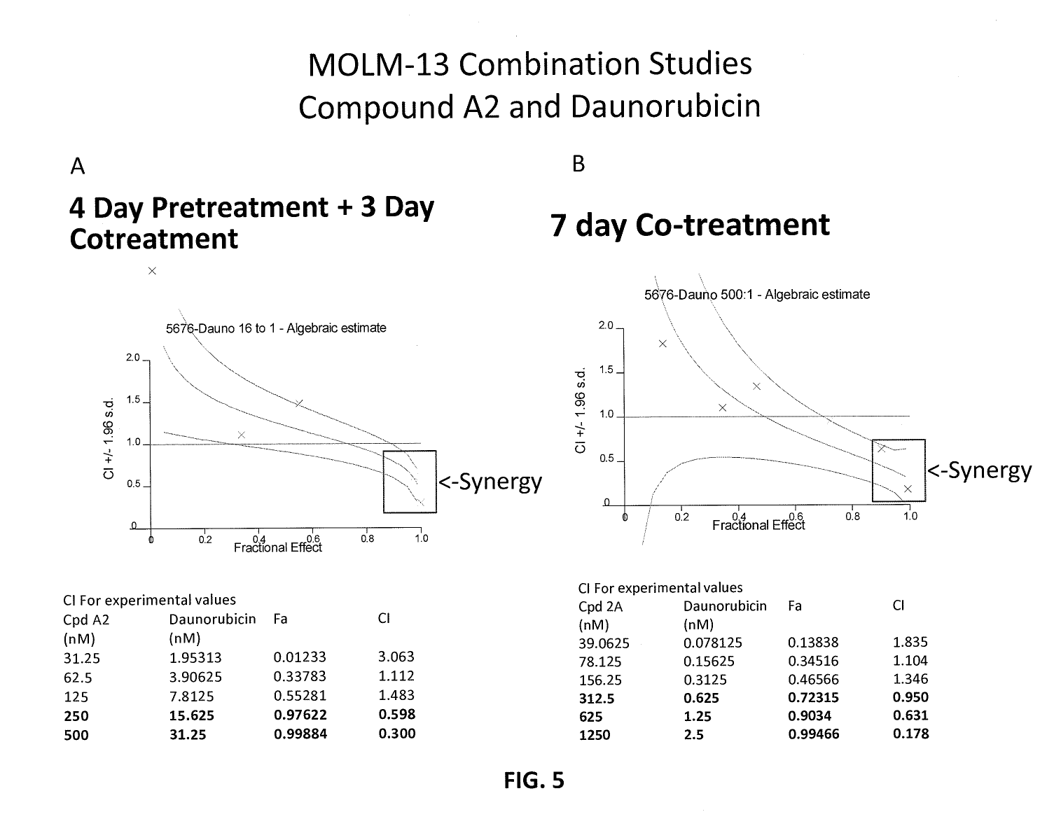

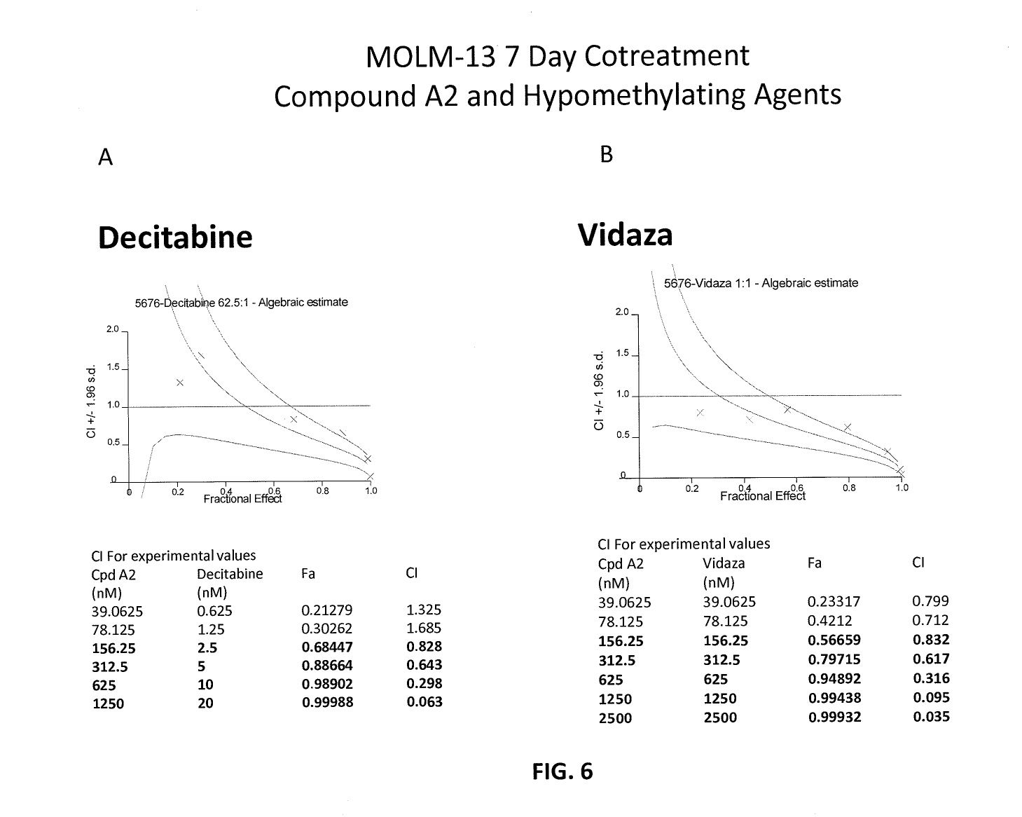

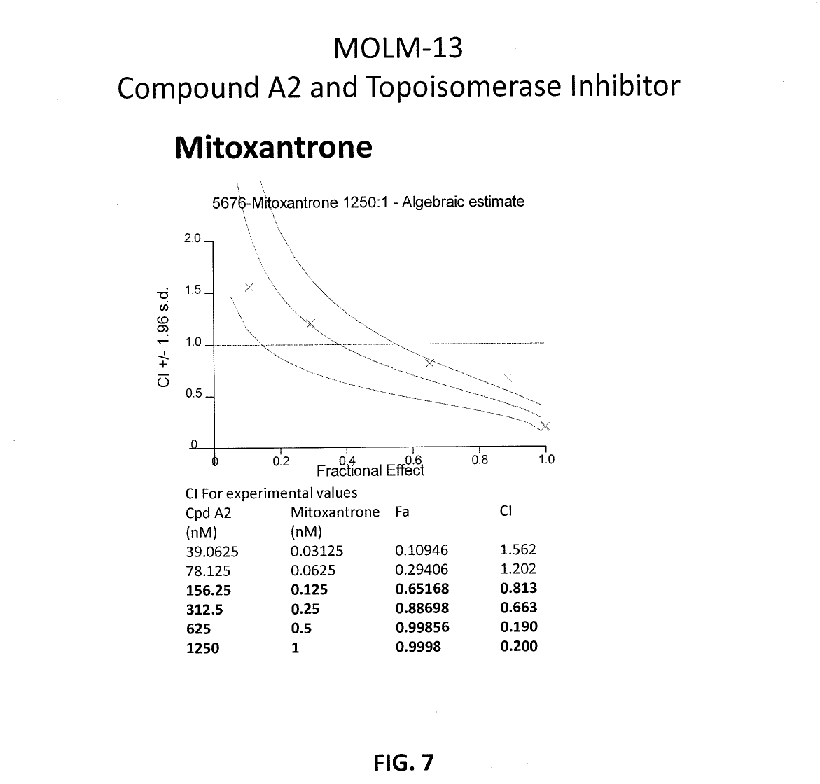

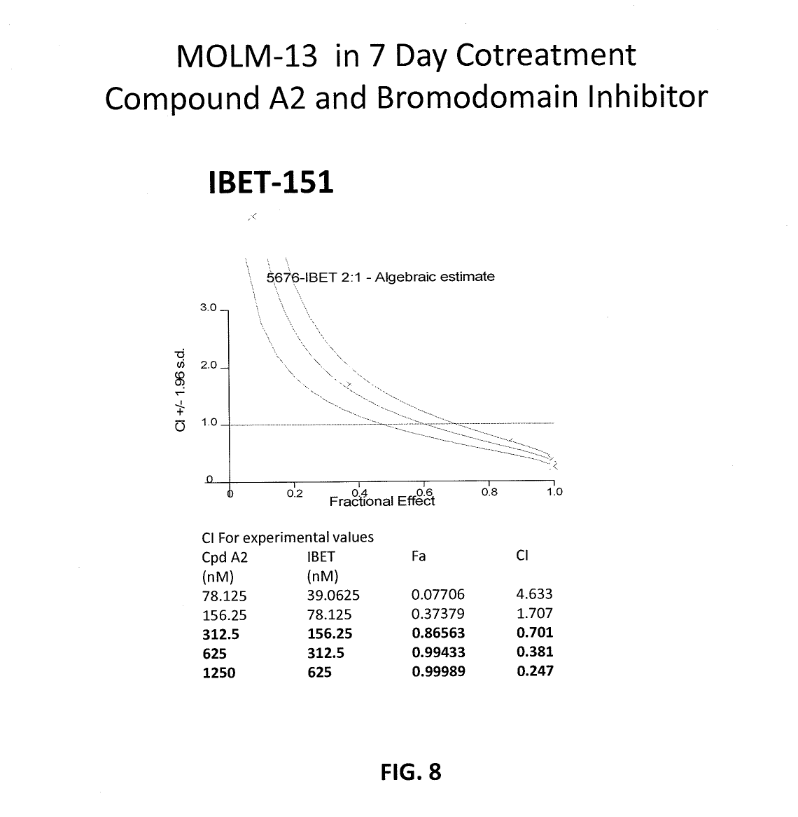

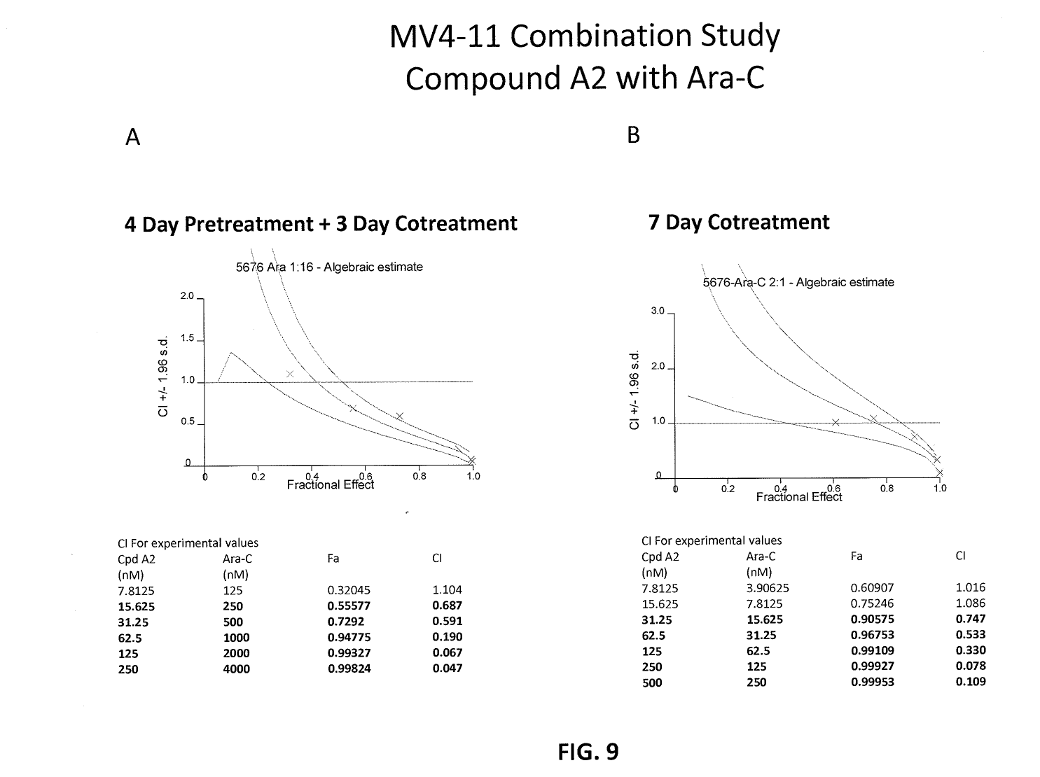

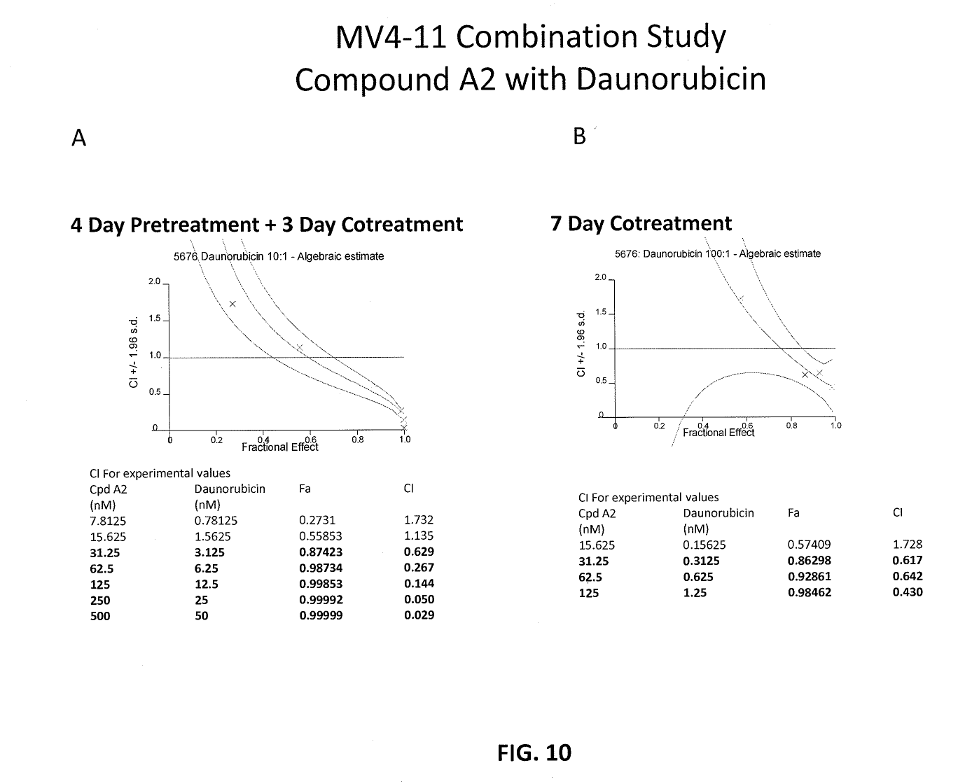

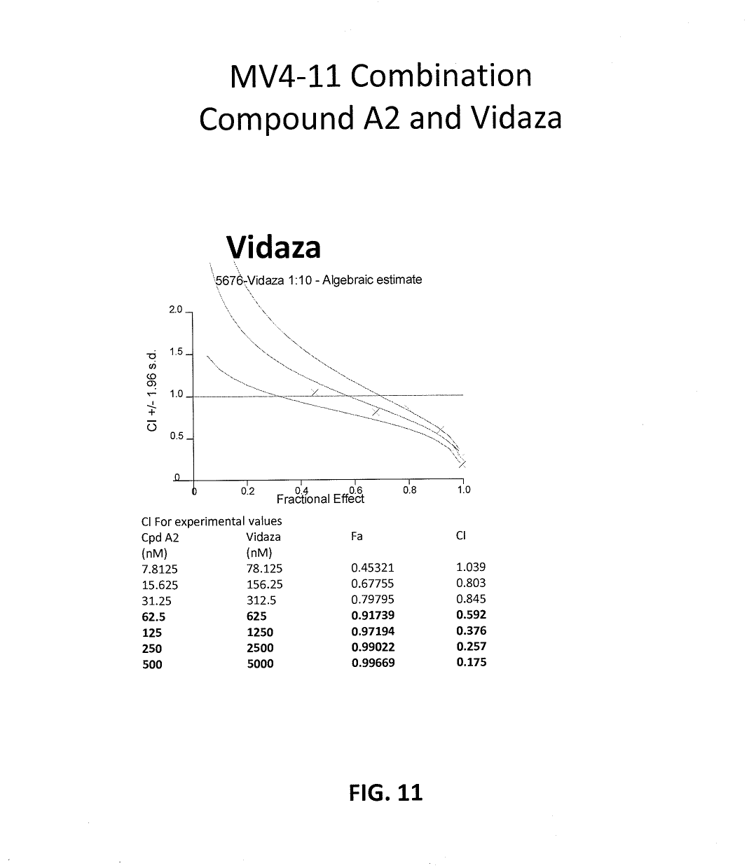

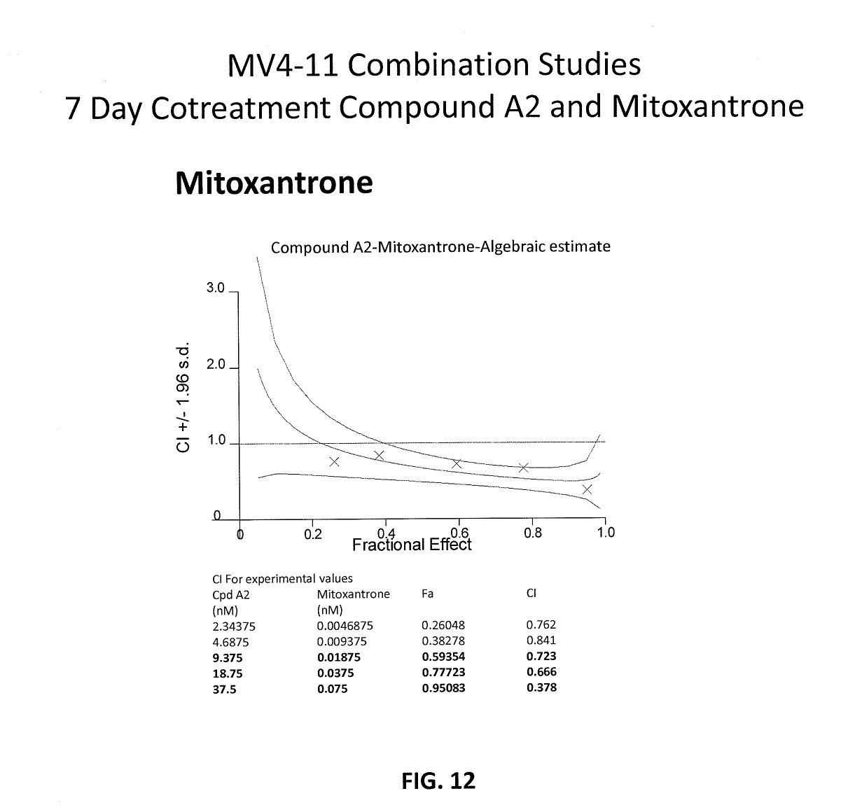

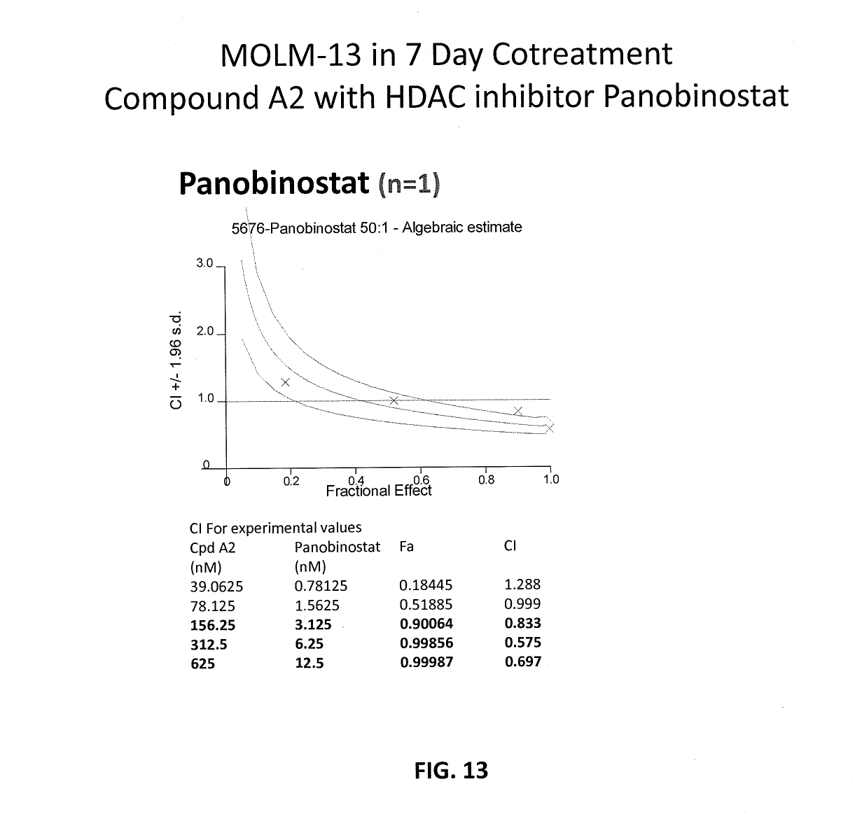

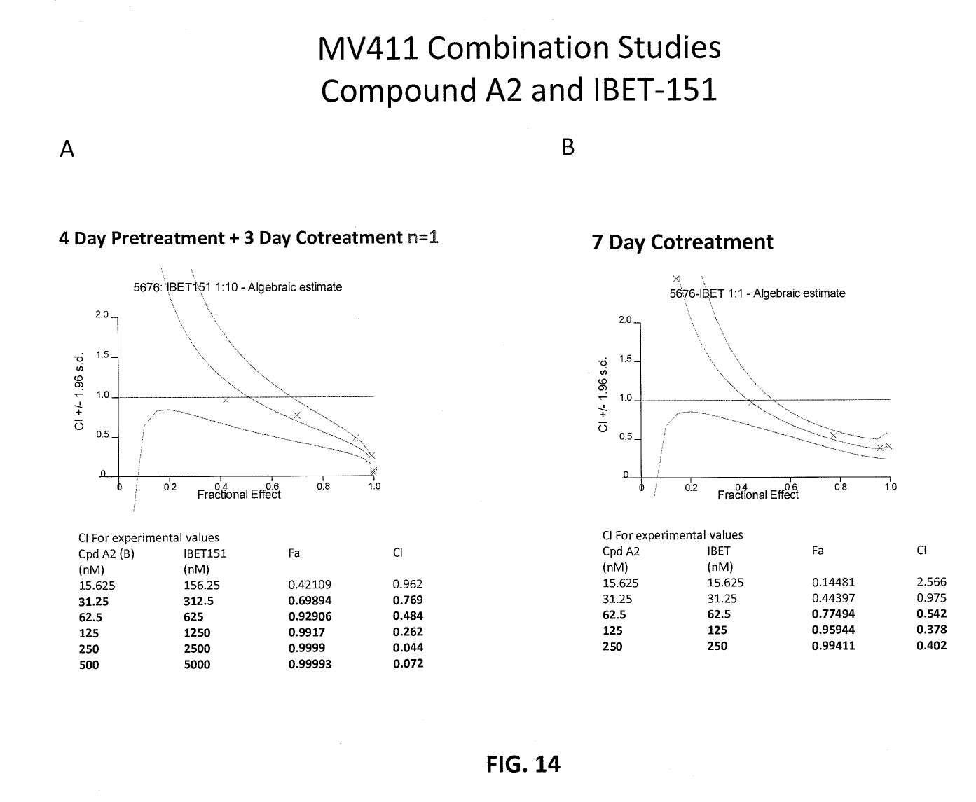

In a seven day cotreatment model, synergy with Compound A2 has been shown with the following drugs in the MOLM-13 (MLL-AF9 rearranged) cell line: Ara-C (FIG. 4), Daunorubicin (FIG. 5) Decitabine (strong) (FIG. 6), Vidaza (strong) (FIG. 6), Mitoxantrone (FIG. 7), IBET-151 (FIG. 8). Synergy with Compound A2 has been shown with the following drugs in MV4-11 (MLL-AF4) cell line: Ara-C (FIG. 9), Daunorubicin (FIG. 10), Vidaza (FIG. 11), Mitoxantrone (FIG. 12), IBET-151 (FIG. 14).

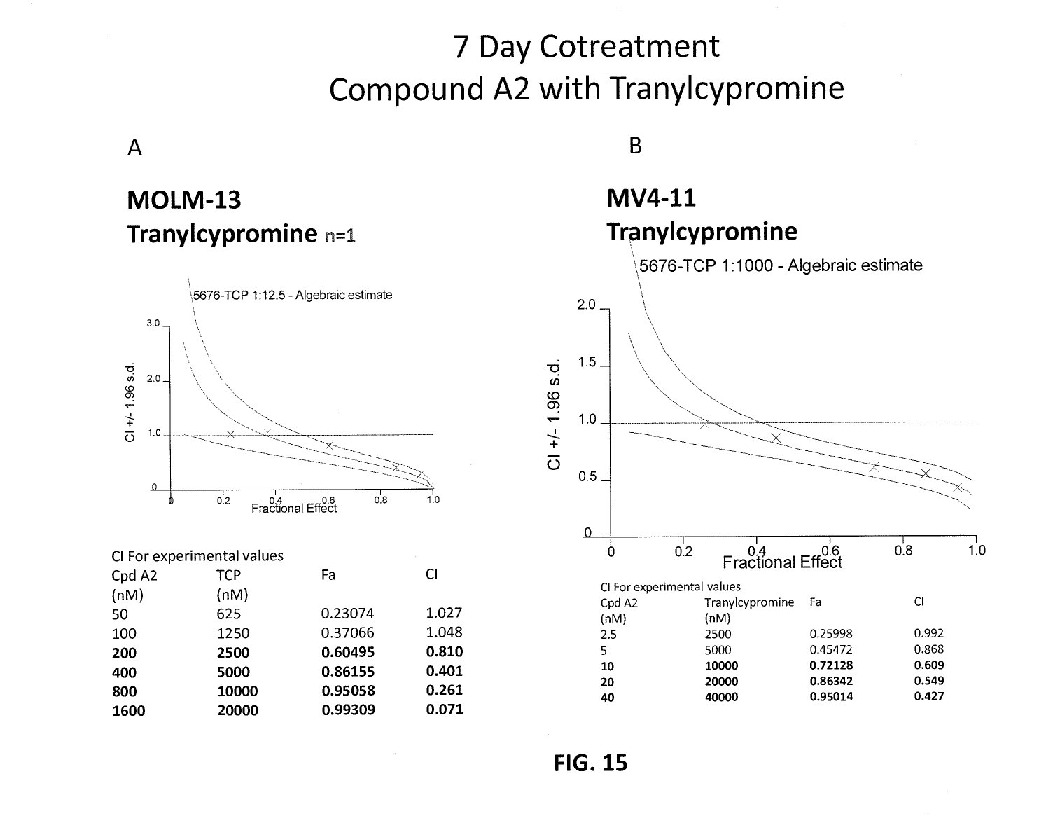

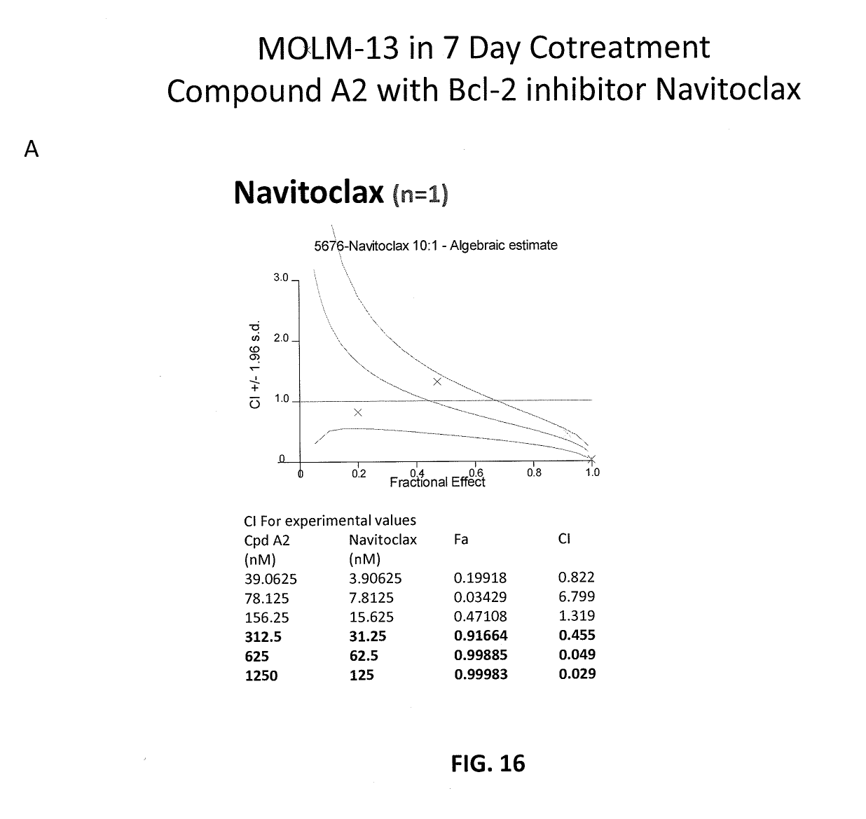

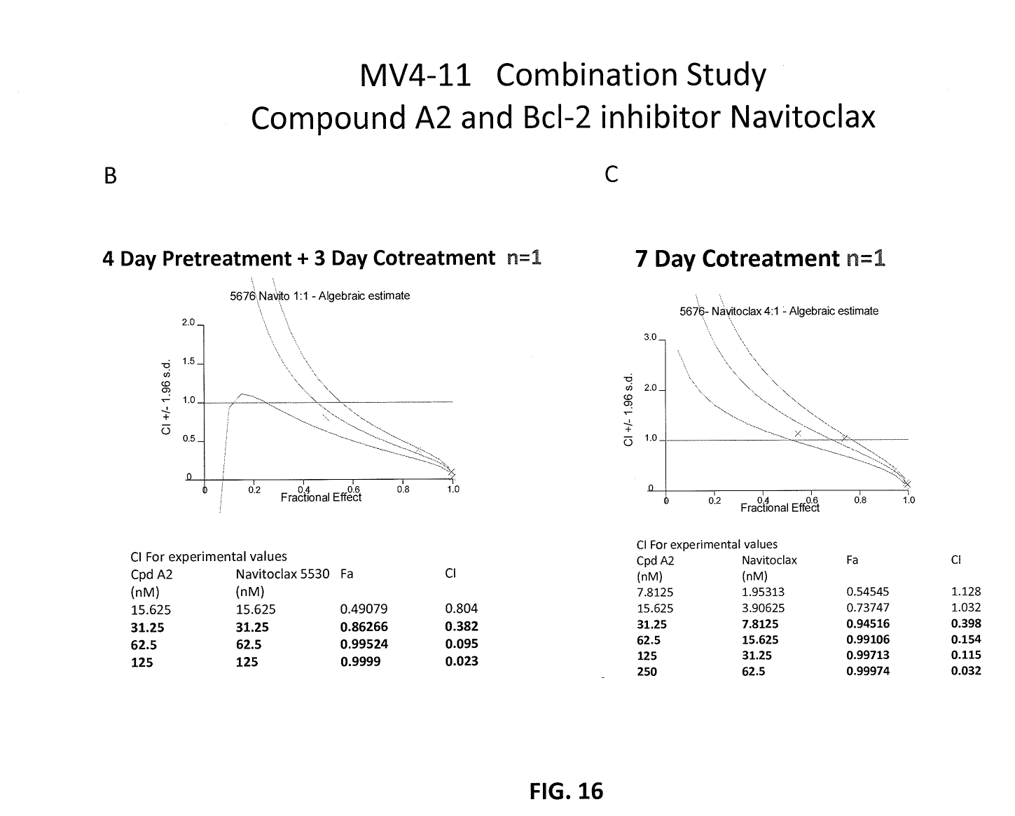

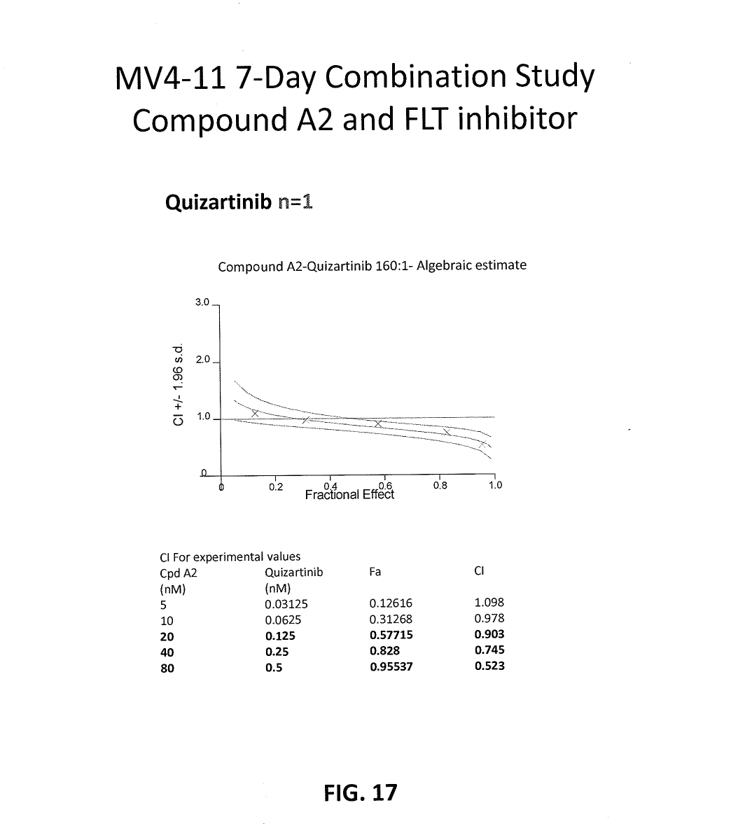

To this end, it has been demonstrated that LSD1 inhibitor, Tranylcypromine (FIG. 15) and Bcl-2 inhibitor, Navitoclax (FIG. 16) show synergy with Compound A2 in both MOLM (FIGS. 15 and 16) and MV4-11 cell lines (FIGS. 15 and 16). Quizartinib (FIG. 17), a FLT inhibitor has also shown synergy in MV4-11 cells.

TABLE 5
Summary table for combination studies of Compound
A2 and exemplary anti-cancer agents.

| | |
|---|---|
| MOLM-13 | MV4-11 |

4 + 3 Model

| | | |
|---|---|---|
| Ara-C | Synergy | Synergy |
| Daunorubicin | Synergy | Synergy |

7 Day Cotreatment

| | | |
|---|---|---|
| Ara-C | Synergy | Synergy |
| Daunorubicin | Synergy | Synergy |
| Decitabine | Strong Synergy | Additive |
| | | (no data shown) |
| Vidaza | Strong Synergy | Synergy |
| Mitoxantrone | Synergy | Synergy |
| IBET-151 | Synergy | Synergy |

### Example 2

### DOT1L Inhibitor Compound A2 Displays Synergistic Antiproliferative Activity in Combination with Standard of Care Drugs or DNA Hypomethylating Agents in MLL-Rearranged Leukemia Cells

The activity of Compound A2 in combination with current standard of care agents for acute leukemias as well as other chromatin modifying drugs was evaluated in cell proliferation assays with three human acute leukemia cell lines; Molm-13 (MLL-AF9 expressing acute myeloid leukemia (AML)), MV4-11 (MLL-AF4 expressing acute biphenotypic leukemia cell line) and SKM-1 (non-MLL-rearranged AML). A high density combination platform suitable for testing the antiproliferative activity of a complete titration matrix of two agents with multiple replicate points was established to enable generation of statistically meaningful results. This platform was used to evaluate the anti-proliferative effects of Compound A2 combinations tested in a co-treatment model in which the second agent was added along with Compound A2 at the beginning of the assay, or in a pre-treatment model in which cells were incubated for several days in the presence of Compound A2 prior to the addition of the second agent. The drug combination analysis was performed using the Chou-Talalay method [Chou T C Pharmacological Reviews 2006]. Graphs representing values of combination index (CI) versus Fractional effect (Fa) known as Fa-CI plots were generated and synergy was evaluated. Drug synergy was statistically defined by CI values less than 1, antagonism by CI>1 and additive effect by CI equal to 1.

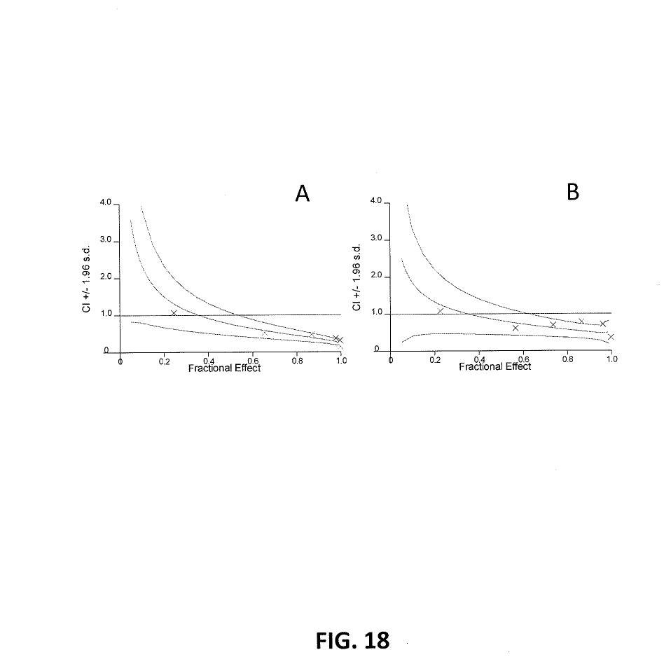

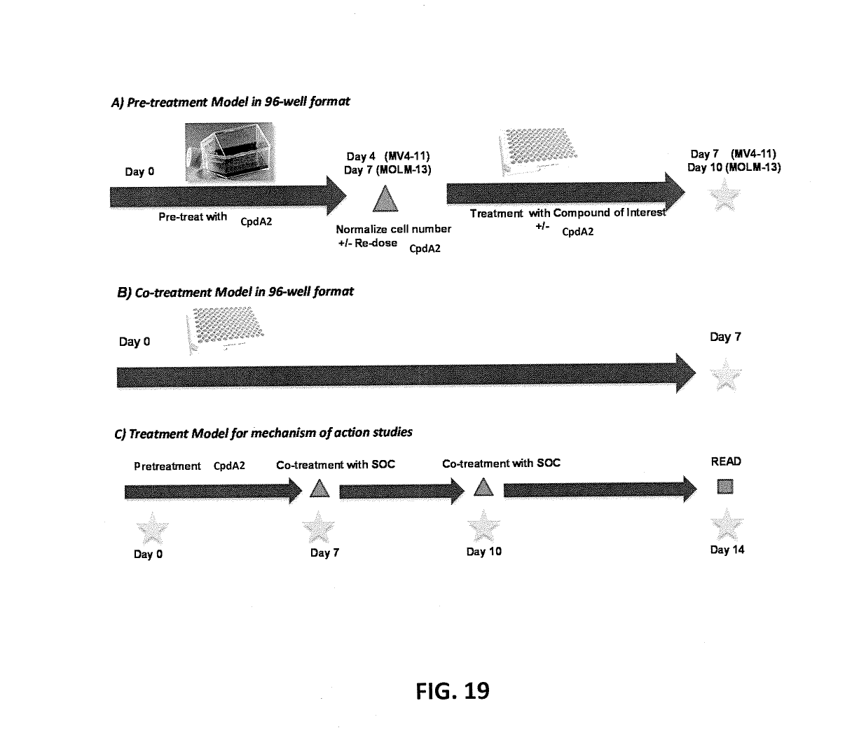

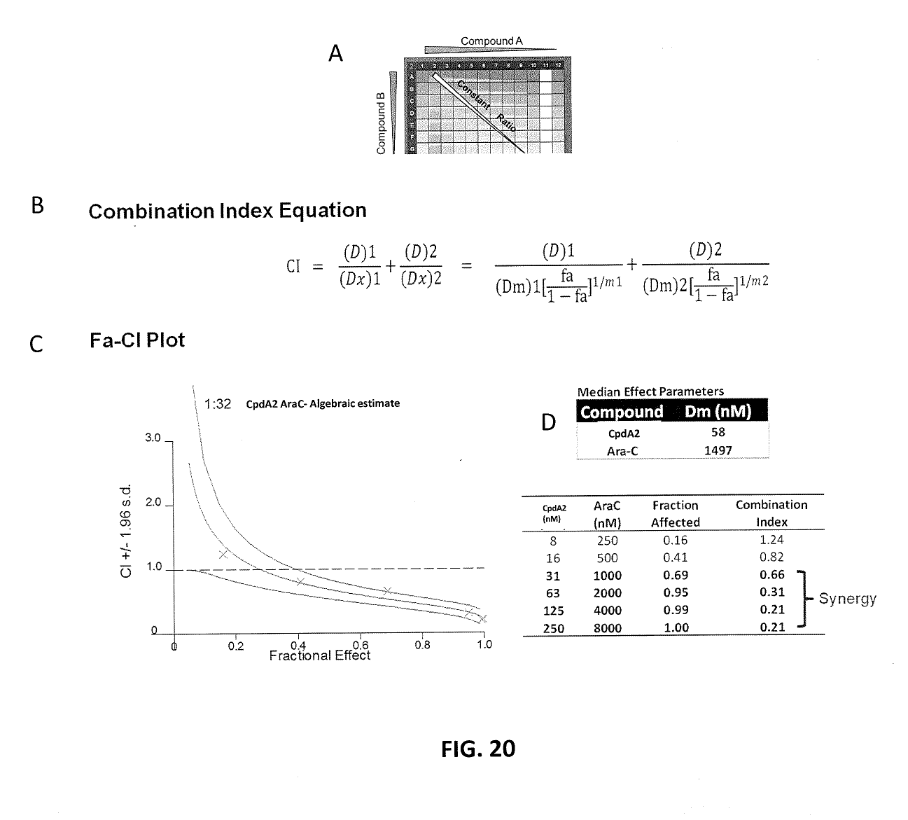

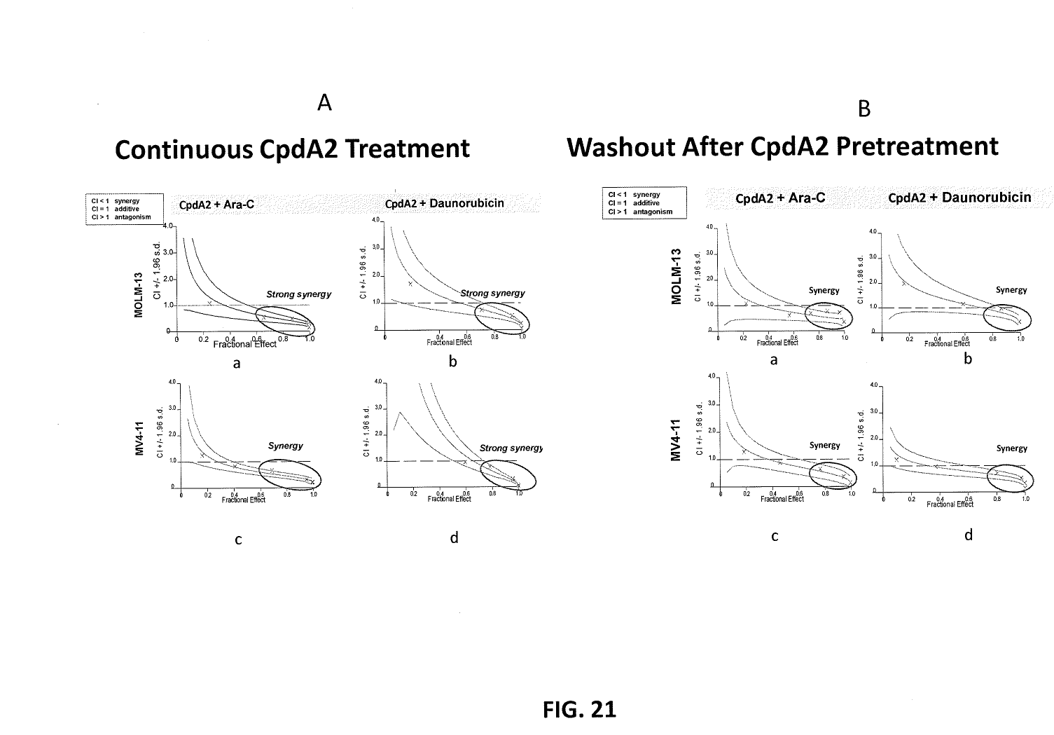

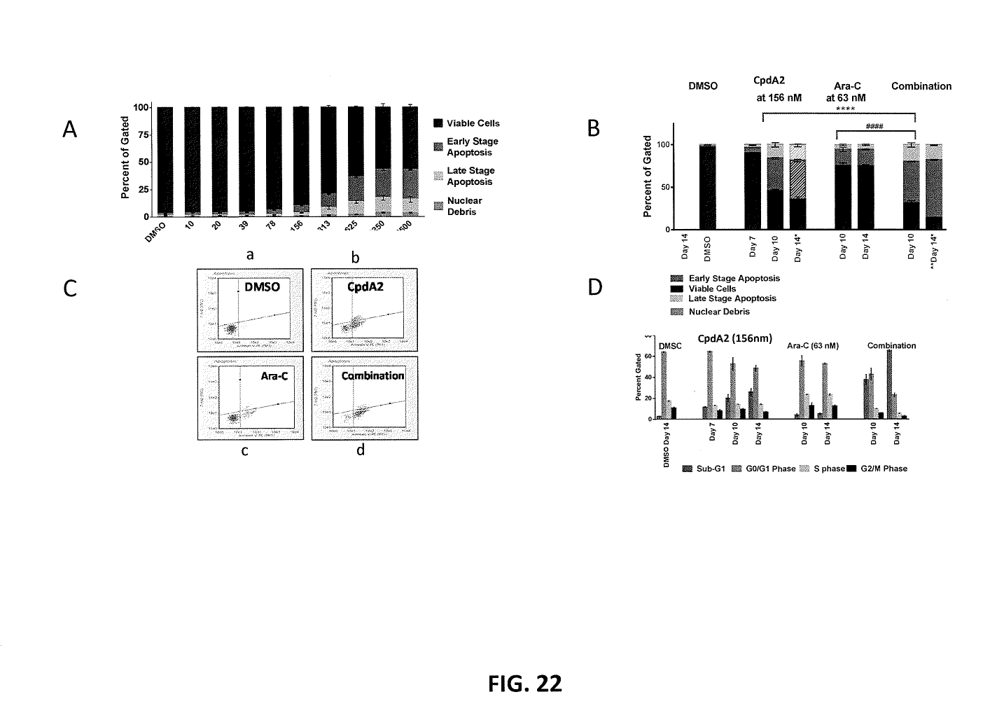

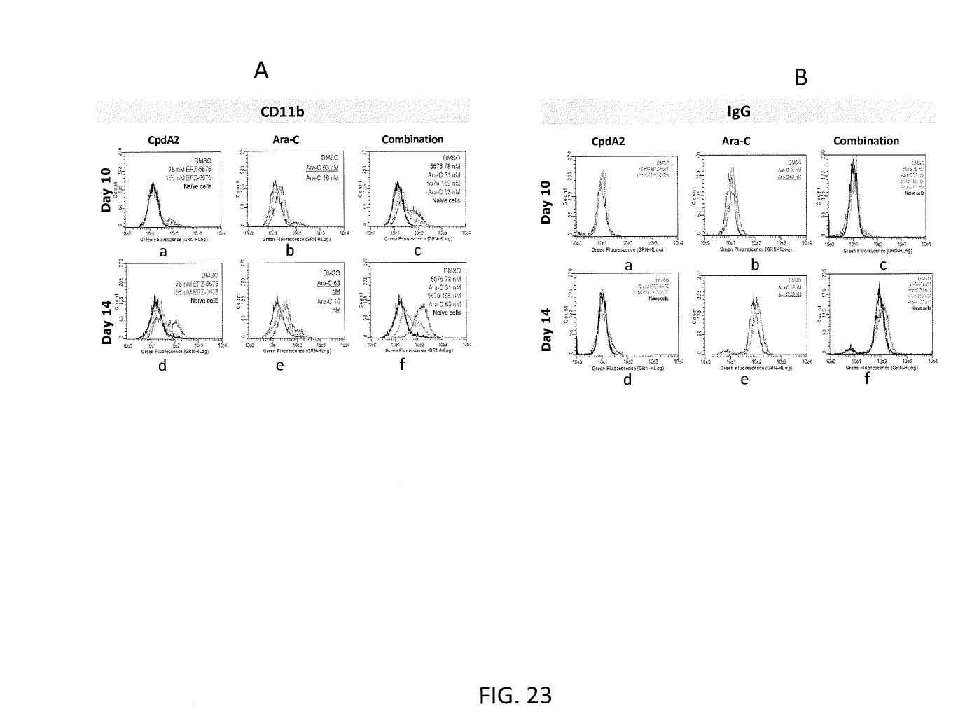

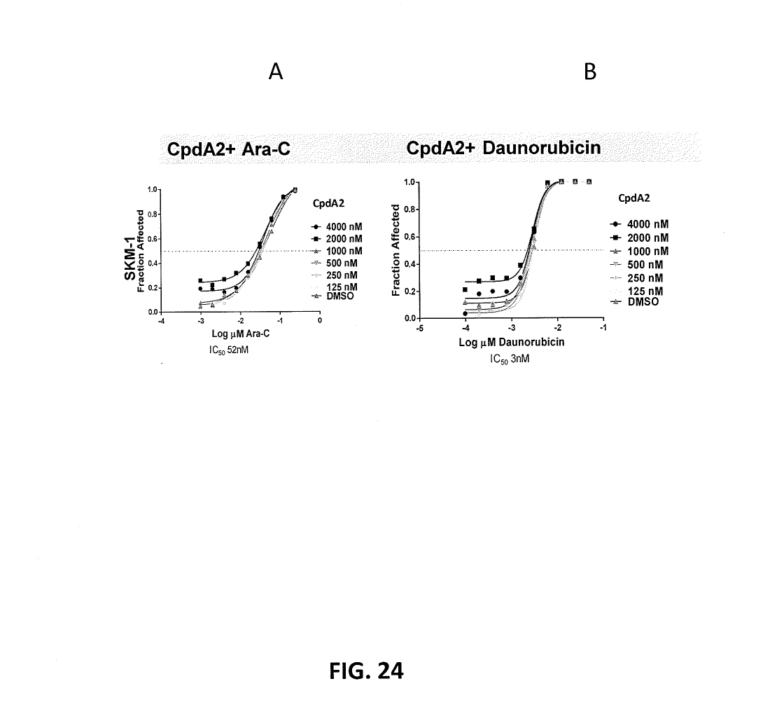

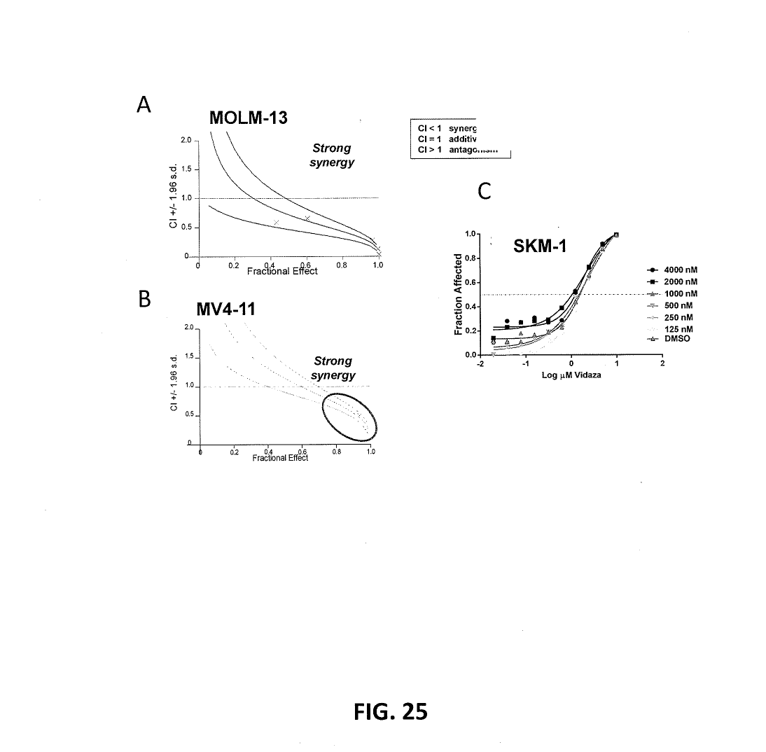

The results showed that Compound A2 acts synergistically with the AML standard of care agents cytarabine and duanorubicin in Molm-13 and MV4-11 MLL-rearranged cell lines. Moreover, a persistent combination benefit was observed even when Compound A2 was washed out prior to the addition of the standard of care agents (FIG. 18), suggesting that Compound A2 sets up a durable altered chromatin state that enhances the effect of chemotherapeutic agents in MLL-rearranged cells. The combination of Compound A2 with other chromatin modifying drugs also revealed a consistent combination benefit including synergy with DNA hypomethylating agents.

In summary, the results indicate that Compound A2 is highly efficacious as a single agent and is synergistic with other anticancer agents including AML standard of care drugs and DNA hypomethylating agents in MLL-rearranged cells.

### Example 3

### Example DOT1L Inhibitor Compound A2 Displays Synergistic Antiproliferative Activity in Combination with Standard of Care Drugs or DNA Hypomethylating Agents in MLL-Rearranged Leukemia Cells

Compound A2 is a small molecule inhibitor of the histone methyltransferase DOT1L that is currently under clinical investigation as a potential therapy for acute leukemias bearing MLL-rearrangements. Gene knockout and small molecule inhibitor studies have demonstrated that DOT is required for MLL-fusion protein—mediated leukemogenesis in model systems. In preclinical studies Compound A2 promoted cell killing of acute leukemia lines bearing MLL translocations in vitro while sparing those without MLL gene translocations and also caused sustained tumor regressions in a rat xenograft model of MLL-rearranged leukemia [Daigle et al. Blood 2013]. To support potential future clinical scenarios, the activity of Compound A2 in combination with current standard of care agents for acute leukemias as well as other chromatin modifying drugs was evaluated in cell proliferation assays with three human acute leukemia cell lines; Molm-13 (MLL-AF9 expressing acute myeloid leukemia (AML)), MV4-11 (MLL-AF4 expressing acute biphenotypic leukemia cell line) and SKM-1 (non-MLL-rearranged AML). We established a high density combination platform suitable for testing the anti-proliferative activity of a complete titration matrix of two agents with multiple replicate points to enable generation of statistically meaningful results. This platform was used to evaluate the anti-proliferative effects of Compound A2 combinations tested in a co-treatment model in which the second agent was added along with Compound A2 at the beginning of the assay, or in a pre-treatment model in which cells were incubated for several days in the presence of Compound A2 prior to the addition of the second agent. The drug combination analysis was performed using the Chou-Talalay method [Chou T C Pharmacological Reviews 2006]. Graphs representing values of combination index (CI) versus Fractional effect (Fa) known as Fa-CI plots were generated and synergy was evaluated. Drug synergy was statistically defined by CI values less than 1, antagonism by CI>1 and additive effect by CI equal to 1.

The results showed that Compound A2 acts synergistically with the AML standard of care agents cytarabine or daunorubicin in Molm-13 and MV4-11 MLL-rearranged cell lines. However, in the non-rearranged SKM-1 cell line Compound A2 had no effect alone and did not act synergistically with cytarabine or daunorubicin.

Moreover, a persistent combination benefit was observed even when Compound A2 was washed out prior to the addition of the standard of care agents suggesting that Compound A2 sets up a durable altered chromatin state that enhances the effect of chemotherapeutic agents in MLL-rearranged cells.

Evaluation of Compound A2 in conjunction with other chromatin modifying drugs also revealed a consistent combination benefit including synergy with DNA hypomethylating agents.

In summary, the results presented herein indicate that Compound A2 is highly efficacious as a single agent and is synergistic with other anticancer agents including AML standard of care drugs and DNA hypomethylating agents in MLL-rearranged cells.

### Methods:

### A) Pre-Treatment Model in 96-Well Format:

Human leukemia cell lines were pretreated in flasks with 7 concentrations of Compound A2 or DMSO for 4 (MV4-11 cells) or 7 days (MOLM-13 cells). Cells were then counted and reseeded with, or without Compound A2 (Compound A2 washout) in 96-well plates at a constant cell density in the presence of increasing concentrations of a second agent for an additional 3 days. The HP-D300 digital dispenser (Tecan) was used to dispense compounds in a combinatorial matrix. Cells were treated with concentrations of Compound A2 and standard of care agent which were bracketed above and below the IC₅₀ of each compound alone. Cell viability was measured via ATP content using CellTiter-Glo® (Promega).

### B) Co-Treatment Model in 96-Well Format:

Human leukemia cell lines were treated with matrix of 7 concentrations of Compound A2 and 9 concentrations of compound of interest for 7 days. Viability was determined using CellTiter-Glo® (Promega).

### C) Pre-Treatment Model for Mechanism of Cell Death Studies:

MOLM-13 cells were pretreated in flasks with 7 concentrations of Compound A2 or DMSO vehicle control for 7 days. Cells were then counted and reseeded in 96-well plates at a constant cell density in the presence of Compound A2 and Ara-C at concentrations previously demonstrated to give synergistic cell killing activity and incubated for an additional 3 or 7 days. A Guava EasyCyte HT™ flow cytometer was used to measure DNA content, Annexin V staining and cell surface expression of CD14 and CD11b markers on Days 10 and 14.

TABLE 6
Summary of Combination Studies with Compound A2 in AML Cell Lines

| | | | | SKM-1 |
|---|---|---|---|---|
| | | MV4-11 | MOLM-13 | AML: Non- |
| | | MLL-AF4 | MLL-AF9 | rearranged |
| AML Standard of | Ara-C | Strong | Synergy | No Combination |
| Care Agents | | Synergy | | Benefit |
| | Daunorubicin | Synergy | Synergy | No Combination |
| | | | | Benefit |
| DNA | Azacitidine | Synergy | Synergy | No Combination |
| Methyltransferase | | | | Benefit |
| Inhibitors | Decitabine | Synergy | Synergy | No Combination |
| | | | | Benefit |
| Bromodomain | IBET-151 | Synergy | Synergy | IC₅₀ not achieved |
| Inhibitors | JQ1 | Additive | Additive | TBD |

Combination benefit with Compound A2 is achieved with all drugs tested in MLL-rearranged leukemia cell lines Molm-13 and MV4-11 sparing the non-rearranged SKM-1 cell line. In summary, the present study demonstrates that:
(1) Compound A2 acts synergistically with the AML SOC drugs Ara-C and daunorubicin to induce a strong antiproliferative response that is selective for MLL-rearranged leukemia cells;
(2) Synergy is observed even when Compound A2 is washed out prior to the addition of Ara-C and daunorubicin;
(3) Initial studies suggest that the concurrent induction of apoptosis and differentiation underlies the combination benefit observed with SOC drugs in the MLL-rearranged leukemia cell line MOLM-13; and
(4) Synergistic anti-proliferative activity in MLL-rearranged leukemia cell lines is also observed when Compound A2 is used in combination with several chromatin modifying agents, including the DNA-methyltransferase inhibitors azacytidine and decitabine and the bromodomain inhibitor i-BET.

Taken together these studies suggest that Compound A2 sets up an altered chromatin and/or gene expression state in MLL-rearranged cells that dramatically potentiates the cytotoxic effects of current AML SOC drugs.

### Example 4

### Synergistic Activity of Ara-C and Compound A2

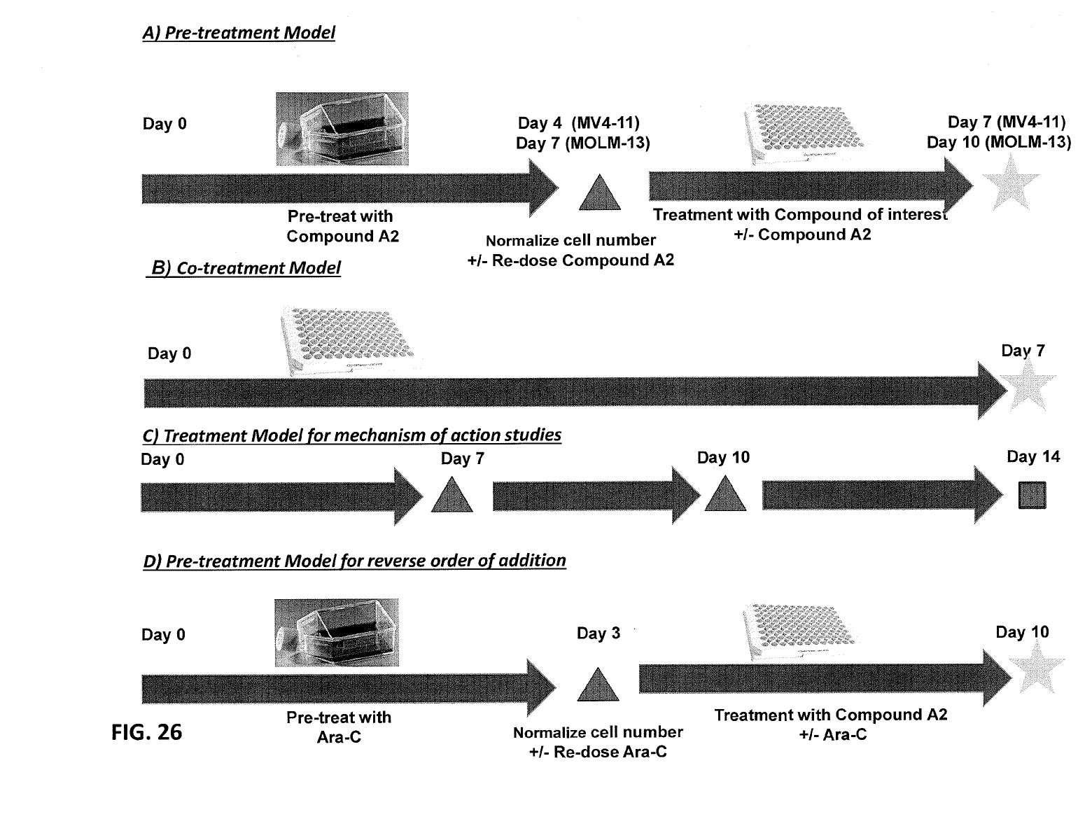

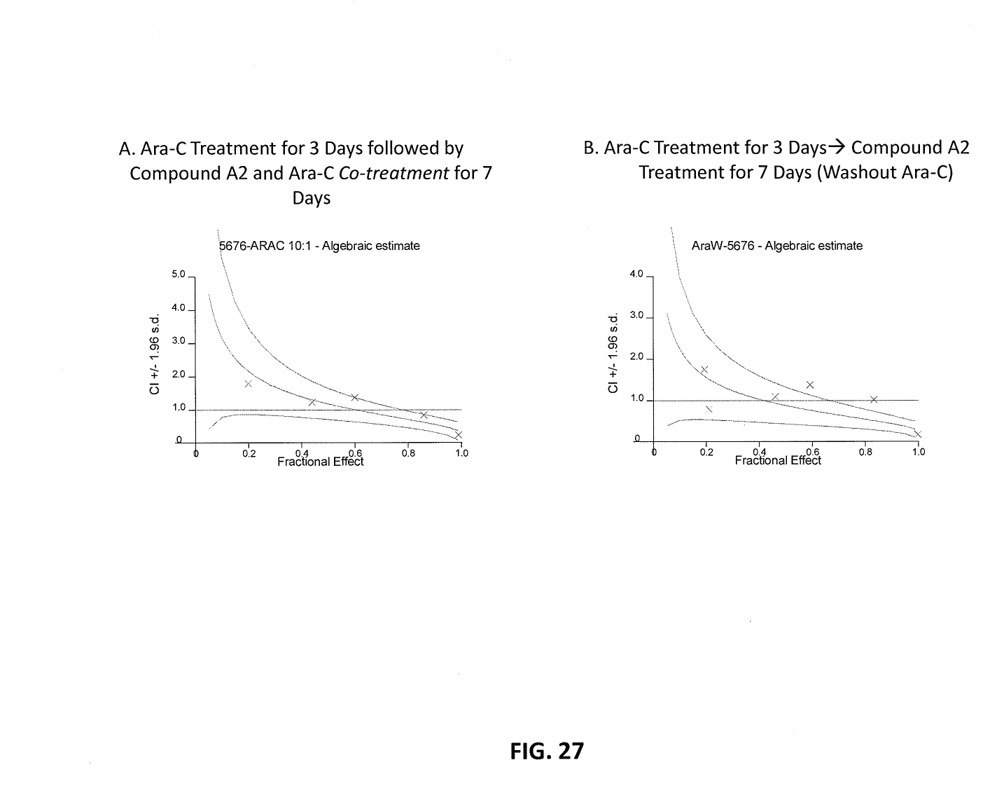

As shown in FIG. 26D, pre-treatment model with reverse order of addition in 96-well format is carried out as follows.

MOLM-13 cells were pretreated with 9 concentrations of Ara-C or DMSO for 3 days. Cells were then counted and reseeded with or without Ara-C (Ara-C washout) in 96-well plates at a constant cell density in the presence of increasing concentrations of Compound A2 for an additional 7 days.

The HP-D300 digital dispenser (Tecan) was used to dispense Compound A2 and Ara-C in a combinatorial matrix. Cells were treated with concentrations of Compound A2 and Ara-C bracketed above and below the IC₅₀ of each compound alone. Cell viability was measured via ATP content using CellTiter-Glo® (Promega).

### Results:

Synergy is observed when cells are pretreated with Ara-C followed by cotreatment with Compound A2. Combination benefit is maintained when Ara-C is washed out prior to treatment with Compound A2.

### Example 5

### Compound A2 Induces a Synergistic and Durable Antiproliferative Effect in Combination with AML Standard of Care Drugs

### Materials and Methods

### Cell Lines

The acute myelogenous leukemia cell line MV4-11 (MLL-AF4) (CRL-9591) was obtained from American Type Culture Collection (ATCC), Manassas, Va. and both MOLM-13 (MLL-AF9) (ACC 554) and SKM-1 (ACC 547) cells were obtained from Leibniz Institute DSMZ-German Collection of Microorganisms and Cell Cultures, Braunschweig, Germany. MV4-11 cells were maintained in IMDM supplemented with 10% fetal bovine serum. MOLM-13 and SKM-1 cells were maintained in Roswell Park Memorial Institute medium (RPMI) supplemented with 10% fetal bovine serum. They were cultured in flasks or plates in a humidified 5% CO₂ atmosphere.

### Proliferation Assays and Calculation of Synergism

Proliferation studies were performed using MOLM-13, MV4-11 and SKM-1 cell lines in vitro to evaluate the cancer cell killing effect of a combination of two agents together on cell growth. Initial proliferation studies were performed to determine the IC₅₀ values of a given compound in each cell line. The cell counts were measured by ATP quantitation using the Promega Cell Titer Glo kit and luminescence values correspond to the amount of ATP in a given well.

These studies were performed to evaluate both the combinatorial effect of compounds on cell killing and the durability of the effect by washing out one of the agents. Compounds were tested in combination with Compound A2 to study their effect on cell proliferation in either a 4+3 model where cells were pretreated with increasing concentrations of Compound A2 for 4 days, followed by a co-treatment with Compound A2 with test article for 3 days or a 7 day co-treatment model.

In addition, the effect of sequence of addition of compounds was studied by measuring the ten day proliferation of cells pretreated with Ara-C in a 3+7 model. This experiment was performed by first pretreating MOLM-13 cells with increasing concentrations of Ara-C for 3 days. Ara-C was then washed out, the cell numbers were normalized and either Compound A2 alone or Compound A2 cotreatment in a matrix format with Ara-C was performed. The cells were then normalized on day 3, followed by washout of Ara-C or cotreatment of cells with Compound A2 and Ara-C for 7 days.

Compounds evaluated for synergy in the co-treatment phase were tested in a range which was bracketed around the IC₅₀'s. The compounds were plated to a 96 well plate in a matrix format which includes increasing concentrations of each drug in the combination in a constant ratio, in addition to the effect of each compound alone in the study. Cells were seeded and grown in the log-linear phase for 3 or 7 days in the cotreatment phase. Maximum and minimum inhibition (DMSO alone) controls were used in each plate to calculate fraction affected (Fa) of a test well. DMSO concentration was kept at 0.1% v/v. The drug combination analysis was performed utilizing the Chou-Talalay method. Synergy was determined using the software package Calcusyn by Biosoft. The combination index (CI) is the term used to describe the level of synergy or antagonism in a given test system. A combination index less than one indicates synergy, and a CI greater than one indicates antagonism.

### Cell Treatment for Analysis of Mechanism of Cell Death Studies

On Day 0 MOLM-13 cells are seeded at 3,000 cells/mL. On Day 7 and Day 10 MOLM-13 cells are counted and reseeded at 50,000 cells/mL. MOLM-13 cells were treated with various concentrations of compounds as a single agent or in combination with AraC or Daunorubicin. Day 1-7 cells were only treated with Compound A2. On Day 7 cells reseeded and redosed with Compound A2 alone or in combination with AraC or Daunorubicin as described below. On Day 10 They were redosed again. On Day 14 the experiment was terminated. Cells were sampled for CD14 and CD11b analysis on Days 7, 10 and 14.

### Flow Cytometric Analysis of Cell Cycle and Annexin V

To evaluate the fraction of cells in each cell cycle, flow cytometric analysis was performed. FACS analysis for detection of cell death by apoptosis, and cell cycle was performed. Cells were treated alone with Compound A2 or in combination. To allow for simultaneous analysis of cell cycle and apoptosis, cells were treated alone or in combination with Compound A2.

Cells were harvested on days 7, 10 and 14 and split to allow simultaneous analysis of cell cycle and Annexin V staining Apoptosis was determined using the Guava Nexin Assay (Millipore 4500-0450) and cells were prepared according to the manufacturer's recommendations. Samples were analyzed using the Guava EasyCyte Plus System (Millipore). Cells for cell cycle analysis were pelleted by centrifugation at 200×g for 5 minutes at 4° C., washed twice with ice cold PBS then fixed with 70% ice cold ethanol. All samples were analyzed together at end of experiment. Following fixation cells were washed with PBS and stained with the Guava cell cycle reagent (Millipore 4500-0220) for 30 minutes. Samples were analyzed using the Guava EasyCyte Plus System (Millipore).

### Analysis of CD11b and CD14 Expression by Flow Cytometry

To analyze the degree of differentiation, MOLM-13 cells were incubated in the presence of 0.1% DMSO or previously stated concentrations of Compound A2, Ara-C, Daunorubicin or in combination. On day 7, 10, and 14, cells were collected for analysis. The cells were prepared by washing twice in PBS, followed by fixation in 4% formaldehyde for ten minutes at 37° C. After fixation cells were washed and blocked with blocking buffer for 10 minutes at room temperature. Cells were then incubated in presence of anti-CD14, anti-CD11b or anti-IgG antibody for 1 hour at room temperature while rotating. Cells were washed, re-suspended in PBS and 5,000 events were analyzed using ExpressPro software on the GuavaCyte Plus System.

### Analysis of CD11b and Caspase Cleavage by High Content Screening

To further analyze the cell population for differentiation or markers of apoptotic cell death, MOLM-13 cells were collected on days 5, 7, 8, 9, 10, 11, 12 and 14 for imaging. Cells were incubated with test articles, and at each time point, cells were collected, washed once in PBS and re-suspended in 0.5% BSA+PBS blocking buffer. CD11b antibody, at a dilution of 1:12.5, was incubated with the cells for 15 minutes at 37° C. in the dark at room temperature while rotating. Medium A was added and the cells were incubated for an additional 15 minutes. After one wash with PBS+0.1% NaN₃+5% FBS cells were re-suspended in Medium B from the Fix and Perm kit. DAPI at a 1:100,000 dilution and second antibody (Caspase-3 or H2A.X) at a 1:50 dilution were added and cells incubated for 20 minutes at room temperature in the dark. After the last incubation, cells were washed one time in PBS+0.1% NaN₃+5% FBS and re-suspended in 150 μL of PBS, allowed to settle on the plate for about 30-60 minutes then imaged.

The drug combination analysis was performed using the Chou-Talalay method. Graphs representing values of combination index (CI) versus Fractional effect (Fa) known as Fa-CI plots were generated and synergy was evaluated. Drug synergy was statistically defined by CI values less than 1, antagonism by CI>1 and additive effect by CI equal to 1.

### Results

Compound A2 Induces a Synergistic and Durable Antiproliferative Effect in Combination with AML Standard of Care Drugs

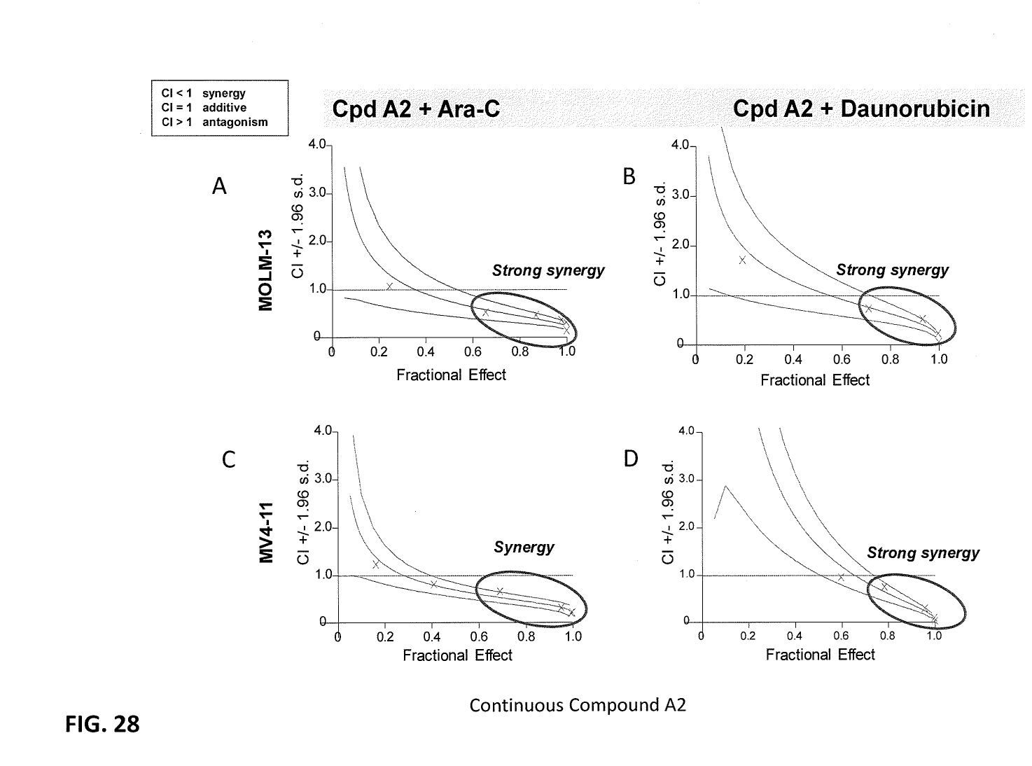

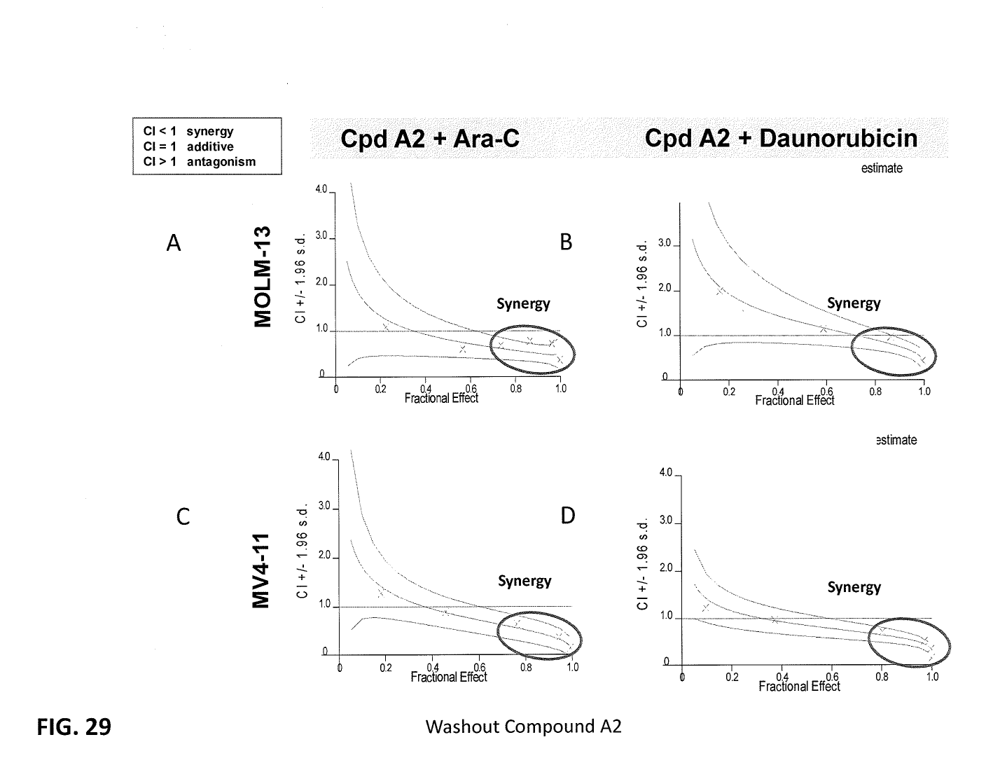

Compound A2 demonstrates synergistic antiproliferative activity in combination with two standard of care (SOC) drugs for AML, cytarabine and daunorubicin in the MLL-rearranged leukemia cell lines MOLM-13 and MV4-11 (FIG. 28). Cells were treated according to the pre-treatment model described in above (i.e., no Compound A2 washout). The synergistic anti-proliferative activity of Compound A2 in combination with AML SOC agents was also observed when cells were treated according to the co-treatment model. Intriguingly, this synergistic anti-proliferative activity was maintained in MOLM-13 and MV4-11 MLL-rearranged cells even when Compound A2 is removed (i.e., washed out) prior to the addition of the SOC agent (FIG. 29). These data are remarkable in that they imply a durable reprogramming of the epigenetic status of these cells by Compound A2 that renders them more acutely sensitive to chemotherapeutic agents, even when the DOT1L inhibitor has been removed from the cellular environment. This result is consistent with the kinetics of Compound A2 effect on histone methylation at the DOT1L substrate site, H3K79 (Daigle et al, 2013). In previous studies, we have shown that four days of treatment with Compound A2 is sufficient to deplete cellular levels of H3K79me2 by >80%. When Compound A2 was then removed, by wash out from these cells, no recovery of H3K79 methylation was observed for 3 days after wash out. After this 3-day latency period, the level of H3K79me2 slowly returned to pretreatment levels over the course of an additional 4 days. Hence, treatment of MLL-rearranged cells with Compound A2 results in durable inhibition of H3K79 methylation which in turn results in sensitization of these cells to chemotherapy-induced cell killing. These results offer the possibility of a highly flexible dosing schedule for combinations of Compound A2 and chemotherapies.

The synergistic effects of Compound A2 and chemotherapeutic agents were very similar in both MLL-rearranged cells tested (MV4-11 and MOLM-13). In the interest of clarity and brevity, below we present representative data for MOLM-13 cells only. In all cases, similar results were observed in the MV4-11 cell line as well.

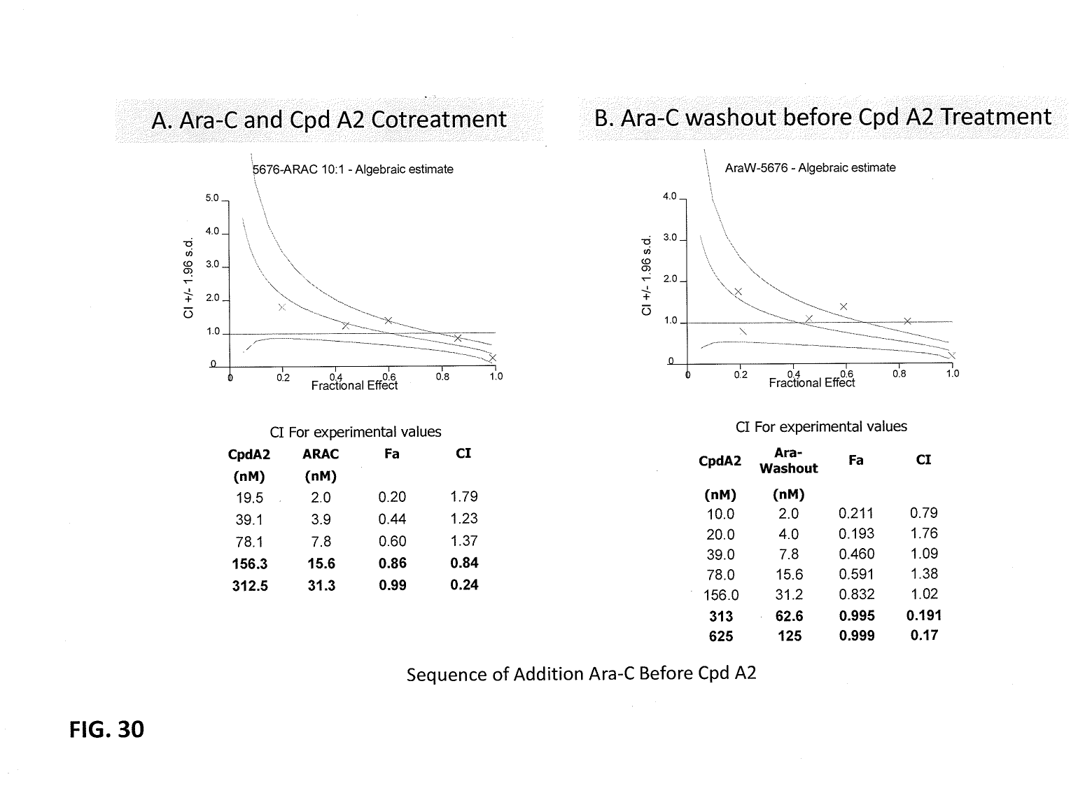

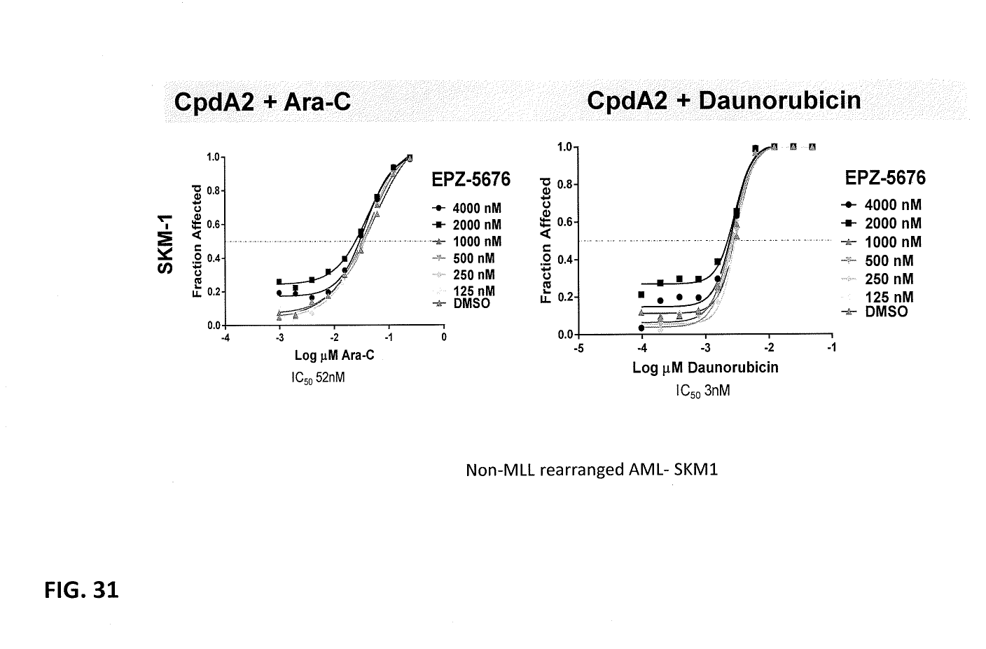

To test further the flexibility of dosing schedules that might afford synergistic cell killing, we pretreated MOLM-13 cells with the chemotherapeutic agent cytarabine for 3 days, washed this drug out and then treated the cells with Compound A2 for an additional 7 days. As illustrated in FIG. 30, this sequential treatment schedule resulted in essentially the same level of synergistic cell killing as seen when both drugs were co-administered to cells simultaneously.

While both single agent activity and strong synergy with cytarabine and daunorubicin were seen for Compound A2 in the MLL-rearranged cell lines MV4-11 and MOLM-13, no effect of Compound A2 was observed in the non-MLL-rearranged leukemia cell line SKM-1. Compound A2 showed no single agent activity in this latter cell line and did not affect the antiproliferative activity of either chemotherapeutic agent in this cell line either (data not shown). The lack of activity of Compound A2 in SKM-1 cells is completely consistent with the proposed mechanism of action of this drug. In previous studies we have demonstrated that while Compound A2 inhibits intracellular DOT1L activity—as evidenced by concentration-dependent inhibition of H3K79 methylation—across a spectrum of AML cell lines, this enzyme inhibition only translates into an antiproliferative effect for those leukemia cells bearing an 11 q23 chromosomal translocation.

Compound A2 Increases Expression of Differentiation Markers and Apoptosis as Single Agent and in Combination with AML Standard of Care Drugs

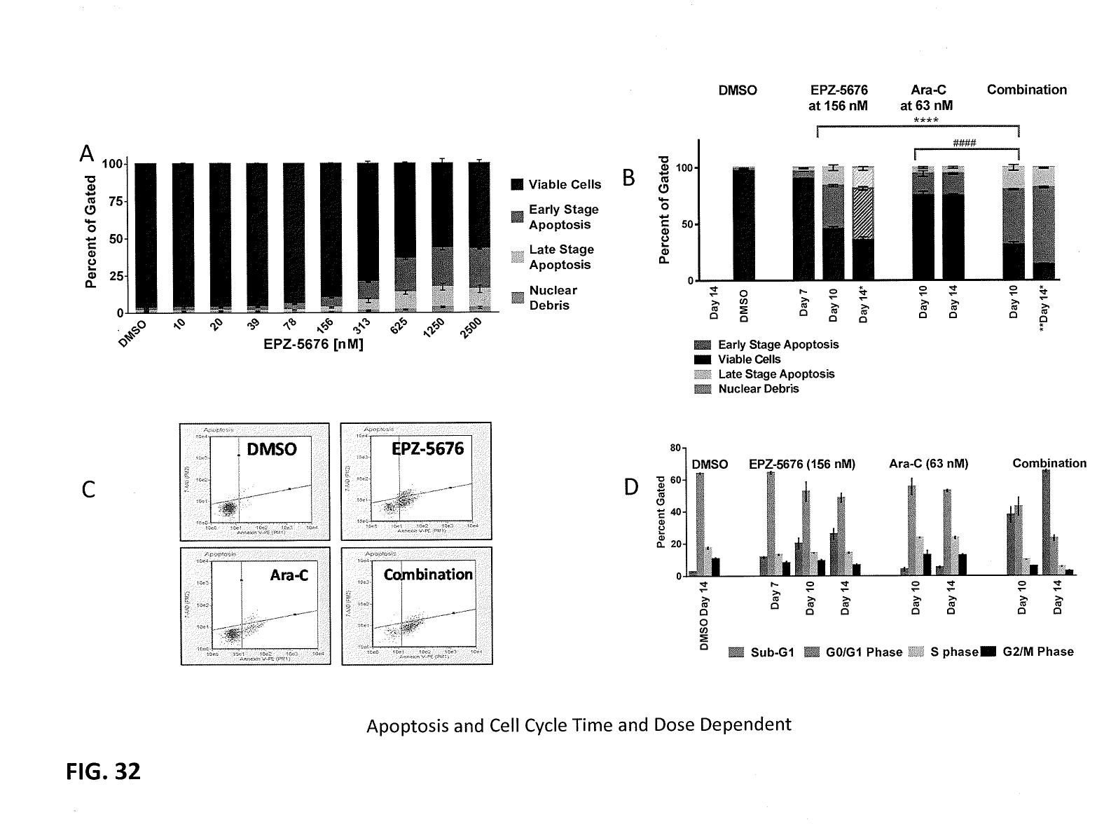

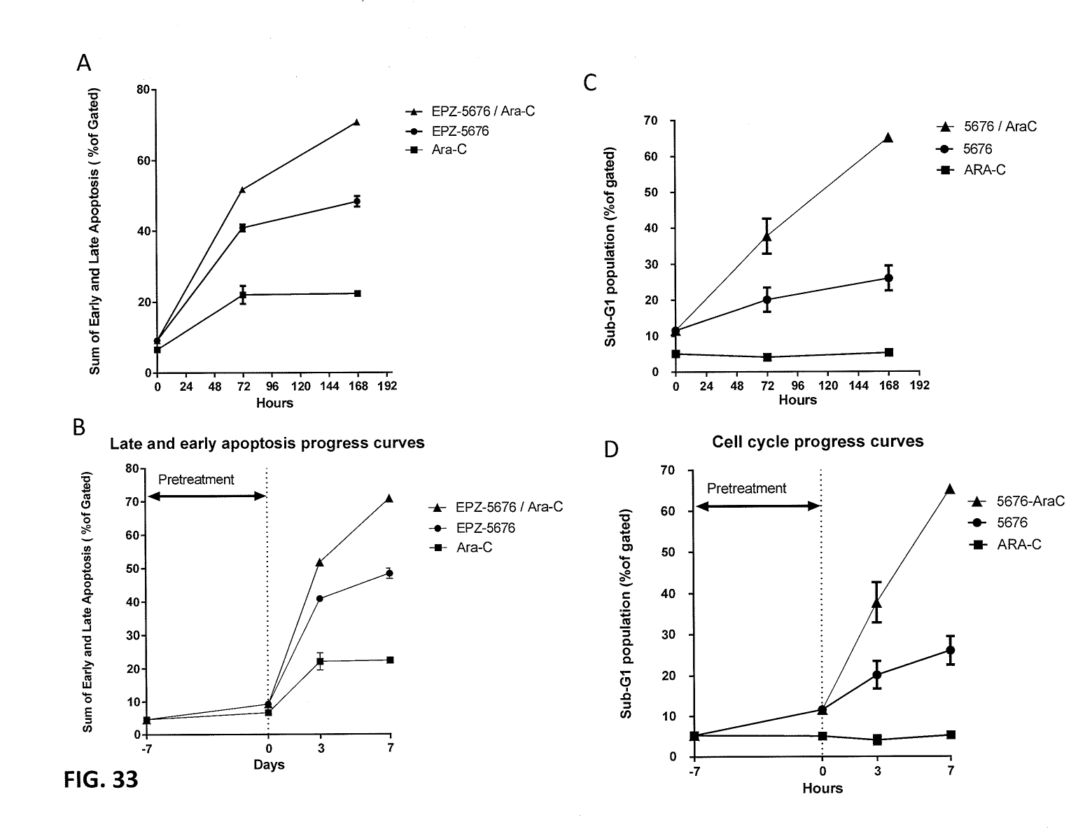

Compound A2 induces a concentration-dependent increase in apoptotic cells (as measured by Annexin-V staining) after 7 days of treatment of MOLM-13 cells as a single agent. As illustrated in FIG. 32A, the total content of viable cells decreases with Compound A2 concentration according to a classic Langmuir isotherm, with a midpoint value (EC₅₀) of 364±18 nM and this trend is exactly mirrored by the increasing content of apoptotic cells (sum of early and late stage apoptosis). The kinetics apoptosis induction was measured at fixed time points over a 14 day course of treatment for MOLM-13 cells treated with DMSO (as a control), 156 nM Compound A2, 63 nM cytarabine (Ara-C) or a combination of Compound A2 and Ara-C (at the same concentrations as for the single agent treatments). Ara-C by itself induced a modest increase in apoptotic cell population over the 14 day treatment period, while Compound A2 lead to much more robust induction of apoptosis over the same time course. The combination of the two drugs led to enhance apoptosis in the MOLM-13 cells (FIG. 32B). Apoptotic cell content was also assessed by measuring the percent of cells in the sub-G1 phase of the cell cycle. FIG. 32C illustrates the distribution of cell cycle stages at various time points for MOLM-13 cells treated with DMSO (control), 156 nM Compound A2, 63 nM Ara-C or a combination of Compound A2 and Ara-C. The data for the sub-G1 cell population is also graphed as a kinetic plot in FIG. 32D. This plot makes clear that Ara-C treatment alone has minimal effect of the sub-GI population of MOLM-13 cells over the 14 day treatment course, while treatment with Compound A2 leads to a moderate, time-dependent increase in sub-G1 population. When Compound A2 and Ara-C are combined, a significant increase in the population of sub-G1 cells at 10 and 14 days is realized with a concomitant increase in the rate of sub-G1 population growth as well. Similar results were observed when Compound A2 was combined with daunorubicin.

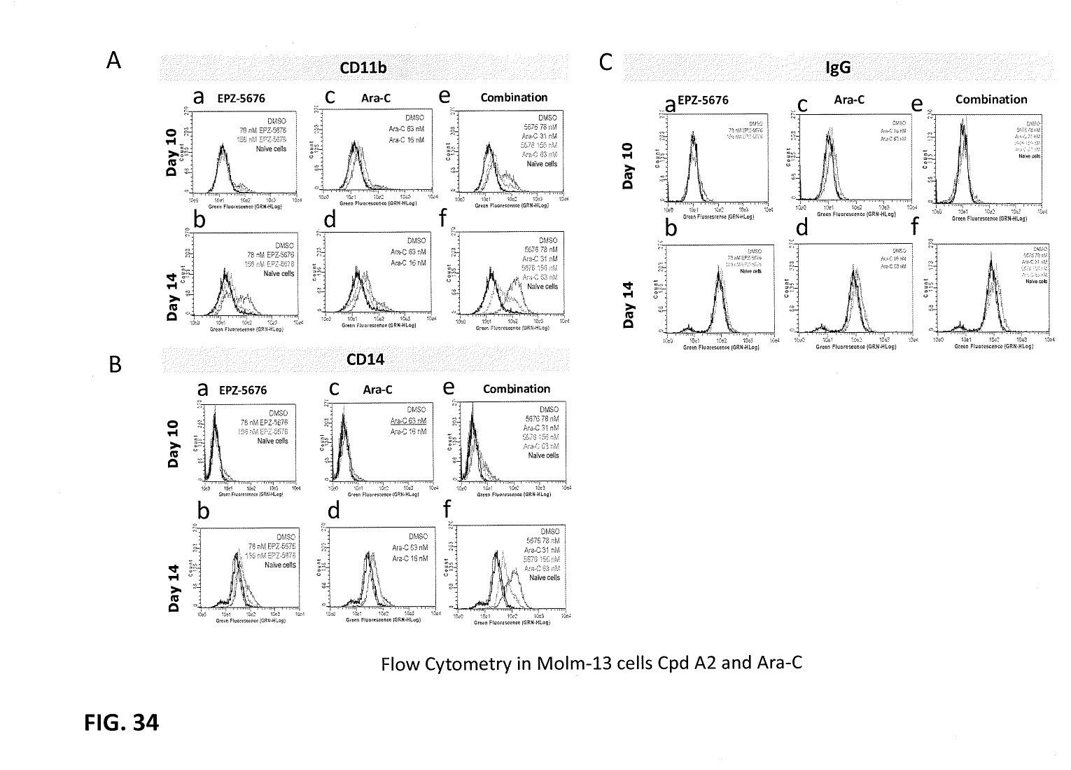

In addition to driving apoptotic cell death, Compound A2, Ara-C as single agents and in combination promote time and concentration dependent up-regulation of the differentiation markers CD11b and CD14 (FIG. 34) in MLL-rearranged MOLM-13 cells. The same effect was observed with daunorubicin as a single agent and in combination with Compound A2.

Compound A2 Demonstrates Strong Synergy with DNMT Inhibitors in MLL-Rearranged Cell Lines

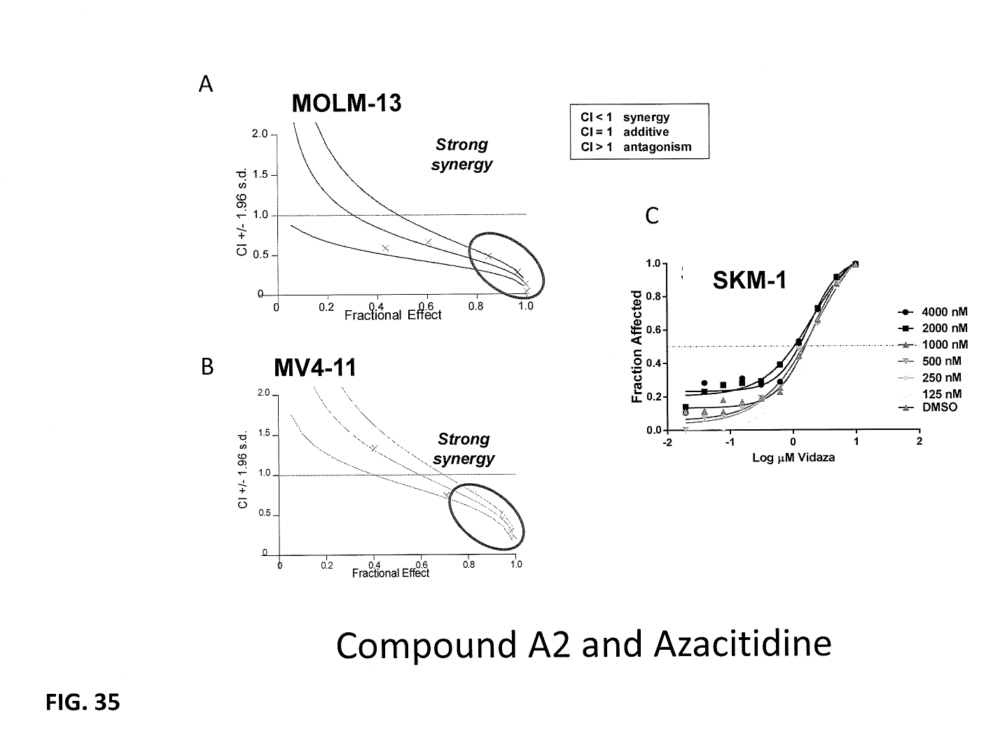

Compound A2 represents the first protein methyltransferase (PMT) inhibitor to be tested in human clinical trials. The PMT target class effects chromatin remodeling and gene transcriptional programming by site-specific methylation of lysine residues on histones H3 and H4; in the case of DOT1L, the enzyme uniquely catalyzes the methylation of a single histone site, H3K79. There is considerable evidence that epigenetic regulation of gene transcriptional results from the combinatorial effects of distinct covalent modifications of chromatin components, including histone methylation, histone acetylation, other covalent histone modifications and direct methylation of chromosomal DNA at CpG islands by the DNA methyltransfersases (DNMTs). Next, the impact of combining the PMT inhibitor Compound A2 in combination with other compounds that affect their pharmacology was tested by inhibition of other chromatin modifying enzymes, such as histone deacetylases (HDAC) histone demethylases (HDMs), acetyl-lysine reader domains (bromodomains) and DNA methyltransferases (DNMTs). The results of these combinations are summarized in Table 7 and demonstrate a range of effects from antagonism with some HDAC inhibitors in the context of MV4-11 cells to synergy. Among these other chromatin modifying enzyme inhibitors, the DNMT inhibitors decitabine and azacytidine demonstrated synergistic anti-proliferative activity in MLL-rearranged cells when combined with Compound A2. In contrast, and again consistent with the mechanism of action of Compound A2, this compound had no impact on the antiproliferative activity of either DNMT inhibitor when tested in the non-MLL-rearranged leukemia cell line SKM-1 (Table 7). FIG. 35 illustrates representative data for the strong synergistic effects of combining azacitidine and Compound A2 in MV4-11 and MOLM-13 cell lines. Similar synergy was also seen in these cell lines when Compound A2 was combined with another DNMT inhibitor, decitabine (Table 7).

TABLE 7
Summary of Combinations Evaluated in 7 Day Cotreatment Model

| Rationale | Compound | MV4-11 (MLL-AF4) | MOLM-13 (MLL-AF9) |
|---|---|---|---|
| AML Standard of Care | Ara-C | Strong Synergy | Synergy |
| | Daunorubicin | Synergy | Synergy |
| DNMTi | Azacitidine | Synergy | Synergy |
| | Decitabine | Synergy | Synergy |
| HDACi | Vorinostat | Antagonistic | Additive/Synergy |
| | Panobinostat | Antagonistic | Synergy |
| HDMi | Tranylcypromine | Synergy | Synergy |
| | LSD1 inhibitor II | Additive | Additive |
| Bromodomain-i | IBET-151 | Synergy | Synergy |
| | JQ1 | Additive | Additive |
| Acute Lymphoblastic | Mitoxantrone | Synergy | Synergy |
| Leukemia | Methotrexate | Additive | Antagonistic/Additive |
| Standard of Care | Mafosfamide | Synergy | Synergy |
| | Prednisolone | Antagonistic | Antagonistic |
| | Vincristine | Additive | Additive |
| Bcl-2i | Navitoclax | Synergy | Synergy |
| Immunomodulatory | Lenalidomide | Combination Benefit | Combination Benefit |
| Proteasome Inhibitor | Velcade | Combination Benefit | Combination Benefit |
| Antimetabolite | Hydroxyurea | Synergy | |
| MLL binding partner | Menin-MLL | Synergy | Synergy |
| inhibitor | inhibitor MI-2 | | |

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A composition comprising a compound of Formula (I): 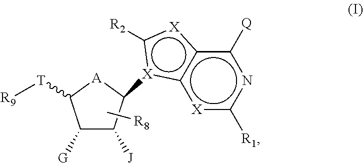
or pharmaceutically acceptable salts thereof, and one or more therapeutic agents, wherein,
T is a linker group of a 6-10 carbon atoms, in which one or more carbon atoms are optionally replaced with a heteroatom and T is optionally substituted;
R₉ comprises a C₆-C₁₀ aryl or 5 to 10-membered heteroaryl optionally substituted with one or more substituents selected from the group consisting of unsubstituted or substituted t-butyl, CF₃, cyclohexyl, C₆-C₁₀ aryl, and 5 to 10-membered heteroaryl;
A is O or CH₂;
each of G and J, independently, is H, halo, C(O)OH, C(O)O—C₁-C₆ alkyl or ORₐ, Rₐ being H, C₁-C₆ alkyl, C(O)—C₁-C₆ alkyl, or silyl, wherein C(O)O—C₁-C₆ alkyl, C₁-C₆ alkyl or C(O)—C₁-C₆ alkyl is optionally substituted with one or more substituents selected from the group consisting of halo, cyano hydroxyl, carboxyl, C₁-C₆ alkoxyl, amino, mono-C₁-C₆ alkylamino, di-C₁-C₆ alkylamino, and C₃-C₈ cycloalkyl;
each X independently is N or CRₓ, in which Rₓ is H, halo, hydroxyl, carboxyl, cyano, or R_{S1}, R_{S1} being amino, C₁-C₆ alkoxyl, C₁-C₆ alkyl, C₂-C₆ alkenyl, C₂-C₆ alkynyl, C₃-C₈ cycloalkyl, C₆-C₁₀ aryl, 4 to 6-membered heterocycloalkyl, or 5 to 6-membered heteroaryl, and R_{S1} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
each of R₁ and R₂, independently is H, halo, hydroxyl, carboxyl, cyano, or R_{S2}, R_{S2} being amino, C₁-C₆ alkoxyl, C₁-C₆ alkyl, C₂-C₆ alkenyl, C₂-C₆ alkynyl, or C₃-C₈ cycloalkyl, and each R_{S2} being optionally substituted with one or more substituents selected from the group consisting of halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl;
R₈ is H, halo or R_{S3}, R_{S3} being C₁-C₆ alkyl, C₂-C₆ alkenyl, or C₂-C₆ alkynyl, and R_{S3} being optionally substituted with one or more substituents selected from the group consisting of halo, hydroxyl, carboxyl, cyano amino, C₁-C₆ alkoxyl, mono-C₁-C₆ alkylamino, di-C₁-C₆ alkylamino, and C₃-C₈ cycloalkyl; and
Q is H, NH₂, NHR_{b}, NR_{b}R_{c}, R_{b}, ═O, OH, or OR_{b}, in which each of R_{b} and Rₑ independently is C₁-C₆ alkyl, C₂-C₆ alkenyl, C₂-C₆ alkynyl, C₃-C₈ cycloalkyl, C₆-C₁₀ aryl, 4 to 7-membered heterocycloalkyl, 5 to 10-membered heteroaryl, or -M₁-T₁ in which M₁ is a bond or C₁-C₆ alkyl linker optionally substituted with halo, cyano, hydroxyl or C₁-C₆ alkoxyl and T₁ is C₃-C₈ cycloalkyl, C₆-C₁₀ aryl, 4 to 6-membered heterocycloalkyl, or 5 to 10-membered heteroaryl, or R_{b} and R_{c}, together with the N atom to which they attach, form 4 to 7-membered heterocycloalkyl having 0 or 1 additional heteroatoms to the N atom optionally substituted with C₁-C₆ alkyl, C₂-C₆ alkenyl, C₂-C₆ alkynyl, halo, hydroxyl, carboxyl, C(O)OH, C(O)O—C₁-C₆ alkyl, OC(O)—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, or 5 to 6-membered heteroaryl, and each of R_{b}, R_{c}, and T₁ is optionally substituted with one or more substituents selected from the group consisting of C₁-C₆ alkyl, C₂-C₆ alkenyl, C₂-C₆ alkynyl, halo, hydroxyl, carboxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl.

2. The composition of claim 1, wherein the compound has formula (IV): 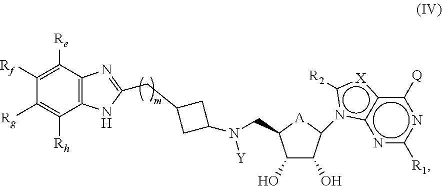
wherein each of Rₑ, R_{f}, R_{g}, and Rₕ, independently is -M₂-T₂, in which M₂ is a bond, SO₂, SO, S, CO, CO₂, O, O—C₁-C₄ alkyl linker, C₁-C₄ alkyl linker, NH, or N(Rₜ), Rₜ being C₁-C₆ alkyl, and T₂ is H, halo, or R_{S4}, R_{S4} being C₁-C₆ alkyl, C₂-C₆ alkenyl, C₂-C₆ alkynyl, C₃-C₈ cycloalkyl, C₆-C₁₀ aryl, 4 to 8-membered heterocycloalkyl, or 5 to 10-membered heteroaryl, and each of O—C₁-C₄ alkyl linker, C₁-C₄ alkyl linker, Rₜ, and R_{S4} being optionally substituted with one or more substituents selected from halo, hydroxyl, carboxyl, cyano, C₁-C₆ alkyl, C₂-C₆ alkenyl, C₂-C₆ alkynyl, C₁-C₆ alkoxyl, amino, mono-C₁-C₆ alkylamino, di-C₁-C₆ alkylamino, C₃-C₈ cycloalkyl, C₆-C₁₀ aryl, 4 to 6-membered heterocycloalkyl, and 5 to 6-membered heteroaryl, and
m is 0, 1, or 2.

3. A composition comprising any one of the compounds listed in Tables 1-4 or pharmaceutically acceptable salts thereof and one or more therapeutic agents.

4. A composition comprising Compound A2, or pharmaceutically acceptable salts thereof, and one or more therapeutic agents.

5. A composition comprising Compound D16, or pharmaceutically acceptable salts thereof, and one or more therapeutic agents.

6. The composition of any one of claims 1-5, wherein the one or more therapeutic agents are anti-cancer agents.

7. The composition of any one of claims 1-5, wherein the one or more therapeutic agents are selected from Ara-C, Daunorubicin, Decitabine, Vidaza, Mitoxantrone, JQ1, IBET151, Panobinostat, Vorinostat, Quizartinib, Midostaurin, Tranylcypromine, LSD1 inhibitor II, Navitoclax, and analogs, derivatives, or combinations thereof.

8. The composition of any one of claims 1-5, wherein the therapeutic agent is Ara-C, Daunorubicin, Vidaza or an analog or derivative thereof.

9. A pharmaceutical composition comprising a therapeutically effective amount of composition of any one of claims 1-8 and a pharmaceutically acceptable carrier.

10. A method of treating or alleviating a symptom of a disease comprising administering to a subject in need thereof a therapeutically effective amount of a composition of claim 1.

11. The method of claim 10, wherein the disease is cancer or a precancerous condition.

12. The method of claim 10, wherein the disease can be influenced by modulating the methylation status of histones or other proteins.

13. A method of claim 12, wherein the methylation status is mediated at least in part by the activity of DOT1L.

14. A method of treating or alleviating a symptom of cancer comprising administering to a subject in need thereof a therapeutically effective dose of a compound of Formula (I) and one or more therapeutic agents, wherein the compound of Formula (I) and the one or more therapeutic agents are administered simultaneously or sequentially.

15. The method of claim 14, wherein a compound of Formula (I) is administered prior to administration of the one or more therapeutic agents.

16. A method of treating or alleviating a symptom of cancer comprising administering to a subject in need thereof a therapeutically effective dose of a compound of Formula (I), or a pharmaceutically acceptable salt thereof, prior to administering a therapeutically effective dose of a composition of claim 1.

17. The method of claim 10 or 16, wherein the composition of claim 1 is administered to the subject in need thereof at a dosage of 0.01 mg/kg per day to about 1000 mg/kg per day.

18. The method of claim 14 or 16, wherein the compound of Formula (I) is administered at a dosage of 0.01 mg/kg per day to about 1000 mg/kg per day.

19. The method of claim 14 or 16, wherein each of the one or more therapeutic agents is administered at a dosage of 0.01 mg/kg per day to about 1000 mg/kg per day.

20. The method of claim 14 or 16, wherein the compound of Formula (I) or a pharmaceutically acceptable salt thereof is administered at a dose of at least 36 mg/m².

21. The method of claim 14 or 16, wherein the compound of Formula (I) or a pharmaceutically acceptable salt thereof is administered at a dose of at least 54 mg/m².

22. The method of claim 14 or 16, wherein the compound of Formula (I) or a pharmaceutically acceptable salt thereof is administered at a dose of at least 80 mg/m².

23. The method of any one of claim 14, 16 or 18-22, wherein the compound of Formula (I) or a pharmaceutically acceptable salt thereof is administered continuously for at least 7, 14, 21, 28, 35, 42, 47, 56, or 64 days.

24. The method of claim 23, wherein continuous administration comprises administration without a drug holiday.

25. The method of any one of claims 10-24, wherein the administration results in maturation or differentiation of leukemic blast cells.

26. The method of claim 25, wherein at least 20% of leukemic blast cells have undergone maturation or differentiation.

27. The method of claim 25, wherein at least 50% of leukemic blast cells have undergone maturation or differentiation.

28. The method of claim 25, wherein at least 80% of leukemic blast cells have undergone maturation or differentiation.

29. The method of any one of claims 10-28, wherein administration results in reduction of H3K79 methyl mark to at least 90%, 80%, 70%, 60%, 50%, 40%, 30%, 20%, 10% or less of untreated control levels.

30. The method of any one of claims 10-29, wherein administration results in the suppression of H3K79 methyl mark rebound.

31. The method of any one of claims 10-30, wherein administration results in at least 20%, 30%, 40%, 50%, 60%, 70%, 80%, or 90% of leukemic blast cells undergoing cell death or apoptosis.

32. The method of any one of claims 10-31, wherein the method of treatment includes resolution of fevers, resolution of cachexia or resolution of leukemia cutis.

33. The method of any one of claims 10-32, wherein the method of treatment includes restoration of normal haematopoiesis.

34. The method of any one of claims 10-33, wherein the subject has demonstrated resistance to any one of the components of the composition of claim 1 when administered as a single agent.

35. The method of any one of claims 10-34, wherein the subject is a pediatric patient aged 3 months to 18 years.

36. A method of inhibiting cancer cell proliferation comprising contacting a cancer cell with the composition of claim 1.

37. A method of inhibiting cancer cell proliferation comprising contacting a cancer cell with a compound of Formula (I) and one or more therapeutic agents, wherein the compound of Formula (I) and the therapeutic agents are delivered simultaneously or sequentially.

38. The method of claim 37, wherein a compound of Formula (I) is administered prior to administration of the therapeutic agents.

39. A method of inhibiting cancer cell proliferation comprising administering a therapeutically effective dose of a compound of Formula (I), or a pharmaceutically acceptable salt thereof, prior to administering a therapeutically effective dose of a composition of claim 1.

40. The method of any one of claims 10-39, wherein the therapeutic agent is Ara-C, Daunorubicin, Vidaza, or an analog or derivative thereof.

41. The method of any one of claims 10-40, wherein the subject has leukemia.

42. The method of claim 41, wherein the leukemia is characterized by a chromosomal rearrangement.

43. The method of claim 42, wherein the chromosomal rearrangement is chimeric fusion of mixed lineage leukemia gene (MLL) or partial tandem duplication of MLL (MLL-PTD).

44. The method of any one of claims 10-43, wherein the subject has an increased level of HOXA9, Fms-like tyrosine kinase 3 (FLT3), MEIS1, and/or DOT1L.

45. The method of any one of claims 10-44, wherein the compound is Compound A2.

46. The method of any one of claims 10-44, wherein the compound is Compound D16.

47. A method of treating or alleviating a symptom of a disease comprising administering to a subject in need thereof a therapeutically effective amount of a compound of Formula I, wherein the therapeutically effective amount is an amount sufficient to sensitize the subject to subsequent treatment with a therapeutic agent.

48. The method of claim 47, further comprising administering to the sensitized subject a therapeutically effective amount of a therapeutic agent.

49. The method of claim 47 or 48, wherein the therapeutic agent is Ara-C, Daunorubicin, Vidaza, or an analog or derivative thereof.

50. The method of any of claims 47-49, wherein the subject has leukemia.

51. The method of claim 50, wherein the leukemia is characterized by a chromosomal rearrangement.

52. The method of claim 51, wherein the chromosomal rearrangement is chimeric fusion of mixed lineage leukemia gene (MLL) or partial tandem duplication of MLL (MLL-PTD).

53. The method of any one of claims 47-52, wherein the subject has an increased level of HOXA9, Fms-like tyrosine kinase 3 (FLT3), MEIS1, and/or DOT1L.

54. The method of any one of claims 47-53, wherein the compound of Formula (I) is Compound A2.

55. The method of any one of claims 47-53, wherein the compound of Formula (I) is Compound D16.

56. The method of any one of claims 47-55, wherein the therapeutic agent is a standard of care agent.

57. The method of any one of claims 47-56, wherein the therapeutic agent is administered at least after one, two, three or more hours following the administration of the compound of Formula I.

58. The method of any one of claims 47-57, wherein the therapeutic agent is administered at least after one, two, three or more days following the administration of the compound of Formula I.

59. The method of any one of claims 47-58, wherein the sensitization is determined by the methylation status of histones or other proteins.

60. The method of any one of claims 47-58, wherein the sensitization is determined by decreased level of methylation of histones or other proteins.

61. The method of any one of claims 47-58, wherein the sensitization is determined by decreased methylation of H3K79.

62. The method of any one of claims 47-58, wherein the therapeutically effective amount of the therapeutic agent is lowered due to the sensitizing effect of compound of Formula I.

63. A method of treating or alleviating a symptom of cancer comprising administering to a subject in need thereof a therapeutically effective dose of a compound of Formula (I) and one or more therapeutic agents, wherein the one or more therapeutic agents is administered prior to administration of the compound of Formula (I).

64. A method of treating or alleviating a symptom of cancer comprising administering to a subject in need thereof a therapeutically effective dose of one or more therapeutic agents prior to administering a therapeutically effective dose of a composition of claim 1.

65. A method of inhibiting cancer cell proliferation comprising contacting a cancer cell with a compound of Formula (I) and one or more therapeutic agents, wherein the one or more therapeutic agents is administered prior to administration of the compound of Formula (I).

66. A method of inhibiting cancer cell proliferation comprising administering a therapeutically effective dose of one or more therapeutic agents prior to administering a therapeutically effective dose of a composition of claim 1.

67. A method of treating or alleviating a symptom of a disease comprising administering to a subject in need thereof a therapeutically effective amount of one or more therapeutic agents, wherein the therapeutically effective amount is an amount sufficient to sensitize the subject to subsequent treatment with a compound of Formula I or a composition of claim 1.

68. The method of any one of claims 63-67, wherein the therapeutic agent is Ara-C.

69. The method of any one of claims 63-67, wherein the compound of Formula (I) or a pharmaceutically acceptable salt thereof is administered at a dose of at least 36 mg/m².

70. The method of any one of claims 63-67, wherein the compound of Formula (I) or a pharmaceutically acceptable salt thereof is administered at a dose of at least 54 mg/m².

71. The method of any one of claims 63-67, wherein the compound of Formula (I) or a pharmaceutically acceptable salt thereof is administered at a dose of at least 80 mg/m².

72. The method of any one of claims 63-67, wherein the compound of Formula (I) or a pharmaceutically acceptable salt thereof is administered continuously for at least 7, 14, 21, 28, 35, 42, 47, 56, or 64 days.

73. The method of claim 72, wherein continuous administration comprises administration without a drug holiday.

74. The method of any one of claims 63-67, wherein the administration results in maturation or differentiation of leukemic blast cells.

75. The method of claim 74, wherein at least 20% of leukemic blast cells have undergone maturation or differentiation.

76. The method of claim 74, wherein at least 50% of leukemic blast cells have undergone maturation or differentiation.

77. The method of claim 74, wherein at least 80% of leukemic blast cells have undergone maturation or differentiation.

78. The method of any one of claims 63-67, wherein administration results in reduction of H3K79 methyl mark to at least 90%, 80%, 70%, 60%, 50%, 40%, 30%, 20%, 10% or less of untreated control levels.

79. The method of any one of claims 63-67, wherein administration results in the suppression of H3K79 methyl mark rebound.

80. The method of any one of claims 63-67, wherein administration results in at least 20%, 30%, 40%, 50%, 60%, 70%, 80%, or 90% of leukemic blast cells undergoing cell death or apoptosis.

81. The method of any one of claims 63-67, wherein the method of treatment includes resolution of fevers, resolution of cachexia or resolution of leukemia cutis.

82. The method of any one of claims 63-67, wherein the method of treatment includes restoration of normal haematopoiesis.

83. The method of any one of claims 63-67, wherein the subject has demonstrated resistance to any one of the components of the composition of claim 1 when administered as a single agent.

84. The method of any one of claims 63-67, wherein the subject is a pediatric patient aged 3 months to 18 years.

85. The method of any one of claims 63-67, wherein the compound of Formula (I) is Compound A2.

86. The method of any one of claims 63-67, wherein the compound of Formula (I) is Compound D16.
